(19) United States
(12) Patent Application Publication
(10) Pub. No.: US 2024/0140958 A1
(43) Pub. Date: May 2, 2024

(54) FUSED QUADRACYCLIC COMPOUNDS, COMPOSITIONS AND USES THEREOF

(71) Applicant: Tabomedex Biosciences, Inc., Boxford, MA (US)

(72) Inventors: Michael H. Serrano-Wu, Belmont, MA (US); Zhixiong Ye, New York, NY (US); Weihong Zheng, Shanghai (CN)

(21) Appl. No.: 18/270,616

(22) PCT Filed: Jan. 10, 2022
(86) PCT No.: PCT/US22/11824

§ 371 Date: Jun. 30, 2023

(30) Foreign Application Priority Data
Jan. 12, 2021 (WO) PCT/CN21/71062

(51) Int. Cl. C07D 487/14; A61K 31/155; A61K 31/5377; A61K 31/551; A61K 33/243; A61P 35/00; C07D 519/00

(52) U.S. Cl. CPC C07D 487/14; A61K 31/155; A61K 31/5377; A61K 31/551; A61K 33/243; A61P 35/00; C07D 519/00

(57) ABSTRACT

Provided herein are substituted fused quadracyclic compounds useful as inhibitors of MK2. The invention further provides pharmaceutical compositions of the compounds of the invention. The invention also provides medical uses of substituted fused quadracyclic compounds.

### RELATED APPLICATION

This application claims benefit of priority to PCT International Application No. PCT/CN21/71062, filed Jan. 11, 2021, which is hereby incorporated by reference herein in its entirety.

### BACKGROUND

p38 mitogen activated protein kinase (p38 MAPK) transduces a range of extracellular signals that result in inflammatory response, cell division and differentiation, apoptosis, and cell motility. p38 MAPK was initially believed to be an ideal target for anti-inflammatory therapeutics. However, the failure of more than a dozen chemically different compounds in the clinical phase suggests that p38 MAPK might be a poor therapeutic target. Many of these compounds were found to be hepatotoxic to various degree and tolerance to the anti-inflammatory effect developed within weeks. In hindsight, the failures in clinical trials due to unwanted side effects is perhaps not unexpected as p38 MAPK regulates the activity for more than 60 substrates.

One of the downstream substrates of p38 MAPK is mitogen-activated protein kinase activated protein kinase-2 (MAPKAPK or MK2). Among other roles, MK2 regulates the biosynthesis of tumor necrosis factor α and other cytokines. In addition, MK2 is activated after DNA damage resulting in cell cycle arrest, such that cells have the capacity to repair their DNA and continue to proliferate. MK2 also phosphorylates heat shock 27 (Hsp27), a prominent biomarker of cancer progression. Thus, MK2 could serve as a potential anti-inflammatory target and an anticancer target to improve the efficacy of chemotherapy without the unwanted side effects that affect targets further upstream (i.e., p38 MAPK).

Therefore, there is a continuing need to discover and develop new compounds that inhibit MK2 and that may be useful therapeutics.

### SUMMARY OF INVENTION

In certain embodiments, the invention relates to compounds having the structure of Formula (I):

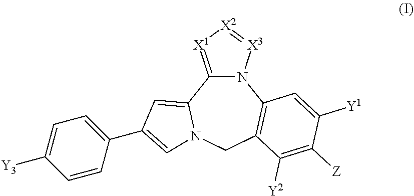

and pharmaceutically acceptable salts thereof, wherein X¹-X³, Y¹-Y³, and Z are as defined in the specification.

In some embodiments, the invention relates to pharmaceutical compositions comprising a compound of Formula (I) and a pharmaceutically acceptable carrier.

The invention also relates to methods of treating a MK2-related disorder, comprising administering to a subject a compound of the invention.

The invention further relates to methods of inhibiting proliferation of a cancer cell comprising contacting a cancer cell with a compound of the invention.

The invention also provides methods of inhibiting MK2 activity in a cell, comprising contacting a cell with a compound of the invention.

The invention also provides methods of treating or preventing a metabolic disorder, comprising administering to a subject a compound of the invention.

### DETAILED DESCRIPTION OF THE INVENTION

In certain aspects, the invention provides substituted fused quadracyclic compounds, and pharmaceutical compositions thereof. In particular, such substituted fused quadracyclic compounds are useful as MK2 inhibitors, and thus can be used as anti-cancer agents, anti-inflammatory agents, or anti-diabetic agents.

### I. Compounds

In certain embodiments, the invention relates to compounds having the structure of Formula (I):

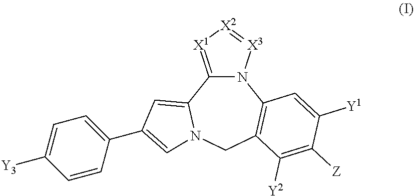

wherein
X¹, X², and X³ are each independently CH, C-(alkyl), or N;
Y¹ and Y² are each independently H or halogen;
Y³ is CN or halogen;
Z is —C(O)NZ¹Z², —NHC(O)NZ³Z⁴, —NHC(O)C(HZ⁵Z⁶), or —NZ⁷Z⁸;
   Z¹ is H or alkyl;
   Z² is optionally substituted alkyl, aminoalkyl, cycloalkyl, heterocyclyl, or heterocycloalkyl; or
   Z¹ and Z² together with the N to which they are bound combine to form an optionally substituted 4-, 5-, or 6-membered heterocyclic ring or an optionally substituted heterobicyclic ring;
   Z³ and Z⁴ together with the N to which they are bound combine to form an optionally substituted 4-, 5-, or 6-membered heterocyclic ring;
   Z⁵ and Z⁶ together with the C to which they are bound combine to form an optionally substituted 4-, 5-, or 6-membered heterocyclic ring; and
   Z⁷ and Z⁸ together with the N to which they are bound combine to form an optionally substituted 5- or 6-membered cyclic amide, cyclic urea, or cyclic carbamate.

In certain embodiments, the invention relates to compounds having the structure:

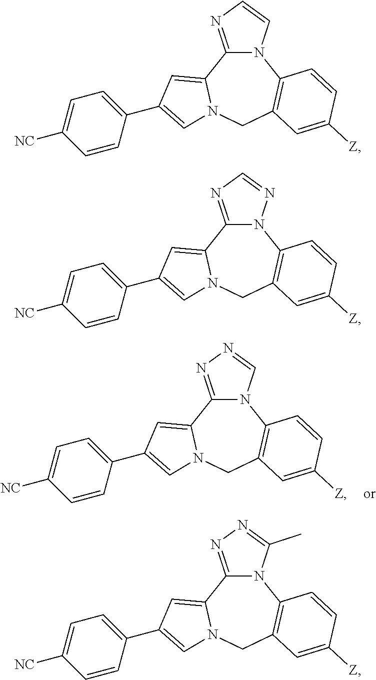

and pharmaceutically acceptable salts thereof, wherein Z is as defined in the specification.

In certain embodiments, one of X¹, X² and X³ is N, e.g., X¹ is N, and X² and X³ are each CH. In other embodiments, two of X¹, X² and X³ are N, e.g., X¹ and X³ are each N, and X² is CH, or X¹ and X² are each N, and X³ is CH, or X¹ and X² are each N, and X³ is CCH₃. In other embodiments, each of X¹, X² and X³ is N.

In certain embodiments, Z is —C(O)NZ¹Z².

In certain embodiments, Z¹ is H or CH₃.

In certain embodiments, Z² is substituted alkyl or substituted aminoalkyl. In other embodiments, Z² is a substituted linear C₁-C₆ alkyl. In other embodiments, Z² is a substituted branched C₂-C₆ alkyl.

In certain embodiments, Z² is substituted with alkylamino, e.g. methylamino or dimethylamino. In other embodiments, Z² is substituted with hydroxyalkyl.

In certain embodiments, Z is

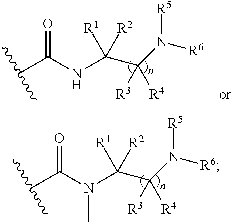

n is 0, 1 or 2; R¹, R², R³, and R⁴ are, independently for each occurrence, H, alkyl, or hydroxyalkyl; and R⁵ and R⁶ are, independently for each occurrence, H or alkyl.

In certain embodiments, R¹, R², R³, and R⁴ are, independently for each occurrence, H or CH₃; and each occurrence of R⁵ and R⁶ is CH₃. In other embodiments, each occurrence of R¹, R², R³, and R⁴ is H; and each occurrence of R⁵ and R⁶ is CH₃.

In certain embodiments, Z is

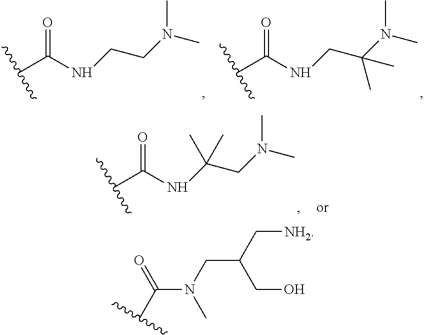

In certain embodiments, Z² is optionally substituted heterocycloalkyl.

In certain embodiments, the heterocycloalkyl comprises an azetidinyl, pyrrolidinyl, or piperidinyl. In other embodiments, the azetidinyl, pyrrolidinyl, or piperidinyl, when substituted, is substituted with alkyl.

In certain embodiments, Z is

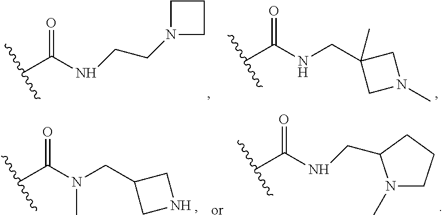

In certain embodiments, Z₂ is optionally substituted cycloalkyl, e.g. optionally substituted cyclobutyl, cyclopentyl, or cyclohexyl. In other embodiments, the cyclobutyl, cyclopentyl or cyclohexyl, when substituted, is substituted with alkylamino, e.g. methylamino or dimethylamino.

In certain embodiments, Z is

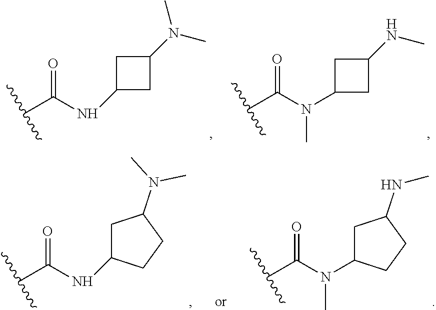 [SMILES: *C(=O)NC1CC(N(C)C)C1 ; *C(=O)N(C)C1CC(NC)C1 ; *C(=O)NC1CCC(N(C)C)C1 ; *C(=O)N(C)C1CCC(NC)C1]

In certain embodiments, Z² is optionally substituted heterocyclyl, e.g. optionally substituted azetidinyl, pyrrolidinyl, or piperidinyl. In other embodiments, the azetidinyl, pyrrolidinyl, or piperidinyl, when substituted, is substituted with alkyl, e.g. methyl or isopropyl.

In certain embodiments, Z is

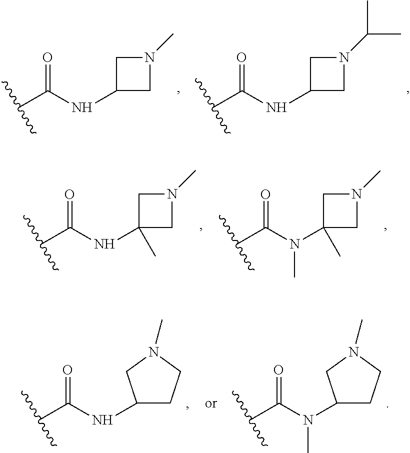

In certain embodiments, Z is —C(O)NZ¹Z²; and Z¹ and Z², together with the N to which they are bound, combine to form an optionally substituted 4-, 5-, or 6-membered heterocyclic ring.

In certain embodiments, Z is

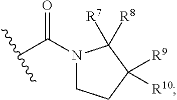

R⁷ and R⁸ are each independently H, CN, halo, alkyl, aminoalkyl, or alkylaminoalkyl; and R⁹ and R¹⁰ are each independently H, CN, halo, hydroxyl, amino, alkyl, aminoalkyl, alkylamino, or alkylaminoalkyl.

In other embodiments, R⁷ and R⁸ are each independently H, alkyl, or alkylaminoalkyl; and R⁹ and R¹⁰ are each independently H, alkyl, alkylamino, or alkylaminoalkyl, e.g. methylamino, dimethylamino, methylaminoalkyl, or dimethylaminoalkyl.

In certain embodiments, Z is

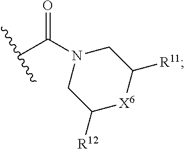

X⁶ is CH₂, NH, or N(alkyl); R¹¹ and R¹² are each independently H, CN, halo, hydroxyl, amino, alkyl, aminoalkyl, alkylamino, or alkylaminoalkyl; and when X⁶ is NH or N(alkyl), then R¹¹ and R¹² are not hydroxyl, amino, or alkylamino. In other embodiments, R¹¹ and R¹² are each independently H, CN, alkyl, alkylamino, or alkylaminoalkyl, e.g. methylamino, dimethylamino, methylaminoalkyl, or dimethylaminoalkyl.

In certain embodiments, Z is

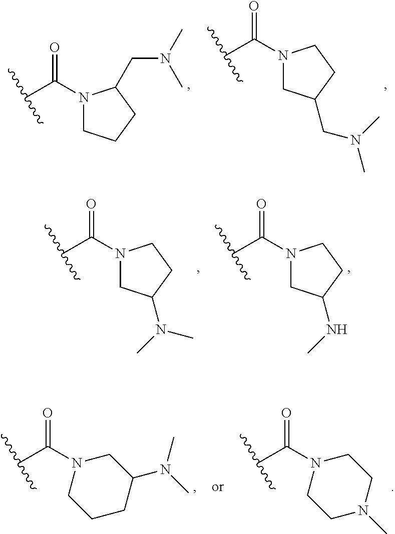

In certain embodiments, Z is

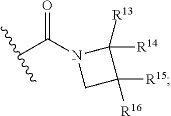

R¹³ and R¹⁴ are each independently H, CN, halo, alkyl, aminoalkyl, or alkylaminoalkyl; R¹⁵ and R¹⁶ are each independently H, CN, halo, hydroxyl, amino, alkyl, aminoalkyl, alkylamino, or alkylaminoalkyl, or R¹⁵ and R¹⁶ combine to form an optionally substituted 4-, 5-, or 6-membered nitrogen containing heterocyclic ring.

In other embodiments, R¹³ and R¹⁴ are each independently H, CN, alkyl, aminoalkyl, or alkylaminoalkyl. In other embodiments, R¹⁵ and R¹⁶ are each independently H, CN, alkyl, aminoalkyl, alkylamino, or alkylaminoalkyl, e.g. methylamino, dimethylamino, methylaminoalkyl, or dimethylaminoalkyl. In other embodiments, R¹³ and R¹⁴ are each H; and R¹⁵ and R¹⁶ combine to form an optionally substituted 4-, 5-, or 6-membered nitrogen containing heterocyclic ring.

In certain embodiments, the optionally substituted 4-, 5-, or 6-membered nitrogen containing heterocyclic ring is an azetidine, pyrrolidine, or piperidine. In other embodiments, the azetidine, pyrrolidine, or piperidine, when substituted, is substituted with alkyl, e.g. methyl.

In certain embodiments, Z is

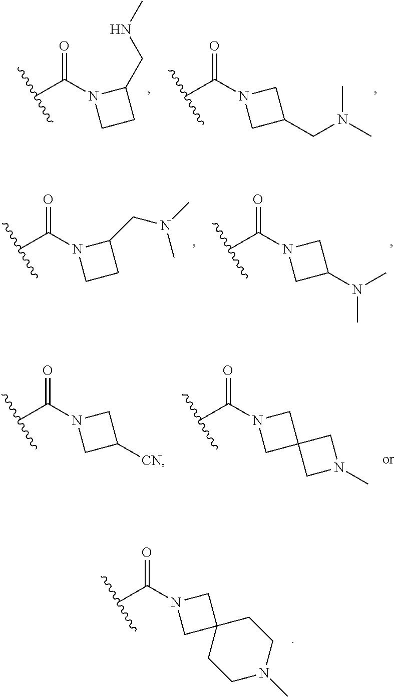

In certain embodiments, Z is —C(O)NZ¹Z²; and Z¹ and Z² together with the N to which they are bound combine to form an optionally substituted heterobicyclic ring.

In certain embodiments, Z is

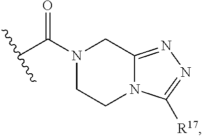

wherein R¹⁷ is H, —CN, halo, hydroxyl, amino, alkyl, aminoalkyl, alkylamino, or alkylaminoalkyl.

In certain embodiments, Z is

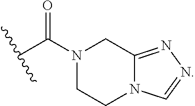 [SMILES: *C(=O)N1CCn2cnnc2C1]

In certain embodiments, Z is —NHC(O)NZ³Z⁴; and Z³ and Z⁴ together with the N to which they are bound combine to form an optionally substituted 5-membered heterocyclic ring.

In certain embodiments, Z is

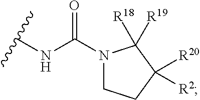

R¹⁸ and R¹⁹ are each independently H, CN, halo, alkyl, aminoalkyl, or alkylaminoalkyl; and R²⁰ and R²¹ are each independently H, CN, halo, hydroxyl, amino, alkyl, aminoalkyl, alkylamino, or alkylaminoalkyl. In other embodiments, R²⁰ and R²¹ are each independently H or alkylamino. In other embodiments, the alkylamino is methylamino or dimethylamino.

In certain embodiments, Z is

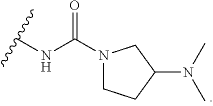 [SMILES: *NC(=O)N1CCC(N(C)C)C1]

In certain embodiments, Z is —NHC(O)C(HZ⁵Z⁶); and Z⁵ and Z⁶ together with the C to which they are bound combine to form an optionally substituted 5-membered heterocyclic ring.

In certain embodiments, Z is

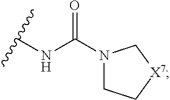

X⁷ is CHR²² or NR²³; R²² is H, —CN, halo, hydroxyl, amino, alkyl, aminoalkyl, alkylamino, or alkylaminoalkyl; and R²³ is H, alkyl, alkylamino, or alkylaminoalkyl. In other embodiments, Z is X⁷ is CHR²²; and R²² is alkylamino, e.g. methylamino or dimethylamino. In other embodiments, X⁷ is NR²³; and R²³ is H or alkyl, e.g. methyl or isopropyl.

In certain embodiments, Z is

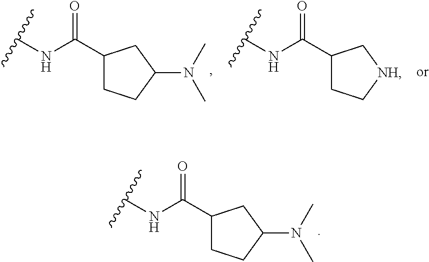

In certain embodiments, Z is —NZ⁷Z⁸; and Z⁷ and Z⁸ together with the N to which they are bound combine to form an optionally substituted 5- or 6-membered cyclic amide, cyclic urea, or cyclic carbamate.

In certain embodiments, Z is

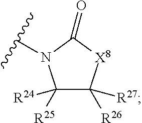

X⁸ is CR²⁸R²⁹, NR³⁰ or O; R²⁴ and R²⁶ are each independently H or alkyl; R²⁵ is H, or an optionally substituted alkyl, aminoalkyl, alkylaminoalkyl, heterocycloalkyl, or heterocyclyl; R²⁷ is H, or an optionally substituted alkyl, amino, alkylamino, aminoalkyl, alkylaminoalkyl, heterocycloalkyl, or heterocyclyl; R²⁸ and R²⁹ each independently H, alkyl, alkylamino, aminoalkyl, or optionally substituted alkylaminoalkyl; R³⁰ is H, alkyl, alkylaminoalkyl, or an optionally substituted cycloalkyl or heterocyclyl; and when X⁸ is NR³⁰ or O, then R³⁰ is not amino, or alkylamino.

In certain embodiments, X⁸ is NH or N—CH₃. In other embodiments, X⁸ is CH₂. In other embodiments, X⁸ is O.

In certain embodiments, R²⁵ and R²⁷ is H and the other of R²⁵ and R²⁷ is

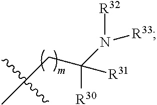

m is 0, 1 or 2; R³⁰ and R³¹ are each independently H or alkyl, or R³⁰ and R³¹ together with the C to which they are bound form a carbocyclic ring; and R³² and R³³ are each independently H, alkyl, hydroxyalkyl, optionally substituted cycloalkyl, or optionally substituted heterocyclyl, or together with the N to which they are bound form an optionally substituted heterocyclic ring.

In certain embodiments, R³⁰ and R³¹ are each H. In other embodiments, R³⁰ and R³¹ are each alkyl, e.g. methyl.

In certain embodiments, R³² and R³³ are each H. In other embodiments, R³² and R³³ are each alkyl, e.g. methyl.

In certain embodiments, R³² is H or CH₃; and R³³ is alkyl, hydroxyalkyl, optionally substituted cycloalkyl, or optionally substituted heterocyclyl, e.g. cyclopropyl, cyclobutyl, azetidinyl, pyrrolidinyl, or piperidinyl. In other embodiments, the cycloalkyl or heterocyclyl, when substituted, is substituted with halo, alkyl, hydroxyl, hydroxyalkyl, or carbamate.

In certain embodiments, R³² and R³³ combine to form an optionally substituted azetidine, pyrrolidine, or piperidine. In other embodiments, the azetidinyl, pyrrolidinyl, or piperidinyl, when substituted, is substituted with halo, alkyl, or hydroxyl.

In certain embodiments, Z is

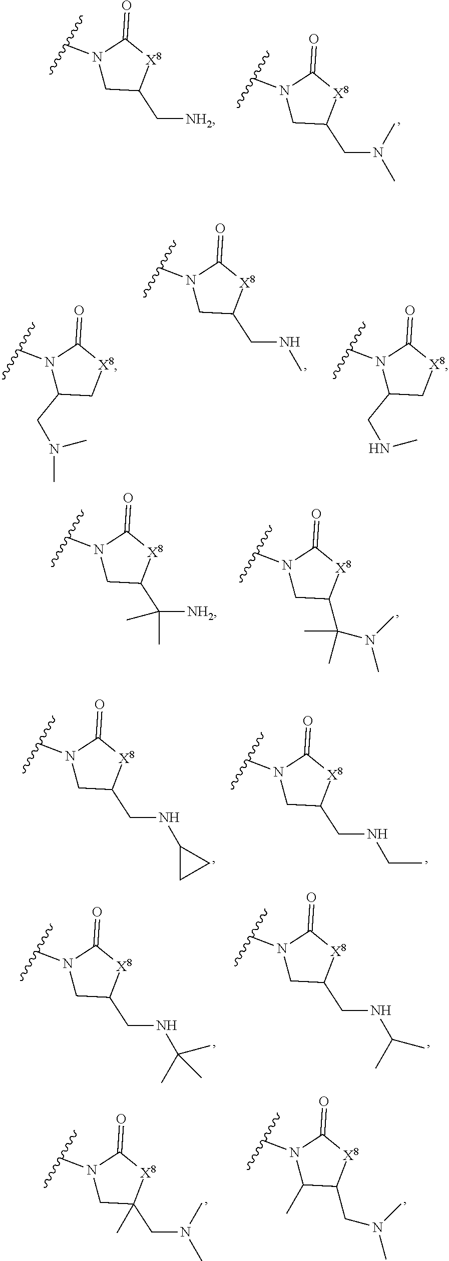

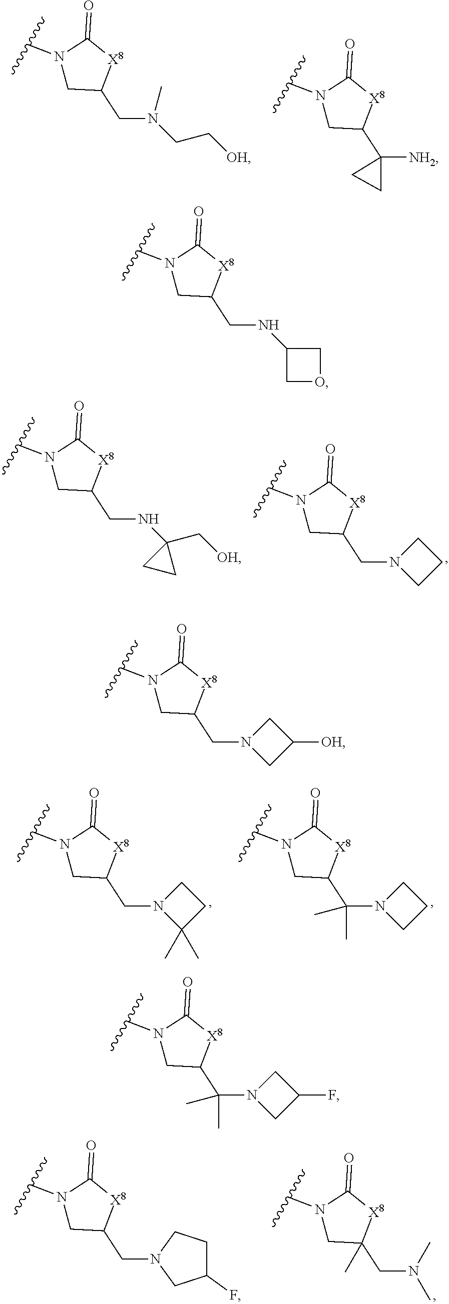

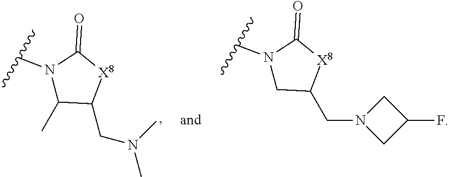

In certain embodiments, R²⁵ is H; and R²⁷ is amino or optionally substituted heterocyclyl. In other embodiments, the optionally substituted heterocyclyl is an optionally substituted oxetanyl, azetidinyl, pyrrolidinyl, piperidinyl, or oxazolidinonyl. In other embodiments, the oxetanyl, azetidinyl, pyrrolidinyl, piperidinyl or oxazolidinonyl, when substituted, is substituted with alkyl, hydroxyalkyl, or carbamate.

In certain embodiments, Z is

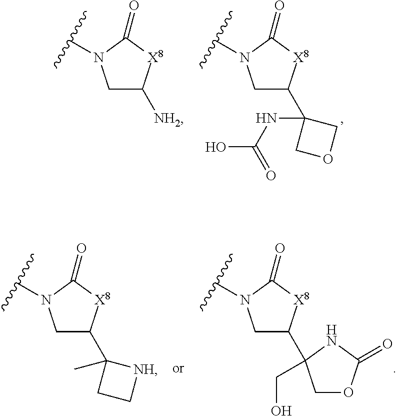

In certain embodiments, Z is

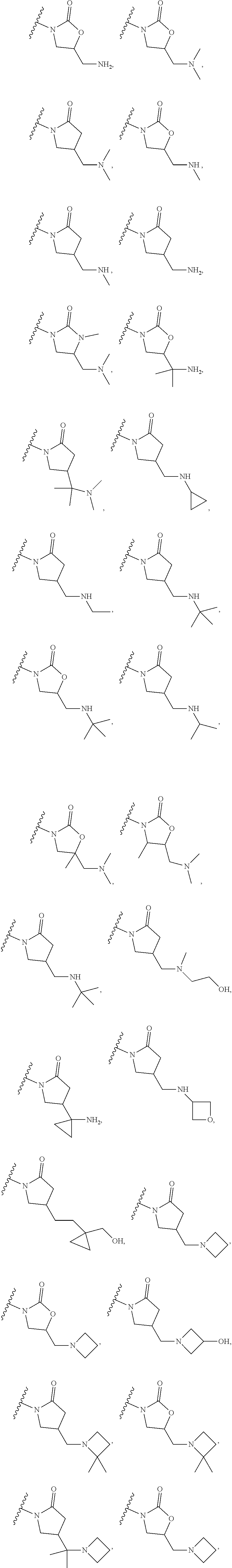

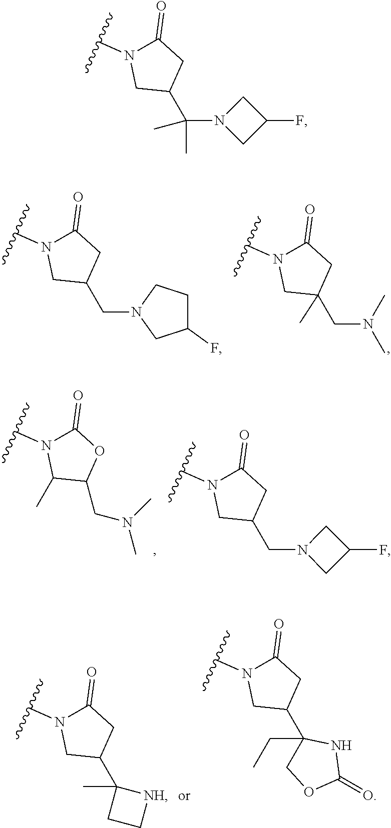

In certain embodiments, X⁸ is CR²⁸R²⁹; and R²⁸ and R²⁹ each independently H, alkyl, alkylamino, aminoalkyl, or optionally substituted alkylaminoalkyl; and R³⁰ is alkyl, alkylaminoalkyl, or an optionally substituted cycloalkyl or heterocyclyl. In other embodiments, one of R²⁸ and R²⁹ is H or CH₃ and the other of R²⁸ and R²⁹ is alkylamino, aminoalkyl, or optionally substituted alkylaminoalkyl. In other embodiments, the alkylaminoalkyl, when substituted, is substituted with a hydroxyl.

In certain embodiments, R²⁴, R²⁵, R²⁶, and R²⁷ are each H.

In certain embodiments, X⁸ is NR³⁰; and R³⁰ is alkylaminoalkyl, or an optionally substituted cycloalkyl or heterocyclyl, e.g. optionally substituted cyclopropyl, cyclobutyl, oxetanyl, azetidinyl, pyrrolidinyl, or piperidinyl. In other embodiments, the cyclopropyl or cyclobutyl, when substituted, is substituted with alkylamino, e.g. methylamino or dimethylamino. In other embodiments, the oxetanyl, azetidinyl, pyrrolidinyl, or piperidinyl, when substituted, is substituted with alkyl, e.g. methyl.

In certain embodiments, Z is

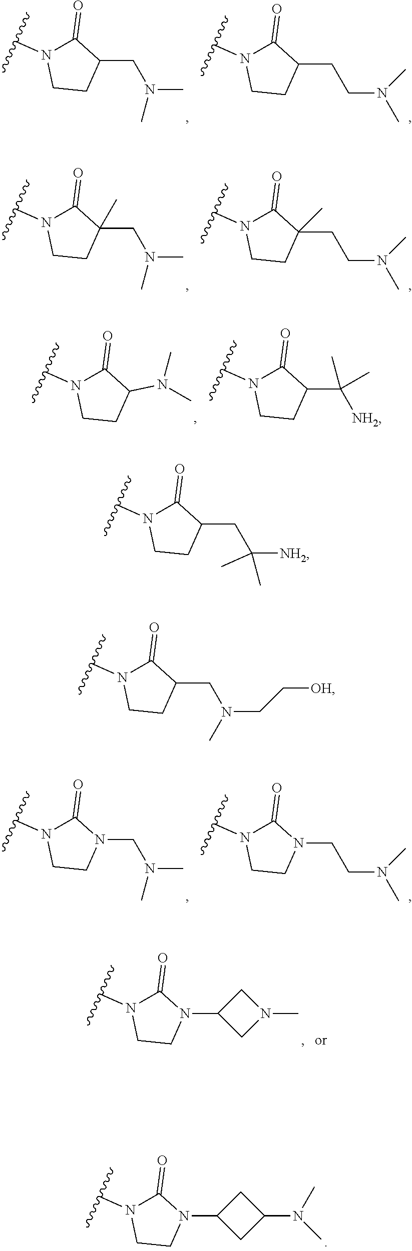

In certain embodiments, Z is

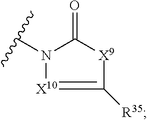

X⁹ is CR³⁶R³⁷ or N³⁸; X¹⁰ is N or CR³⁴; R³⁴ and R³⁵ are each independently H or optionally substituted alkyl; R³⁶ and R³⁷ are each independently H, alkyl, alkylamino, aminoalkyl, alkylaminoalkyl, or optionally substituted heterocyclyl; and R³⁸ is alkyl, aminoalkyl or alkylaminoalkyl.

In other embodiments, Z is

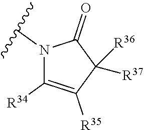

In other embodiments, one of R³⁶ and R³⁷ is H or alkyl and the other of R³⁶ and R³⁷ is alkylaminoalkyl or optionally substituted heterocyclyl. In other embodiments, the optionally substituted heterocyclyl is oxetanyl, azetidinyl, pyrrolidinyl, or piperidinyl. In other embodiments, the oxetanyl, azetidinyl, pyrrolidinyl, or piperidinyl, when substituted, is substituted with alkyl.

In other embodiments, Z is

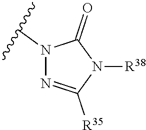

In other embodiments, R³⁸ is alkylaminoalkyl.

In certain embodiments, Z is

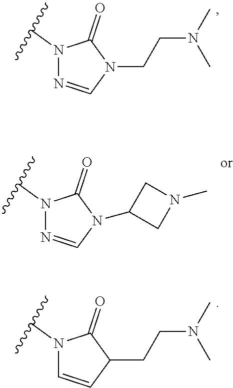 [SMILES: *n1ncn(CCN(C)C)c1=O ; *n1ncn(C2CN(C)C2)c1=O ; *N1C=CC(CCN(C)C)C1=O]

In certain embodiments, Z is

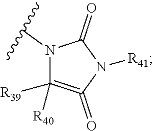

R³⁹ and R⁴⁰ are each independently H or optionally substituted alkyl; and R⁴¹ is aminoalkyl, alkylaminoalkyl, or an optionally substituted heterocyclyl or heterocycloalkyl. In other embodiments, R⁴¹ is aminoalkyl or alkylaminoalkyl. In other embodiments, R⁴¹ is an optionally substituted heterocyclyl or heterocycloalkyl. In other embodiments, the heterocyclyl or heterocycloalkyl substituted alkyl, when substituted, is substituted with alkyl, e.g. methyl.

In certain embodiments, Z is

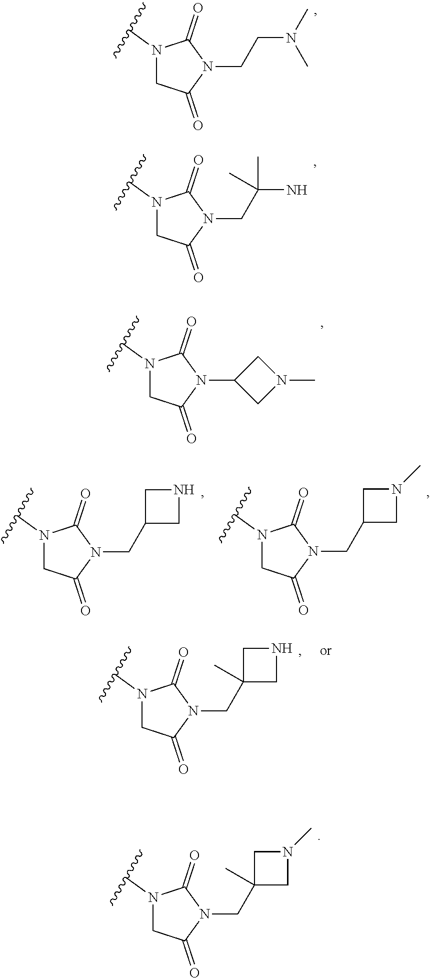

In certain embodiments, Z is

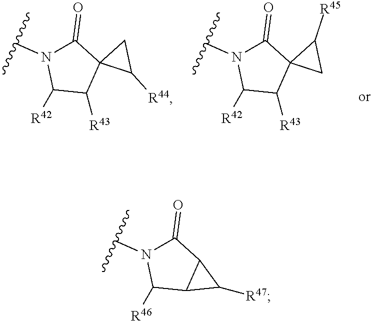

R⁴², R⁴³, and R⁴⁶ are each independently H or optionally substituted alkyl; and R⁴⁴, R⁴⁵, and R⁴⁷ are each independently H, alkylamino, aminoalkyl, or alkylaminoalkyl. In other embodiments, R⁴², R⁴³, and R⁴⁶ are each H. In other embodiments, R⁴⁴, R⁴⁵, and R⁴⁷ are each alkylaminoalkyl.

In certain embodiments, Z is

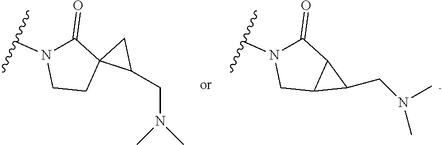 [SMILES: *N1CCC2(CC2CN(C)C)C1=O ; *N1CC2C(CN(C)C)C2C1=O]

In certain embodiments, Z is

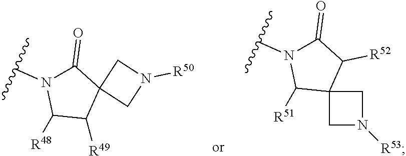

R⁴⁸, R⁴⁹, R⁵¹, and R⁵² are each independently H or optionally substituted alkyl; and R⁵⁰ and R⁵³ are each independently H, alkyl, aminoalkyl, or alkylaminoalkyl. In other embodiments, R⁴⁸, R⁴⁹, R⁵¹, and R⁵² are each H. In other embodiments, R⁵⁰ and R⁵³ are each alkyl.

In certain embodiments, Z is

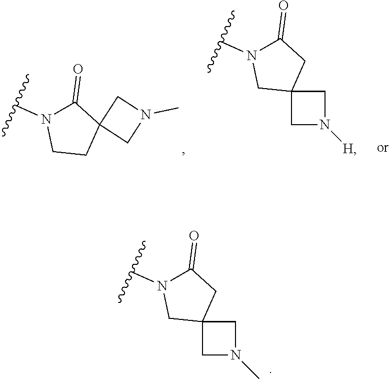

In certain embodiments, Z is

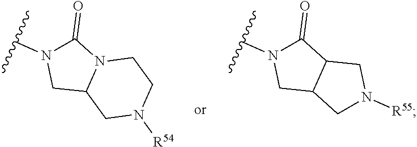

and R⁵⁴ and R⁵⁵ are each independently H, alkyl, aminoalkyl, or alkylaminoalkyl. In other embodiments, R⁵⁴ and R⁵⁵ are each alkyl, e.g. methyl.

In certain embodiments, Z is

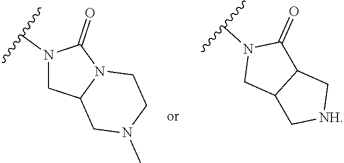 [SMILES: *N1CC2CN(C)CCN2C1=O ; "or" ; *N1CC2CNCC2C1=O]

In certain embodiments, Z is

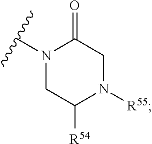

R⁵⁴ is H, or an optionally substituted alkyl or hydroxyalkyl; and R⁵⁵ is H, alkyl, aminoalkyl, alkylaminoalkyl, or cycloalkyl. In other embodiments, R⁵⁴ is H or hydroxyalkyl. In other embodiments, R⁵⁵ is H or cycloalkyl, e.g. cyclopropyl.

In certain embodiments, Z is

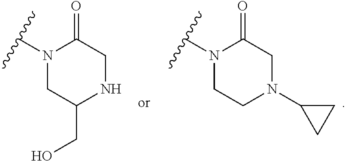 [SMILES: *N1CC(CO)NCC1=O ; *N1CCN(C2CC2)CC1=O]

Exemplary compounds of Formula I are depicted in Table 1. The compounds of Table 1 are understood to encompass both the free base and the conjugate acid. For example, the compounds in Table 1 may be depicted as the free base forms, but the compounds in their corresponding complexes or salts with trifluoroacetic acid or hydrochloric acid or as salts with other acids are equally within the scope of the invention. Compounds may be isolated in either the free base form, as a salt (e.g., a hydrochloride salt) or in both forms. In the chemical structures shown below, standard chemical abbreviations are sometimes used.

TABLE 1
Exemplary Compounds of the Invention

| Structure | Ex. # |
|---|---|
| 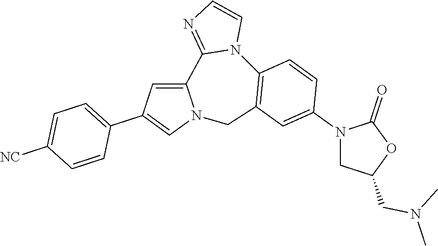 [SMILES: CN(C)C[C@@H]1CN(c2ccc3c(c2)Cn2cc(-c4ccc(C#N)cc4)cc2-c2nccn2-3)C(=O)O1] | 1 |
| 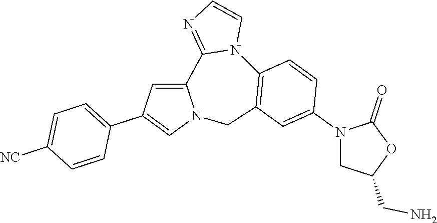 [SMILES: N#Cc1ccc(-c2cc3n(c2)Cc2cc(N4C[C@@H](CN)OC4=O)ccc2-n2ccnc2-3)cc1] | 2 |
| 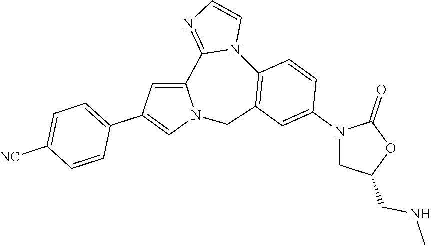 [SMILES: CNC[C@@H]1CN(c2ccc3c(c2)Cn2cc(-c4ccc(C#N)cc4)cc2-c2nccn2-3)C(=O)O1] | 3 |
| 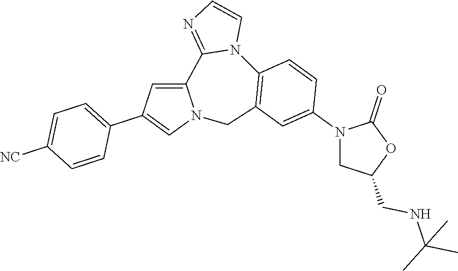 [SMILES: CC(C)(C)NC[C@@H]1CN(c2ccc3c(c2)Cn2cc(-c4ccc(C#N)cc4)cc2-c2nccn2-3)C(=O)O1] | 4 |
| 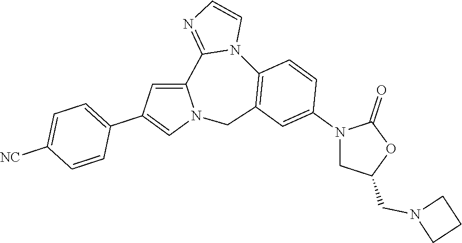 [SMILES: N#Cc1ccc(-c2cc3n(c2)Cc2cc(N4C[C@@H](CN5CCC5)OC4=O)ccc2-n2ccnc2-3)cc1] | 5 |
| 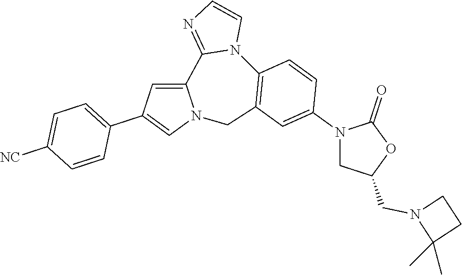 [SMILES: CC1(C)CCN1C[C@@H]1CN(c2ccc3c(c2)Cn2cc(-c4ccc(C#N)cc4)cc2-c2nccn2-3)C(=O)O1] | 6 |
| 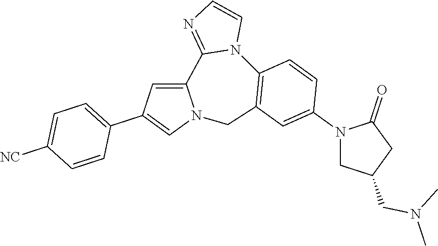 [SMILES: CN(C)C[C@H]1CC(=O)N(c2ccc3c(c2)Cn2cc(-c4ccc(C#N)cc4)cc2-c2nccn2-3)C1] | 7 |
| 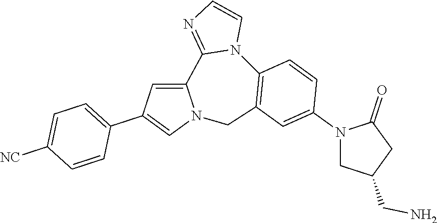 [SMILES: N#Cc1ccc(-c2cc3n(c2)Cc2cc(N4C[C@@H](CN)CC4=O)ccc2-n2ccnc2-3)cc1] | 8 |
| 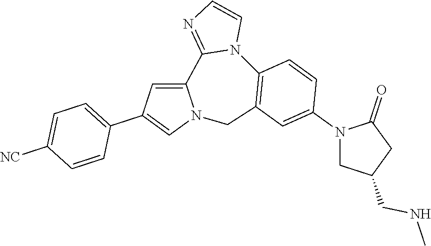 [SMILES: CNC[C@H]1CC(=O)N(c2ccc3c(c2)Cn2cc(-c4ccc(C#N)cc4)cc2-c2nccn2-3)C1] | 9 |
| 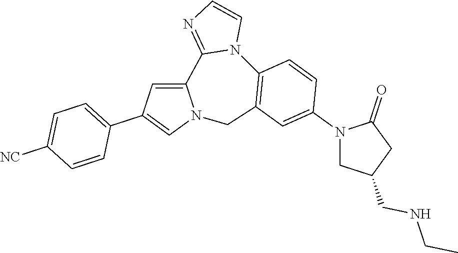 [SMILES: CCNC[C@H]1CC(=O)N(c2ccc3c(c2)Cn2cc(-c4ccc(C#N)cc4)cc2-c2nccn2-3)C1] | 10 |
| 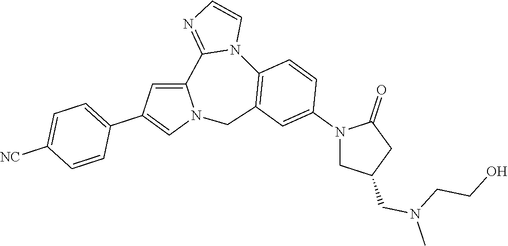 [SMILES: CN(CCO)C[C@H]1CC(=O)N(c2ccc3c(c2)Cn2cc(-c4ccc(C#N)cc4)cc2-c2nccn2-3)C1] | 11 |
| 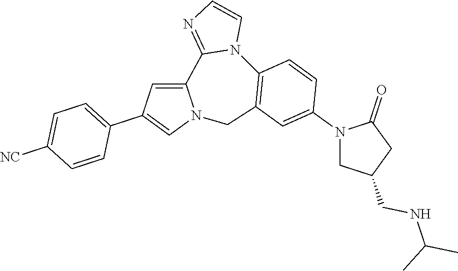 [SMILES: CC(C)NC[C@H]1CC(=O)N(c2ccc3c(c2)Cn2cc(-c4ccc(C#N)cc4)cc2-c2nccn2-3)C1] | 12 |
| 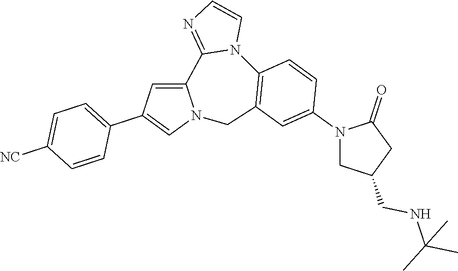 [SMILES: CC(C)(C)NC[C@H]1CC(=O)N(c2ccc3c(c2)Cn2cc(-c4ccc(C#N)cc4)cc2-c2nccn2-3)C1] | 13 |
| 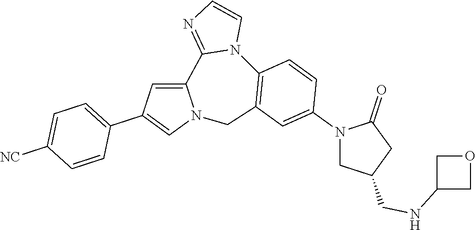 [SMILES: N#Cc1ccc(-c2cc3n(c2)Cc2cc(N4C[C@@H](CNC5COC5)CC4=O)ccc2-n2ccnc2-3)cc1] | 14 |
| 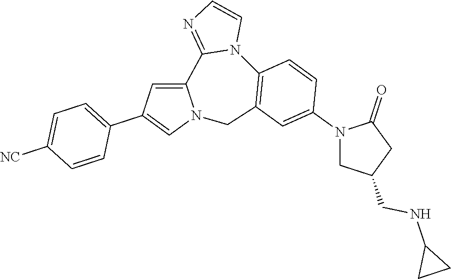 [SMILES: N#Cc1ccc(-c2cc3n(c2)Cc2cc(N4C[C@@H](CNC5CC5)CC4=O)ccc2-n2ccnc2-3)cc1] | 15 |
| 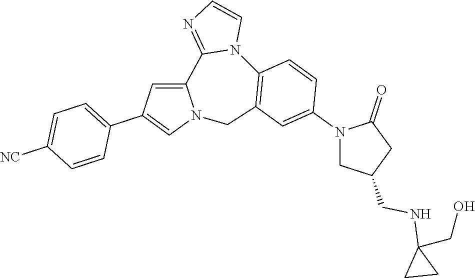 [SMILES: N#Cc1ccc(-c2cc3n(c2)Cc2cc(N4C[C@@H](CNC5(CO)CC5)CC4=O)ccc2-n2ccnc2-3)cc1] | 16 |
| 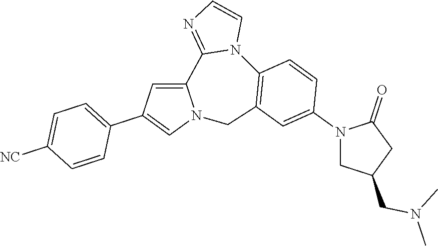 [SMILES: CN(C)C[C@@H]1CC(=O)N(c2ccc3c(c2)Cn2cc(-c4ccc(C#N)cc4)cc2-c2nccn2-3)C1] | 17 |
| 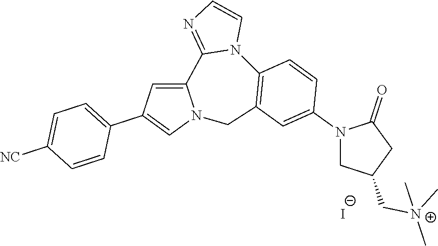 [SMILES: C[N+](C)(C)C[C@H]1CC(=O)N(c2ccc3c(c2)Cn2cc(-c4ccc(C#N)cc4)cc2-c2nccn2-3)C1 ; [I-]] | 18 |
| 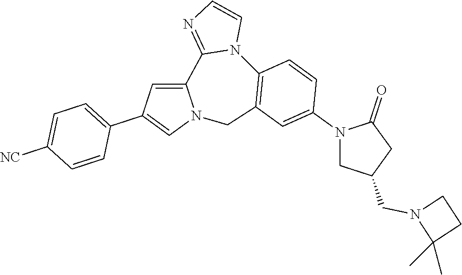 [SMILES: CC1(C)CCN1C[C@H]1CC(=O)N(c2ccc3c(c2)Cn2cc(-c4ccc(C#N)cc4)cc2-c2nccn2-3)C1] | 19 |
| 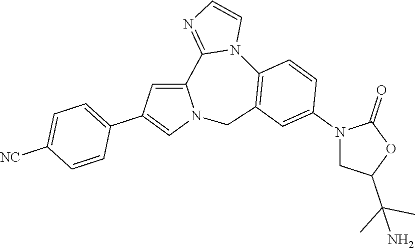 [SMILES: CC(C)(N)C1CN(c2ccc3c(c2)Cn2cc(-c4ccc(C#N)cc4)cc2-c2nccn2-3)C(=O)O1] | 20 |
| 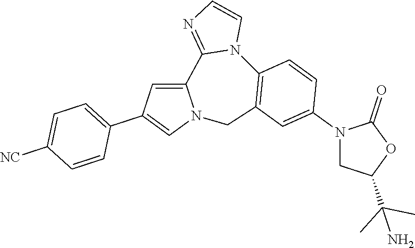 [SMILES: CC(C)(N)[C@@H]1CN(c2ccc3c(c2)Cn2cc(-c4ccc(C#N)cc4)cc2-c2nccn2-3)C(=O)O1] | 21 |
| 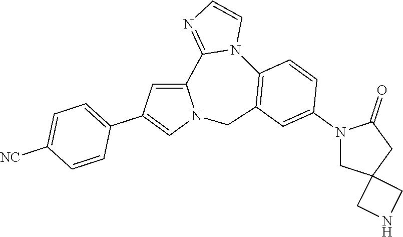 [SMILES: N#Cc1ccc(-c2cc3n(c2)Cc2cc(N4CC5(CNC5)CC4=O)ccc2-n2ccnc2-3)cc1] | 22 |
| 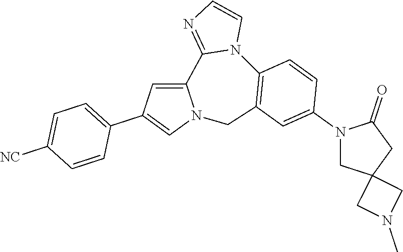 [SMILES: CN1CC2(CC(=O)N(c3ccc4c(c3)Cn3cc(-c5ccc(C#N)cc5)cc3-c3nccn3-4)C2)C1] | 23 |
| 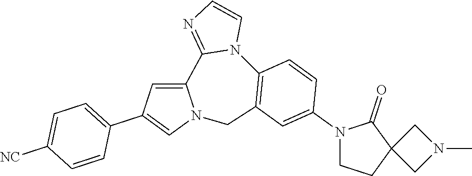 [SMILES: CN1CC2(CCN(c3ccc4c(c3)Cn3cc(-c5ccc(C#N)cc5)cc3-c3nccn3-4)C2=O)C1] | 24 |
| 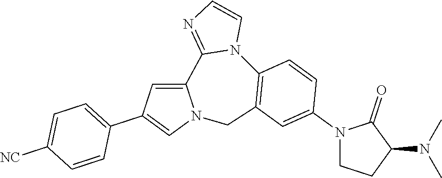 [SMILES: CN(C)[C@H]1CCN(c2ccc3c(c2)Cn2cc(-c4ccc(C#N)cc4)cc2-c2nccn2-3)C1=O] | 25 |
| 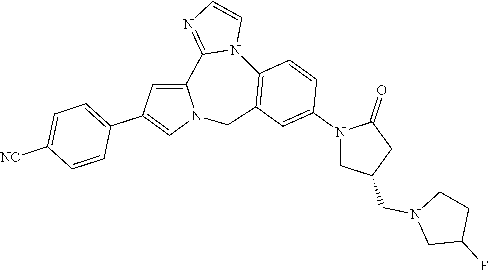 [SMILES: N#Cc1ccc(-c2cc3n(c2)Cc2cc(N4C[C@@H](CN5CCC(F)C5)CC4=O)ccc2-n2ccnc2-3)cc1] | 26 |
| 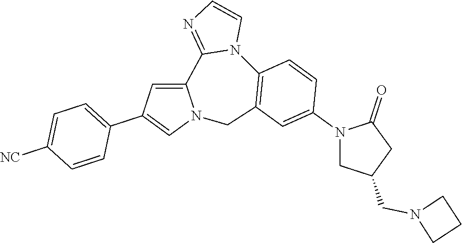 [SMILES: N#Cc1ccc(-c2cc3n(c2)Cc2cc(N4C[C@@H](CN5CCC5)CC4=O)ccc2-n2ccnc2-3)cc1] | 27 |
| 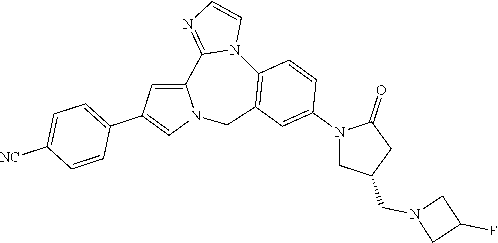 [SMILES: N#Cc1ccc(-c2cc3n(c2)Cc2cc(N4C[C@@H](CN5CC(F)C5)CC4=O)ccc2-n2ccnc2-3)cc1] | 28 |
| 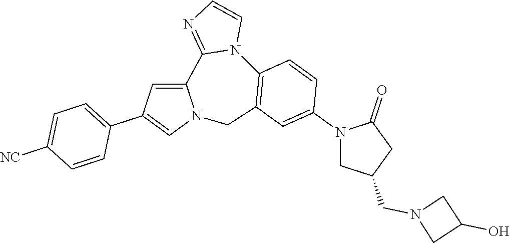 [SMILES: N#Cc1ccc(-c2cc3n(c2)Cc2cc(N4C[C@@H](CN5CC(O)C5)CC4=O)ccc2-n2ccnc2-3)cc1] | 29 |
| 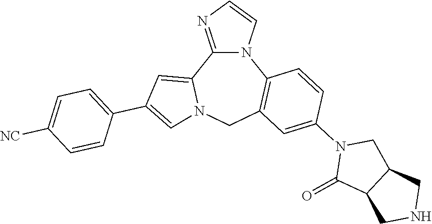 [SMILES: N#Cc1ccc(-c2cc3n(c2)Cc2cc(N4C[C@H]5CNC[C@H]5C4=O)ccc2-n2ccnc2-3)cc1] | 30 |
| 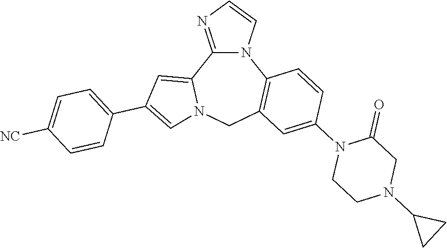 [SMILES: N#Cc1ccc(-c2cc3n(c2)Cc2cc(N4CCN(C5CC5)CC4=O)ccc2-n2ccnc2-3)cc1] | 31 |
| 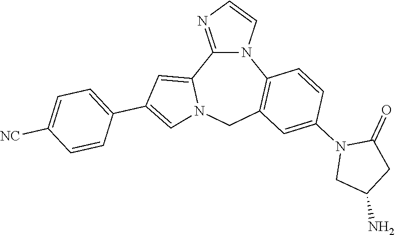 [SMILES: N#Cc1ccc(-c2cc3n(c2)Cc2cc(N4C[C@@H](N)CC4=O)ccc2-n2ccnc2-3)cc1] | 32 |
| 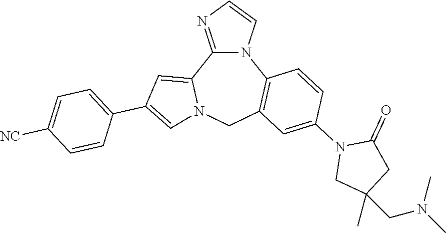 [SMILES: CN(C)CC1(C)CC(=O)N(c2ccc3c(c2)Cn2cc(-c4ccc(C#N)cc4)cc2-c2nccn2-3)C1] | 33 |
| 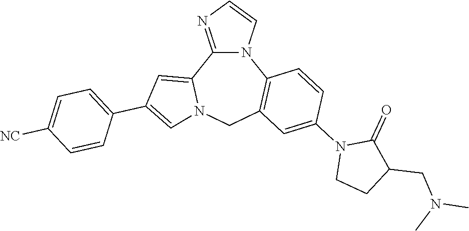 [SMILES: CN(C)CC1CCN(c2ccc3c(c2)Cn2cc(-c4ccc(C#N)cc4)cc2-c2nccn2-3)C1=O] | 34 |
| 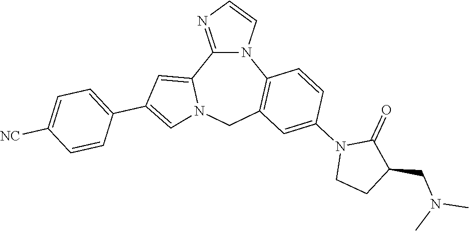 [SMILES: CN(C)C[C@H]1CCN(c2ccc3c(c2)Cn2cc(-c4ccc(C#N)cc4)cc2-c2nccn2-3)C1=O] | 35 |
| 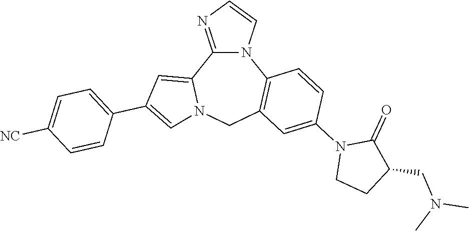 [SMILES: CN(C)C[C@@H]1CCN(c2ccc3c(c2)Cn2cc(-c4ccc(C#N)cc4)cc2-c2nccn2-3)C1=O] | 36 |
| 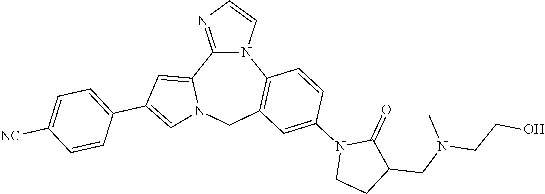 [SMILES: CN(CCO)CC1CCN(c2ccc3c(c2)Cn2cc(-c4ccc(C#N)cc4)cc2-c2nccn2-3)C1=O] | 37 |
| 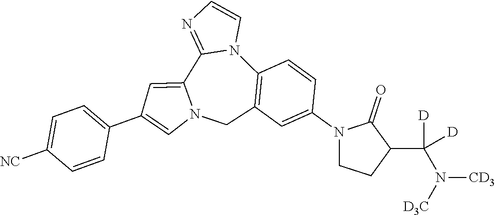 [SMILES: [2H]C([2H])([2H])N(C([2H])([2H])[2H])C([2H])([2H])C1CCN(c2ccc3c(c2)Cn2cc(-c4ccc(C#N)cc4)cc2-c2nccn2-3)C1=O] | 38 |
| 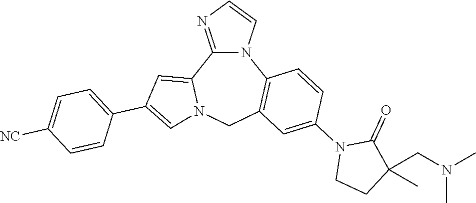 [SMILES: CN(C)CC1(C)CCN(c2ccc3c(c2)Cn2cc(-c4ccc(C#N)cc4)cc2-c2nccn2-3)C1=O] | 39 |
| 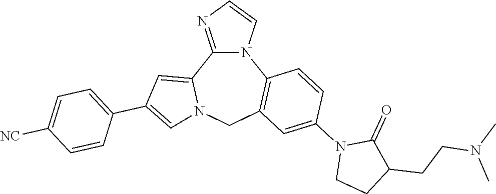 [SMILES: CN(C)CCC1CCN(c2ccc3c(c2)Cn2cc(-c4ccc(C#N)cc4)cc2-c2nccn2-3)C1=O] | 40 |
| 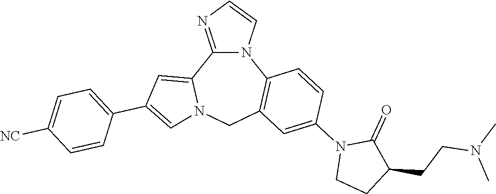 [SMILES: CN(C)CC[C@H]1CCN(c2ccc3c(c2)Cn2cc(-c4ccc(C#N)cc4)cc2-c2nccn2-3)C1=O] | 41 |
| 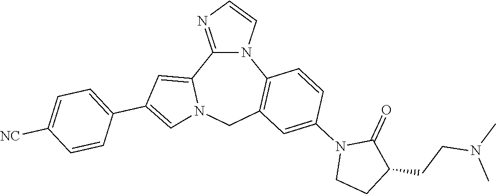 [SMILES: CN(C)CC[C@@H]1CCN(c2ccc3c(c2)Cn2cc(-c4ccc(C#N)cc4)cc2-c2nccn2-3)C1=O] | 42 |
| 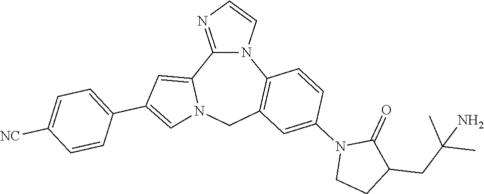 [SMILES: CC(C)(N)CC1CCN(c2ccc3c(c2)Cn2cc(-c4ccc(C#N)cc4)cc2-c2nccn2-3)C1=O] | 43 |
| 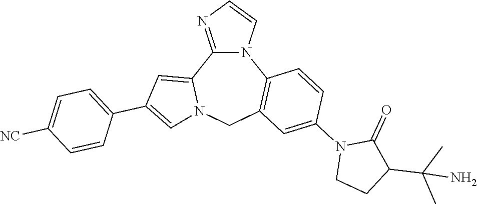 [SMILES: CC(C)(N)C1CCN(c2ccc3c(c2)Cn2cc(-c4ccc(C#N)cc4)cc2-c2nccn2-3)C1=O] | 44 |
| 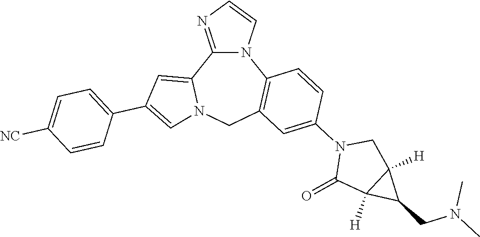 [SMILES: CN(C)C[C@@H]1[C@@H]2CN(c3ccc4c(c3)Cn3cc(-c5ccc(C#N)cc5)cc3-c3nccn3-4)C(=O)[C@H]12] | 45 |
| 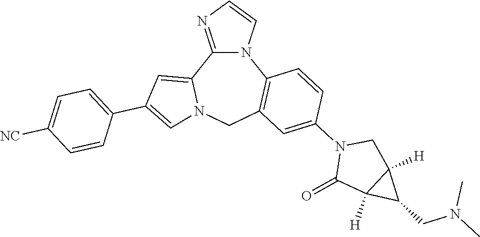 [SMILES: CN(C)C[C@H]1[C@@H]2CN(c3ccc4c(c3)Cn3cc(-c5ccc(C#N)cc5)cc3-c3nccn3-4)C(=O)[C@H]12] | 46 |
| 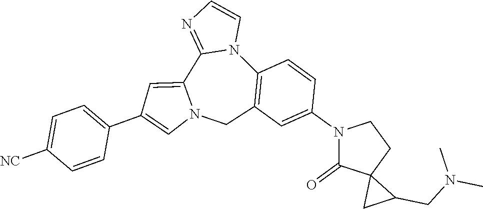 [SMILES: CN(C)CC1CC12CCN(c1ccc3c(c1)Cn1cc(-c4ccc(C#N)cc4)cc1-c1nccn1-3)C2=O] | 47 |
| 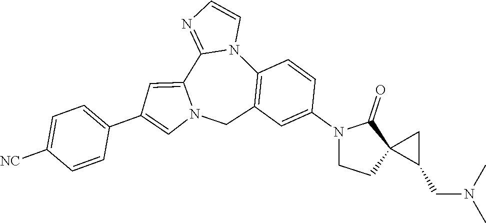 [SMILES: CN(C)C[C@H]1C[C@@]12CCN(c1ccc3c(c1)Cn1cc(-c4ccc(C#N)cc4)cc1-c1nccn1-3)C2=O] | 48 |
| 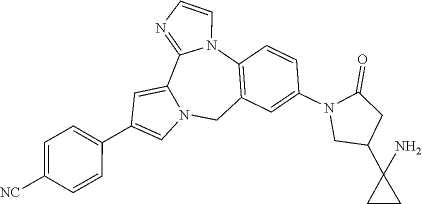 [SMILES: N#Cc1ccc(-c2cc3n(c2)Cc2cc(N4CC(C5(N)CC5)CC4=O)ccc2-n2ccnc2-3)cc1] | 49 |
| 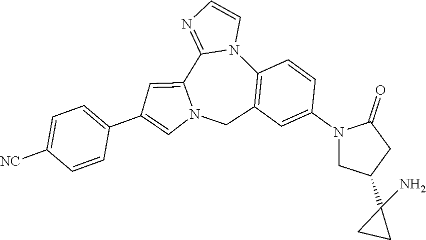 [SMILES: N#Cc1ccc(-c2cc3n(c2)Cc2cc(N4C[C@@H](C5(N)CC5)CC4=O)ccc2-n2ccnc2-3)cc1] | 50 |
| 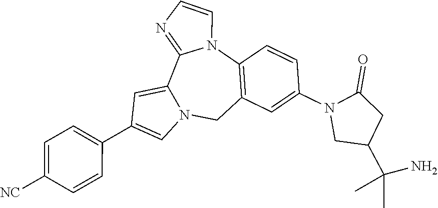 [SMILES: CC(C)(N)C1CC(=O)N(c2ccc3c(c2)Cn2cc(-c4ccc(C#N)cc4)cc2-c2nccn2-3)C1] | 51 |
| 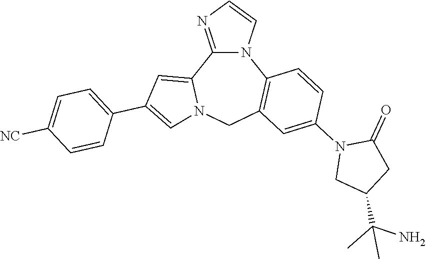 [SMILES: CC(C)(N)[C@H]1CC(=O)N(c2ccc3c(c2)Cn2cc(-c4ccc(C#N)cc4)cc2-c2nccn2-3)C1] | 52 |
| 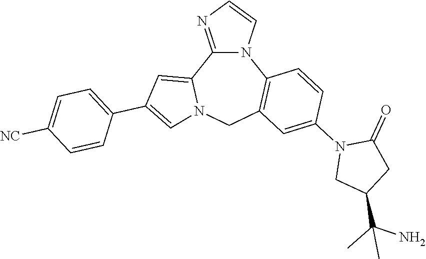 [SMILES: CC(C)(N)[C@@H]1CC(=O)N(c2ccc3c(c2)Cn2cc(-c4ccc(C#N)cc4)cc2-c2nccn2-3)C1] | 53 |
| 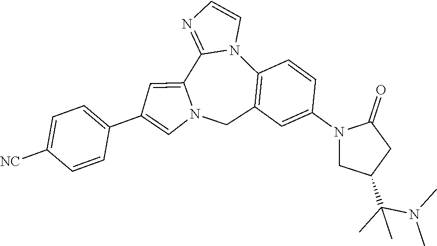 [SMILES: CN(C)C(C)(C)[C@H]1CC(=O)N(c2ccc3c(c2)Cn2cc(-c4ccc(C#N)cc4)cc2-c2nccn2-3)C1] | 54 |
| 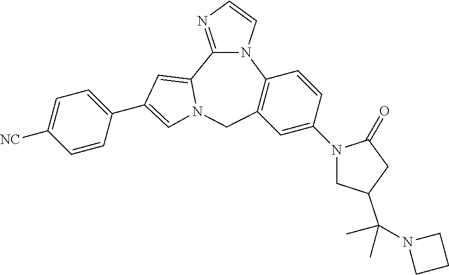 [SMILES: CC(C)(C1CC(=O)N(c2ccc3c(c2)Cn2cc(-c4ccc(C#N)cc4)cc2-c2nccn2-3)C1)N1CCC1] | 55 |
| 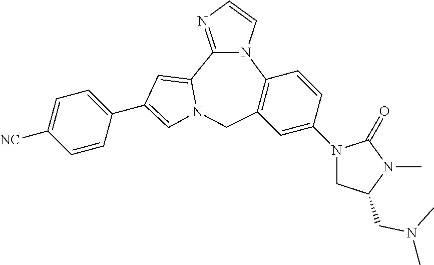 [SMILES: CN(C)C[C@@H]1CN(c2ccc3c(c2)Cn2cc(-c4ccc(C#N)cc4)cc2-c2nccn2-3)C(=O)N1C] | 56 |
| 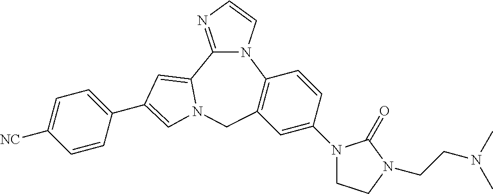 [SMILES: CN(C)CCN1CCN(c2ccc3c(c2)Cn2cc(-c4ccc(C#N)cc4)cc2-c2nccn2-3)C1=O] | 57 |
| 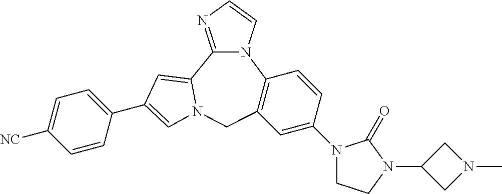 [SMILES: CN1CC(N2CCN(c3ccc4c(c3)Cn3cc(-c5ccc(C#N)cc5)cc3-c3nccn3-4)C2=O)C1] | 58 |
| 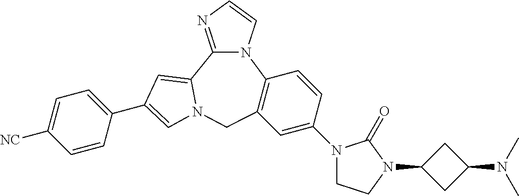 [SMILES: CN(C)[C@H]1C[C@@H](N2CCN(c3ccc4c(c3)Cn3cc(-c5ccc(C#N)cc5)cc3-c3nccn3-4)C2=O)C1] | 59 |
| 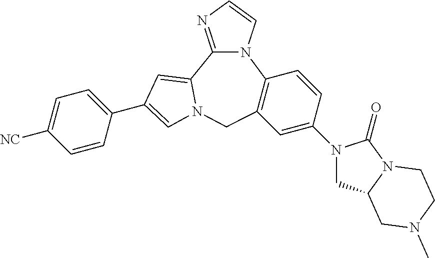 [SMILES: CN1CCN2C(=O)N(c3ccc4c(c3)Cn3cc(-c5ccc(C#N)cc5)cc3-c3nccn3-4)C[C@H]2C1] | 60 |
| 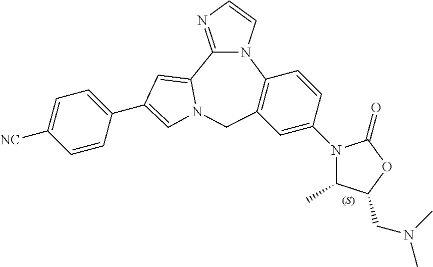 | 61 |
| 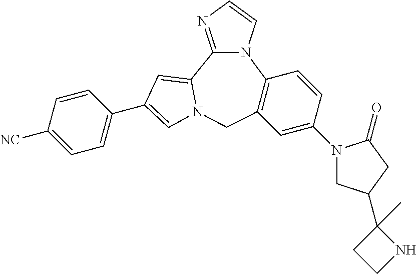 [SMILES: CC1(C2CC(=O)N(c3ccc4c(c3)Cn3cc(-c5ccc(C#N)cc5)cc3-c3nccn3-4)C2)CCN1] | 62 |
| 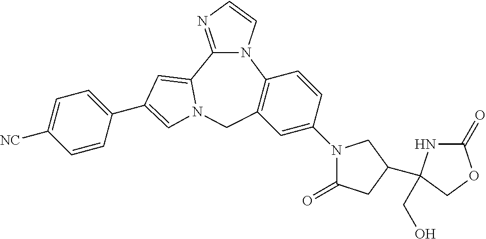 [SMILES: N#Cc1ccc(-c2cc3n(c2)Cc2cc(N4CC(C5(CO)COC(=O)N5)CC4=O)ccc2-n2ccnc2-3)cc1] | 63 |
| 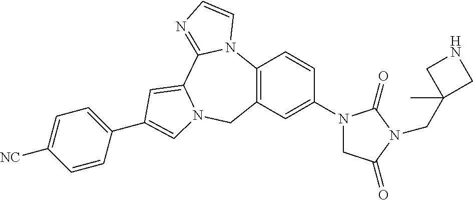 [SMILES: CC1(CN2C(=O)CN(c3ccc4c(c3)Cn3cc(-c5ccc(C#N)cc5)cc3-c3nccn3-4)C2=O)CNC1] | 64 |
| 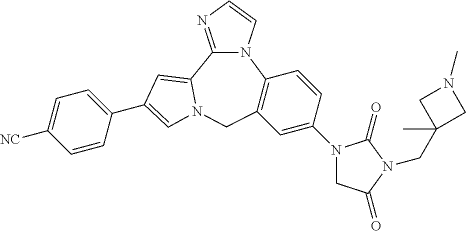 [SMILES: CN1CC(C)(CN2C(=O)CN(c3ccc4c(c3)Cn3cc(-c5ccc(C#N)cc5)cc3-c3nccn3-4)C2=O)C1] | 65 |
| 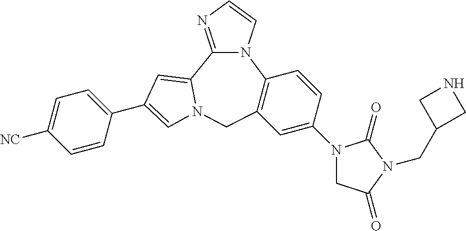 [SMILES: N#Cc1ccc(-c2cc3n(c2)Cc2cc(N4CC(=O)N(CC5CNC5)C4=O)ccc2-n2ccnc2-3)cc1] | 66 |
| 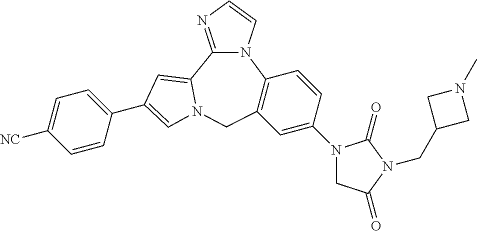 [SMILES: CN1CC(CN2C(=O)CN(c3ccc4c(c3)Cn3cc(-c5ccc(C#N)cc5)cc3-c3nccn3-4)C2=O)C1] | 67 |
| 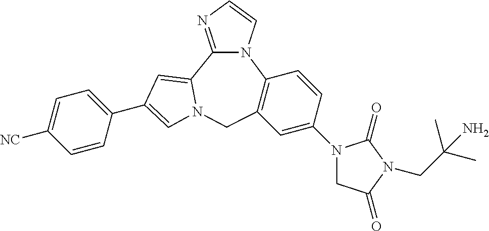 [SMILES: CC(C)(N)CN1C(=O)CN(c2ccc3c(c2)Cn2cc(-c4ccc(C#N)cc4)cc2-c2nccn2-3)C1=O] | 68 |
| 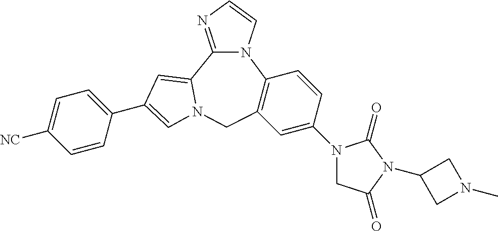 [SMILES: CN1CC(N2C(=O)CN(c3ccc4c(c3)Cn3cc(-c5ccc(C#N)cc5)cc3-c3nccn3-4)C2=O)C1] | 69 |
| 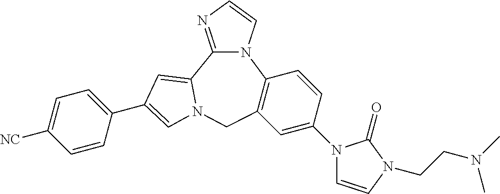 [SMILES: CN(C)CCn1ccn(-c2ccc3c(c2)Cn2cc(-c4ccc(C#N)cc4)cc2-c2nccn2-3)c1=O] | 70 |
| 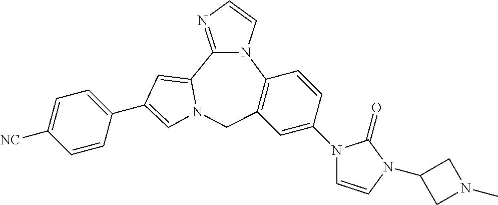 [SMILES: CN1CC(n2ccn(-c3ccc4c(c3)Cn3cc(-c5ccc(C#N)cc5)cc3-c3nccn3-4)c2=O)C1] | 71 |
| 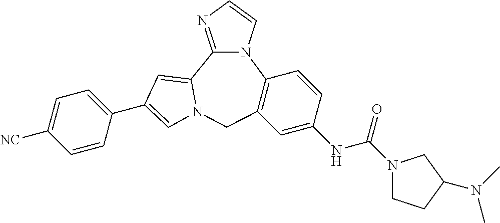 [SMILES: CN(C)C1CCN(C(=O)Nc2ccc3c(c2)Cn2cc(-c4ccc(C#N)cc4)cc2-c2nccn2-3)C1] | 72 |
| 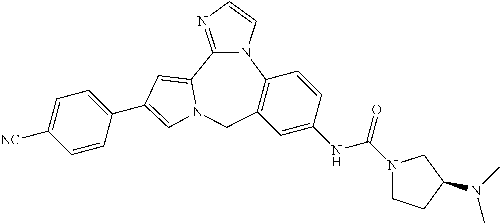 [SMILES: CN(C)[C@H]1CCN(C(=O)Nc2ccc3c(c2)Cn2cc(-c4ccc(C#N)cc4)cc2-c2nccn2-3)C1] | 73 |
| 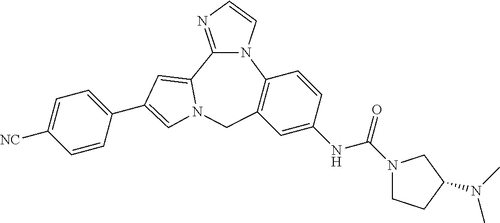 [SMILES: CN(C)[C@@H]1CCN(C(=O)Nc2ccc3c(c2)Cn2cc(-c4ccc(C#N)cc4)cc2-c2nccn2-3)C1] | 74 |
| 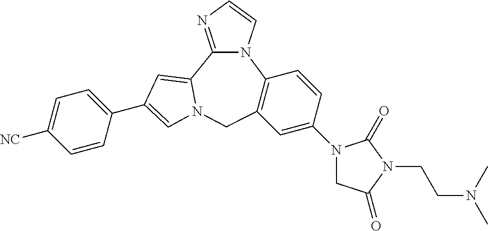 [SMILES: CN(C)CCN1C(=O)CN(c2ccc3c(c2)Cn2cc(-c4ccc(C#N)cc4)cc2-c2nccn2-3)C1=O] | 75 |
| 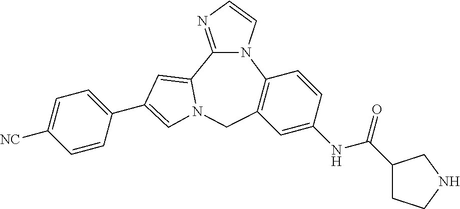 [SMILES: N#Cc1ccc(-c2cc3n(c2)Cc2cc(NC(=O)C4CCNC4)ccc2-n2ccnc2-3)cc1] | 76 |
| 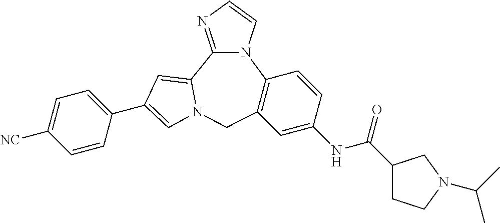 [SMILES: CC(C)N1CCC(C(=O)Nc2ccc3c(c2)Cn2cc(-c4ccc(C#N)cc4)cc2-c2nccn2-3)C1] | 77 |
| 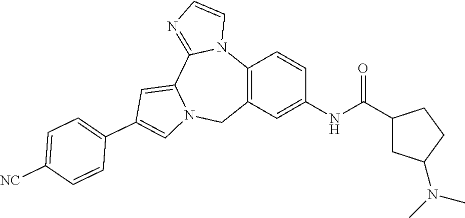 [SMILES: CN(C)C1CCC(C(=O)Nc2ccc3c(c2)Cn2cc(-c4ccc(C#N)cc4)cc2-c2nccn2-3)C1] | 78 |
| 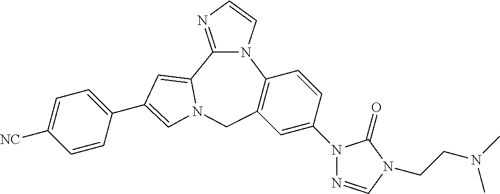 [SMILES: CN(C)CCn1cnn(-c2ccc3c(c2)Cn2cc(-c4ccc(C#N)cc4)cc2-c2nccn2-3)c1=O] | 79 |
| 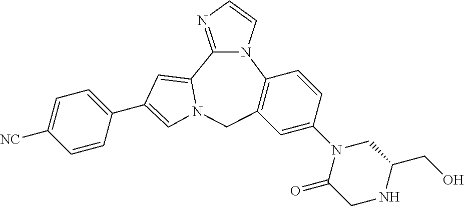 [SMILES: N#Cc1ccc(-c2cc3n(c2)Cc2cc(N4C[C@H](CO)NCC4=O)ccc2-n2ccnc2-3)cc1] | 80 |
| 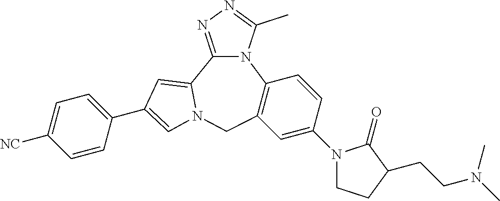 [SMILES: Cc1nnc2n1-c1ccc(N3CCC(CCN(C)C)C3=O)cc1Cn1cc(-c3ccc(C#N)cc3)cc1-2] | 81 |
| 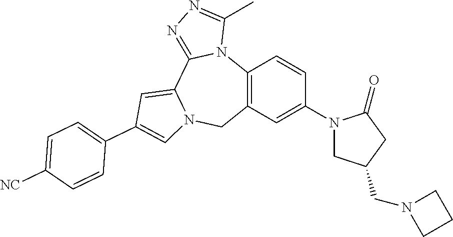 [SMILES: Cc1nnc2n1-c1ccc(N3C[C@@H](CN4CCC4)CC3=O)cc1Cn1cc(-c3ccc(C#N)cc3)cc1-2] | 82 |
| 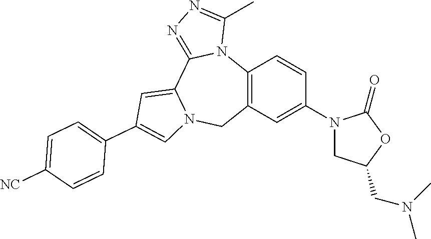 [SMILES: Cc1nnc2n1-c1ccc(N3C[C@@H](CN(C)C)OC3=O)cc1Cn1cc(-c3ccc(C#N)cc3)cc1-2] | 83 |
| 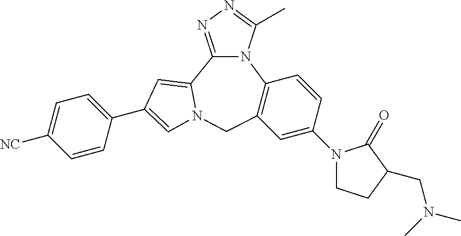 [SMILES: Cc1nnc2n1-c1ccc(N3CCC(CN(C)C)C3=O)cc1Cn1cc(-c3ccc(C#N)cc3)cc1-2] | 84 |
| 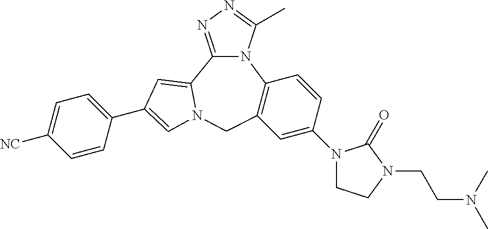 [SMILES: Cc1nnc2n1-c1ccc(N3CCN(CCN(C)C)C3=O)cc1Cn1cc(-c3ccc(C#N)cc3)cc1-2] | 85 |
| 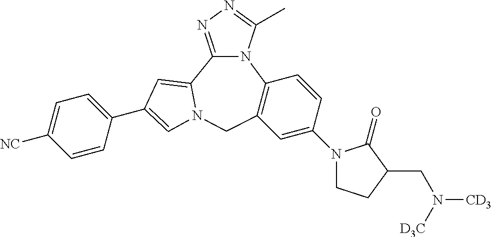 [SMILES: [2H]C([2H])([2H])N(CC1CCN(c2ccc3c(c2)Cn2cc(-c4ccc(C#N)cc4)cc2-c2nnc(C)n2-3)C1=O)C([2H])([2H])[2H]] | 86 |
| 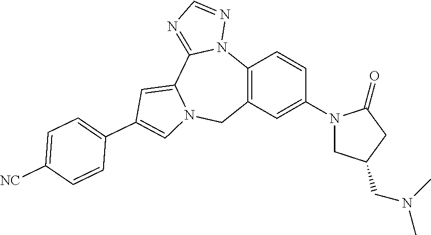 [SMILES: CN(C)C[C@H]1CC(=O)N(c2ccc3c(c2)Cn2cc(-c4ccc(C#N)cc4)cc2-c2ncnn2-3)C1] | 87 |
| 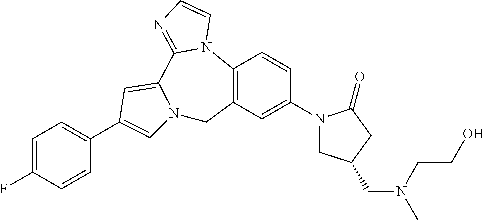 [SMILES: CN(CCO)C[C@H]1CC(=O)N(c2ccc3c(c2)Cn2cc(-c4ccc(F)cc4)cc2-c2nccn2-3)C1] | 89 |
| 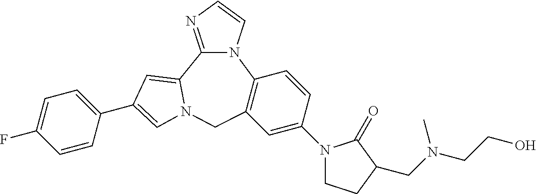 [SMILES: CN(CCO)CC1CCN(c2ccc3c(c2)Cn2cc(-c4ccc(F)cc4)cc2-c2nccn2-3)C1=O] | 90 |
| 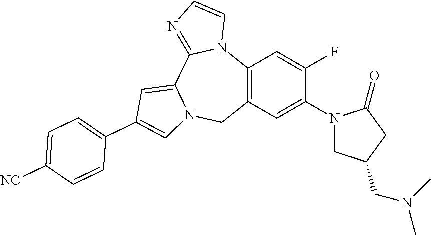 [SMILES: CN(C)C[C@H]1CC(=O)N(c2cc3c(cc2F)-n2ccnc2-c2cc(-c4ccc(C#N)cc4)cn2C3)C1] | 91 |
| 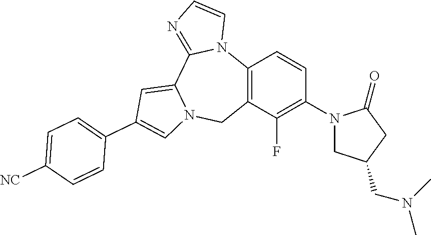 [SMILES: CN(C)C[C@H]1CC(=O)N(c2ccc3c(c2F)Cn2cc(-c4ccc(C#N)cc4)cc2-c2nccn2-3)C1] | 92 |
| 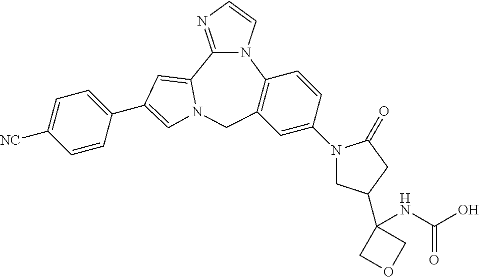 [SMILES: N#Cc1ccc(-c2cc3n(c2)Cc2cc(N4CC(C5(NC(=O)O)COC5)CC4=O)ccc2-n2ccnc2-3)cc1] | 94 |
| 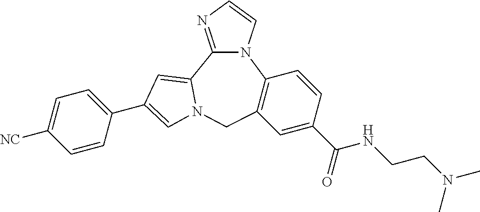 [SMILES: CN(C)CCNC(=O)c1ccc2c(c1)Cn1cc(-c3ccc(C#N)cc3)cc1-c1nccn1-2] | 101 |
| 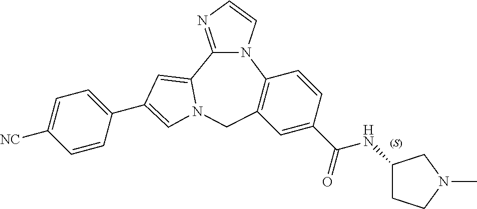 | 102 |
| 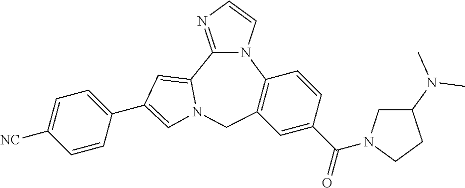 [SMILES: CN(C)C1CCN(C(=O)c2ccc3c(c2)Cn2cc(-c4ccc(C#N)cc4)cc2-c2nccn2-3)C1] | 103 |
| 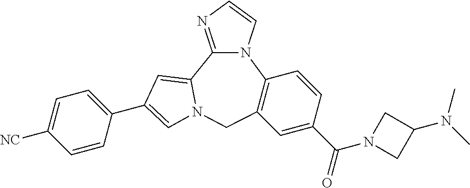 [SMILES: CN(C)C1CN(C(=O)c2ccc3c(c2)Cn2cc(-c4ccc(C#N)cc4)cc2-c2nccn2-3)C1] | 104 |
| 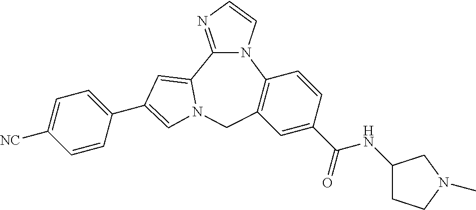 [SMILES: CN1CCC(NC(=O)c2ccc3c(c2)Cn2cc(-c4ccc(C#N)cc4)cc2-c2nccn2-3)C1] | 105 |
| 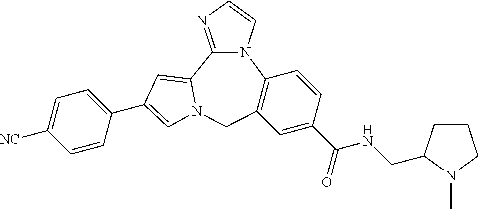 [SMILES: CN1CCCC1CNC(=O)c1ccc2c(c1)Cn1cc(-c3ccc(C#N)cc3)cc1-c1nccn1-2] | 106 |
| 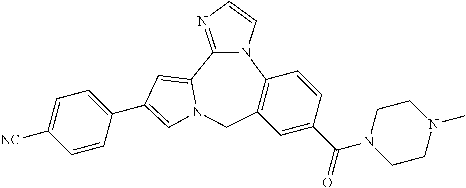 [SMILES: CN1CCN(C(=O)c2ccc3c(c2)Cn2cc(-c4ccc(C#N)cc4)cc2-c2nccn2-3)CC1] | 107 |
| 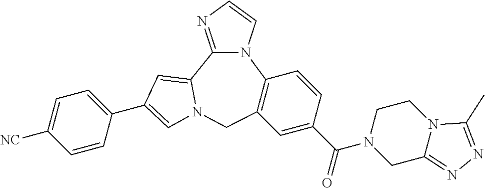 [SMILES: Cc1nnc2n1CCN(C(=O)c1ccc3c(c1)Cn1cc(-c4ccc(C#N)cc4)cc1-c1nccn1-3)C2] | 108 |
| 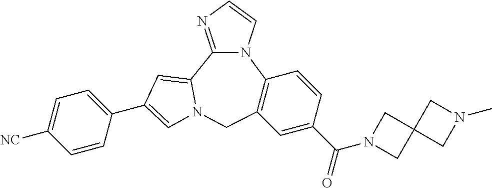 [SMILES: CN1CC2(C1)CN(C(=O)c1ccc3c(c1)Cn1cc(-c4ccc(C#N)cc4)cc1-c1nccn1-3)C2] | 109 |
| 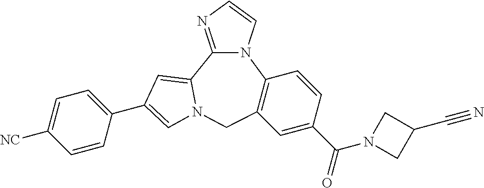 [SMILES: N#Cc1ccc(-c2cc3n(c2)Cc2cc(C(=O)N4CC(C#N)C4)ccc2-n2ccnc2-3)cc1] | 110 |
| 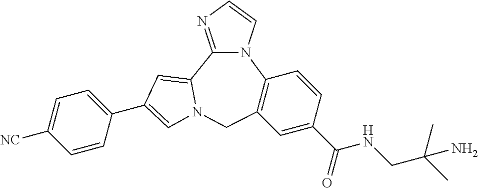 [SMILES: CC(C)(N)CNC(=O)c1ccc2c(c1)Cn1cc(-c3ccc(C#N)cc3)cc1-c1nccn1-2] | 111 |
| 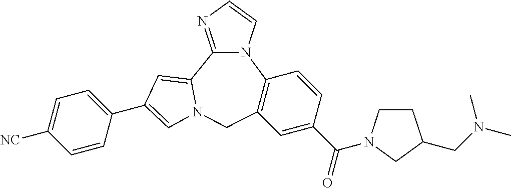 [SMILES: CN(C)CC1CCN(C(=O)c2ccc3c(c2)Cn2cc(-c4ccc(C#N)cc4)cc2-c2nccn2-3)C1] | 112 |
| 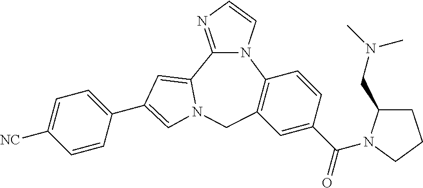 [SMILES: CN(C)C[C@H]1CCCN1C(=O)c1ccc2c(c1)Cn1cc(-c3ccc(C#N)cc3)cc1-c1nccn1-2] | 113 |
| 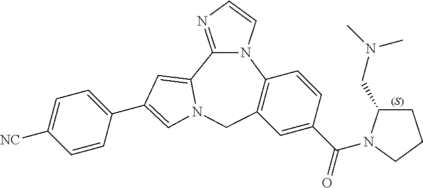 | 114 |
| 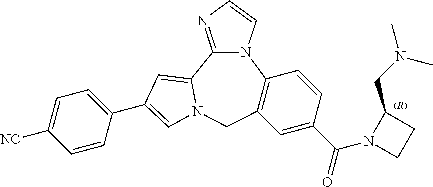 | 115 |
| 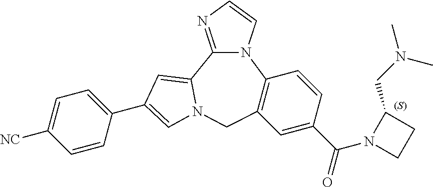 | 116 |
| 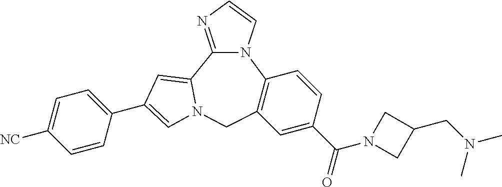 [SMILES: CN(C)CC1CN(C(=O)c2ccc3c(c2)Cn2cc(-c4ccc(C#N)cc4)cc2-c2nccn2-3)C1] | 117 |
| 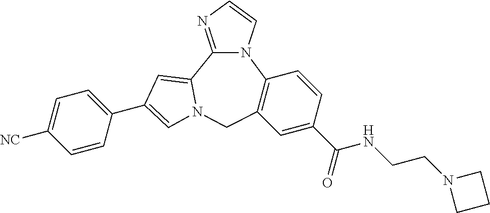 [SMILES: N#Cc1ccc(-c2cc3n(c2)Cc2cc(C(=O)NCCN4CCC4)ccc2-n2ccnc2-3)cc1] | 118 |
| 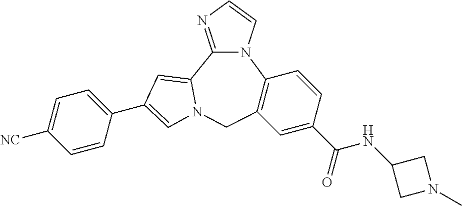 [SMILES: CN1CC(NC(=O)c2ccc3c(c2)Cn2cc(-c4ccc(C#N)cc4)cc2-c2nccn2-3)C1] | 119 |
| 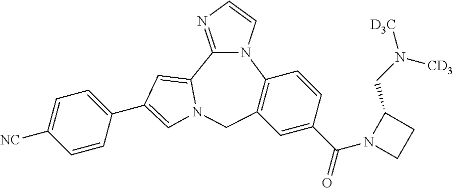 [SMILES: [2H]C([2H])([2H])N(C[C@@H]1CCN1C(=O)c1ccc2c(c1)Cn1cc(-c3ccc(C#N)cc3)cc1-c1nccn1-2)C([2H])([2H])[2H]] | 120 |
| 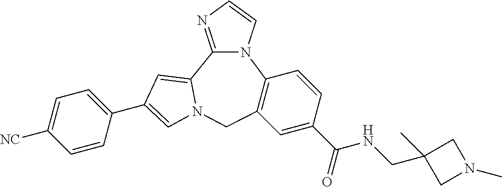 [SMILES: CN1CC(C)(CNC(=O)c2ccc3c(c2)Cn2cc(-c4ccc(C#N)cc4)cc2-c2nccn2-3)C1] | 121 |
| 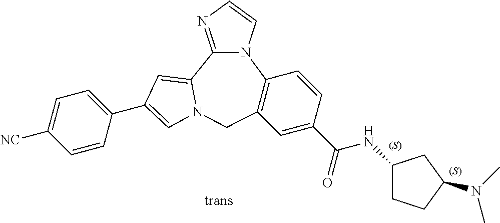 | 122 |
| 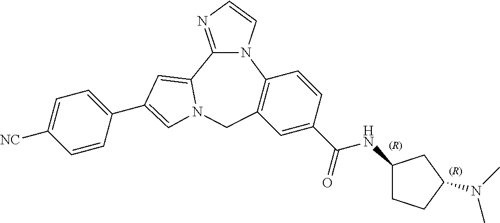 | 123 |
| 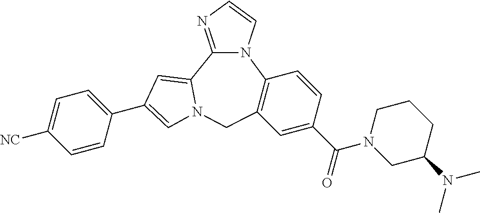 [SMILES: CN(C)[C@@H]1CCCN(C(=O)c2ccc3c(c2)Cn2cc(-c4ccc(C#N)cc4)cc2-c2nccn2-3)C1] | 124 |
| 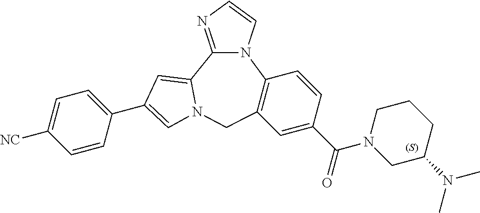 | 125 |
| 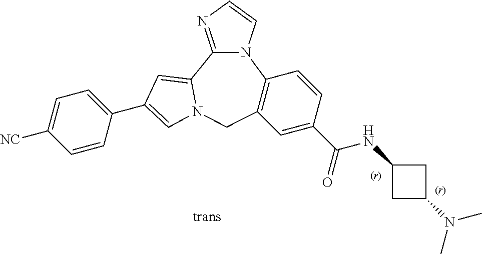 [SMILES: CN(C)[C@H]1C[C@H](NC(=O)c2ccc3c(c2)Cn2cc(-c4ccc(C#N)cc4)cc2-c2nccn2-3)C1] | 126 |
| 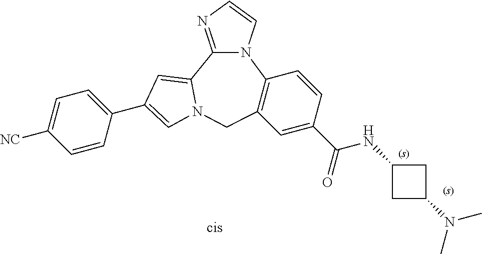 [SMILES: CN(C)[C@H]1C[C@@H](NC(=O)c2ccc3c(c2)Cn2cc(-c4ccc(C#N)cc4)cc2-c2nccn2-3)C1] | 127 |
| 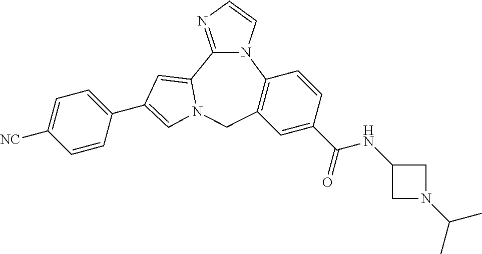 [SMILES: CC(C)N1CC(NC(=O)c2ccc3c(c2)Cn2cc(-c4ccc(C#N)cc4)cc2-c2nccn2-3)C1] | 128 |
| 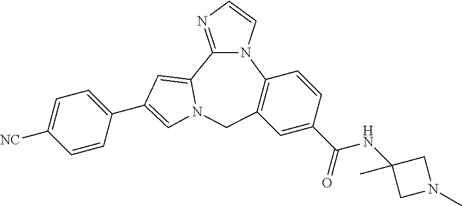 [SMILES: CN1CC(C)(NC(=O)c2ccc3c(c2)Cn2cc(-c4ccc(C#N)cc4)cc2-c2nccn2-3)C1] | 129 |
| 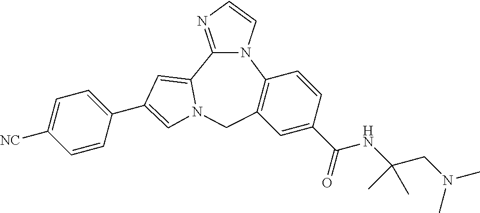 [SMILES: CN(C)CC(C)(C)NC(=O)c1ccc2c(c1)Cn1cc(-c3ccc(C#N)cc3)cc1-c1nccn1-2] | 130 |
| 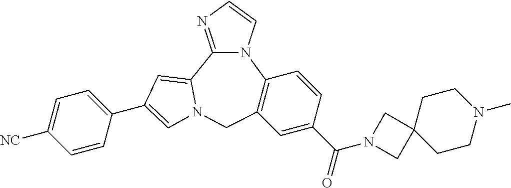 [SMILES: CN1CCC2(CC1)CN(C(=O)c1ccc3c(c1)Cn1cc(-c4ccc(C#N)cc4)cc1-c1nccn1-3)C2] | 131 |
| 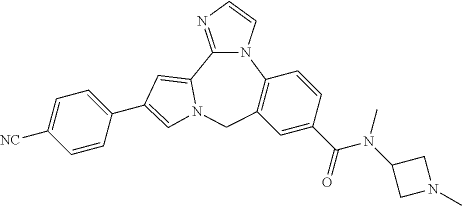 [SMILES: CN1CC(N(C)C(=O)c2ccc3c(c2)Cn2cc(-c4ccc(C#N)cc4)cc2-c2nccn2-3)C1] | 132 |
| 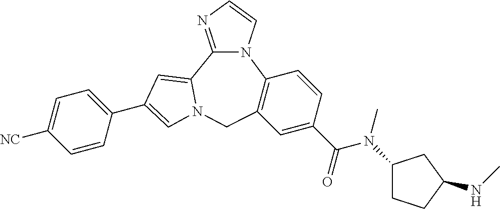 [SMILES: CN[C@H]1CC[C@H](N(C)C(=O)c2ccc3c(c2)Cn2cc(-c4ccc(C#N)cc4)cc2-c2nccn2-3)C1] | 133 |
| 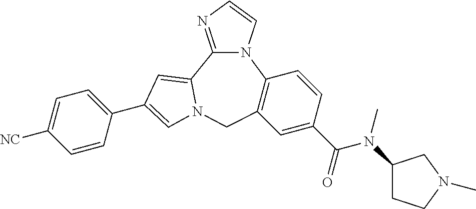 [SMILES: CN1CC[C@@H](N(C)C(=O)c2ccc3c(c2)Cn2cc(-c4ccc(C#N)cc4)cc2-c2nccn2-3)C1] | 134 |
| 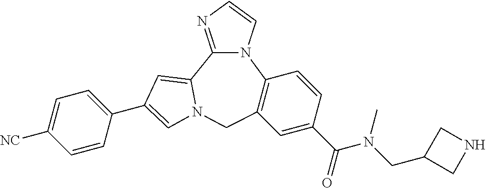 [SMILES: CN(CC1CNC1)C(=O)c1ccc2c(c1)Cn1cc(-c3ccc(C#N)cc3)cc1-c1nccn1-2] | 135 |
| 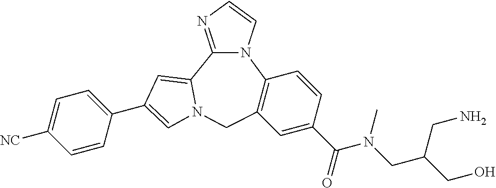 [SMILES: CN(CC(CN)CO)C(=O)c1ccc2c(c1)Cn1cc(-c3ccc(C#N)cc3)cc1-c1nccn1-2] | 136 |
| 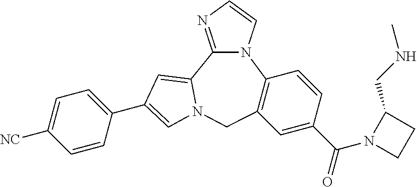 [SMILES: CNC[C@@H]1CCN1C(=O)c1ccc2c(c1)Cn1cc(-c3ccc(C#N)cc3)cc1-c1nccn1-2] | 137 |

In certain embodiments, compounds of the invention may be prodrugs of the compounds of Formula I, e.g., wherein a hydroxyl in the parent compound is presented as an ester or a carbonate, or carboxylic acid present in the parent compound is presented as an ester. In certain such embodiments, the prodrug is metabolized to the active parent compound in vivo (e.g., the ester is hydrolyzed to the corresponding hydroxyl, or carboxylic acid).

In certain embodiments, compounds of the invention may be racemic. In certain embodiments, compounds of the invention may be enriched in one enantiomer. For example, a compound of the invention may have greater than 30% ee, 40% ee, 50% ee, 60% ee, 70% ee, 80% ee, 90% ee, or even 95% or greater ee. Certain compounds of the invention have more than one stereocenter. Consequently, such compounds may be enriched in one or more diastereomer. For example, a compound of the invention may have greater than 30% de, 40% de, 50% de, 60% de, 70% de, 80% de, 90% de, or even 95% or greater de.

In certain embodiments, as will be described in detail below, the present invention relates to methods of treating or preventing cancer or an inflammatory disorder with a compound of Formula I, or a pharmaceutically acceptable salt thereof. In certain embodiments, the therapeutic preparation to be administered may be enriched to provide predominantly one enantiomer of a compound (e.g., of Formula I). An enantiomerically enriched mixture may comprise, for example, at least 60 mol percent of one enantiomer, or more preferably at least 75, 90, 95, or even 99 mol percent. In certain embodiments, the compound enriched in one enantiomer is substantially free of the other enantiomer, wherein substantially free means that the substance in question makes up less than 10%, or less than 5%, or less than 4%, or less than 3%, or less than 2%, or less than 1% as compared to the amount of the other enantiomer, e.g., in the composition or compound mixture. For example, if a composition or compound mixture contains 98 grams of a first enantiomer and 2 grams of a second enantiomer, it would be said to contain 98 mol percent of the first enantiomer and only 2% of the second enantiomer.

In certain embodiments, the therapeutic preparation may be enriched to provide predominantly one diastereomer of a compound (e.g., of Formula I). A diastereomerically enriched mixture may comprise, for example, at least 60 mol percent of one diastereomer, or more preferably at least 75, 90, 95, or even 99 mol percent.

In certain embodiments, the present invention provides a pharmaceutical preparation suitable for use in a human patient in the treatment of cancer an inflammatory disorder, comprising an effective amount of any compound of Formula I and one or more pharmaceutically acceptable excipients. In certain embodiments, the pharmaceutical preparations may be for use in treating or preventing a condition or disease as described herein. In certain embodiments, the pharmaceutical preparations have a low enough pyrogen activity to be suitable for use in a human patient.

Compounds of any of the above structures may be used in the manufacture of medicaments for the treatment of any diseases or conditions disclosed herein.

The subject invention is also intended to include all isotopes of atoms occurring on the compounds disclosed herein. Isotopes include those atoms having the same atomic number but different mass numbers. By way of general example and without limitation, isotopes of hydrogen include deuterium (D). Any notation of a carbon in structures throughout this application, when used without further notation, are intended to represent all isotopes of carbon, such as ¹²C, ¹³C, or ¹⁴C. Any notation of a hydrogen in structures throughout this application, when used without further notation, are intended to represent all isotopes of hydrogen, such as ¹H, ²H, or ³H. Isotopically-labeled compounds can generally be prepared by conventional techniques known to those skilled in the art using appropriate isotopically-labeled reagents in place of the non-labeled reagents employed.

Exemplary compounds of Formula I are depicted in Examples 1-93 and 101-137. The compounds of Examples 1-93 and 101-137 are understood to encompass both the free base and the conjugate acid. For example, the compounds in Examples 1-93 and 101-137 may be depicted as complexes or salts with trifluoroacetic acid or hydrochloric acid, but the compounds in their corresponding free base forms or as salts with other acids are equally within the scope of the invention. Compounds may be isolated in either the free base form, as a salt (e.g., a hydrochloride salt) or in both forms. In the chemical structures shown below, standard chemical abbreviations are sometimes used.

### II. Uses of MK2 Inhibitors

In certain aspects, the invention provides methods of treating or preventing a mitogen-activated protein kinase activated protein kinase-2 (MK2) related disorder, comprising administering to a subject a compound of Formula I, e.g., in a therapeutically effective amount.

In certain embodiments, the MK2 related disorder an inflamatory disorder. In other embodiments, the MK2 related disorder is a cancer.

In certain aspects, the invention provides methods of treating cancer, comprising administering to a subject a compound of Formula I, e.g., in a therapeutically effective amount.

In certain embodiments, the cancer may be one or a variant of Acute Lymphoblastic Leukemia (ALL), Acute Myeloid Leukemia (AML), Adrenocortical Carcinoma, AIDS-Related Cancers (Kaposi Sarcoma and Lymphoma), Anal Cancer, Appendix Cancer, Atypical Teratoid/Rhabdoid Tumor, Basal Cell Carcinoma, Bile Duct Cancer (including Extrahepatic), Bladder Cancer, Bone Cancer (including Osteosarcoma and Malignant Fibrous Histiocytoma), Brain Tumor (such as Astrocytomas, Brain and Spinal Cord Tumors, Brain Stem Glioma, Central Nervous System Atypical Teratoid/Rhabdoid Tumor, Central Nervous System Embryonal Tumors, Craniopharyngioma, Ependymoblastoma, Ependymoma, Medulloblastoma, Medulloepithelioma, Pineal Parenchymal Tumors of Intermediate Differentiation, Supratentorial Primitive Neuroectodermal Tumors and Pineoblastoma), Breast Cancer, Bronchial Tumors, Burkitt Lymphoma, Basal Cell Carcinoma, Bile Duct Cancer (including Extrahepatic), Bladder Cancer, Bone Cancer (including Osteosarcoma and Malignant Fibrous Histiocytoma), Carcinoid Tumor, Carcinoma of Unknown Primary, Central Nervous System (such as Atypical Teratoid/Rhabdoid Tumor, Embryonal Tumors and Lymphoma), Cervical Cancer, Childhood Cancers, Chordoma, Chronic Lymphocytic Leukemia (CLL), Chronic Myelogenous Leukemia (CML), Chronic Myeloproliferative Disorders, Colon Cancer, Colorectal Cancer, Craniopharyngioma, Cutaneous T-Cell Lymphoma (Mycosis Fungoides and Sezary Syndrome), Duct, Bile (Extrahepatic), Ductal Carcinoma In Situ (DCIS), Embryonal Tumors (Central Nervous System), Endometrial Cancer, Ependymoblastoma, Ependymoma, Esophageal Cancer, Esthesioneuroblastoma, Ewing Sarcoma Family of Tumors, Extracranial Germ Cell Tumor, Extragonadal Germ Cell Tumor, Extrahepatic Bile Duct Cancer, Eye Cancer (like Intraocular Melanoma, Retinoblastoma), Fibrous Histiocytoma of Bone (including Malignant and Osteosarcoma) Gallbladder Cancer, Gastric (Stomach) Cancer, Gastrointestinal Carcinoid Tumor, Gastrointestinal Stromal Tumors (GIST), Germ Cell Tumor (Extracranial, Extragonadal, Ovarian), Gestational Trophoblastic Tumor, Glioblastoma, Glioma, Hairy Cell Leukemia, Head and Neck Cancer, Heart Cancer, Hepatocellular (Liver) Cancer, Histiocytosis, Langerhans Cell, Hodgkin Lymphoma, Hypopharyngeal Cancer, Intraocular Melanoma, Islet Cell Tumors (Endocrine, Pancreas), Kaposi Sarcoma, Kidney (including Renal Cell), Langerhans Cell Histiocytosis, Laryngeal Cancer, Leukemia (including Acute Lymphoblastic (ALL), Acute Myeloid (AML), Chronic Lymphocytic (CLL), Chronic Myelogenous (CML), Hairy Cell), Lip and Oral Cavity Cancer, Liver Cancer (Primary), Lobular Carcinoma In Situ (LCIS), Lung Cancer (Non-Small Cell and Small Cell), Lymphoma (AIDS-Related, Burkitt, Cutaneous T-Cell (Mycosis Fungoides and Sezary Syndrome), Hodgkin, Non-Hodgkin, Primary Central Nervous System (CNS), Male Breast Cancer, Malignant Fibrous Histiocytoma of Bone and Osteosarcoma, Medulloblastoma, Medulloepithelioma, Melanoma (including Intraocular (Eye)), Merkel Cell Carcinoma, Mesothelioma (Malignant), Metastatic Squamous Neck Cancer with Occult Primary, Midline Tract Carcinoma Involving NUT Gene, Mouth Cancer, Multiple Endocrine Neoplasia Syndromes, Multiple Myeloma/Plasma Cell Neoplasm, Mycosis Fungoides, Myelodysplastic Syndromes, Myelodysplastic/Myeloproliferative Neoplasms, Chronic Myeloid Leukemia (CML), Acute Myelogenous Leukemia (AML), Myeloma and Multiple Myeloma, Myeloproliferative Disorders (Chronic), Nasal Cavity and Paranasal Sinus Cancer, Nasopharyngeal Cancer, Neuroblastoma, Non-Hodgkin Lymphoma, Non-Small Cell Lung Cancer, Oral Cancer, Oral Cavity Cancer, Lip and Oropharyngeal Cancer, Osteosarcoma and Malignant Fibrous Histiocytoma of Bone, Ovarian Cancer (such as Epithelial, Germ Cell Tumor, and Low Malignant Potential Tumor), Pancreatic Cancer (including Islet Cell Tumors), Papillomatosis, Paraganglioma, Paranasal Sinus and Nasal Cavity Cancer, Parathyroid Cancer, Penile Cancer, Pharyngeal Cancer, Pheochromocytoma, Pineal Parenchymal Tumors of Intermediate Differentiation, Pineoblastoma and Supratentorial Primitive Neuroectodermal Tumors, Pituitary Tumor, Plasma Cell Neoplasm/Multiple Myeloma, Pleuropulmonary Blastoma, Pregnancy and Breast Cancer, Primary Central Nervous System (CNS) Lymphoma, Prostate Cancer, Rectal Cancer, Renal Cell (Kidney) Cancer, Renal Pelvis and Ureter, Retinoblastoma, Rhabdomyosarcoma, Salivary Gland Cancer, Sarcoma (such as Ewing Sarcoma Family of Tumors, Kaposi, Soft Tissue, Uterine), Sezary Syndrome, Skin Cancer (such as Melanoma, Merkel Cell Carcinoma, Nonmelanoma), Small Cell Lung Cancer, Small Intestine Cancer, Soft Tissue Sarcoma, Squamous Cell Carcinoma, Squamous Neck Cancer with Occult Primary, Metastatic Stomach (Gastric) Cancer, Supratentorial Primitive Neuroectodermal Tumors, T-Cell Lymphoma (Cutaneous, Mycosis Fungoides and Sezary Syndrome), Testicular Cancer, Throat Cancer, Thymoma and Thymic Carcinoma, Thyroid Cancer, Transitional Cell Cancer of the Renal Pelvis and Ureter, Trophoblastic Tumor (Gestational), Unknown Primary, Unusual Cancers of Childhood, Ureter and Renal Pelvis, Transitional Cell Cancer, Urethral Cancer, Uterine Cancer, Endometrial, Uterine Sarcoma, Waldenström Macroglobulinemia and Wilms Tumor.

In certain embodiments, the cancer is a KRAS- or BRAF-dependent cancer.

In certain embodiments, the cancer is a solid tumor. The subject is generally one who has been diagnosed as having a cancerous tumor or one who has been previously treated for a cancerous tumor (e.g., where the tumor has been previously removed by surgery). The cancerous tumor may be a primary tumor and/or a secondary (e.g., metastatic) tumor.

In certain embodiments, the subject is a mammal, e.g., a human.

In certain embodiments, the cancer is associated with tissue of the bladder, bone marrow, breast, colon, kidney, liver, lung, ovary, pancreas, prostate, skin or thyroid.

In certain embodiments, the method of treating cancer further comprises conjointly administering radiation therapy.

In some embodiments, the method of treating cancer further comprises conjointly administering one or more additional chemotherapeutic agents.

Chemotherapeutic agents that may be conjointly administered with compounds of the invention include: ABT-263, aminoglutethimide, amsacrine, anastrozole, asparaginase, AZD5363, Bacillus Calmette-Guerin vaccine (bcg), bicalutamide, bleomycin, bortezomib, buserelin, busulfan, campothecin, capecitabine, carboplatin, carfilzomib, carmustine, chlorambucil, chloroquine, cisplatin, cladribine, clodronate, cobimetinib, colchicine, cyclophosphamide, cyproterone, cytarabine, dacarbazine, dactinomycin, daunorubicin, demethoxyviridin, dexamethasone, dichloroacetate, dienestrol, diethylstilbestrol, docetaxel, doxorubicin, epirubicin, estradiol, estramustine, etoposide, everolimus, exemestane, filgrastim, fludarabine, fludrocortisone, fluorouracil and 5-fluorouracil, fluoxymesterone, flutamide, gemcitabine, genistein, goserelin, hydroxyurea, idarubicin, ifosfamide, imatinib, interferon, irinotecan, lenalidomide, letrozole, leucovorin, leuprolide, levamisole, lomustine, lonidamine, LY2603618, mechlorethamine, medroxyprogesterone, megestrol, melphalan, mercaptopurine, mesna, metformin (e.g., metformin HCl), methotrexate, miltefosine, mitomycin, mitotane, mitoxantrone, MK2206, nilutamide, nocodazole, octreotide, oxaliplatin, paclitaxel, pamidronate, pentostatin, pazopanib, perifosine, PF-04691502, PF477736, plicamycin, pomalidomide, porfimer, procarbazine, raltitrexed, rituximab, romidepsin, selumetinib, sorafenib, streptozocin, sunitinib, suramin, tamoxifen, temozolomide, temsirolimus, teniposide, testosterone, thalidomide, thioguanine, thiotepa, titanocene dichloride, topotecan, trametinib, trastuzumab, tretinoin, vinblastine, vincristine, vindesine, vinorelbine, and vorinostat (SAHA).

For example, chemotherapeutic agents that may be conjointly administered with compounds of the invention include: aminoglutethimide, amsacrine, anastrozole, asparaginase, bcg, bicalutamide, bleomycin, bortezomib, buserelin, busulfan, campothecin, capecitabine, carboplatin, carfilzomib, carmustine, chlorambucil, chloroquine, cisplatin, cladribine, clodronate, colchicine, cyclophosphamide, cyproterone, cytarabine, dacarbazine, dactinomycin, daunorubicin, demethoxyviridin, dichloroacetate, dienestrol, diethylstilbestrol, docetaxel, doxorubicin, epirubicin, estradiol, estramustine, etoposide, everolimus, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, lenalidomide, letrozole, leucovorin, leuprolide, levamisole, lomustine, lonidamine, mechlorethamine, medroxyprogesterone, megestrol, melphalan, mercaptopurine, mesna, metformin (e.g., metformin HCl), methotrexate, mitomycin, mitotane, mitoxantrone, nilutamide, nocodazole, octreotide, oxaliplatin, paclitaxel, pamidronate, pentostatin, perifosine, plicamycin, pomalidomide, porfimer, procarbazine, raltitrexed, rituximab, sorafenib, streptozocin, sunitinib, suramin, tamoxifen, temozolomide, temsirolimus, teniposide, testosterone, thalidomide, thioguanine, thiotepa, titanocene dichloride, topotecan, trastuzumab, tretinoin, vinblastine, vincristine, vindesine, and vinorelbine. In certain embodiments, the chemotherapeutic agent is cisplatin. In certain embodiments, the additional chemotherapeutic agent is an CHK1 inhibitor. In certain embodiments, the additional chemotherapeutic agent is an alkylating agent.

The “PF477736” refers to the following structure which is commercially available at least from Sigma-Aldrich (Catalog No. PZ0186):

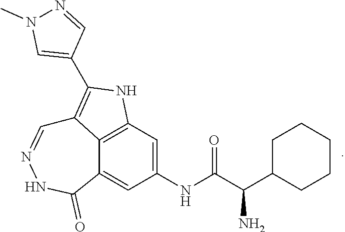 [SMILES: Cn1cc(-c2[nH]c3cc(NC(=O)[C@H](N)C4CCCCC4)cc4c3c2C=NNC4=O)cn1]

“LY2603618” refers to the following structure which is commercially available at least from Sigma-Aldrich (Catalog No. SML2855):

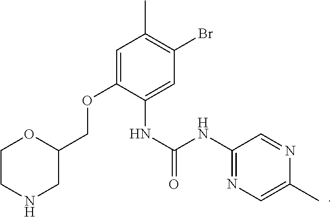 [SMILES: Cc1cnc(NC(=O)Nc2cc(Br)c(C)cc2OCC2CNCCO2)cn1]

Many combination therapies have been developed for the treatment of cancer. In certain embodiments, compounds of the invention may be conjointly administered with a combination therapy. Examples of combination therapies with which compounds of the invention may be conjointly administered are included in Table 2.

TABLE 2
Exemplary combinatorial therapies for the treatment of cancer.

| Name | Therapeutic agents |
|---|---|
| ABV | Doxorubicin, Bleomycin, Vinblastine |
| ABVD | Doxorubicin, Bleomycin, Vinblastine, Dacarbazine |
| AC (Breast) | Doxorubicin, Cyclophosphamide |
| AC (Sarcoma) | Doxorubicin, Cisplatin |
| AC (Neuroblastoma) | Cyclophosphamide, Doxorubicin |
| ACE | Cyclophosphamide, Doxorubicin, Etoposide |
| ACe | Cyclophosphamide, Doxorubicin |
| AD | Doxorubicin, Dacarbazine |
| AP | Doxorubicin, Cisplatin |
| ARAC-DNR | Cytarabine, Daunorubicin |
| B-CAVe | Bleomycin, Lomustine, Doxorubicin, Vinblastine |
| BCVPP | Carmustine, Cyclophosphamide, Vinblastine, Procarbazine, |
| | Prednisone |
| BEACOPP | Bleomycin, Etoposide, Doxorubicin, Cyclophosphamide, |
| | Vincristine, Procarbazine, Prednisone, Filgrastim |
| BEP | Bleomycin, Etoposide, Cisplatin |
| BIP | Bleomycin, Cisplatin, Ifosfamide, Mesna |
| BOMP | Bleomycin, Vincristine, Cisplatin, Mitomycin |
| CA | Cytarabine, Asparaginase |
| CABO | Cisplatin, Methotrexate, Bleomycin, Vincristine |
| CAF | Cyclophosphamide, Doxorubicin, Fluorouracil |
| CAL-G | Cyclophosphamide, Daunorubicin, Vincristine, Prednisone, |
| | Asparaginase |
| CAMP | Cyclophosphamide, Doxorubicin, Methotrexate, |
| | Procarbazine |
| CAP | Cyclophosphamide, Doxorubicin, Cisplatin |
| CaT | Carboplatin, Paclitaxel |
| CAV | Cyclophosphamide, Doxorubicin, Vincristine |
| CAVE ADD | CAV and Etoposide |
| CA-VP16 | Cyclophosphamide, Doxorubicin, Etoposide |
| CC | Cyclophosphamide, Carboplatin |
| CDDP/VP-16 | Cisplatin, Etoposide |
| CEF | Cyclophosphamide, Epirubicin, Fluorouracil |
| CEPP(B) | Cyclophosphamide, Etoposide, Prednisone, with or without/ |
| | Bleomycin |
| CEV | Cyclophosphamide, Etoposide, Vincristine |
| CF | Cisplatin, Fluorouracil or Carboplatin Fluorouracil |
| CHAP | Cyclophosphamide or Cyclophosphamide, Altretamine, |
| | Doxorubicin, Cisplatin |
| ChlVPP | Chlorambucil, Vinblastine, Procarbazine, Prednisone |
| CHOP | Cyclophosphamide, Doxorubicin, Vincristine, Prednisone |
| CHOP-BLEO | Add Bleomycin to CHOP |
| CISCA | Cyclophosphamide, Doxorubicin, Cisplatin |
| CLD-BOMP | Bleomycin, Cisplatin, Vincristine, Mitomycin |
| CMF | Methotrexate, Fluorouracil, Cyclophosphamide |
| CMFP | Cyclophosphamide, Methotrexate, Fluorouracil, Prednisone |
| CMFVP | Cyclophosphamide, Methotrexate, Fluorouracil, Vincristine, |
| | Prednisone |
| CMV | Cisplatin, Methotrexate, Vinblastine |
| CNF | Cyclophosphamide, Mitoxantrone, Fluorouracil |
| CNOP | Cyclophosphamide, Mitoxantrone, Vincristine, Prednisone |
| COB | Cisplatin, Vincristine, Bleomycin |
| CODE | Cisplatin, Vincristine, Doxorubicin, Etoposide |
| COMLA | Cyclophosphamide, Vincristine, Methotrexate, Leucovorin, |
| | Cytarabine |
| COMP | Cyclophosphamide, Vincristine, Methotrexate, Prednisone |
| Cooper Regimen | Cyclophosphamide, Methotrexate, Fluorouracil, Vincristine, |
| | Prednisone |
| COP | Cyclophosphamide, Vincristine, Prednisone |
| COPE | Cyclophosphamide, Vincristine, Cisplatin, Etoposide |
| COPP | Cyclophosphamide, Vincristine, Procarbazine, Prednisone |
| CP(Chronic lymphocytic | Chlorambucil, Prednisone |
| leukemia) | |
| CP (Ovarian Cancer) | Cyclophosphamide, Cisplatin |
| CT | Cisplatin, Paclitaxel |
| CVD | Cisplatin, Vinblastine, Dacarbazine |
| CVI | Carboplatin, Etoposide, Ifosfamide, Mesna |
| CVP | Cyclophosphamide, Vincristine, Prednisome |
| CVPP | Lomustine, Procarbazine, Prednisone |
| CYVADIC | Cyclophosphamide, Vincristine, Doxorubicin, Dacarbazine |
| DA | Daunorubicin, Cytarabine |
| DAT | Daunorubicin, Cytarabine, Thioguanine |
| DAV | Daunorubicin, Cytarabine, Etoposide |
| DCT | Daunorubicin, Cytarabine, Thioguanine |
| DHAP | Cisplatin, Cytarabine, Dexamethasone |
| DI | Doxorubicin, Ifosfamide |
| DTIC/Tamoxifen | Dacarbazine, Tamoxifen |
| DVP | Daunorubicin, Vincristine, Prednisone |
| EAP | Etoposide, Doxorubicin, Cisplatin |
| EC | Etoposide, Carboplatin |
| EFP | Etoposie, Fluorouracil, Cisplatin |
| ELF | Etoposide, Leucovorin, Fluorouracil |
| EMA 86 | Mitoxantrone, Etoposide, Cytarabine |
| EP | Etoposide, Cisplatin |
| EVA | Etoposide, Vinblastine |
| FAC | Fluorouracil, Doxorubicin, Cyclophosphamide |
| FAM | Fluorouracil, Doxorubicin, Mitomycin |
| FAMTX | Methotrexate, Leucovorin, Doxorubicin |
| FAP | Fluorouracil, Doxorubicin, Cisplatin |
| F-CL | Fluorouracil, Leucovorin |
| FEC | Fluorouracil, Cyclophosphamide, Epirubicin |
| FED | Fluorouracil, Etoposide, Cisplatin |
| FL | Flutamide, Leuprolide |
| FZ | Flutamide, Goserelin acetate implant |
| HDMTX | Methotrexate, Leucovorin |
| Hexa-CAF | Altretamine, Cyclophosphamide, Methotrexate, Fluorouracil |
| ICE-T | Ifosfamide, Carboplatin, Etoposide, Paclitaxel, Mesna |
| IDMTX/6-MP | Methotrexate, Mercaptopurine, Leucovorin |
| IE | Ifosfamide, Etoposie, Mesna |
| IfoVP | Ifosfamide, Etoposide, Mesna |
| IPA | Ifosfamide, Cisplatin, Doxorubicin |
| M-2 | Vincristine, Carmustine, Cyclophosphamide, Prednisone, |
| | Melphalan |
| MAC-III | Methotrexate, Leucovorin, Dactinomycin, |
| | Cyclophosphamide |
| MACC | Methotrexate, Doxorubicin, Cyclophosphamide, Lomustine |
| MACOP-B | Methotrexate, Leucovorin, Doxorubicin, Cyclophosphamide, |
| | Vincristine, Bleomycin, Prednisone |
| MAID | Mesna, Doxorubicin, Ifosfamide, Dacarbazine |
| m-BACOD | Bleomycin, Doxorubicin, Cyclophosphamide, Vincristine, |
| | Dexamethasone, Methotrexate, Leucovorin |
| MBC | Methotrexate, Bleomycin, Cisplatin |
| MC | Mitoxantrone, Cytarabine |
| MF | Methotrexate, Fluorouracil, Leucovorin |
| MICE | Ifosfamide, Carboplatin, Etoposide, Mesna |
| MINE | Mesna, Ifosfamide, Mitoxantrone, Etoposide |
| mini-BEAM | Carmustine, Etoposide, Cytarabine, Melphalan |
| MOBP | Bleomycin, Vincristine, Cisplatin, Mitomycin |
| MOP | Mechlorethamine, Vincristine, Procarbazine |
| MOPP | Mechlorethamine, Vincristine, Procarbazine, Prednisone |
| MOPP/ABV | Mechlorethamine, Vincristine, Procarbazine, Prednisone, |
| | Doxorubicin, Bleomycin, Vinblastine |
| MP (multiple myeloma) | Melphalan, Prednisone |
| MP (prostate cancer) | Mitoxantrone, Prednisone |
| MTX/6-MO | Methotrexate, Mercaptopurine |
| MTX/6-MP/VP | Methotrexate, Mercaptopurine, Vincristine, Prednisone |
| MTX-CDDPAdr | Methotrexate, Leucovorin, Cisplatin, Doxorubicin |
| MV (breast cancer) | Mitomycin, Vinblastine |
| MV (acute myelocytic | Mitoxantrone, Etoposide |
| leukemia) | |
| M-VAC Methotrexate | Vinblastine, Doxorubicin, Cisplatin |
| MVP Mitomycin | Vinblastine, Cisplatin |
| MVPP | Mechlorethamine, Vinblastine, Procarbazine, Prednisone |
| NFL | Mitoxantrone, Fluorouracil, Leucovorin |
| NOVP | Mitoxantrone, Vinblastine, Vincristine |
| OPA | Vincristine, Prednisone, Doxorubicin |
| OPPA | Add Procarbazine to OPA. |
| PAC | Cisplatin, Doxorubicin |
| PAC-I | Cisplatin, Doxorubicin, Cyclophosphamide |
| PA-CI | Cisplatin, Doxorubicin |
| PC | Paclitaxel, Carboplatin or Paclitaxel, Cisplatin |
| PCV | Lomustine, Procarbazine, Vincristine |
| PE | Paclitaxel, Estramustine |
| PFL | Cisplatin, Fluorouracil, Leucovorin |
| POC | Prednisone, Vincristine, Lomustine |
| ProMACE | Prednisone, Methotrexate, Leucovorin, Doxorubicin, |
| | Cyclophosphamide, Etoposide |
| ProMACE/cytaBOM | Prednisone, Doxorubicin, Cyclophosphamide, Etoposide, |
| | Cytarabine, Bleomycin, Vincristine, Methotrexate, |
| | Leucovorin, Cotrimoxazole |
| PRoMACE/MOPP | Prednisone, Doxorubicin, Cyclophosphamide, Etoposide, |
| | Mechlorethamine, Vincristine, Procarbazine, Methotrexate, |
| | Leucovorin |
| Pt/VM | Cisplatin, Teniposide |
| PVA | Prednisone, Vincristine, Asparaginase |
| PVB | Cisplatin, Vinblastine, Bleomycin |
| PVDA | Prednisone, Vincristine, Daunorubicin, Asparaginase |
| SMF | Streptozocin, Mitomycin, Fluorouracil |
| TAD | Mechlorethamine, Doxorubicin, Vinblastine, Vincristine, |
| | Bleomycin, Etoposide, Prednisone |
| TCF | Paclitaxel, Cisplatin, Fluorouracil |
| TIP | Paclitaxel, Ifosfamide, Mesna, Cisplatin |
| TTT | Methotrexate, Cytarabine, Hydrocortisone |
| Topo/CTX | Cyclophosphamide, Topotecan, Mesna |
| VAB-6 | Cyclophosphamide, Dactinomycin, Vinblastine, Cisplatin, |
| | Bleomycin |
| VAC | Vincristine, Dactinomycin, Cyclophosphamide |
| VACAdr | Vincristine, Cyclophosphamide, Doxorubicin, Dactinomycin, |
| | Vincristine |
| VAD | Vincristine, Doxorubicin, Dexamethasone |
| VATH | Vinblastine, Doxorubicin, Thiotepa, Flouxymesterone |
| VBAP | Vincristine, Carmustine, Doxorubicin, Prednisone |
| VBCMP | Vincristine, Carmustine, Melphalan, Cyclophosphamide, |
| | Prednisone |
| VC | Vinorelbine, Cisplatin |
| VCAP | Vincristine, Cyclophosphamide, Doxorubicin, Prednisone |
| VD | Vinorelbine, Doxorubicin |
| VelP | Vinblastine, Cisplatin, Ifosfamide, Mesna |
| VIP | Etoposide, Cisplatin, Ifosfamide, Mesna |
| VM | Mitomycin, Vinblastine |
| VMCP | Vincristine, Melphalan, Cyclophosphamide, Prednisone |
| VP | Etoposide, Cisplatin |
| V-TAD | Etoposide, Thioguanine, Daunorubicin, Cytarabine |
| 5 + 2 | Cytarabine, Daunorubicin, Mitoxantrone |
| 7 + 3 | Cytarabine with/, Daunorubicin or Idarubicin or |
| | Mitoxantrone |
| “8 in 1” | Methylprednisolone, Vincristine, Lomustine, Procarbazine, |
| | Hydroxyurea, Cisplatin, Cytarabine, Dacarbazine |

In some embodiments, the conjointly administered chemotherapeutic agent is an immune-oncology therapeutic, such as an inhibitor of CTLA-4, indoleamine 2,3-dioxygenase, and/or PD-1/PD-L1.

In certain embodiments, conjoint administration of the MK2 inhibitor(s) of Formula I with one or more additional therapeutic agent(s) (e.g., one or more additional chemotherapeutic agent(s)) provides improved efficacy relative to each individual administration of the MK2 inhibitor (e.g., a compound of Formula I) or the one or more additional therapeutic agent(s). In certain such embodiments, the conjoint administration provides an additive effect, wherein an additive effect refers to the sum of each of the effects of individual administration of the MK2 inhibitor and the one or more additional therapeutic agent(s). In certain embodiments, coadministration produces a synergistic effect.

In certain embodiments, the MK2 inhibitor and the one or more additional chemotherapeutic agents are administered simultaneously. In alternative embodiments, the one or more additional chemotherapeutic agents are administered within about 5 minutes to within about 168 hours prior to or after administration of the MK2 inhibitor.

In certain embodiments, the invention provides methods of inhibiting proliferation of a cancerous cell comprising contacting a cancerous cell with an effective amount of a compound of Formula I.

The invention also provides methods of inhibiting MK2 activity in a cell, comprising contacting a cell with a compound of Formula I. In certain embodiments, the cell is a cancer cell. Such methods may be performed in vivo or in vitro.

The invention also provides a method of treating or preventing a metabolic disorder, comprising administering to a subject a compound of Formula I. In certain embodiments, the metabolic disorder is diabetes, insulin resistance, obesity, or metabolic syndrome. In some embodiments, the diabetes is Type I, Type II, or gestational diabetes. In some embodiments, the treating or preventing affects glycogenolysis or gluceoneogenesis in the subject. In some embodiments, the treating or preventing reduces hepatic glucose production, hyperglycemis, fatty liver, insulin resistance, insulin-resistance-associated inflammation, insuling resistance-associated dyslipidemia, or any combination thereof, in the subject.

In certain embodiments, e.g., of methods of treating diabetes, the method further comprises conjointly administering one or more additional antidiabetic agents. Anti-diabetic agents that may be conjointly administered with compounds of the invention include, but not limited to, sulfonylurea, biguanides, alpha-glucosidase inhibitors, thiazolidinediones (TZDs), dipeptidyl peptidase-inhibitors (DPP-4 inhibitors), nonsulfonylurea insulin secretagogues, glucagon-like peptide-1 analogs (GLP-1 analogs) and insulin. More specifically, the antidiabetic drugs include, but are not limited to metformin (metformin HCl), glyburide, glimepiride, glipyride, glipizide, chlorpropamide, gliclazide, acarbose, miglitol, pioglitazone, troglitazone, rosiglitazone, isaglitazone, muraglitizar, peliglitazar, sitagliptin, saxagliptin, vildagliptin, alogliptin, linagliptin, dutogliptin, dutogliptin, repaglinide, nateglinide, mitiglindine, exenatide, liraglutide, albiglutide and insulin. In certain preferred embodiments, the one or more additional antidiabetic agents comprises metformin.

### III. Pharmaceutical Compositions

In certain embodiments, the present invention provides pharmaceutical compositions comprising a compound of Formula I and a pharmaceutically acceptable carrier.

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In a preferred embodiment, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as an eye drop.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a selfemulsifying drug delivery system or a selfmicroemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin, or as an eye drop). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof, (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions for rectal, vaginal, or urethral administration may be presented as a suppository, which may be prepared by mixing one or more active compounds with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the pharmaceutical compositions for administration to the mouth may be presented as a mouthwash, or an oral spray, or an oral ointment.

Alternatively or additionally, compositions can be formulated for delivery via a catheter, stent, wire, or other intraluminal device. Delivery via such devices may be especially useful for delivery to the bladder, urethra, ureter, rectum, or intestine.

Formulations which are suitable for vaginal administration also include pessaries, tampons, creams, gels, pastes, foams or spray formulations containing such carriers as are known in the art to be appropriate.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention. Exemplary ophthalmic formulations are described in U.S. Publication Nos. 2005/0080056, 2005/0059744, 2005/0031697 and 2005/004074 and U.S. Pat. No. 6,583,124, the contents of which are incorporated herein by reference. If desired, liquid ophthalmic formulations have properties similar to that of lacrimal fluids, aqueous humor or vitreous humor or are compatable with such fluids. A preferred route of administration is local administration (e.g., topical administration, such as eye drops, or administration via an implant).

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the amount of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including primates, in particular humans, and other mammals such as equines, cattle, swine and sheep; and poultry and pets in general.

In certain embodiments, compounds of the invention may be used alone or conjointly administered with another type of therapeutic agent. As used herein, the phrase “conjoint administration” refers to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week of one another. Thus, an individual who receives such treatment can benefit from a combined effect of different therapeutic compounds.

In certain embodiments, conjoint administration of compounds of the invention with one or more additional therapeutic agent(s) (e.g., one or more additional chemotherapeutic agent(s)) provides improved efficacy relative to each individual administration of the compound of the invention (e.g., compound of Formula I) or the one or more additional therapeutic agent(s). In certain such embodiments, the conjoint administration provides an additive effect, wherein an additive effect refers to the sum of each of the effects of individual administration of the compound of the invention and the one or more additional therapeutic agent(s).

This invention includes the use of pharmaceutically acceptable salts of compounds of the invention in the compositions and methods of the present invention. The term “pharmaceutically acceptable salt” as used herein includes salts derived from inorganic or organic acids including, for example, hydrochloric, hydrobromic, sulfuric, nitric, perchloric, phosphoric, formic, acetic, lactic, maleic, fumaric, succinic, tartaric, glycolic, salicylic, citric, methanesulfonic, benzenesulfonic, benzoic, malonic, trifluoroacetic, trichloroacetic, naphthalene-2-sulfonic, and other acids. Pharmaceutically acceptable salt forms can include forms wherein the ratio of molecules comprising the salt is not 1:1. For example, the salt may comprise more than one inorganic or organic acid molecule per molecule of base, such as two hydrochloric acid molecules per molecule of compound of Formula I. As another example, the salt may comprise less than one inorganic or organic acid molecule per molecule of base, such as two molecules of compound of Formula I per molecule of tartaric acid.

In further embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### IV. Definitions

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group, preferably a lower alkyl group, having an oxygen attached thereto. Representative alkoxy groups include methoxy, —OCF₃, ethoxy, propoxy, tert-butoxy and the like.

The term “cycloalkyloxy” refers to a cycloakyl group having an oxygen attached thereto.

The term “alkoxyalkyl” refers to an alkyl group substituted with an alkoxy group and may be represented by the general formula alkyl-O-alkyl.

The term “alkylaminoalkyl” refers to an alkyl group substituted with an alkylamino group.

The term “alkenyl”, as used herein, refers to an aliphatic group containing at least one double bond and is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed below, except where stability is prohibitive. For example, substitution of alkenyl groups by one or more alkyl, carbocyclyl, aryl, heterocyclyl, or heteroaryl groups is contemplated.

An “alkyl” group or “alkane” is a straight chained or branched non-aromatic hydrocarbon which is completely saturated. Typically, a straight chained or branched alkyl group has from 1 to about 20 carbon atoms, preferably from 1 to about 10 unless otherwise defined. Examples of straight chained and branched alkyl groups include methyl, ethyl, n-propyl, iso-propyl, n-butyl, sec-butyl, tert-butyl, pentyl, hexyl, pentyl and octyl. A C₁-C₆ straight chained or branched alkyl group is also referred to as a “lower alkyl” group.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents, if not otherwise specified, can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

The term “C_{x-y}” when used in conjunction with a chemical moiety, such as, acyl, acyloxy, alkyl, alkenyl, alkynyl, or alkoxy is meant to include groups that contain from x to y carbons in the chain. For example, the term “C_{x-y}alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-trifluoroethyl, etc. C₀ alkyl indicates a hydrogen where the group is in a terminal position, a bond if internal. The terms “C_{2-y}alkenyl” and “C_{2-y}alkynyl” refer to substituted or unsubstituted unsaturated aliphatic groups analogous in length and possible substitution to the alkyls described above, but that contain at least one double or triple bond respectively.

The term “alkylamino”, as used herein, refers to an amino group substituted with at least one alkyl group.

The term “alkylthio”, as used herein, refers to a thiol group substituted with an alkyl group and may be represented by the general formula alkyl-S—.

The term “alkynyl”, as used herein, refers to an aliphatic group containing at least one triple bond and is intended to include both “unsubstituted alkynyls” and “substituted alkynyls”, the latter of which refers to alkynyl moieties having substituents replacing a hydrogen on one or more carbons of the alkynyl group. Such substituents may occur on one or more carbons that are included or not included in one or more triple bonds. Moreover, such substituents include all those contemplated for alkyl groups, as discussed above, except where stability is prohibitive. For example, substitution of alkynyl groups by one or more alkyl, carbocyclyl, aryl, heterocyclyl, or heteroaryl groups is contemplated.

The term “amide”, as used herein, refers to a group

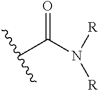

wherein each R independently represent a hydrogen or hydrocarbyl group, or two R are taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

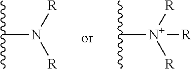

wherein each R independently represents a hydrogen or a hydrocarbyl group, or two R are taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

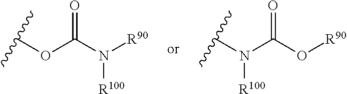

wherein R⁹⁰ and R¹⁰⁰ independently represent hydrogen or a hydrocarbyl group, such as an alkyl group, or R⁹⁰ and R¹⁰⁰ taken together with the intervening atom(s) complete a heterocycle having from 4 to 8 atoms in the ring structure. The terms “carbocycle”, and “carbocyclic”, as used herein, refers to a saturated or unsaturated ring in which each atom of the ring is carbon. The term carbocycle includes both aromatic carbocycles and non-aromatic carbocycles. Non-aromatic carbocycles include both cycloalkane rings, in which all carbon atoms are saturated, and cycloalkene rings, which contain at least one double bond. “Carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

A “cycloalkyl” group is a cyclic hydrocarbon which is completely saturated. “Cycloalkyl” includes monocyclic and bicyclic rings. Typically, a monocyclic cycloalkyl group has from 3 to about 10 carbon atoms, more typically 3 to 8 carbon atoms unless otherwise defined. The second ring of a bicyclic cycloalkyl may be selected from saturated, unsaturated and aromatic rings. Cycloalkyl includes bicyclic molecules in which one, two or three or more atoms are shared between the two rings. The term “fused cycloalkyl” refers to a bicyclic cycloalkyl in which each of the rings shares two adjacent atoms with the other ring. The second ring of a fused bicyclic cycloalkyl may be selected from saturated, unsaturated and aromatic rings. A “cycloalkenyl” group is a cyclic hydrocarbon containing one or more double bonds.

The term “carbocyclylalkyl”, as used herein, refers to an alkyl group substituted with a carbocycle group.

The term “carbonate” is art-recognized and refers to a group —OCO₂—R¹⁰, wherein R¹⁰ represents a hydrocarbyl group.

The term “carboxy”, as used herein, refers to a group represented by the formula —CO₂H.

The term “ester”, as used herein, refers to a group —C(O)OR¹⁰ wherein R¹⁰ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The term “heteroalkyl”, as used herein, refers to a saturated or unsaturated chain of carbon atoms and at least one heteroatom, wherein no two heteroatoms are adjacent.

The term “heteroalkylamino”, as used herein, refers to an amino group substituted with a heteroalkyl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like. Heterocyclyl groups can also be substituted by oxo groups. For example, “heterocyclyl” encompasses both pyrrolidine and pyrrolidinone.

The term “heterocycloalkyl”, as used herein, refers to an alkyl group substituted with a heterocycle group.

The term “heterocycloalkylamino”, as used herein refers to an amino group substituted with a heterocycloalkyl group.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocyclyl, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer non-hydrogen atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

As used herein, the term “oxo” refers to a carbonyl group. When an oxo substituent occurs on an otherwise saturated group, such as with an oxo-substituted cycloalkyl group (e.g., 3-oxo-cyclobutyl), the substituted group is still intended to be a saturated group. When a group is referred to as being substituted by an “oxo” group, this can mean that a carbonyl moiety (i.e., —C(═O)—) replaces a methylene unit (i.e., —CH₂—).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “silyl” refers to a silicon moiety with three hydrocarbyl moieties attached thereto.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, alkly, alkylamino, aminoalkyl, alkylaminoalkyl, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that substituents can themselves be substituted, if appropriate. Unless specifically stated as “unsubstituted,” references to chemical moieties herein are understood to include substituted variants. For example, reference to an “aryl” group or moiety implicitly includes both substituted and unsubstituted variants.

The term “sulfate” is art-recognized and refers to the group —OSO₃H, or a pharmaceutically acceptable salt thereof.

The term “sulfonamide” is art-recognized and refers to the group represented by the general formulae

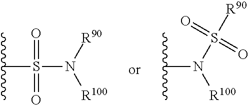

wherein R⁹⁰ and R¹⁰⁰ independently represents hydrogen or hydrocarbyl, such as alkyl, or R⁹⁰ and R¹⁰⁰ taken together with the intervening atom(s) complete a heterocycle having from 4 to 8 atoms in the ring structure.

The term “sulfoxide” is art-recognized and refers to the group —S(O)—R, wherein R represents a hydrocarbyl.

The term “sulfonate” is art-recognized and refers to the group SO₃H, or a pharmaceutically acceptable salt thereof.

The term “sulfone” is art-recognized and refers to the group —S(O)₂—R, wherein R represents a hydrocarbyl.

The term “thioalkyl”, as used herein, refers to an alkyl group substituted with a thiol group.

The term “thioester”, as used herein, refers to a group —C(O)SR or —SC(O)R wherein R¹⁰ represents a hydrocarbyl.

The term “thioether”, as used herein, is equivalent to an ether, wherein the oxygen is replaced with a sulfur.

The term “urea” is art-recognized and may be represented by the general formula

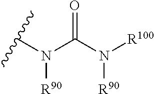

wherein R⁹⁰ and R¹⁰⁰ independently represent hydrogen or a hydrocarbyl, such as alkyl, or either occurrence of R⁹⁰ and R¹⁰⁰ taken together with the intervening atom(s) complete a heterocycle having from 4 to 8 atoms in the ring structure.

“Protecting group” refers to a group of atoms that, when attached to a reactive functional group in a molecule, mask, reduce or prevent the reactivity of the functional group. Typically, a protecting group may be selectively removed as desired during the course of a synthesis. Examples of protecting groups can be found in Greene and Wuts, Protective Groups in Organic Chemistry, 3^{rd} Ed., 1999, John Wiley & Sons, NY and Harrison et al., Compendium of Synthetic Organic Methods, Vols. 1-8, 1971-1996, John Wiley & Sons, NY. Representative nitrogen protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“TES”), trityl and substituted trityl groups, allyloxycarbonyl, 9-fluorenylmethyloxycarbonyl (“FMOC”), nitro-veratryloxycarbonyl (“NVOC”) and the like. Representative hydroxylprotecting groups include, but are not limited to, those where the hydroxyl group is either acylated (esterified) or alkylated such as benzyl and trityl ethers, as well as alkyl ethers, tetrahydropyranyl ethers, trialkylsilyl ethers (e.g., TMS or TIPS groups), glycol ethers, such as ethylene glycol and propylene glycol derivatives and allyl ethers.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

The term “treating” includes prophylactic and/or therapeutic treatments. The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The term “prodrug” is intended to encompass compounds which, under physiologic conditions, are converted into the therapeutically active agents of the present invention (e.g., a compound of Formula I). A common method for making a prodrug is to include one or more selected moieties which are hydrolyzed under physiologic conditions to reveal the desired molecule. In other embodiments, the prodrug is converted by an enzymatic activity of the host animal. For example, esters or carbonates (e.g., esters or carbonates of alcohols or carboxylic acids) are preferred prodrugs of the present invention. In certain embodiments, some or all of the compounds of formula I in a formulation represented above can be replaced with the corresponding suitable prodrug, e.g., wherein a hydroxyl in the parent compound is presented as an ester or a carbonate or carboxylic acid present in the parent compound is presented as an ester. The term “MK2-related disorder” is a disorder or condition that MK2 plays a role in the morbidity or symptoms of the disease or disorder. For example, an MK2-related disorder includes, but is not limited to, inflammatory diseases, autoimmune diseases, destructive bone disorders, proliferative disorders, angiogenic disorders, infectious diseases, neurodegenerative diseases, and viral diseases.

As used herein, the term “inflammatory disorder” or “inflammatory disease” includes diseases and disorders that are caused or primarily caused by inflammation, as well as diseases and disorders in which inflammation plays a role in the morbidity or symptoms of the disease or disorder, the propagation of the disease or disorder, the worsening of symptoms of a disease or disorder and/or the worsening of a patient's prognosis or survival time due to a disease or disorder.

### EXAMPLES

A. Chemical Syntheses

The general procedures used in the methods to prepare the compounds of the present invention are described below.

### General Methods

All solvents and reagents were obtained from commercial sources and used without further purification unless indicated otherwise. NMR spectra were obtained on a Bruker Neo 400M spectrometer operating at 400 MHz. Chemical shifts are reported in parts per million (δ) from the tetramethysilane resonance in the indicated solvent. LC-Mass spectra were taken with Agilent 1260-6125B single quadrupole mass spectrometer using a Welch Biomate column (C18, 2.7 um, 4.6*50 mm) eluting with a mixture of solvents A (ACN with 0.05% TFA) and B (Water with 0.05% TFA) using a gradient elution. Detection was by DAD (254 nm and 210 nm). Ionization was by ESI. The spectra were analyzed using Chemstation software. Analytical HPLC was performed on the Waters ARC system either under acid-containing condition on a YMC Pack Pro column (C18 S-3 um, 12 nm, 150*2.0 mm) eluting with a mixture of solvents A (ACN with 0.05% TFA) and B (Water with 0.05% TFA); or under base-containing condition on a Agilent Poroshell HPH C18 column (2.7 um, 2.1*150 mm), eluting with a mixture of solvents C (Water with 0.1% NH40H) and D (ACN with 0.1% NH40H) using a gradient elution. The detection was by DAD (254 nm and 210 nm). Preparative HPLC was performed on Waters AutoP system that is coupled with single quadrupole mass spectrometer using a Welch C18 column (5 um, 25*150 mm), eluting with a mixture of solvents A and B. Flash chromatography was carried out on Biotage Isolera Prime system using Welch WelFlash flash columns (40-63 um) eluting with a mixture of solvents as indicated in the experimental procedures.

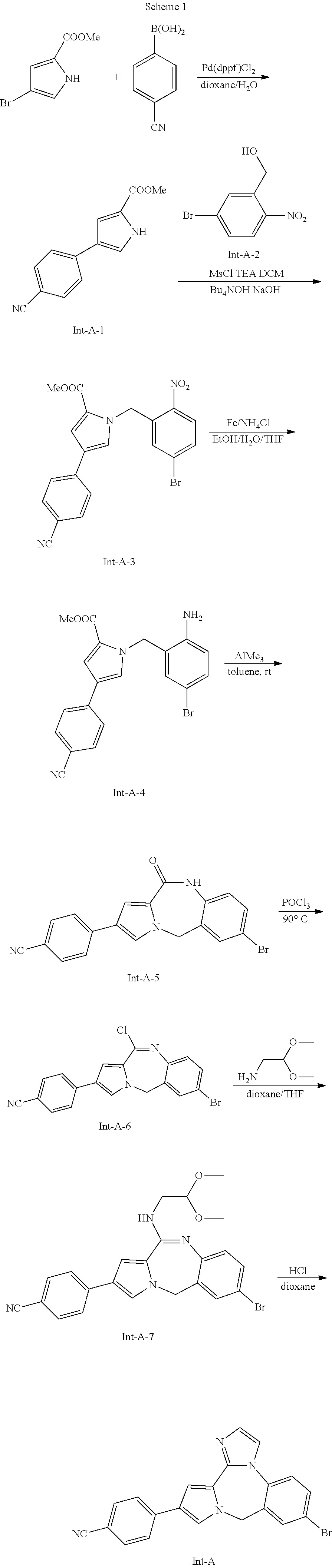

### Methyl 4-(4-cyanophenyl)-1H-pyrrole-2-carboxylate (Int-A-1)

A suspension of methyl 4-bromo-1H-pyrrole-2-carboxylate (100 g, 489 mmol), ((4-cyanophenyl)boronic acid (108 g, 734.6 mmol), K₂CO₃ (202 g, 1467 mmol, 3.0 eq.), Pd(dppf)Cl₂ (17.9 g, 24.5 mmol, 0.05 eq.) in dioxane/H₂O (1.8 L, 5:1) was de-gassed and then heated to 100° C. for 20 h under N₂. The reaction mixture was then cooled to room temperature and poured into ice water (2 L) with stirring. The precipitate formed was collected by filtration, which was re-dissolved in DCM (˜3 L) and washed with brine. The organic solvent was passed through a short silica gel column and eluted with PE:DCM (1:1). The product-containing fractions were combined and concentrated under reduced pressure. The residue was re-dissolved in DCM (˜1 L), and to which 10 L of petroleum ether was slowly added to precipitate the product out. The solid was collected by filtration after stirring for 1 h, and dried to give desired product (92 g, 93% purity) as a pale yellow solid. ESI: [M+H]⁺=227.1; ¹H NMR (400 MHz, DMSO-d₆): δ12.30 (br. s., 1H), 7.84 (d, J=8.4 Hz, 2H), 7.79-7.69 (m, 3H), 7.33 (br. s., 1H), 3.80 (s, 3H).

### (5-bromo-2-nitrophenyl) methanol (Int-A-2)

To a solution of 5-bromo-2-nitrobenzaldehyde (250 g, 1.086 mol) in MeOH (1.0 L) at 0° C., NaBH₄ (12.4 g, 326 mmol) was added batch-wise over 30 minutes. After addition, the mixture was allowed to warm to room temperature and continued to stir for 10 min. The mixture was then quenched by slowly addition of sat'd NH₄Cl (1 L) and concentrated under reduced pressure to remove volatile solvent. The residue was extracted with EA (1 L×3), and the combined organic layers were washed with brine, dried over Na₂SO₄, then concentrated under reduced pressure to give the desired product (252 g, 1086 mmol) as a white solid. ESI: [M+H]⁺=232.2/234.2; ¹H NMR (400 MHz, CDCl₃): δ 7.98-7.91 (m, 2H), 7.55 (dd, J=1.8, 8.8 Hz, 1H), 4.97 (br. s., 2H), 2.77 (br. s, 1H).

### Methyl 1-(5-bromo-2-nitrobenzyl)-4-(4-cyanophenyl)-1H-pyrrole-2-carboxylate (Int-A-3)

To a solution of compound Int-A-2 (105 g, 442.5 mmol) and TEA (123 mL, 885 mmol, 2.0 eq.) in DCM (1.5 L) was added MsCl (37.6 mL, 486.7 mmol, 1.1 eq.) at 0° C. and the mixture was stirred for 0.5 h at 20° C. Compound Int-A-1 (100 g, 442.5 mmol) was added to the solution followed by tetrabutylammonium hydroxide (45 g, 44.2 mmol, 0.1 eq.) and a solution of NaOH (2M, 1.1 L, 5.0 eq.) at 0° C. The mixture was then allowed to stir at room temperature for 2 h. The resulting yellow solid was collected by filtration. The residue was added EtOH (200 mL) and stirred for 1 h. The precipitate was collected by filtration to give the desired product (280 g, 636 mmol, 71.9% yield) as an off-white solid. ESI: [M+H]⁺=440.1/442.1; ¹H NMR (400 MHz, DMSO-d₆): δ 8.08 (d, J=8.8 Hz, 1H), 8.00 (d, J=1.8 Hz, 1H), 7.84-7.76 (m, 5H), 7.55 (d, J=1.8 Hz, 1H), 6.71 (d, J=1.3 Hz, 1H), 5.86 (s, 2H), 3.29 (s, 3H).

### Methyl 1-(2-amino-5-bromobenzyl)-4-(4-cyanophenyl)-1H-pyrrole-2-carboxylate (Int-A-4)

A suspension of compound Int-A-3 (186 g, 423.4 mmol), Fe (118.2 g, 2.1 mol) and NH₄Cl (226.5 g, 4.2 mol, 10.0 eq.) in EtOH (2 L), H₂O (1 L) and THE (2 L) was heated to 80° C. for 2 h. The mixture was then concentrated to dryness and the residue was re-dissolved in hot THE (20 L*5). The mixture was filtered through a pad of celite, and the filtrate was concentrated. The residue was slurried with EtOH (1 L) and stirred for 1 h. The solid was collected by filtration to give the desired product (138 g). ESI: [M+H]⁺=410.2/412.2; ¹H NMR (400 MHz, DMSO-d₆): δ 7.88 (br. s., 1H), 7.85-7.68 (m, 4H), 7.50 (br. s., 1H), 7.09 (d, J=7.9 Hz, 1H), 6.62 (d, J=8.4 Hz, 1H), 6.33 (br. s., 1H), 5.37 (br. s., 4H), 3.73 (br. s., 3H).

### 4-(7-Bromo-11-oxo-10,11-dihydro-5H-benzo[e]pyrrolo[1,2-a][1,4]diazepin-2-yl) benzonitrile (Int-A-5)

To a suspension of compound Int-A-4 (100 g, 244 mmol) in toluene (1 L) was added Me₃Al (2 M in toluene, 609 mL) dropwise at 0° C. and the mixture was stirred for 10 h at 20° C. under N₂. The mixture was then poured into 1M iced HCl (2M, 500 mL). The resulting mixture was filtered and the filter cake was washed with NaHCO₃ (1 L). The solid was slurried with EtOH (700 mL) and stirred for 2 h and then collected by filtration. After dried under vacuum, the desired product was obtained (86.2 g, 166 mmol, 93.7% yield) as a yellow solid. ESI: [M+H]⁺=378.2/380.2; ¹H NMR (400 MHz, DMSO-d₆): δ 10.30 (s, 1H), 7.73 (s, 4H), 7.66 (dd, J=2.0, 4.2 Hz, 2H), 7.50 (dd, J=2.2, 8.4 Hz, 1H), 7.27 (d, J=1.8 Hz, 1H), 7.13 (d, J=8.8 Hz, 1H), 5.21 (s, 2H).

### 4-(7-Bromo-11-chloro-5H-benzo[e]pyrrolo[1,2-a][1,4]diazepin-2-yl)benzonitrile (Int-A-6)

A solution of compound Int-A-5 (80 g, 211 mol) in POCl₃ (300 mL) was stirred for 18 h at 90° C. The solution was concentrated under reduced pressure and the residue was dissolved in EA (3 L). The organic solution was poured into iced-NaHCO₃ solution (2 L) and stirred for 30 min. The organic layer was separated and washed with brine, dry over Na₂SO₄. After concentration under reduced pressure, the crude product (76 g, 93.7% yield) was used immediately in the next step. ESI: [M+H]⁺=396.2/398.2

### 4-(7-Bromo-11-((2,2-dimethoxyethyl) amino)-5H-benzo[e]pyrrolo[1,2-a][1,4]diazepin-2-yl)benzonitrile (Int-A-7)

To a solution of compound Int-A-6 (75 g, 190 mmol) in THF/dioxane (500 mL/1 L) was added 2, 2-dimethoxyethanamine (100 g, 949 mmol). The resulting solution was stirred at 100° C. for 18 h. The mixture was then concentrated under reduced pressure. The residue was re-dissolved in EA (300 mL) and HCl (1M, 1.2 L) was added to above solution slowly. A precipitate formed during the addition, which was collected by filtration. The filter cake was washed with EA several times and dried under vacuum to give the desired product (wet solid), which was used to the next step without further purification. ESI: [M+H]⁺=465.2/467.2

### 4-(7-Bromo-9H-benzo[e]imidazo[2,1-c]pyrrolo[1,2-a][1,4]diazepin-12-yl) benzonitrile (Int-A)

To the solution of above solid in dioxane (800 mL) was added HCl (1M, 400 mL), and the resulting mixture was stirred at 80° C. for 20 h. The reaction mixture was then cooled to room temperature and filtered. The filter cake was washed with sat'd NaHCO₃ solution (1 L) followed by deionized water (1 L). The solid was then slurried again with MeOH (300 mL). After filtration, the filter cake was collected and dried under vacuum to give the desired product as a white solid (36 g). ESI: [M+H]⁺=401.2/403.2; ¹H NMR (400 MHz, DMSO-d₆): δ 7.92 (d, J=2.3 Hz, 1H), 7.84 (d, J=1.4 Hz, 1H), 7.75-7.774 (m, 4H), 7.65 (d, J=1.9 Hz, 1H), 7.56 (d, J=8.6 Hz, 1H), 7.25 (d, J=1.4 Hz, 1H), 7.06 (d, J=1.9 Hz, 1H), 5.24 (s, 2H).

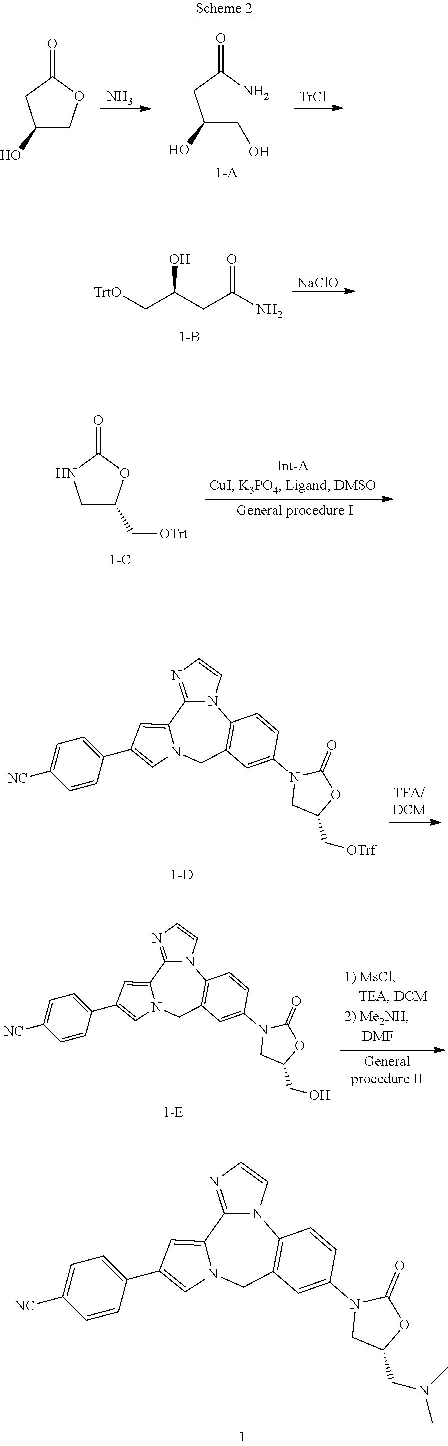

### (S)-3,4-Dihydroxybutanamide (1-A)

A solution of (S)-4-hydroxydihydrofuran-2(3H)-one (5 g, 49 mmol) in NH₃ solution in MeOH (7 mol/L, 12 mL) was stirred at 25° C. for 19 h. The mixture was concentrated to give the desired product (6 g, yield 100%). ESI: [M+H]⁺=120.1

### (S)-3-Hydroxy-4-(trityloxy)butanamide (1-B)

To a solution of compound 011-A (6 g, 50.39 mmol) and pyridine (6 mL) in THE (25 mL) and DMF (25 mL) was added trityl chloride (15.45 g, 55.43 mmol) and stirred at 25° C. for 18 h. To the mixture was then added NH₄Cl aq. (200 mL) and EA (200 mL). After stirring for 30 min, the organic layer in the mixture was separated and washed with brine, dried over anhydrous Na₂SO₄, filtered and concentrated to give the desired compound as a yellow solid (5.3 g, yield: 29%). ESI: [M+H]=362.1

### (S)-4-(((2-Oxooxazolidin-5-yl)methoxy)diphenylmethyl)benzene-1-ylium (1-C)

To a solution of compound 1-B (5.67 g, 15.7 mmol) and 13% NaClO (25.2 mL) in THE (47 mL) was added NaOH (2.52 g 63.5 mmol) in H₂O (15.75 mL). The resulting mixture was stirred at 60° C. for 1 h before the addition of water (200 mL) and EA (200 mL). The organic layer in the mixture was separated and washed with brine, dried over anhydrous Na₂SO₄. After filtration and concentration under reduced pressure, the residue was purified by chromatography on silica gel (DCM:MeOH=40:1) to give the desired compound (3.87 g, yield: 68.6%). ESI: [M+H]⁺=360.2

General Procedure I—Ullmann Reaction:

### (S)-4-(7-(2-Oxo-5-((trityloxy)methyl)oxazolidin-3-yl)-9H-benzo[e]imidazo [2,1-c]pyrrolo[1,2-a][1,4]diazepin-12-yl)benzonitrile (1-D)

The solution of Int-A (100 mg, 0.25 mmol), compound 1-C (134.25 mg, 0.37 mmol), CuI (4.75 mg, 0.025 mmol), Cs₂CO₃ (162.4 mg, 0.5 mmol), trans-N,N′-dimethylcyclohexane-1,2-diamine (7.09 mg, 0.05 mmol) in anhydrous DMSO (2 mL) was stirred at 115° C. under N₂ for 16 h. The reaction mixture was then cooled to room temperature and H₂O (10 mL), EA (10 mL), and NH₄OH:NH₄Cl=1:10 (30 mL) were added. The resulting mixture was stirred and then filtered. The organic layer in the filtrate was separated and washed with brine, dried over anhydrous Na₂SO₄. After filtration and concentration, the residue was purified by chromatography on silica gel (DCM:MeOH=30:1) to give the desired compound as a brown solid (230 mg, yield>99%). ESI: [M+H]⁺=680.3

### (S)-4-(7-(5-(Hydroxymethyl)-2-oxooxazolidin-3-yl)-9H-benzo[e]imidazo [2,1-c]pyrrolo[1,2-a][1,4]diazepin-12-yl)benzonitrile (1-E)

To the solution of compound 1-D (230 mg, 0.34 mmol) in DCM (10 mL) was added TFA (0.5 mL). After stirring at 25° C. for 30 min, to the solution was added NaHCO₃ (20 mL) and DCM (20 mL). The organic layer was separated and washed with brine and dried over anhydrous Na₂SO₄. After filtration and concentration, the residue was purified by chromatography on silica gel (DCM:MeOH=100:1) to give the desired product as a brown solid (117.4 mg, yield: 73%). ESI [M+H]⁺=348.1

General Procedure II:

### (R)-4-(7-(5-((Dimethylamino)methyl)-2-oxooxazolidin-3-yl)-9H-benzo [e]imidazo[2,1-c]pyrrolo[1,2-a][1,4]diazepin-12-yl)benzonitrile (1)

To a solution of compound 1-E (100 mg, 0.23 mmol), TEA (46.25 mg, 0.46 mmol) in DCM (50 mL) was added MsCl (0.1 mL, 1.26 mmol) at 0° C., and the resulting solution was stirred at 0° C. for 3.5 h. The mixture was diluted with ice-water (100 mL) and extracted with DCM (100 mL). The organic layer was washed with brine, dried over anhydrous Na₂SO₄, and filtered. After filtration and concentration, the crude compound was obtained as a brown solid (115 mg), which was used directly in next step. ESI: [M+H]⁺=516.1. A solution of above compound (50 mg, 0.1 mmol), Me₂NH in THE (2 mL, 2 mmol) in DMF (2 mL) was placed at 100° C. in a microwave reactor for 2 h. The mixture was diluted with H₂O (50 mL) and extracted with DCM (50 mL). The organic layer was washed with brine, dried over anhydrous Na₂SO₄, filtered, and concentrated. The resulting residue was purified by Prep-HPLC to give the desired compound as a white solid (11.1 mg, yield: 23%). ESI: [M+H]⁺=465.2; ¹H-NMR (400 MHz, DMSO-d₆): δ 9.9 (brs, 1H), 7.6-8.0 (m, 10H), 7.55 (s, 1H), 5.36 (s, 2H), 5.20 (s, 1H), 4.30 (t, 1H), 3.88 (s, 2H), 2.89 (s, 6H), 2.68 (s, 1H), 2.38 (s, 1H).

Using the procedures described above, the following examples in Table 3 were synthesized.

TABLE 3

| | Ex. | | LC-MS |
|---|---|---|---|
| Structure | # | ¹H NMR | (M + H)⁺ |
| 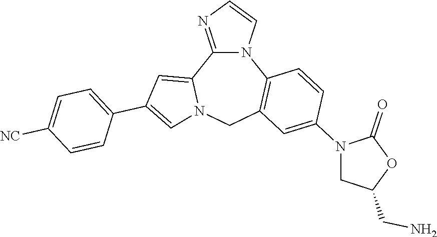 [SMILES: N#Cc1ccc(-c2cc3n(c2)Cc2cc(N4C[C@@H](CN)OC4=O)ccc2-n2ccnc2-3)cc1] | 2 | (400 MHz, MeOH-d₄): δ 8.03 (d, J = 2.2 Hz, 1H), 7.99 (s, 1H), 7.79 (dd, J = 8.9, 2.3 Hz, 1H), 7.77 − 7.72 (m, 4H), 7.70 (d, J = 8.5 Hz, 2H), 7.64 (s, 1H), 7.27 (s, 1H), 5.37 (s, 2H), 5.05 − 4.97 (m, 1H), 4.37 (t, J = 9.1 Hz, 1H), 3.94 (dd, J = 9.2, 6.5 Hz, 1H), 3.50 − 3.43 (m, 1H), 3.42 − 3.39 (m, 1H). | 437.2 |
| 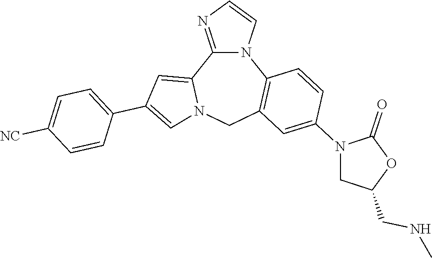 [SMILES: CNC[C@@H]1CN(c2ccc3c(c2)Cn2cc(-c4ccc(C#N)cc4)cc2-c2nccn2-3)C(=O)O1] | 3 | (400 MHz, MeOH-d₄): δ 8.06 − 7.95 (m, 2H), 7.80 (dd, J = 8.9, 2.5 Hz, 1H), 7.75 (dd, J = 5.7, 3.8 Hz, 4H), 7.70 (t, J = 6.7 Hz, 3H), 7.65 (d, J = 1.7 Hz, 1H), 7.27 (d, J = 1.8 Hz, 1H), 5.37 (s, 2H), 4.38 (t, J = 9.2 Hz, 1H), 3.93 (dd, J = 9.4, 6.4 Hz, 2H), 3.50 − 3.45 (m, 3H), 2.82 (s, 3H). | 451.2 |
| 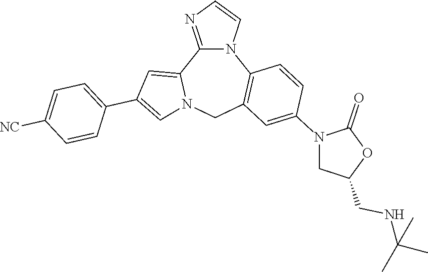 [SMILES: CC(C)(C)NC[C@@H]1CN(c2ccc3c(c2)Cn2cc(-c4ccc(C#N)cc4)cc2-c2nccn2-3)C(=O)O1] | 4 | (400 MHz, MeOH-d₄): δ 8.01 (dd, J = 13.0, 2.1 Hz, 2H), 7.81 (dd, J = 9.0, 2.5 Hz, 1H), 7.78 - 7.73 (m, 4H), 7.69 (d, J = 8.6 Hz, 2H), 7.65 (d, J = 1.8 Hz, 1H), 7.27 (d, J = 1.8 Hz, 1H), 5.38 (s, 2H), 4.39 (t, J = 9.1 Hz, 1H), 3.95 (dd, J = 9.3, 6.5 Hz, 1H), 3.49 (dt, J = 23.0, 11.9 Hz, 3H), 1.43 (s, 9H). | 493.2 |
| 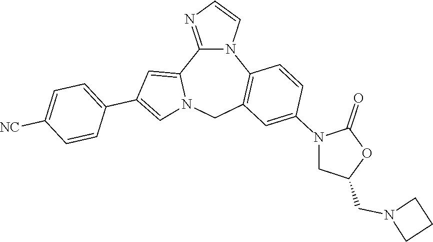 [SMILES: N#Cc1ccc(-c2cc3n(c2)Cc2cc(N4C[C@@H](CN5CCC5)OC4=O)ccc2-n2ccnc2-3)cc1] | 5 | (400 MHz, MeOH-d₄): δ 8.02 (dd, J = 5.5, 2.2 Hz, 2H), 7.81 (dd, J = 9.0, 2.5 Hz, 1H), 7.79 - 7.73 (m, 4H), 7.73 − 7.67 (m, 3H), 7.30 (d, J = 1.7 Hz, 1H), 5.39 (s, 2H), 5.06 − 4.96 (m, 4H), 4.32 (dd, J = 17.1, 8.5 Hz, 4H), 3.91 (dd, J = 9.4, 6.3 Hz, 1H), 3.75 − 3.59 (m, 2H). | 477.2 |

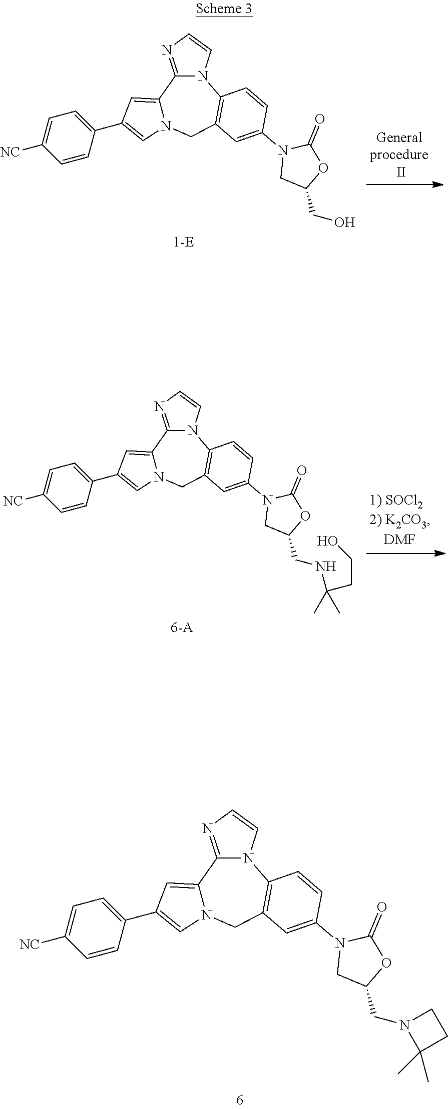 [SMILES: N#Cc1ccc(-c2cc3n(c2)Cc2cc(N4C[C@@H](CO)OC4=O)ccc2-n2ccnc2-3)cc1 ; CC(C)(CCO)NC[C@@H]1CN(c2ccc3c(c2)Cn2cc(-c4ccc(C#N)cc4)cc2-c2nccn2-3)C(=O)O1 ; CC1(C)CCN1C[C@@H]1CN(c2ccc3c(c2)Cn2cc(-c4ccc(C#N)cc4)cc2-c2nccn2-3)C(=O)O1]

### (R)-4-(7-(5-(((4-Hydroxy-2-methylbutan-2-yl)amino)methyl)-2-oxooxazolidin-3-yl)-9H-benzo[e]imidazo[2,1-c]pyrrolo[1,2-a][1,4]diazepin-12-yl)benzonitrile (6-A)

Using the procedures described above, compound 6-A was synthesized. ESI: [M+H]⁺=523.2.

### (R)-4-(7-(5-((2,2-Dimethylazetidin-1-yl)methyl)-2-oxooxazolidin-3-yl)-9H-benzo[e]imidazo[2,1-c]pyrrolo[1,2-a][1,4]diazepin-12-yl)benzonitrile (6)

To a solution of compound 6-A in dry DCM (1 mL) was added SOCl₂ (1 drop), and the resulting solution was stirred at 40° C. for 30 min. After the reaction was completed, the mixture was concentrated to give the crude compound (11 mg). ESI: [M+H]⁺=541.2. To a solution of above compound (11 mg, 0.02 mmol) in dry DMF (1 mL) was added K₂CO₃ (10 mg), the mixture was placed at 80° C. in a microwave reactor for 2 h. The reaction mixture was concentrated and purified by Prep-HPLC to give the desired compound (2 mg, yield: 20%). ESI: [M+H]⁺=505.2; ¹H NMR (400 MHz, MeOH-d₄): δ 8.01 (dd, J=10.2, 2.2 Hz, 2H), 7.80 (dd, J=8.9, 2.5 Hz, 1H), 7.78-7.73 (m, 4H), 7.72-7.68 (m, 2H), 7.65 (d, J=1.8 Hz, 1H), 7.27 (d, J=1.8 Hz, 1H), 5.37 (s, 2H), 5.03 (s, 1H), 4.36 (t, J=9.1 Hz, 1H), 4.16 (s, 3H), 3.90 (dd, J=9.2, 6.4 Hz, 1H), 3.59 (s, 2H), 2.57 (s, 1H), 2.29 (s, 1H), 1.69 (d, J=6.3 Hz, 6H).

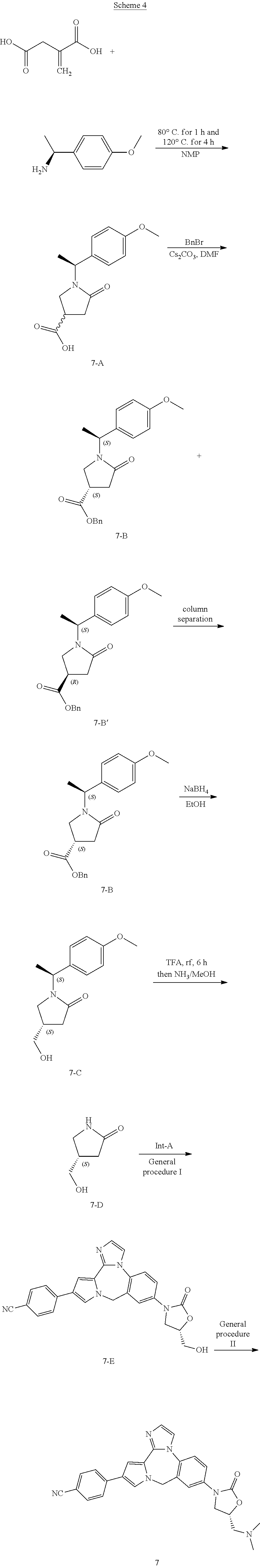

### 1-((S)-1-(4-Methoxyphenyl)ethyl)-5-oxopyrrolidine-3-carboxylic acid (7-A)

A suspension of (S)-1-(4-methoxyphenyl)ethanamine (13.5 g, 99.15 mmol) and itaconic acid (13 g, 98.2 mmol) in 1-methyl-2-pyrrolidinone (80 mL) was heated to 80° C. for 1 hour. The solution was stirred for additional 4 h at 120° C. The reaction mixture was cooled to 25° C. and poured into 500 mL of demineralized water. The precipitate formed was filtered, washed with demineralized water and dried at 50° C. to give the desired compound as white solid (21 g, 79.8 mmol, 89% yield).

### (S)-Benzyl-1-((S)-1-(4-methoxyphenyl)ethyl)-5-oxopyrrolidine-3-carboxylate (7-B)

A solution of compound 7-A (21 g, 79.8 mmol), benzyl bromide (10.34 mL, 87.8 mmol, 1.1 eq.) and cesium carbonate (28.55 g, 87.8 mmol, 1.1 eq.) in DMF (100 mL) was stirred at room temperature for 45 min. Water and ethyl acetate was added to the reaction mixture. The organic layer was separated and the aqueous layer was extracted with EtOAc (200 mL*2). The combined organic layers were dried over anhydrous Na₂SO₄ and concentrated under reduce pressure. The obtained residue was purified by silica gel column chromatography (solvent gradient: 5 percent to 50 percent petroleum ether/ethyl acetate) to give (S)-benzyl 1-((S)-1-(4-methoxyphenyl)ethyl)-5-oxopyrrolidine-3-carboxylate (11.2 g, 31.73 mmol, 39.7% yield) as first eluent and (R)-benzyl 1-((S)-1-(4-methoxyphenyl)ethyl)-5-oxopyrrolidine-3-carboxylate (11.6 g, 32.86 mmol, 41.2% yield) as second eluent.

7-B: ¹H NMR (400 MHz, CDCl₃): δ 7.42-7.28 (m, 5H), 7.20 (t, J=5.7 Hz, 2H), 6.88-6.82 (m, 2H), 5.44 (q, J=7.1 Hz, 1H), 5.14 (s, 2H), 3.79 (s, 3H), 3.53 (dd, J=9.4, 5.8 Hz, 1H), 3.22-3.05 (m, 2H), 2.73 (qd, J=17.1, 8.4 Hz, 2H), 1.48 (d, J=7.1 Hz, 3H)

7-B′: ¹H NMR (400 MHz, CDCl₃): δ 7.38-7.30 (m, 3H), 7.27-7.22 (m, 2H), 7.18 (t, J=5.7 Hz, 2H), 6.88-6.80 (m, 2H), 5.44 (q, J=7.1 Hz, 1H), 5.06 (d, J=1.5 Hz, 2H), 3.77 (d, J=9.0 Hz, 3H), 3.58-3.47 (m, 1H), 3.28-3.14 (m, 2H), 2.80-2.61 (m, 2H), 1.49 (d, J=7.1 Hz, 3H). ESI: [M+H]⁺=354

### (S)-4-(Hydroxymethyl)-1-((S)-1-(4-methoxyphenyl)ethyl)pyrrolidin-2-one (007-C)

To a solution of compound 7-B (11.2 g, 31.7 mmol) in EtOH (75 mL) was added NaBH₄ (4.2 g, 111 mmol) at 0° C. The mixture was stirred at room temperature for 8 h. The reaction was quenched by addition of NH₄Cl. The EtOAc was added and the organic layer was separated. The aqueous layer was extracted with EtOAc (3*200 mL). The combined organic layer was washed with saturated salt, dried over anhydrous Na₂SO₄ and concentrated under reduced pressure. The resulting residue was purified by silica gel chromatography (PE:EA=5:1 to DCM:MeOH=100:1) to give the desired alcohol (7.5 g, 30.12 mmol, 95% yield). ESI: [M+H]⁺=250

### (S)-4-(Hydroxymethyl) pyrrolidin-2-one-D)

A solution of compound 7-C (7.5 g, 30.12 mmol, 1.0 eq) in TFA (40 mL) was heated to 80° C. and stirred for 16 h. The volatile solvent was removed under reduced pressure. The residue was re-dissolved in MeOH (50 mL), and NH₃·H₂O was added to above solution at room temperature. After stirring for 1 h, the reaction mixture was concentrated, and the residue was purified by silica gel column chromatography (DCM:MeOH=100:1 to 10:1) to give the desired product (3.0 g, 26.1 mmol). ESI: [M+H]⁺=116; ¹H NMR (400 MHz, MeOH-d₄): δ 3.58-3.51 (m, 2H), 3.51-3.45 (m, 1H), 3.20 (dd, J=10.1, 5.3 Hz, 1H), 2.72-2.56 (m, 1H), 2.42 (dd, J=17.1, 9.1 Hz, 1H), 2.24-2.05 (m, 1H).

### (S)-4-(7-(4-(Hydroxymethyl)-2-oxopyrrolidin-1-yl)-9H-benzo[e]imidazo[2,1-c]pyrrolo[1,2-a][1,4]diazepin-12-yl)benzonitrile 07-E)

Using the procedures described above, compound 7-E was synthesized. ESI: [M+H]⁺=436

### (R)-4-(7-(4-((Dimethylamino)methyl)-2-oxopyrrolidin-1-yl)-9H-benzo[e]imidazo[2,1-c]pyrrolo[1,2-a][1,4]diazepin-12-yl)benzonitrile (7)

Using the procedures described above, compound 7 was synthesized. ESI: [M+H]⁺=436.3; ¹H NMR (400 MHz, MeOH-d₄): δ 7.97 (d, J=2.5 Hz, 1H), 7.78 (dd, J=8.8, 2.5 Hz, 1H), 7.74-7.67 (m, 3H), 7.66-7.62 (m, 2H), 7.58 (d, J=8.8 Hz, 1H), 7.53 (d, J=1.9 Hz, 1H), 7.25 (d, J=1.3 Hz, 1H), 7.05 (d, J=1.9 Hz, 1H), 5.18 (s, 2H), 4.05 (dd, J=9.7, 7.8 Hz, 1H), 3.76-3.67 (m, 1H), 2.83-2.71 (m, 2H), 2.51 (dd, J=12.3, 7.7 Hz, 1H), 2.47-2.37 (m, 2H), 2.30 (s, 6H).

Using the procedures described above, the following examples in Table 4 were synthesized.

TABLE 4

| | Ex. | | LC-MS |
|---|---|---|---|
| Structure | # | ¹H NMR | (M + H)⁺ |
| 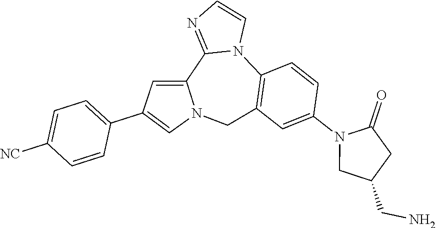 [SMILES: N#Cc1ccc(-c2cc3n(c2)Cc2cc(N4C[C@@H](CN)CC4=O)ccc2-n2ccnc2-3)cc1] | 8 | (400 MHz, MeOH-d₄): δ 8.01 (d, J = 2.4 Hz, 1H), 7.75-7.66 (m, 8H), 7.53 (d, J = 1.8 Hz, 1H), 7.26 (s, 1H), 7.06 (d, J = 1.8 Hz, 1H), 5.19 (s, 2H), 4.12 (dd, J = 9.7, 7.9 Hz, 1H), 3.76 (dd, J = 9.9, 6.3 Hz, 1H), 3.09 (t, J = 13.6 Hz, 2H), 2.89 − 2.75 (m, 2H), 2.51 (dd, J = 15.5, 6.0 Hz, 1H). | 435.5 |
| 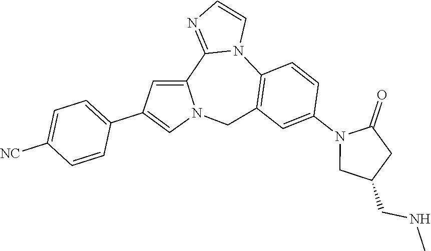 [SMILES: CNC[C@H]1CC(=O)N(c2ccc3c(c2)Cn2cc(-c4ccc(C#N)cc4)cc2-c2nccn2-3)C1] | 9 | (400 MHz, MeOH-d₄): δ 8.12 (s, 2H), 7.97 − 7.63 (m, 8H), 7.38 (s, 1H), 5.42 (s, 2H), 4.17 (s, 1H), 3.81 (s, 1H), 3.26 (s, 2H), 3.03 − 2.82 (m, 3H), 2.78 (s, 3H), 2.57 (dd, J = 16.8, 6.8 Hz, 1H). | 449.2 |
| 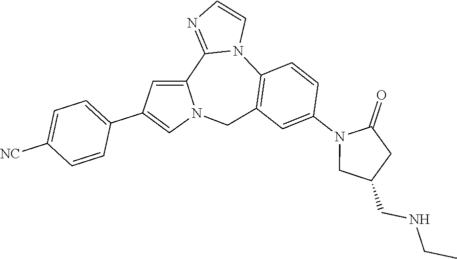 [SMILES: CCNC[C@H]1CC(=O)N(c2ccc3c(c2)Cn2cc(-c4ccc(C#N)cc4)cc2-c2nccn2-3)C1] | 10 | (400 MHz, MeOH-d₄): δ 8.00 (d, J = 2.4 Hz, 1H), 7.76 − 7.57 (m, 7H), 7.53 (d, J = 1.9 Hz, 1H), 7.26 (d, J = 1.4 Hz, 1H), 7.05 (d, J = 1.9 Hz, 1H), 5.19 (s, 2H), 4.11 (dd, J = 9.7, 7.8 Hz, 1H), 3.76 (dd, J = 9.9, 6.2 Hz, 1H), 3.08 − 2.74 (m, 6H), 2.51 (q, J = 10.8 Hz, 1H), 1.26 (t, J = 7.2 Hz, 3H). | 463.2 |
| 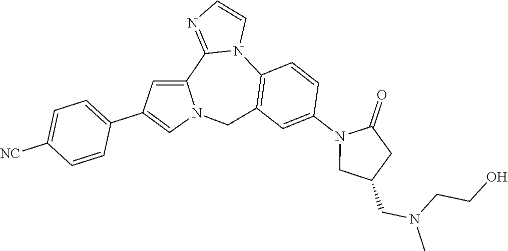 [SMILES: CN(CCO)C[C@H]1CC(=O)N(c2ccc3c(c2)Cn2cc(-c4ccc(C#N)cc4)cc2-c2nccn2-3)C1] | 11 | (400 MHz, MeOH-d₄): δ 8.10 (d, J = 3.5 Hz, 1H), 8.07 (d, J = 5.5 Hz, 1H), 7.85 (dd, J = 8.8, 2.4 Hz, 1H), 7.80 − 7.66 (m, 7H), 7.32 (d, J = 4.5 Hz, 1H), 5.38 (s, 2H), 4.20 (dt, J = 13.6, 6.8 Hz, 1H), 3.97 − 3.88 (m, 2H), 3.82 (dt, J = 14.1, 7.1 Hz, 1H), 3.43 (ddd, J = 16.6, 8.0, 5.4 Hz, 3H), 3.12- 3.08 (m, 1H), 3.02 (s, 3H), 2.99 − 2.88 (m, 1H), 2.67 − 2.52 (m, 1H). | 493.2 |
| 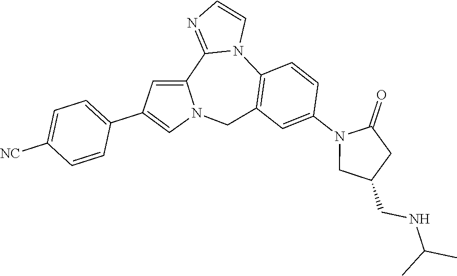 [SMILES: CC(C)NC[C@H]1CC(=O)N(c2ccc3c(c2)Cn2cc(-c4ccc(C#N)cc4)cc2-c2nccn2-3)C1] | 12 | (400 MHz, MeOH-d₄): δ 8.07 (d, J = 2.3 Hz, 1H), 7.84 (s, 1H), 7.79 − 7.71 (m, 3H), 7.67 (dd, J = 13.1, 5.9 Hz, 4H), 7.45 (s, 1H), 7.16 (s, 1H), 5.28 (s, 2H), 4.21 − 4.12 (m, 1H), 3.80 (dd, J = 9.6, 6.1 Hz, 1H), 3.45 (dd, J = 13.5, 6.6 Hz, 2H), 3.27 (d, J = 1.5 Hz, 1H), 2.88 (t, J = 9.7 Hz, 2H), 2.58 (dd, J = 20.2, 11.0 Hz, 1H), 1.36 (d, J = 8.0 Hz, 6H). | 477.2 |
| 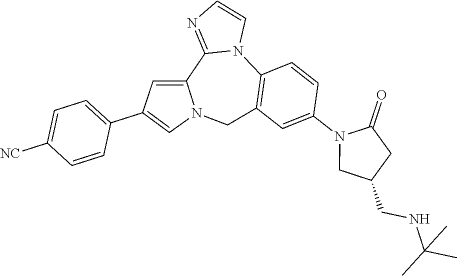 [SMILES: CC(C)(C)NC[C@H]1CC(=O)N(c2ccc3c(c2)Cn2cc(-c4ccc(C#N)cc4)cc2-c2nccn2-3)C1] | 13 | (400 MHz, MeOH-d₄): δ 8.12 (d, J = 2.3 Hz, 1H), 8.06 (s, 1H), 7.85 (dd, J = 8.9, 2.4 Hz, 1H), 7.79-7.69 (m, 7H), 7.31 (s, 1H), 5.39 (s, 2H), 4.19 (dd, J = 9.6, 8.0 Hz, 1H), 3.83 (dd, J = 9.9, 6.5 Hz, 1H), 3.29 − 3.19 (m, 2H), 2.97 − 2.84 (m, 2H), 2.59 (q, J = 11.3 Hz, 1H), 1.42 (s, 9H). | 491.2 |
| 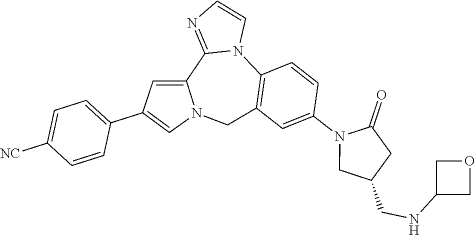 [SMILES: N#Cc1ccc(-c2cc3n(c2)Cc2cc(N4C[C@@H](CNC5COC5)CC4=O)ccc2-n2ccnc2-3)cc1] | 14 | (400 MHz, MeOH-d₄): δ 8.12 (d, J = 2.3 Hz, 1H), 8.05 (d, J = 2.0 Hz, 1H), 7.87 − 7.81 (m, 1H), 7.77 − 7.69 (m, 7H), 7.30 (d, J = 1.8 Hz, 1H), 5.39 (s, 2H), 4.94 (t, J = 7.5 Hz, 2H), 4.70 (dd, J = 8.0, 5.2 Hz, 2H), 4.51 − 4.42 (m, 1H), 4.18 (t, J = 8.7 Hz, 1H), 3.84 − 3.73 (m, 1H), 3.40 (d, J = 7.3 Hz, 1H), 3.20 (d, J = 5.3 Hz, 2H), 3.15 − | 491.2 |
| | | 3.04 (m, 1H), 2.95 − | |
| | | 2.86 (m, 2H), 2.58 (q, | |
| | | J = 8.0 Hz, 1H). | |
| 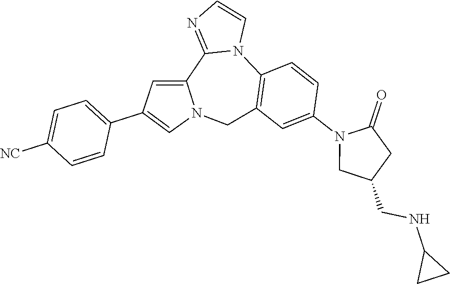 [SMILES: N#Cc1ccc(-c2cc3n(c2)Cc2cc(N4C[C@@H](CNC5CC5)CC4=O)ccc2-n2ccnc2-3)cc1] | 15 | (400 MHz, MeOH-d₄): δ 7.98 (d, J = 2.5 Hz, 1H), 7.77 (dd, J = 8.8, 2.5 Hz, 1H), 7.70 − 7.59(m, 6H), 7.54 (d, J = 1.8 Hz, 1H), 7.26 (s, 1H), 7.06 (d, J = 1.8 Hz, 1H), 5.19 (s, 2H), 4.06 (dd, J = 9.7, 7.8 Hz, 1H), 3.72 (dd, J = 9.7, 6.0 Hz, 1H), 2.78 (ddd, J = 26.0, 21.3, 9.1 Hz, 4H), 2.44 (dd, J = 15.3, 5.5 Hz, 1H), 2.23 (dd, J = 6.8, 3.4 Hz, 1H), 0.53(d, J = 4.7Hz, 2H), 0.47 (d, J = 4.7 Hz, 2H). | 475.2 |
| 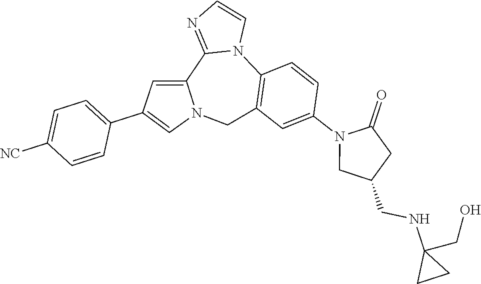 [SMILES: N#Cc1ccc(-c2cc3n(c2)Cc2cc(N4C[C@@H](CNC5(CO)CC5)CC4=O)ccc2-n2ccnc2-3)cc1] | 16 | (400 MHz, MeOH-d₄): δ 8.12 (d, J = 2.3 Hz, 1H), 8.04 (s, 1H), 7.84 (dd, J = 8.9, 2.5 Hz, 1H), 7.76 − 7.70 (m, 7H), 7.30 (s, 1H), 5.39 (s, 2H), 4.27 − 4.08 (m, 1H), 3.92 − 3.77 (m, 2H), 3.73 − 3.68 (m, 1H), 3.51 − 3.40 (m, 1H), 2.92 (ddd, J = 15.5, 13.5, 10.2 Hz, 2H), 2.66 − 2.52 (m, 1H), 1.80-1.76 (m, 1H), 1.19 − 0.89 (m, 4H). | 505.2 |

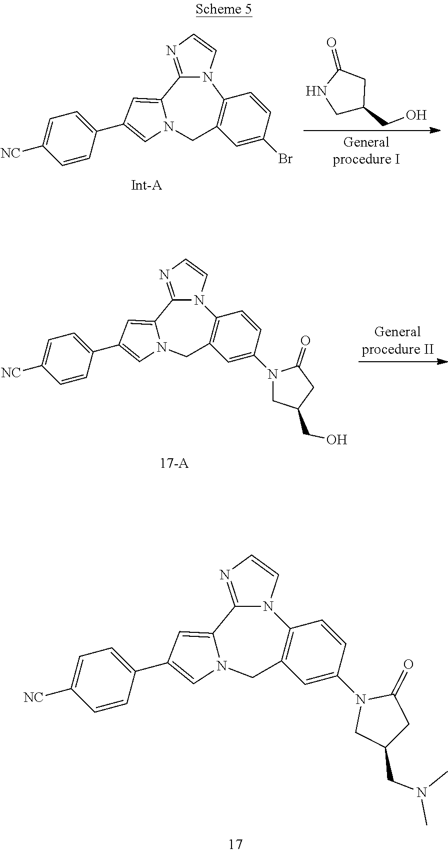 [SMILES: N#Cc1ccc(-c2cc3n(c2)Cc2cc(Br)ccc2-n2ccnc2-3)cc1 ; O=C1C[C@@H](CO)CN1 ; N#Cc1ccc(-c2cc3n(c2)Cc2cc(N4C[C@H](CO)CC4=O)ccc2-n2ccnc2-3)cc1 ; CN(C)C[C@@H]1CC(=O)N(c2ccc3c(c2)Cn2cc(-c4ccc(C#N)cc4)cc2-c2nccn2-3)C1]

### (S)-4-(7-(4-((Dimethylamino)methyl)-2-oxopyrrolidin-1-yl)-9H-benzo[e]imidazo[2,1-c]pyrrolo[1,2-a][1,4]diazepin-12-yl)benzonitrile (17)

Using the procedures described above, compound 17 was synthesized: ESI: [M+H]⁺=463.2; ¹H NMR (400 MHz, MeOH-d₄): δ 8.10 (dd, J=15.3, 2.3 Hz, 2H), 7.87 (dd, J=8.9, 2.5 Hz, 1H), 7.80 (d, J=1.8 Hz, 1H), 7.78 (s, 1H), 7.77-7.73 (m, 4H), 7.71 (d, J=2.1 Hz, 2H), 7.70 (d, J=2.0 Hz, 1H), 7.33 (d, J=1.8 Hz, 1H), 5.40 (s, 2H), 4.19 (dd, J=9.7, 8.1 Hz, 1H), 3.80 (dd, J=9.8, 7.3 Hz, 1H), 3.41 (dd, J=7.3, 1.1 Hz, 2H), 3.14-3.06 (m, 1H), 2.99 (d, J=3.3 Hz, 6H), 2.91 (dd, J=16.9, 8.7 Hz, 1H), 2.58 (dd, J=17.0, 8.5 Hz, 1H).

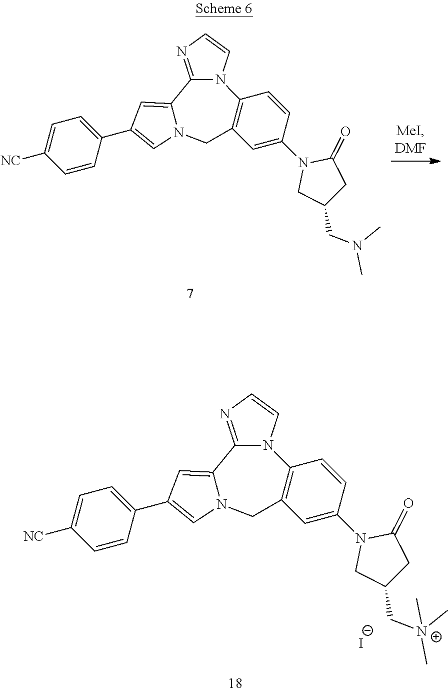 [SMILES: CN(C)C[C@H]1CC(=O)N(c2ccc3c(c2)Cn2cc(-c4ccc(C#N)cc4)cc2-c2nccn2-3)C1 ; C[N+](C)(C)C[C@H]1CC(=O)N(c2ccc3c(c2)Cn2cc(-c4ccc(C#N)cc4)cc2-c2nccn2-3)C1 ; [I-]]

### (S)-1-(1-(12-(4-cyanophenyl)-9H-benzo[e]imidazo[2,1-c]pyrrolo[1,2-a][1,4]diazepin-7-yl)-5-oxopyrrolidin-3-yl)-N,N,N-trimethylmethanaminium iodide (18)

To a solution of compound 007 (10 mg, 0.02 mmol) in DMF (2 mL) was added iodomethane (0.5 mL) at room temperature. The mixture was stirred at 20° C. for 2 h. The solvent was then removed in vacuo. The residue was purified by Prep-HPLC to give the desired product (1 mg, yield: 8%). ESI: [M+H]⁺=477.2; ¹H NMR (400 MHz, DMSO-d₆): δ 8.04 (d, J=2.2 Hz, 1H), 7.96 (s, 1H), 7.76 (dd, J=16.3, 8.6 Hz, 6H), 7.67 (d, J=8.8 Hz, 1H), 7.45 (s, 1H), 7.15 (s, 1H), 5.31 (s, 2H), 4.15 (t, J=9.0 Hz, 1H), 3.72 (t, J=9.1 Hz, 1H), 3.57 (d, J=5.9 Hz, 2H), 3.13 (s, 9H), 2.85 (dd, J=16.6, 8.7 Hz, 1H), 2.69-2.65 (m, 1H), 2.34-2.31 (m, 2H).

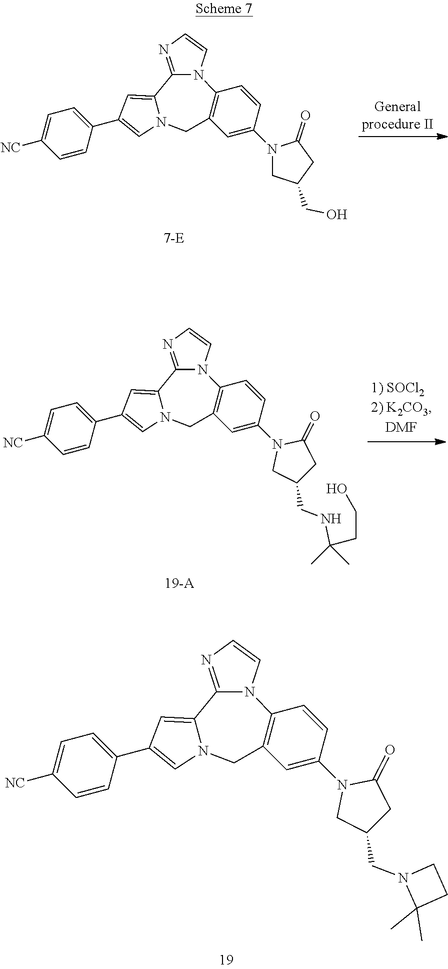 [SMILES: N#Cc1ccc(-c2cc3n(c2)Cc2cc(N4C[C@@H](CO)CC4=O)ccc2-n2ccnc2-3)cc1 ; CC(C)(CCO)NC[C@H]1CC(=O)N(c2ccc3c(c2)Cn2cc(-c4ccc(C#N)cc4)cc2-c2nccn2-3)C1 ; CC1(C)CCN1C[C@H]1CC(=O)N(c2ccc3c(c2)Cn2cc(-c4ccc(C#N)cc4)cc2-c2nccn2-3)C1]

Using the procedures described above, compound 19 was synthesized. ESI: [M+H]⁺=503.2; ¹H NMR (400 MHz, MeOH-d₄): δ 7.97 (d, J=2.4 Hz, 1H), 7.77 (dd, J=8.8, 2.5 Hz, 1H), 7.74-7.67 (m, 3H), 7.67-7.62 (m, 2H), 7.58 (d, J=8.8 Hz, 1H), 7.54 (d, J=1.9 Hz, 1H), 7.25 (d, J=1.4 Hz, 1H), 7.05 (d, J=1.9 Hz, 1H), 5.19 (s, 2H), 4.58 (s, 1H), 4.03 (dd, J=9.7, 7.4 Hz, 1H), 3.70 (dd, J=9.8, 5.9 Hz, 1H), 2.74 (dd, J=16.9, 8.1 Hz, 1H), 2.56 (dd, J=16.7, 9.7 Hz, 3H), 2.41 (dd, J=16.9, 6.9 Hz, 1H), 1.93 (t, J=7.0 Hz, 2H), 1.25 (d, J=2.5 Hz, 6H).

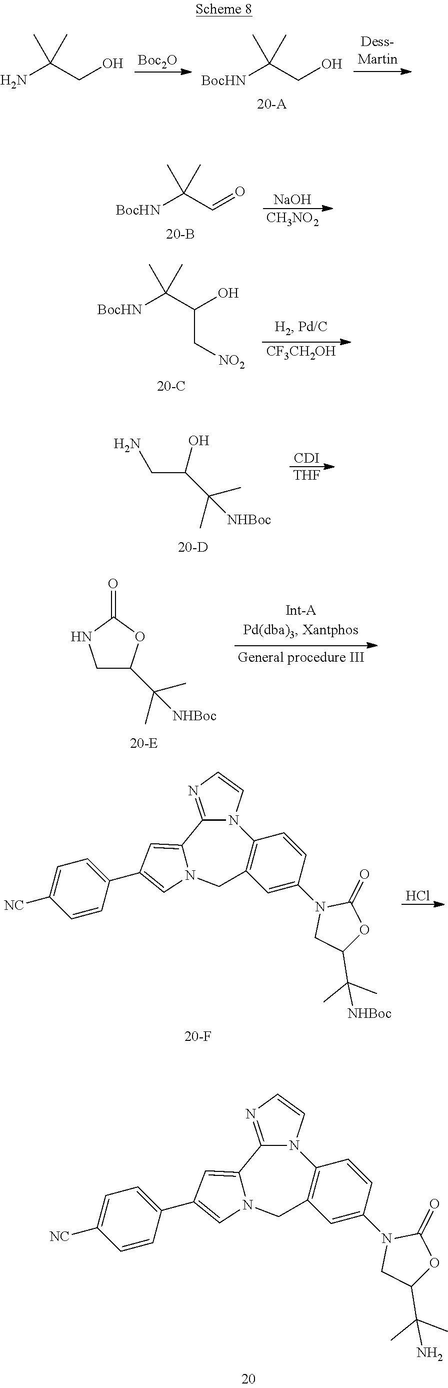

### tert-butyl (1-hydroxy-2-methylpropan-2-yl)carbamate (20-A)

To a solution of 2-amino-2-methylpropan-1-ol (44.5 g, 0.5 mol) and TEA (60.7 g, 0.6 mol) in DCM (200 mL) was added Boc₂O (109.1 g, 0.5 mol) portion-wise at 0° C., then the mixture was stirred at 20° C. for 16 h. The mixture was concentrated under reduced pressure, and the residue was purified by silica gel chromatography eluting with PE/EA (10/1-3/1) to give the desired product (94.6 g, yield: 94%). ESI: [M+H]⁺=190

### tert-butyl (2-methyl-1-oxopropan-2-yl)carbamate (20-B)

To a solution of compound 20-A (20 g, 105.7 mmol) in DCM (200 mL) was added DMP (53.8 g, 126.8 mmol) slowly at 0° C., then the mixture was stirred at 20° C. for 3 h. The mixture was then quenched by saturated NaHCO₃ aqueous solution and Na₂SO₃ solution. The mixture was then stirred for 0.5 h, extracted with DCM, washed with brine, concentrated under reduced pressure. The resulting residue was purified by silica gel chromatography eluting with PE/EA (10/1-3/1) to give the desired compound (15.8 g, yield: 79.9%). ESI: [M+H]⁺=188; ¹H NMR (400 MHz, CDCl₃): δ 9.42 (s, 1H), 1.43 (s, 9H), 1.32 (s, 6H).

### tert-butyl (3-hydroxy-2-methyl-4-nitrobutan-2-yl)carbamate (20-C)

To a solution of compound 20-B (8 g, 42.7 mmol) and nitromethane (2.6 g, 42.7 mmol) in EtOH (80 mL) was added NaOH (10 mol/L, 4.3 mL) dropwise at 0° C., then the mixture was stirred at 20° C. for 16 h. The mixture was quenched with HOAc to pH<6 and extracted with EA. The organic phase was washed with brine, dried over Na₂SO₄, concentrated under reduced pressure. The residue was purified by silica gel chromatography eluting with PE/EA (10/1-1/1) to give the desired product (9.2 g, yield: 86.7%). ESI: [M+H]⁺=249; ¹H NMR (400 MHz, CDCl₃): δ 4.60 (s, 1H), 4.55 (dd, J=12.3, 2.4 Hz, 1H), 4.38 (dd, J=12.3, 9.5 Hz, 1H), 4.27 (d, J=9.3 Hz, 1H), 1.44 (s, 9H), 1.42 (s, 3H), 1.29 (s, 3H).

### tert-butyl (4-amino-3-hydroxy-2-methylbutan-2-yl)carbamate (20-D)

To a solution of compound 20-C (4.0 g, 16.1 mmol) in CF₃CH₂OH (100 mL) was added Pd/C (0.4 g, 10%) and the mixture was stirred at 50° C. for 18 h under hydrogen atmosphere at 50 psi. The mixture was cooled to room temperature and filtered through a pad of celite; the filtrate was concentrated under reduced pressure to give the desired product (3.2 g, yield: 91%). ESI: [M+H]⁺=219.

### tert-butyl (2-(2-oxooxazolidin-5-yl)propan-2-yl)carbamate (20-E)

To a solution of compound 20-D (2.2 g, 9.6 mmol) in THF (30 mL) was added CDI (4.7 g, 28.9 mmol) at 0° C. under N₂ and the mixture was stirred at 70° C. for 18 h under N₂. The mixture was cooled to room temperature and washed with 0.5N HCl (100 mL) to adjust pH<7. The resulting mixture was extracted with EA. The organic phase was washed with brine and concentrated under reduced pressure. The resulting residue was purified by silica gel chromatography eluting with DCM/MeOH (100/1-20/1) to give the desired product (1.6 g, yield: 60.8%). ESI: [M+H]⁺=245; ¹H NMR (400 MHz, CDCl₃): δ 5.41 (s, 1H), 5.00 (dd, J=8.9, 7.4 Hz, 1H), 4.62 (s, 1H), 3.55 (dt, J=16.2, 8.7 Hz, 2H), 1.42 (s, 9H), 1.38 (s, 3H), 1.29 (s, 3H).

General Procedure III:

### tert-butyl (2-(3-(12-(4-cyanophenyl)-9H-benzo[e]imidazo[2,1-c]pyrrolo[1,2-a][1,4] diazepin-7-yl)-2-oxooxazolidin-5-yl)propan-2-yl)carbamate (20-F)

To a suspension of compound Int-A (2.0 g, 5 mmol), compound 20-E (1.50 g, 6 mmol) in DMA (30 mL) was added Pd₂(dba)₃ (0.92 g, 1.0 mmol), Xantphos (1.16 g, 2 mmol) and Cs₂CO₃ (3.25 g, 10 mmol) at 20° C., and the mixture was stirred at 80° C. for 24 h under N₂. The reaction mixture was cooled to room temperature, diluted with DCM/MeOH (20/1, 200 mL) and filtered. The filtrate was concentrated in vacuo, and the residue was poured into iced water. The solid formed was collected by filtration, which was purified by silica gel chromatography eluting with DCM/MeOH (100/1-20/1) first, then re-purified by flash chromatography eluting with DCM/EA (100/1-1/1) to give the desired compound (2.0 g, yield: 71.4%) ESI: [M+H]⁺=565.2

### tert-butyl (2-(3-(12-(4-cyanophenyl)-9H-benzo[e]imidazo[2,1-c]pyrrolo[1,2-a][1,4]diazepin-7-yl)-2-oxooxazolidin-5-yl)propan-2-yl)carbamate (20)

To a solution of compound 20-F (0.12 g, 0.2 mmol) in DCM/MeOH (5 mL/5 mL) was added HCl in dioxane (4N, 2 mL) at 0° C., and the mixture was stirred at 20° C. for 2 h. The reaction mixture was concentrated in vacuo to give the desired compound as HCl salt. Then the product was purified by Prep-HPLC to give the desired product as a white solid. (50 mg, yield:54%). ESI: [M+H]⁺=465.2; ¹H-NMR (400 MHz, CD₃OD): δ 8.09 (s, 1H), 8.05 (d, J=2.5 Hz, 1H), 7.88 (dd, J=9.0, 2.5 Hz, 1H), 7.83-7.72 (m, 5H), 7.70 (d, J=8.5 Hz, 2H), 7.34 (s, 1H), 5.42 (s, 2H), 4.84-4.67 (m, 1H), 4.35 (t, J=9.6 Hz, 1H), 4.12 (dd, J=9.8, 6.9 Hz, 1H), 1.43 (d, J=21.4 Hz, 6H).

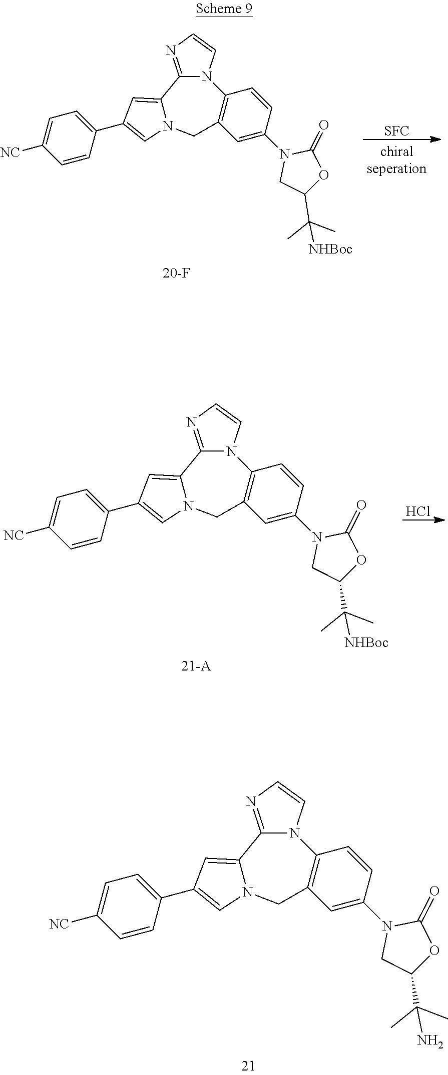

### (S)-tert-butyl (2-(3-(12-(4-cyanophenyl)-9H-benzo[e]imidazo[2,1-c]pyrrolo [1,2-a][1,4]diazepin-7-yl)-2-oxooxazolidin-5-yl)propan-2-yl)carbamate (21-A)

The compound 20-F was separated by chiral-HPLC under below condition to afford peak1 (2.12 g, ee: 99.7%) and peak 2 (2.2 g, ee: 98.5%):
Column: IG-H
Column size: 0.46 cm I.D.*15 cmL
Mobile phase: CO₂:EtOH (0.1% DEA)=60:40
Flow rate: 2.5 mL
Wavelength: UV254 nm
Temperature: 25° C.
Peak 1 was arbitrarily assigned as S-configuration.

### (S)-4-(7-(5-(2-Aminopropan-2-yl)-2-oxooxazolidin-3-yl)-9H-benzo[e] imidazo [2,1-c]pyrrolo[1,2-a][1,4]diazepin-12-yl)benzonitrile (21)

To a solution of compound 021-A (2.12 g, 3.75 mmol) in DCM/MeOH (20 mL/30 mL) was added HCl in dioxane (4N, 20 mL) at 0° C., and the mixture was stirred at 20° C. for 8 h. The reaction mixture was concentrated in vacuo to give the desired compound as HCl salt (1.89 g, purity>95%). Then the product was slurried with THF/MeOH (20 mL/20 mL) to give the desired product as a pale white solid. (1.12 g, yield:59%). ESI: [M+H]⁺=465.2; ¹H NMR (400 MHz, MeOH-d₄): δ 8.17 (d, J=2.2 Hz, 1H), 8.10 (d, J=2.5 Hz, 1H), 7.97-7.69 (m, 8H), 7.39 (d, J=1.8 Hz, 1H), 5.49 (s, 2H), 4.37 (t, J=9.6 Hz, 1H), 4.17 (dd, J=9.9, 6.9 Hz, 1H), 1.47 (s, 3H), 1.41 (s, 3H).

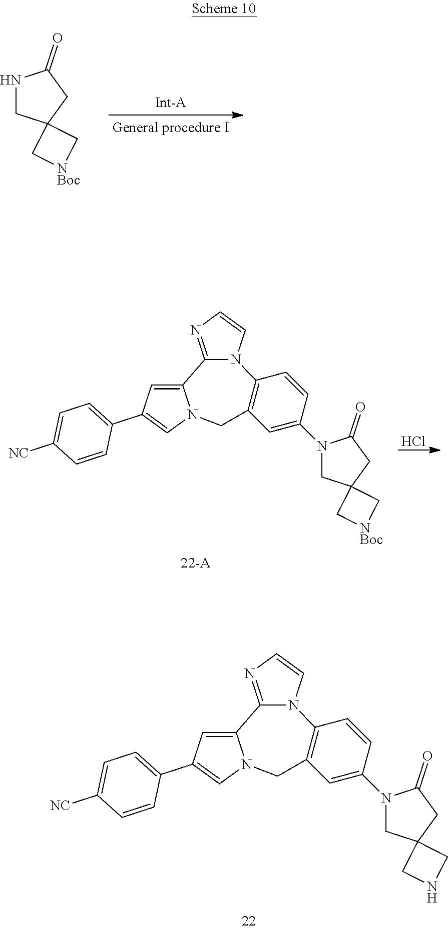 [SMILES: CC(C)(C)OC(=O)N1CC2(CNC(=O)C2)C1 ; CC(C)(C)OC(=O)N1CC2(CC(=O)N(c3ccc4c(c3)Cn3cc(-c5ccc(C#N)cc5)cc3-c3nccn3-4)C2)C1 ; Cl ; N#Cc1ccc(-c2cc3n(c2)Cc2cc(N4CC5(CNC5)CC4=O)ccc2-n2ccnc2-3)cc1]

### tert-butyl 6-(12-(4-cyanophenyl)-9H-benzo[e]imidazo[2,1-c]pyrrolo[1,2-a][1,4]diazepin-7-yl)-7-oxo-2,6-diazaspiro[3.4]octane-2-carboxylate (22-A)

Using the procedures described above, compound 21-A was synthesized. ESI: [M+H]⁺=547.2

### 4-(7-(7-Oxo-2,6-diazaspiro[3.4]octan-6-yl)-9H-benzo[e]imidazo[2,1-c]pyrrolo [1,2-a][1,4]diazepin-12-yl)benzonitrile (22)

To a solution of compound 21-A (100 mg, 0.18 mmol) in MeOH (5 mL) was added HCl-dioxane (4M, 2 mL). The resulting solution was stirred at rt for 1 h and then concentrated to dryness. The residue was purified by Prep-HPLC to give the desired product (50 mg, yield: 62.1%). ESI: [M+H]⁺=447.2; ¹H-NMR (400 MHz, MeOH-d₄): δ 8.11-8.05 (m, 2H), 7.87 (dd, J=8.9, 2.5 Hz, 1H), 7.76-7.73 (m, 5H), 7.69 (d, J=8.5 Hz, 2H), 7.32 (s, 1H), 5.39 (s, 2H), 4.28 (t, J=5.6 Hz, 4H), 4.21 (d, J=11.3 Hz, 2H), 3.08 (s, 2H).

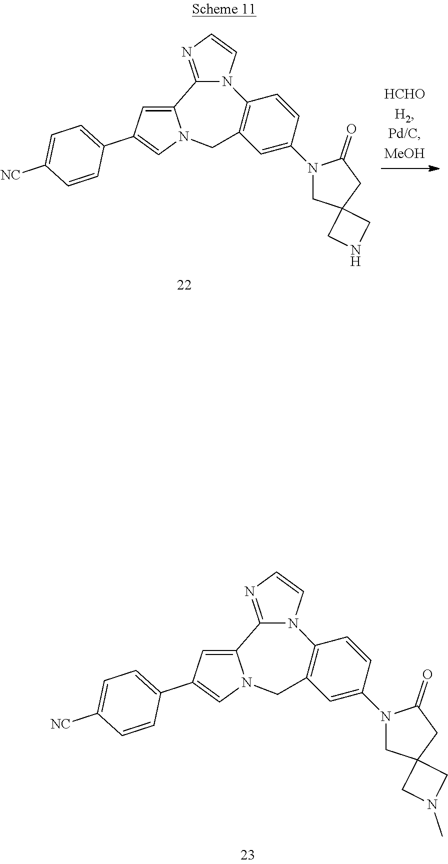 [SMILES: N#Cc1ccc(-c2cc3n(c2)Cc2cc(N4CC5(CNC5)CC4=O)ccc2-n2ccnc2-3)cc1 ; CN1CC2(CC(=O)N(c3ccc4c(c3)Cn3cc(-c5ccc(C#N)cc5)cc3-c3nccn3-4)C2)C1]

### 4-(7-(2-Methyl-7-oxo-2,6-diazaspiro[3.4]octan-6-yl)-9H-benzo[e]imidazo [2,1-c]pyrrolo[1,2-a][1,4]diazepin-12-yl)benzonitrile (23)

To a solution of compound 022 (50 mg, 0.11 mmol) in MeOH (5 mL) was added 10% Pd/C (100 mg), HCHO in H₂O (2 mL). The resulting mixture was stirred at room temperature under hydrogen atmosphere overnight. After the reaction was completed, the mixture was filtered through a pad of celite. The filtrate was concentrated and purified by Prep-HPLC to give the desired product (7 mg, yield: 13.4%). ESI: [M+H]⁺=461.2; ¹H NMR (400 MHz, MeOH-d₄): δ 8.08 (d, J=15.3 Hz, 2H), 7.85 (dd, J=8.9, 2.3 Hz, 1H), 7.81-7.67 (m, 7H), 7.32 (s, 1H), 5.39 (s, 2H), 4.36-4.28 (m, 6H), 3.08 (s, 2H), 2.99 (s, 3H).

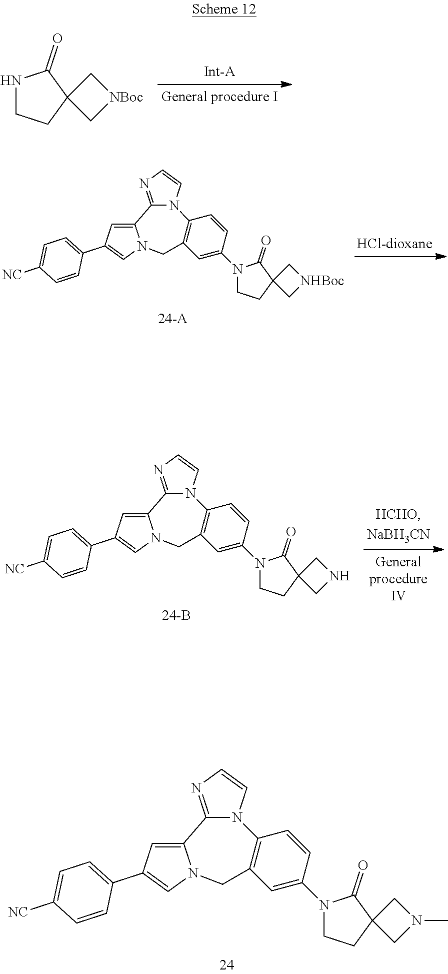

### tert-butyl-6-(12-(4-cyanophenyl)-9H-benzo[e]imidazo[2,1-c]pyrrolo[1,2-a][1,4] diazepin-7-yl)-5-oxo-2,6-diazaspiro[3.4]octane-2-carboxylate (24-A)

Using the procedures described above, compound 24-A was synthesized. ESI: [M+H]⁺=547.2.

### 4-(7-(5-Oxo-2,6-diazaspiro[3.4]octan-6-yl)-9H-benzo[e]imidazo[2,1-c]pyrrolo [1,2-a][1,4]diazepin-12-yl)benzonitrile (24-B)

To a solution of compound 24-A (70 mg, 0.13 mmol) in MeOH (5 mL) was added HCl-dioxane (4M, 2 mL) and stirred at room temperature overnight. After the reaction was completed, the mixture was concentrated, basified with basic resin, filtered, and concentrated to give the crude product (65 mg, yield: 99%). ESI: [M+H]⁺=447.2.

General Procedure IV

### 4-(7-(2-Methyl-5-oxo-2,6-diazaspiro[3.4]octan-6-yl)-911-benzo[e]imidazo [2,1-c]pyrrolo[1,2-a][1,4]diazepin-12-yl)benzonitrile (24)

A solution of compound 24-B (65 mg, 0.15 mmol) in MeOH (5 mL) was charged with NaBH₃CN (27 mg, 0.45 mmol), HCHO (30 mg) and stirred at 40° C. overnight. After the reaction was complete, the mixture was quenched with NH₄Cl (aq.) and extracted with DCM. The organic layer was separated, dried over anhydrous Na₂SO₄, filtered, and concentrated to dryness. The residue was purified by Prep-HPLC to give the desired product (35 mg, yield: 52.4%). ESI: [M+H]⁺=461.2; ¹H NMR (400 MHz, MeOH-d₄): δ 8.14 (d, J=2.4 Hz, 1H), 8.08 (d, J=2.1 Hz, 1H), 8.02 (dd, J=8.9, 2.0 Hz, 1H), 7.82-7.73 (m, 5H), 7.73-7.68 (m, 2H), 7.33 (d, J=1.8 Hz, 1H), 5.42 (s, 2H), 4.59 (dd, J=28.8, 16.8 Hz, 1H), 4.42 (s, 1H), 4.20 (d, J=27.9 Hz, 2H), 3.96 (t, J=7.0 Hz, 2H), 3.08 (s, 1.5H), 3.02 (s, 1.5H), 2.61 (s, 2H).

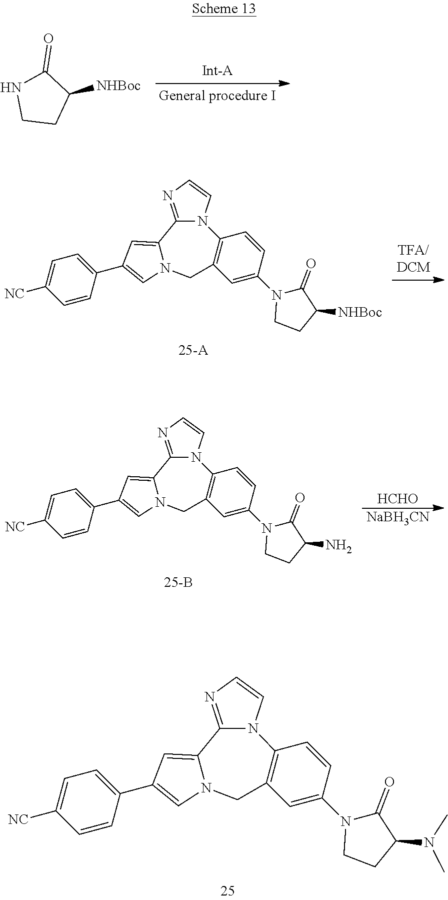 [SMILES: CC(C)(C)OC(=O)N[C@H]1CCNC1=O ; CC(C)(C)OC(=O)N[C@H]1CCN(c2ccc3c(c2)Cn2cc(-c4ccc(C#N)cc4)cc2-c2nccn2-3)C1=O ; N#Cc1ccc(-c2cc3n(c2)Cc2cc(N4CC[C@H](N)C4=O)ccc2-n2ccnc2-3)cc1 ; "HCHO" ; "NaBH3CN" ; CN(C)[C@H]1CCN(c2ccc3c(c2)Cn2cc(-c4ccc(C#N)cc4)cc2-c2nccn2-3)C1=O]

### (S)-tert-butyl (1-(12-(4-cyanophenyl)-9H-benzo[e]imidazo[2,1-c]pyrrolo [1,2-a][1,4]diazepin-7-yl)-2-oxopyrrolidin-3-yl)carbamate (25-A)

Using the procedures described above, 25-A was synthesized. ESI: [M+H]⁺=521.2

### (S)-4-(7-(3-Amino-2-oxopyrrolidin-1-yl)-9H-benzo[e]imidazo[2,1-c]pyrrolo [1,2-a][1,4]diazepin-12-yl)benzonitrile (25-B)

To a solution of compound 25-A (60 mg, 0.115 mmol) in dry DCM (2 mL) was added TFA (0.5 mL). The resulting mixture was stirred at room temperature for 2 h and then evaporated to dryness. The residue was redissolved with DCM/MeOH (10:1, 60 mL), washed with sat'd NaHCO₃ aqueous solution, dried over Na₂SO₄, filtered and concentrated. The crude product was purified by chromatography on silica gel to give the desired product (30 mg, yield: 62.5%). ESI: [M+H]⁺=421.2

### (S)-4-(7-(3-(Dimethylamino)-2-oxopyrrolidin-1-yl)-9H-benzo[e]imidazo [2,1-c]pyrrolo[1,2-a][1,4]diazepin-12-yl)benzonitrile (25)

To a solution of compound 25-B (30 mg, 0.07 mmol) in MeOH/DCE (0.5 mL/0.5 mL) was added HCHO (21 mg, 0.71 mmol) and one drop of AcOH. The solution was stirred for 30 min; then NaBH₃CN (11 mg, 0.18 mmol) was added. The resulting mixture was stirred at room temperature for additional 16 h before dilution with DCM/MeOH (10:1, 60 mL). The solution was washed with water, brine, dried over Na₂SO₄, filtered and concentrated to dryness. The residue was purified by Prep-HPLC to give the desired compound (8 mg, yield: 32%). ESI: [M+H]⁺=449.2; ¹H NMR (400 MHz, MeOH-d₄): δ 8.14 (d, J=2.5 Hz, 1H), 8.03 (d, J=1.9 Hz, 1H), 7.98 (dd, J=8.9, 2.5 Hz, 1H), 7.79-7.65 (m, 5H), 7.29 (d, J=1.7 Hz, 1H), 5.40 (s, 2H), 4.60 (dd, J=11.2, 8.7 Hz, 1H), 4.05 (dq, J=16.3, 9.8 Hz, 2H), 3.03 (s, 6H), 2.79-2.61 (m, 1H), 2.57-2.39 (m, 1H).

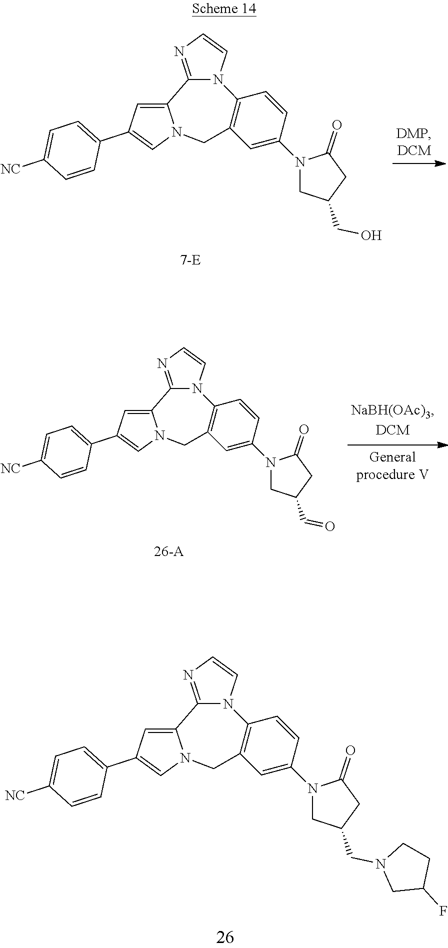 [SMILES: N#Cc1ccc(-c2cc3n(c2)Cc2cc(N4C[C@@H](CO)CC4=O)ccc2-n2ccnc2-3)cc1 ; N#Cc1ccc(-c2cc3n(c2)Cc2cc(N4C[C@@H](C=O)CC4=O)ccc2-n2ccnc2-3)cc1 ; N#Cc1ccc(-c2cc3n(c2)Cc2cc(N4C[C@@H](CN5CCC(F)C5)CC4=O)ccc2-n2ccnc2-3)cc1]

### (S)-4-(7-(4-Formyl-2-oxopyrrolidin-1-yl)-9H-benzo[e]imidazo[2,1-c]pyrrolo[1,2-a][1,4]diazepin-12-yl)benzonitrile 26-A)

To a solution of compound 7-E (870 mg, 2.0 mmol) in DCM (20 mL) was added DMP (1.70 g, 4.0 mmol) portion-wise at 0° C., then the mixture was stirred at 20° C. for 3 h. The mixture was then quenched by saturated NaHCO₃ aqueous solution and Na₂SO₃ solution; and then was stirred for 0.5 hour, extracted with DCM. The combined organic layers were washed with brine, concentrated under reduced pressure. The resulting residue was purified by silica gel chromatography eluting with PE/EA (10/1-3/1) to give the desired compound.

General Procedure V

### 4-(7-((4R)-4-((3-Fluoropyrrolidin-1-yl)methyl)-2-oxopyrrolidin-1-yl)-9H-benzo[e]imidazo[2,1-c]pyrrolo[1,2-a][1,4]diazepin-12-yl)benzonitrile (26)

To a solution of compound 26-A (50 mg, 0.12 mmol), TEA (3 drops) and 3-fluoropyrrolidine hydrochloride (60 mg, 0.475 mmol) in dry DCM (5 mL) was added NaBH(OAc)₃ (48.7 mg, 0.23 mmol), and the resulting mixture was stirred at rt overnight. After the reaction was complete, the mixture was diluted with DCM (30 mL) and washed with aqueous NaHCO₃ and brine. The solution was dried over anhydrous Na₂SO₄ and filtered; the residue was purified by Prep-HPLC to give the desired product (20 mg, yield: 34%). ESI: [M+H]⁺=507.2; ¹H NMR (400 MHz, MeOH-d₄): δ 8.11 (d, J=2.4 Hz, 1H), 8.04 (d, J=2.0 Hz, 1H), 7.85 (dd, J=8.9, 2.4 Hz, 1H), 7.80-7.68 (m, 7H), 7.30 (d, J=1.8 Hz, 1H), 5.39 (s, 2H), 4.20 (ddd, J=11.2, 8.2, 3.2 Hz, 1H), 3.83 (ddd, J=9.8, 7.4, 4.9 Hz, 2H), 3.65-3.50 (m, 4H), 3.11-2.80 (m, 4H), 2.61 (ddd, J=16.9, 8.6, 2.6 Hz, 2H), 1.42-1.31 (m, 1H), 1.17 (t, J=7.0 Hz, 1H).

Using the procedures described above, the following examples in Table 5 were synthesized.

TABLE 5

| | Ex. | | LC-MS |
|---|---|---|---|
| Structure | # | ¹H NMR | (M + H)⁺ |
| 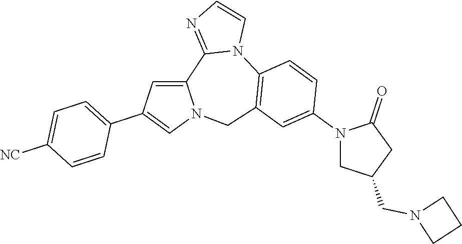 [SMILES: N#Cc1ccc(-c2cc3n(c2)Cc2cc(N4C[C@@H](CN5CCC5)CC4=O)ccc2-n2ccnc2-3)cc1] | 27 | (400 MHz, CDCl3): δ 8.02 (d, J = 2.4 Hz, 1H), 7.63-7.52 (m, 5H), 7.37 (dd, J = 4.9, 3.8 Hz, 2H), 7.28 (d, J = 0.9 Hz, 1H), 7.17 (d, J = 1.7 Hz, 1H), 7.07 (d, J = 1.7 Hz, 1H), 5.03 (s, 2H), 3.95 (dd, J = 9.6, 7.8 Hz, 1H), 3.74 (dd, J =9.7, 6.1 Hz, 1H), 3.45-3.27 (m, 4H), 2.76 (dd, J = 17.0, 8.5 Hz, 1H), 2.64 (dt, J = 11.7, 8.9 Hz, 2H), 2.52 (dt, J = 13.7, 6.8 Hz, 1H), | 475.2 |
| | | 2.36 (dd, J = 17.0, 7.2 | |
| | | Hz, 1H), 2.18-2.15 (m, 2H). | |
| 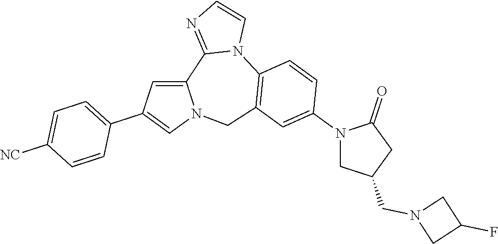 [SMILES: N#Cc1ccc(-c2cc3n(c2)Cc2cc(N4C[C@@H](CN5CC(F)C5)CC4=O)ccc2-n2ccnc2-3)cc1] | 28 | (400 MHz, CDCl₃): δ 8.05 (d, J = 2.3 Hz, 1H), 7.63-7.51 ( (m, 5H), 7.38-7.36 (m, 2H), 7.30 (s, 1H), 7.17 (d, J = 1.8 Hz, 1H), 7.08 (d, J = 1.7 Hz, 1H), 5.10-5.00 (m, 2H), 3.93 (dd, J = 9.6, 7.7 Hz, 1H), 3.79-3.59 (m, 3H), 3.21 (tdd, J = 13.6, 8.9, 5.3 Hz, 2H), 2.76 (dd, J = 16.9, 8.4 Hz, 1H), 2.63 (ddd, J = 17.3, 11.6, 7.4 Hz, 2H), 2.55-2.44 (m, 1H), | 493.2 |
| | | 2.43-2.32 (m, 1H), | |
| | | 1.73-1.68 (m, 1H). | |
| 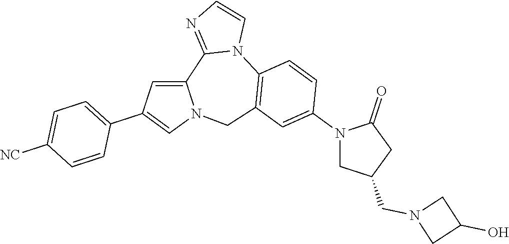 [SMILES: N#Cc1ccc(-c2cc3n(c2)Cc2cc(N4C[C@@H](CN5CC(O)C5)CC4=O)ccc2-n2ccnc2-3)cc1] | 29 | (400 MHz, MeOH-d4): δ 8.08 (d, J = 2.2 Hz, 1H), 8.03(d, J = 2.2 Hz, 1H), 7.84 (dd, J = 8.9, 2.4 Hz, 1H), 7.74- 7.66(m, 7H), 7.28 (d, J = 1.7 Hz, 1H), 5.35 (s, 2H), 4.75-4.65 (m, 1H), 4.49 (s, 2H), 4.11 (t, J = 8.0 Hz, 1H), 4.04 (s, 2H), 3.77 (dd, J = 9.8, 7.0 Hz, 1H), 3.59-3.41 (m, 2H), 2.95-2.76 (m, 2H), 2.62 −2.45 (m, 1H). | 491.2 |

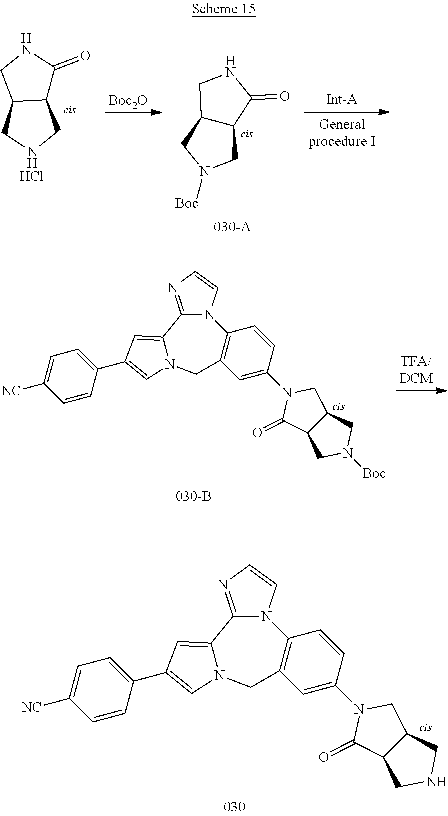 [SMILES: "Boc2O" ; O=C1NC[C@@H]2CNC[C@H]12 ; Cl ; CC(C)(C)OC(=O)N1C[C@H]2CNC(=O)[C@H]2C1 ; CC(C)(C)OC(=O)N1C[C@@H]2CN(c3ccc4c(c3)Cn3cc(-c5ccc(C#N)cc5)cc3-c3nccn3-4)C(=O)[C@@H]2C1 ; N#Cc1ccc(-c2cc3n(c2)Cc2cc(N4C[C@H]5CNC[C@H]5C4=O)ccc2-n2ccnc2-3)cc1]

### cis-tert-butyl 4-oxohexahydropyrrolo[3,4-c]pyrrole-2(1H)-carboxylate (30-A)

To a solution of hexahydropyrrolo[3,4-c]pyrrol-1(2H)-one hydrochloride (50 mg, 0.31 mmol) and TEA (101 mg, 1.0 mmol) in DCM (10 mL) at 0° C. was added Boc₂O (65 mg, 0.31 mmol) slowly. The resulting mixture was stirred at 20° C. for 2 h. The mixture was diluted by water and extracted with EA. The organic layer was dried over anhydrous Na₂SO₄. After filtration, the filtrate was concentrated under reduce pressure to dryness. The residue was purified by silica gel chromatography eluting with PE/EA (10/1-1/10) to give the desired product (25 mg, yield: 36.7%). ESI: [M+H]⁺=227.1

### cis-tert-butyl 5-(12-(4-cyanophenyl)-9H-benzo[e]imidazo[2,1-c]pyrrolo[1,2-a][1,4] diazepin-7-yl)-4-oxohexahydropyrrolo[3,4-c]pyrrole-2(1H)-carboxylate (30-B)

Using the procedures described above, compound 30-B was synthesized. ESI: [M+H]⁺=547.2

### cis-4-(7-(1-oxohexahydropyrrolo[3,4-c]pyrrol-2(1H)-yl)-9H-benzo[e]imidazo [2,1-c]pyrrolo[1,2-a][1,4]diazepin-12-yl)benzonitrile (30)

To a solution of compound 30-B (40 mg, 0.07 mmol) in anhydrous DCM (10 mL) was added TFA (2 mL). The reaction was stirred at 25° C. for 3 h. After starting material was consumed, the reaction was quenched with saturated NaHCO₃ solution to adjust pH>7. The resulting mixture was extracted with DCM. The organic phase was washed with brine, dried over Na₂SO₄, filtered and concentrated to dryness in vacuo. The residue was purified by Prep-HPLC to give the desired compound (20 mg, yield: 64.5%). ESI: [M+H]⁺=447.2; ¹H NMR (400 MHz, MeOH-d₄): δ 8.15 (d, J=2.5 Hz, 1H), 8.07 (d, J=1.8 Hz, 1H), 7.97 (dd, J=8.9, 2.5 Hz, 1H), 7.85-7.66 (m, 7H), 7.32 (d, J=1.5 Hz, 1H), 5.41 (s, 2H), 4.30 (dd, J=10.6, 7.5 Hz, 1H), 3.91 (dd, J=10.7, 1.5 Hz, 1H), 3.79-3.56 (m, 4H), 3.45-3.35 (m, 2H).

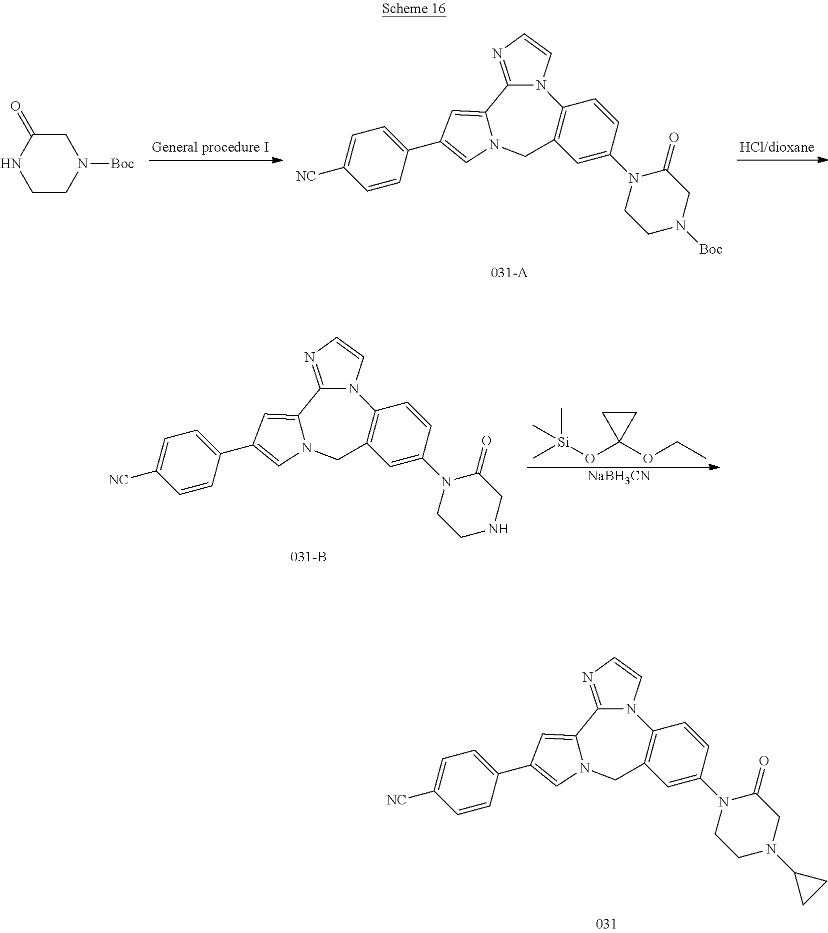

### tert-butyl 4-(12-(4-cyanophenyl)-9H-benzo[e]imidazo[2,1-c]pyrrolo[1,2-a][1,4]diazepin-7-yl)-3-oxopiperazine-1-carboxylate (31-A)

Using the procedures described above, compound 30-A was synthesized. ESI: [M+H]⁺=521.2

### 4-(7-(2-Oxopiperazin-1-yl)-9H-benzo[e]imidazo[2,1-c]pyrrolo[1,2-a][1,4]diazepin-12-yl)benzonitrile (31-B)

To a solution of compound 31-A (78 mg, 0.15 mmol) in MeOH (10 mL) was added HCl in dioxane (2M, 5 mL), and the mixture was stirred at 25° C. overnight. The reaction mixture was then concentrated in vacuo to give the desired product (50 mg, yield: 79.4%). ESI: [M+H]⁺=421.2

### 4-(7-(4-Cyclopropyl-2-oxopiperazin-1-yl)-9H-benzo[e]imidazo[2,1-c]pyrrolo [1,2-a][1,4]diazepin-12-yl)benzonitrile (31)

To a solution of compound 31-B (50 mg, 0.12 mmol), TFA (0.5 mL) in DCE (10 mL) was added (1-ethoxycyclopropoxy)trimethylsilane (62.4 mg, 0.35 mmol) and NaBH₃CN (8.8 mg, 0.14 mmol). The mixture was stirred at 40° C. for 4 h and then concentrated under reduced pressure to dryness. The residue was purified by Prep-HPLC to give the desired product. (10 mg, yield: 18.2%). ESI: [M+H]⁺=461.2; ¹H NMR (400 MHz, MeOH-d₄): δ 8.14 (d, J=2.1 Hz, 1H), 7.82-7.80 (m, 3H), 7.77-7.69 (m, 5H), 7.63 (dd, J=8.7, 2.3 Hz, 1H), 7.35 (d, J=1.8 Hz, 1H), 5.42 (s, 2H), 3.83-3.78 (m, 2H), 3.58 (s, 2H), 3.21-3.16 (m, 2H), 2.06-1.99 (m, 1H), 0.68-0.62 (m, 2H), 0.60 (dd, J=7.4, 3.7 Hz, 2H).

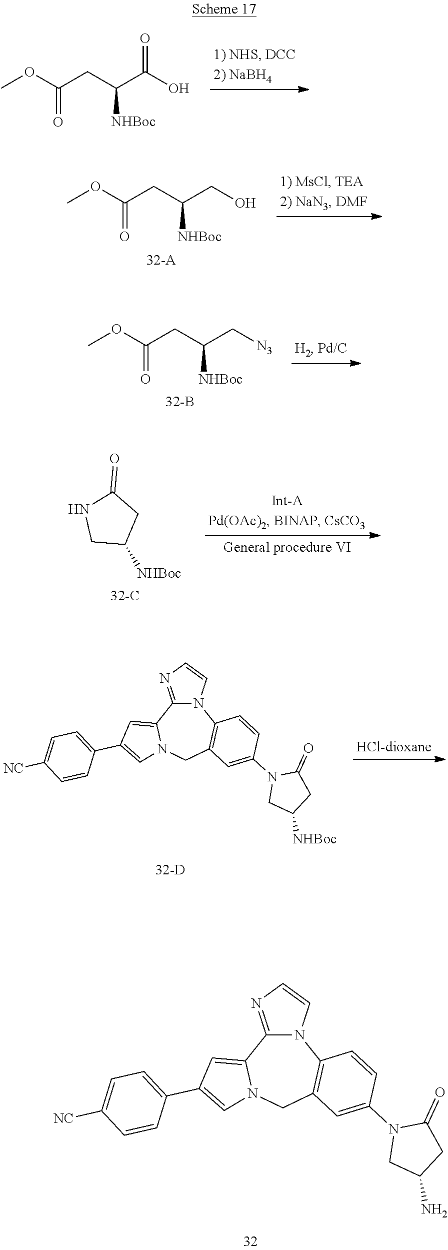 [SMILES: COC(=O)C[C@H](NC(=O)OC(C)(C)C)C(=O)O ; COC(=O)C[C@@H](CO)NC(=O)OC(C)(C)C ; COC(=O)C[C@@H](CN=[N+]=[N-])NC(=O)OC(C)(C)C ; CC(C)(C)OC(=O)N[C@@H]1CNC(=O)C1 ; CC(C)(C)OC(=O)N[C@H]1CC(=O)N(c2ccc3c(c2)Cn2cc(-c4ccc(C#N)cc4)cc2-c2nccn2-3)C1 ; N#Cc1ccc(-c2cc3n(c2)Cc2cc(N4C[C@@H](N)CC4=O)ccc2-n2ccnc2-3)cc1]

### Methyl (S)-3-((tert-butoxycarbonyl)amino)-4-hydroxybutanoate (32-A)

To a solution of (S)-2-((tert-butoxycarbonyl)amino)-4-methoxy-4-oxobutanoic acid (4.95 g, 20 mmol) in 50 mL of EA was added NHS (2.44 g, 21.2 mmol) at 0° C. A solution of DCC (4.17 g, 20.2 mmol) in EA (20 mL) was added slowly to above solution. The mixture was then stirred at 30° C. for 15 h. After the reaction was completed, the mixture was washed with saturated aqueous NaHCO₃, dried over anhydrous Na₂SO₄, filtered and concentrated to dryness. To a solution of NaBH₄ (1.25 g, 32.6 mmol) in THE (60 mL) and H₂O (8 mL) at 0° C., the solution of above residue in THE (10 mL) was added dropwise. The resulting mixture was stirred at 0° C. for 30 min. After the reaction was complete, the mixture was quenched with NH₄Cl aqueous solution and EA. The organic layer was separated, dried over anhydrous Na₂SO₄, filtered and concentrated to dryness. The residue was purified by chromatography on silica gel eluting with PE/EA (3:1) to give the desired alcohol (3.3 g, yield: 50%). ESI: [M+H]⁺=234.1

### Methyl (S)-4-azido-3-((tert-butoxycarbonyl)amino)butanoate (32-B)

To a solution of the compound 31-A (2 g, 8.6 mmol) in DCM (30 mL) was added TEA (0.85 mL, 6.1 mmol) and MsCl (0.76 mL, 9.86 mmol) at 0° C., and the resulting solution was stirred for 30 min at this temperature. After the reaction was complete, the mixture was washed with H₂O, dried over anhydrous Na₂SO₄, filtered, concentrated in vacuo to give the crude product (2 g). To a solution of above product (6.4 mmol) in DMF (30 mL) was added NaN₃ (583 mg, 8.97 mmol), and the resulting mixture was stirred at 80° C. under N₂ overnight. After the reaction was complete, the mixture was diluted with EA, which was then washed with H₂O, dried over anhydrous Na₂SO₄, filtered and concentrated to dryness. The residue was purified by chromatography on silica gel eluting with PE/EA (8/1) to give desired azido compound (500 mg, yield: 19.5% over 2 steps). ESI: [M+H]⁺=259.1

### tert-butyl (S)-(5-oxopyrrolidin-3-yl)carbamate 32-C)

To a solution of compound 32-B (500 mg, 1.9 mmol) in MeOH (10 mL) was added Pd/C (50 mg, 10%) and stirred at 35° C. under H2 for 5 h. After the reaction was complete, the mixture was filtered through a pad of celite. The filtrate was concentrated, and the residue was purified by chromatography on silica gel (DMC:MeOH=30:1) to give the desired product (250 mg, yield: 65%). ESI: [M+H]⁺=201.1

General Procedure VI:

### tert-butyl (S)-(1-(12-(4-cyanophenyl)-9H-benzo[e]imidazo[2,1-c]pyrrolo [1,2-a][1,4]diazepin-7-yl)-5-oxopyrrolidin-3-yl)carbamate (32-D)

To a solution of compound 32-C (50 mg, 0.25 mmol), the compound Int-A (100 mg, 0.25 mmol), Pd(OAc)₂ (5.6 mg, 0.025 mmol), BINAP (31 mg, 0.05 mmol) in toluene (4 mL) was added Cs₂CO₃ (243.7 mg, 0.75 mmol). The resulting mixture was purged with nitrogen and stirred at 110° C. under N₂ overnight. The mixture was cooled to room temperature and diluted with DCM/MeOH (20/1, 200 mL), then filtered. The filtrate was concentrated in vacuo, the resulting residue was purified by chromatography on silica gel (DCM:MeOH=20:1) to give the desired product as a solid (20 mg, yield: 15%). ESI: [M+H]⁺=521.2

### (S)-4-(7-(4-Amino-2-oxopyrrolidin-1-yl)-9H-benzo[e]imidazo[2,1-c]pyrrolo [1,2-a][1,4]diazepin-12-yl)benzonitrile (32)

To a solution of compound 32-D (20 mg, 0.04 mmol) in MeOH (5 mL) was added HCl-dioxane (4M, 3 mL), and the resulting solution was stirred at rt for 3 h. After the reaction was complete, the reaction solution was concentrated under reduced pressure, the residue was purified by Prep-HPLC to give the desired product (16 mg, yield: 99%). ESI: [M+H]⁺=421.2; ¹H NMR (400 MHz, CD₃OD): δ 8.02 (d, J=2.2 Hz, 1H), 8.07 (d, J=2.2 Hz, 1H), 7.91 (dd, J=8.9, 2.5 Hz, 1H), 7.82-7.67 (m, 7H), 7.32 (d, J=1.7 Hz, 1H), 5.41 (s, 2H), 4.43 (dd, J=11.3, 7.0 Hz, 1H), 4.28-4.18 (m, 1H), 3.99 (dd, J=11.3, 2.6 Hz, 1H), 3.23-3.17 (m, 1H), 2.71 (dd, J=18.1, 3.1 Hz, 1H).

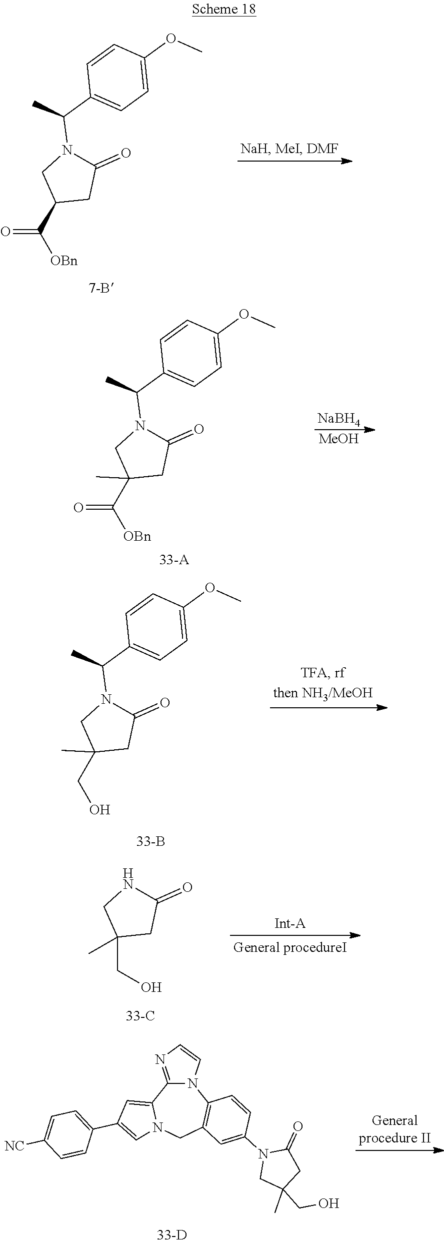

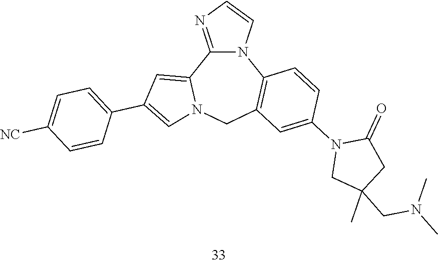 [SMILES: CN(C)CC1(C)CC(=O)N(c2ccc3c(c2)Cn2cc(-c4ccc(C#N)cc4)cc2-c2nccn2-3)C1]

### Benzyl-1-((S)-1-(4-methoxyphenyl)ethyl)-3-methyl-5-oxopyrrolidine-3-carboxylate (33-A)

To a solution of compound 7-B′ (500 mg, 1.41 mmol) in dry DMF (5 mL) was added 60% NaH (227 mg, 5.67 mmol) and MeI (0.9 mL, 14.16 mmol at 0° C. under N₂. The resulting mixture was stirred at 40° C. for 18 h. Sat'd aqueous NH₄Cl solution (20 mL) was added slowly to above mixture at 0° C. to quench the reaction. The resulting mixture was extracted with EA (30 mL×3). The combined organic layers were washed with brine, dried over Na₂SO₄, filtered and concentrated to dryness. The residue was purified by chromatography on silica gel eluting with PE:EA (10:1-3:1) to give the desired product (480 mg, yield: 92.6%). ESI: [M+H]⁺=368.2

### 4-(Hydroxymethyl)-1-((S)-1-(4-methoxyphenyl)ethyl)-4-methylpyrrolidin-2-one (33-B)

To a solution of compound 33-A (480 mg, 1.65 mmol) in dry MeOH (5 mL) was added NaBH₄ (219 mg, 5.77 mmol) at 0° C. The resulting mixture was stirred at room temperature for 18 h. Sat'd aqueous NH₄Cl solution (20 mL) was added slowly to above mixture at 0° C. to quench the reaction. The resulting mixture was extracted with EA (30 mL×3). The combined organic layers were washed with brine, dried over Na₂SO₄, filtered and concentrated to dryness to give the desired product (380 mg, yield: 87.7%) as an off-white solid. ESI: [M+H]⁺=264.2

### 4-(Hydroxymethyl)-4-methylpyrrolidin-2-one (33-C)

A solution of compound 33-B (380 mg, 1.45 mmol) in TFA (5 mL) was heated to 80° C. and stirred at this temperature for 18 h. The volatile solvent was removed under reduced pressure. The residue was re-dissolved in MeOH (50 mL), and NH₃·H₂O was added to above solution at room temperature. After stirring for 2 h, the reaction mixture was concentrated, and the residue was purified by silica gel column chromatography with PE:EA (10:1-1:1) to give the desired product (158 mg, yield: 68.7%). ESI: [M+H]⁺=130.1

### 4-(7-(4-(Hydroxymethyl)-4-methyl-2-oxopyrrolidin-1-yl)-9H-benzo[e]imidazo [2,1-c]pyrrolo[1,2-a][1,4]diazepin-12-yl)benzonitrile (33-D)

Using the procedures described above, compound 33-D was synthesized as a off-white solid. ESI: [M+H]⁺=450.2

### 4-(7-(4-((Dimethylamino)methyl)-4-methyl-2-oxopyrrolidin-1-yl)-9H-benzo [e]imidazo[2,1-c]pyrrolo[1,2-a][1,4]diazepin-12-yl)benzonitrile 33)

Using the procedures described above, compound 33 was synthesized. ESI: [M+H]⁺=477.2; ¹H NMR (400 MHz, CD₃OD): δ 7.97 (d, J=2.4 Hz, 1H), 7.77 (dd, J=8.8, 2.5 Hz, 1H), 7.73-7.70 (m, 3H), 7.65 (d, J=8.5 Hz, 2H), 7.59 (d, J=8.8 Hz, 1H), 7.54 (d, J=1.6 Hz, 1H), 7.26 (s, 1H), 7.06 (d, J=1.6 Hz, 1H), 5.20 (s, 2H), 3.87 (d, J=9.7 Hz, 1H), 3.64 (d, J=9.7 Hz, 1H), 2.65 (d, J=16.8 Hz, 1H), 2.48 (d, J=2.7 Hz, 2H), 2.40 (s, 1H), 2.35 (s, 1H), 2.34 (s, 6H), 1.27 (s, 3H).

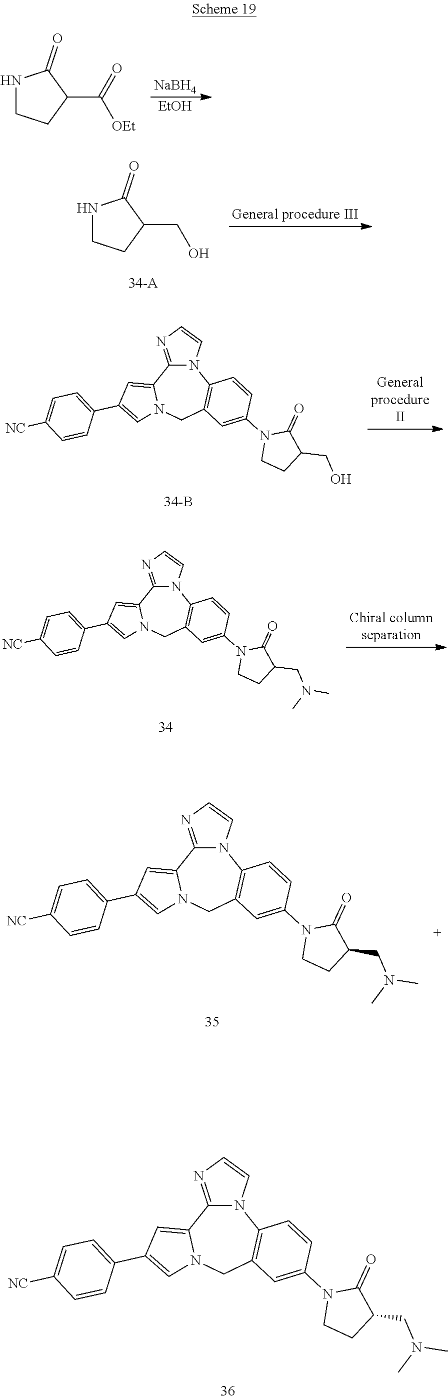

### 3-(Hydroxymethyl)pyrrolidin-2-one (34-A)

To a solution of ethyl 2-oxopyrrolidine-3-carboxylate (1.5 g, 10.0 mmol) in EtOH (100 mL) was added NaBH₄ (2.0 g, 50.0 mmol), and the mixture was stirred at 25° C. for 3 h. 4N HCl in MeOH (30 mL) was then added to quench the reaction, the resulting solution was stirred for 0.5 h and then concentrated to dryness. The residue was purified by silica gel chromatography to give the desired product (1.0 g, yield: 86.9%). ESI: [M+H]⁺=116.1

### 4-(7-(3-(Hydroxymethyl)-2-oxopyrrolidin-1-yl)-9H-benzo[e]imidazo[2,1-c]pyrrolo[1,2-a][1,4]diazepin-12-yl)benzonitrile (34-B)

Using the procedures described above, compound 34-B was synthesized. ESI: [M+H]⁺=436.2; ¹H NMR (400 MHz, CD₃OD): δ 8.12 (d, J=2.5 Hz, 2H), 7.96 (dd, J=8.9, 2.5 Hz, 1H), 7.83 (s, 1H), 7.80 (s, 1H), 7.76 (d, J=9.1 Hz, 3H), 7.71 (d, J=8.6 Hz, 2H), 7.35 (s, 1H), 5.42 (s, 2H), 3.95 (dt, J=6.1, 3.9 Hz, 3H), 3.82 (dd, J=10.9, 3.7 Hz, 1H), 2.86 (dq, J=13.0, 4.2 Hz, 1H), 2.37 (ddd, J=15.0, 12.2, 5.8 Hz, 1H), 2.22 (dq, J=12.7, 8.3 Hz, 1H).

### 4-(7-(3-((Dimethylamino)methyl)-2-oxopyrrolidin-1-yl)-9H-benzo[e]imidazo [2,1-c]pyrrolo[1,2-a][1,4]diazepin-12-yl)benzonitrile (34)

Using the procedures described above, compound 34 was synthesized. ESI: [M+H]⁺=463.2; ¹H NMR (400 MHz, CD₃OD): δ 8.08 (d, J=2.5 Hz, 2H), 8.03 (dd, J=8.9, 2.5 Hz, 1H), 7.81-7.69 (m, 7H), 7.34 (s, 1H), 5.41 (s, 2H), 4.06-3.99 (m, 2H), 3.69-3.59 (m, 1H), 3.38 (dd, J=13.5, 5.0 Hz, 2H), 2.58-2.48 (m, 1H), 2.01 (tt, J=12.6, 9.0 Hz, 1H). Compound 34 was separated by SFC on chiral preparative column to give two enantiomers 35 and 36, their stereochemistry was not determined. The first eluate was arbitrarily assigned as S-isomer (740 mg) and the second eluate was assigned as R-isomer (700 mg). ESI: [M+H]⁺=463.2; Peak 1 ¹H NMR (400 MHz, CD₃OD): δ 7.97 (d, J=2.5 Hz, 1H), 7.79 (dd, J=8.8, 2.5 Hz, 1H), 7.73-7.67 (m, 3H), 7.67-7.62 (m, 2H), 7.58 (d, J=8.8 Hz, 1H), 7.53 (d, J=1.9 Hz, 1H), 7.25 (d, J=1.5 Hz, 1H), 7.05 (d, J=1.9 Hz, 1H), 5.18 (s, 2H), 3.96-3.85 (m, 2H), 2.93 (ddd, J=18.3, 9.6, 3.9 Hz, 1H), 2.84 (dd, J=12.5, 3.9 Hz, 1H), 2.58 (dd, J=12.4, 9.7 Hz, 1H), 2.52-2.41 (m, 1H), 2.33 (s, 6H), 2.01 (dq, J=12.8, 9.0 Hz, 1H).

A solution of compound 35 (400 mg, 0.86 mmol) in hydrochloride in dioxane (5 mL, 5 mmol) was stirred at room temperature for 1 hour. Then reaction mixture was concentrated in vacuum to give its hydrochloride salt as a white solid (440 mg, yield: 99%)

¹H NMR (400 MHz, CD₃OD): δ 8.17 (d, J=2.2 Hz, 1H), 8.12 (d, J=2.5 Hz, 1H), 8.07 (dd, J=8.9, 2.5 Hz, 1H), 7.87 (dd, J=3.9, 2.0 Hz, 2H), 7.77 (dt, J=19.5, 8.8 Hz, 5H), 7.39 (d, J=1.8 Hz, 1H), 5.46 (d, J=9.7 Hz, 2H), 4.10-3.98 (m, 2H), 3.76-3.69 (m, 1H), 3.69-3.62 (m, 2H), 3.58 (m, 1H), 3.00 (d, J=9.9 Hz, 6H), 2.55 (d, J=3.9 Hz, 1H), 2.10-1.94 (m, 1H).

Peak 2 ¹H NMR (400 MHz, CD₃OD): δ 7.97 (d, J=2.5 Hz, 1H), 7.80 (dd, J=8.8, 2.5 Hz, 1H), 7.73-7.67 (m, 3H), 7.67-7.62 (m, 2H), 7.53 (d, J=1.9 Hz, 1H), 7.25 (d, J=1.5 Hz, 1H), 7.05 (d, J=1.9 Hz, 1H), 5.18 (s, 2H), 3.97-3.86 (m, 2H), 2.93 (ddd, J=18.3, 9.6, 3.9 Hz, 1H), 2.84 (dd, J=12.5, 4.0 Hz, 1H), 2.58 (dd, J=12.4, 9.7 Hz, 1H), 2.52-2.41 (m, 1H), 2.33 (s, 6H), 2.01 (dq, J=12.6, 8.9 Hz, 1H).

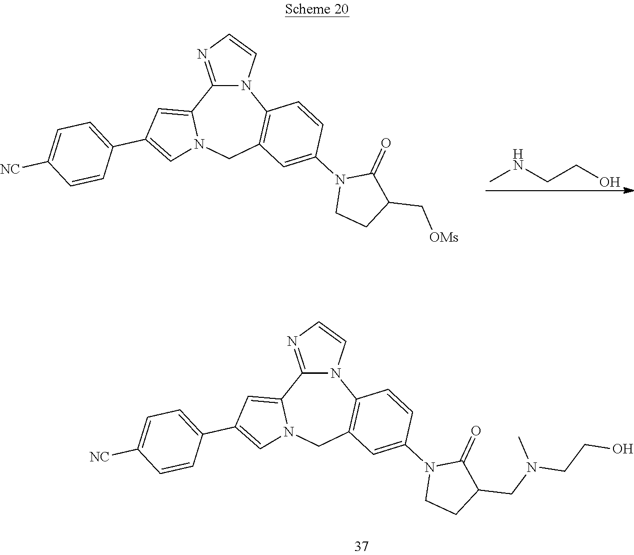

Using the procedures described above, compound 37 was synthesized. ESI: [M+H]⁺=493.2; ¹H-NMR (400 MHz, CD₃OD): δ 8.10 (d, J=2.4 Hz, 1H), 8.03 (s, 1H), 7.98 (dd, J=8.9, 2.4 Hz, 1H), 7.77-7.70 (m, 7H), 7.30 (s, 1H), 5.39 (s, 2H), 4.02 (d, J=7.9 Hz, 2H), 3.93 (t, J=5.0 Hz, 2H), 3.80-3.70 (m, 1H), 3.60-3.50 (m, 1H), 3.43-3.41 (m, 2H), 3.06 (s, 3H), 2.55-2.53 (m, 1H), 2.13-1.90 (m, 1H).

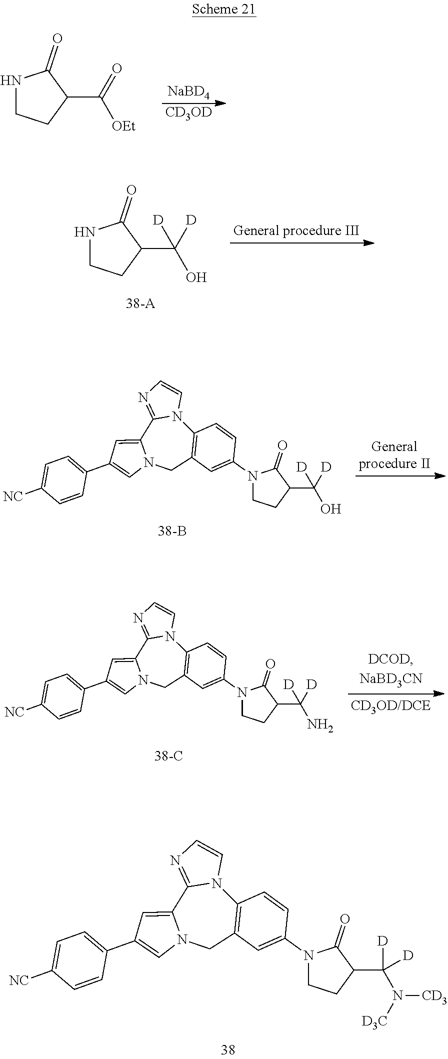

### 3-(Hydroxydeuteratedmethyl)pyrrolidin-2-one (38-A)

To a solution of ethyl 2-oxopyrrolidine-3-carboxylate (300 mg, 1.91 mmol) in CD₃OD (3 mL) was added NaBD₄ (280 mg, 6.69 mmol) at 0° C. The resulting mixture was stirred at room temperature for 16 h under argon atmosphere. The reaction solution was added sat. NH₄Cl (5 mL) at 0° C., and then extracted with DCM/MeOH (10:1, 30 mL*3). The combined organic layers were washed with brine (5 mL), dried over Na₂SO₄, filtered and concentrated. The residue was purified by chromatography on silica gel to give the desired compound as an off-white solid (160 mg, yield: 71.4%). ESI: [M+H]⁺=118.1; ¹H-NMR (400 MHz, CDCl₃): δ 5.94 (s, 1H), 3.48-3.31 (m, 2H), 2.90 (s, 1H), 2.34-2.17 (m, 1H), 2.07-1.85 (m, 1H).

### 4-(7-(3-(Hydroxydeuteratedmethyl)-2-oxopyrrolidin-1-yl)-9H-benzo[e]imidazo[2,1-c]pyrrolo[1,2-a][1,4]diazepin-12-yl)benzonitrile (38-B)

Using the procedures described above, compound 38-B was synthesized. ESI: [M+H]⁺=438.2

### 4-(7-(3-(Aminodeuteratedmethyl)-2-oxopyrrolidin-1-yl)-9H-benzo[e]imidazo[2,1-c]pyrrolo[1,2-a][1,4]diazepin-12-yl)benzonitrile (38-C)

Using the procedures described above, compound 33-C was synthesized. ESI: [M+H]⁺=437.2

### 4-(7-(3-((Dideuteratedmethylamino) deuteratedmethyl)-2-oxopyrrolidin-1-yl)-9H-benzo[e]imidazo[2,1-c]pyrrolo[1,2-a][1,4]diazepin-12-yl)benzonitrile (038)

To a solution of compound 38-C (18 mg, 0.12 mmol) in dry CD₃OD/DCE (0.3 mL/0.3 mL) was added CD20 (13 mg, 0.41 mmol) and cat.AcOH (2 mg). The resulting solution was stirred 30 minutes before the addition of NaBD₃CN (8 mg, 0.12 mmol). The resulting mixture was stirred at room temperature for 4 h and then diluted with DCM/MeOH (10:1, 60 mL). The solution was washed with water, brine, dried over Na₂SO₄, filtered and concentrated. The residue was purified by Prep-TLC to give the desired product (15 mg, yield: 78.9%). ESI: [M+H]⁺=471.3; ¹H NMR (400 MHz, MeOH-d₄): δ 8.06 (d, J=2.4 Hz, 1H), 8.02-7.99 (m, 2H), 7.75-7.65 (m, 7H), 7.30 (d, J=1.6 Hz, 1H), 5.39 (s, 2H), 4.18-3.86 (m, 2H), 3.42-3.35 (m, 1H), 2.53 (ddd, J=8.8, 7.5, 2.9 Hz, 1H), 2.17-1.87 (m, 1H).

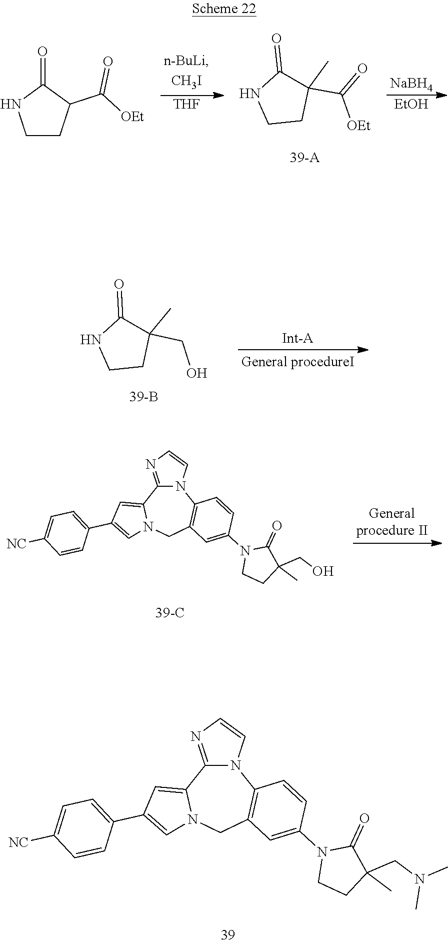

### Ethyl 3-methyl-2-oxopyrrolidine-3-carboxylate 39-A)

To a solution of ethyl 2-oxopyrrolidine-3-carboxylate (500 mg, 3.2 mmol) in dry THE (5 mL) was added dropwise n-BuLi (2.5 M in THF, 1.4 mL, 3.5 mmol) at 0° C. and the resulting mixture allowed to warm slowly to room temperature before cooling down to −78° C. again. CH₃I (497 mg, 3.5 mmol) was added dropwise to above solution at −78° C. The reaction solution was allowed to warm to room temperature and continued to stir for another 16 h under argon atmosphere. Sat'd aqueous NH₄Cl solution was added drop-wise at 0° C. to above solution. The resulting mixture was extracted with DCM/MeOH (10:1, 30 mL*3). The combined organic layers were washed with brine (10 mL), dried over Na₂SO₄, filtered and concentrated to dryness. The residue was purified by chromatography on silica gel to give the desired compound (350 mg, yield: 64.2%) as an oil. ESI: [M+H]⁺=172.1; ¹H-NMR (400 MHz, CDCl₃): δ 6.00 (s, 1H), 4.26-4.16 (m, 2H), 3.48 (dt, J=9.1, 7.4 Hz, 1H), 3.35 (td, J=8.8, 4.0 Hz, 1H), 2.64 (ddd, J=12.9, 7.8, 4.0 Hz, 1H), 2.04 (ddd, J=13.0, 8.4, 7.0 Hz, 1H), 1.45 (d, J=5.0 Hz, 3H), 1.28 (dd, J=9.5, 4.8 Hz, 3H).

### 3-(Hydroxymethyl)-3-methylpyrrolidin-2-one (39-B)

To a solution of compound 39-A (350 mg, 2.05 mmol) in EtOH (10 mL) was added NaBH₄ (272 mg, 7.16 mmol) at 0° C. The resulting mixture was stirred at room temperature for 48 h under argon gas. Sat'd aqueous NH₄Cl solution was added drop-wise at 0° C. to above solution. The resulting mixture was extracted with DCM (30 mL*3). The combined organic layers were washed with brine (10 mL), dried over Na₂SO₄, filtered and concentrated to dryness. The residue was purified by chromatography on silica gel to give the desired compound (234 mg, yield:88.6%) as an oil. ESI: [M+H]⁺=130.1; ¹H NMR (400 MHz, CDCl₃): δ 5.97 (s, 1H), 3.67 (d, J=10.9 Hz, 1H), 3.55 (d, J=10.9 Hz, 1H), 3.44-3.30 (m, 2H), 2.50 (s, 1H), 2.23 (dt, J=12.7, 8.2 Hz, 1H), 1.86 (ddd, J=12.7, 7.1, 4.3 Hz, 1H), 1.20 (s, 3H).

### 4-(7-(3-(Hydroxymethyl)-3-methyl-2-oxopyrrolidin-1-yl)-9H-benzo[e]imidazo [2,1-c]pyrrolo[1,2-a][1,4]diazepin-12-yl)benzonitrile (39-C)

Using the procedures described above, compound 39-C was synthesized. ESI: [M+H]⁺=450.2

### 4-(7-(3-((Dimethylamino)methyl)-3-methyl-2-oxopyrrolidin-1-yl)-9H-benzo[e]imidazo[2,1-c]pyrrolo[1,2-a][1,4]diazepin-12-yl)benzonitrile (39)

Using the procedures described above, compound 39 was synthesized. ESI: [M+H]⁺=477.2; ¹H NMR (400 MHz, CD₃OD): δ 8.12 (d, J=2.4 Hz, 1H), 8.06 (s, 1H), 7.99 (dd, J=8.9, 2.5 Hz, 1H), 7.79-7.67 (m, 7H), 7.31 (d, J=1.3 Hz, 1H), 5.40 (s, 2H), 4.05 (ddd, J=18.7, 13.0, 5.1 Hz, 2H), 3.52 (q, J=13.8 Hz, 2H), 3.00 (s, 6H), 2.37-2.14 (m, 2H), 1.42 (s, 3H).

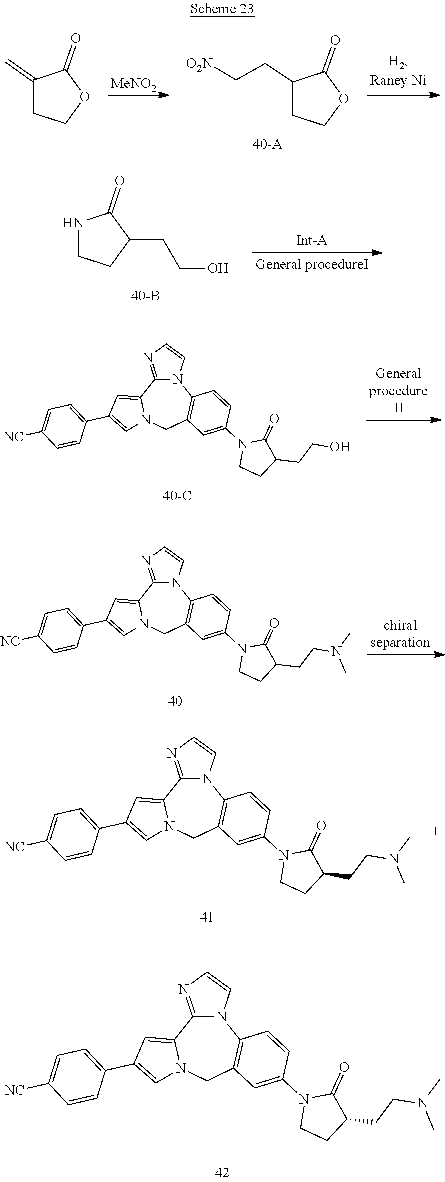

### 3-(2-Nitroethyl)dihydrofuran-2(3H)-one (40-A)

Into a 250-mL round-bottom flask was placed 3-methylideneoxolan-2-one (5 g, 50.97 mmol), DBU (1 g, 6.57 mmol), and CH₃NO₂ (100 mL). The resulting solution was stirred at 25° C. until completion. The resulting mixture was concentrated under vacuum, then taken up in 100 mL of DCM and washed with 3.0 M HCl, water, saturated aqueous NaHCO₃, and brine in turn. The organic solution was dried over anhydrous sodium sulfate, then concentrated under vacuum to deliver the desired compound (5.8 g, yield: 99%). ESI: [M+H]⁺=160.1

### 3-(2-Hydroxyethyl)pyrrolidin-2-one (40-B)

Into a 250-mL round-bottom flask was placed compound 40-A (5.8 g, 36.47 mmol), Raney Ni (4.44 g), methanol (100 mL), and magnesium sulfate (4 g). The resulting solution was stirred under hydrogen atmosphere at room temperature until completion. The solids were filtered out, and the filtrate was concentrated under vacuum to deliver the desired compound (2.3 g, yield: 48.5%). ESI: [M+H]⁺=130.1

### 4-(7-(3-(2-Hydroxyethyl)-2-oxopyrrolidin-1-yl)-9H-benzo[e]imidazo[2,1-c]pyrrolo[1,2-a][1,4]diazepin-12-yl)benzonitrile (40-C)

Using the procedures described above, compound 40-C was synthesized. ESI: [M+H]⁺=450.2

### 4-(7-(3-(2-(Dimethylamino)ethyl)-2-oxopyrrolidin-1-yl)-9H-benzo[e]imidazo [2,1-c]pyrrolo[1,2-a][1,4]diazepin-12-yl)benzonitrile (40)

Using the procedures described above, compound 40 was synthesized. ESI: [M+H]⁺=477.2; ¹H NMR (400 MHz, CD₃OD): δ 8.09-8.08 (m, 2H), 7.98 (dd, J=8.9, 2.5 Hz, 1H), 7.80 (d, J=1.7 Hz, 1H), 7.79-7.73 (m, 4H), 7.70 (d, J=8.5 Hz, 2H), 7.34 (d, J=1.7 Hz, 1H), 5.41 (s, 2H), 3.98 (dd, J=9.3, 4.7 Hz, 2H), 3.44 (ddd, J=12.8, 9.7, 6.5 Hz, 1H), 3.27 (dd, J=8.9, 4.1 Hz, 1H), 2.95 (s, 6H), 2.91-2.79 (m, 1H), 2.47 (qd, J=8.8, 4.5 Hz, 1H), 2.26 (ddt, J=13.7, 9.8, 6.8 Hz, 1H), 2.08-1.84 (m, 2H). The compound 40 was separated by SFC on chiral preparative column to give two enantiomers 41 and 42, their stereochemistry was not determined. The first eluate was assigned as S-isomer and the second eluate was assigned as R-isomer.

### (S)-4-(7-(3-(2-(Dimethylamino)ethyl)-2-oxopyrrolidin-1-yl)-9H-benzo[e]imidazo [2,1-c]pyrrolo[1,2-a][1,4]diazepin-12-yl)benzonitrile (41)

¹H NMR (400 MHz, CD₃OD): δ 7.98 (d, J=2.5 Hz, 1H), 7.82 (dd, J=8.8, 2.5 Hz, 1H), 7.73-7.62 (m, 5H), 7.59 (d, J=8.8 Hz, 1H), 7.53 (d, J=1.9 Hz, 1H), 7.26 (d, J=1.5 Hz, 1H), 7.06 (d, J=1.9 Hz, 1H), 5.19 (s, 2H), 3.99-3.84 (m, 2H), 3.04-2.92 (m, 1H), 2.91-2.72 (m, 2H), 2.62 (s, 6H), 2.50-2.36 (m, 1H), 2.18 (ddt, J=12.8, 9.9, 6.3 Hz, 1H), 2.00-1.70 (m, 2H).

### (R)-4-(7-(3-(2-(Dimethylamino)ethyl)-2-oxopyrrolidin-1-yl)-9H-benzo[e]imidazo [2,1-c]pyrrolo[1,2-a][1,4]diazepin-12-yl)benzonitrile (42)

¹H NMR (400 MHz, CD₃OD): δ 7.98 (d, J=2.4 Hz, 1H), 7.79 (dd, J=8.8, 2.5 Hz, 1H), 7.69-7.66 (m, 5H), 7.58 (d, J=8.8 Hz, 1H), 7.54 (d, J=1.8 Hz, 1H), 7.25 (d, J=1.4 Hz, 1H), 7.05 (d, J=1.9 Hz, 1H), 5.19 (s, 2H), 3.97-3.83 (m, 2H), 2.71 (ddd, J=18.4, 9.1, 4.8 Hz, 1H), 2.62-2.37 (m, 4H), 2.31 (s, 6H), 2.14 (ddd, J=21.3, 10.7, 5.9 Hz, 1H), 1.87 (dq, J=12.4, 8.9 Hz, 1H), 1.73-1.53 (m, 1H).

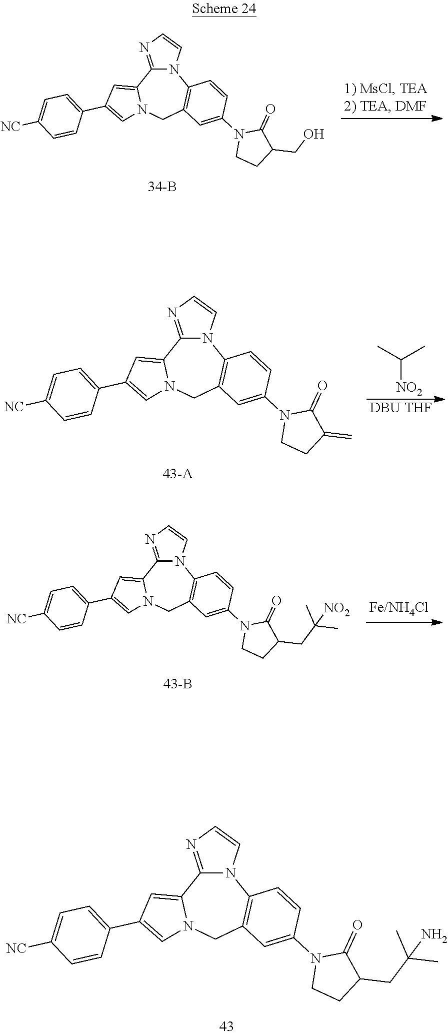

### 4-(7-(3-Methylene-2-oxopyrrolidin-1-yl)-9H-benzo[e]imidazo[2,1-c]pyrrolo[1,2-a][1,4]diazepin-12-yl)benzonitrile (43-A)

To a 0° C. solution of compound 34-B (1.31 g, 3.0 mmol), TEA (909 mg, 9.0 mmol) in DCM (15 mL) was added MsCl (689 mg, 6.00 mmol) and stirred for 1 h. The reaction was quenched with aqueous NaHCO₃ solution; the resulting mixture was extracted with DCM three times. The combined organic layers were dried over Na₂SO₄, filtered and concentrated to dryness. The residue was then purified by chromatography on silica gel to get the desired product (1.22 g, yield: 79.3%), which was used directly. At room temperature, the solution of above compound (0.85 g, 1.66 mmol), TEA (3 mL) in DMF (10 mL) was stirred for 5 h. The reaction solution was dried under the reduced pressure, and the residue was purified by chromatography on silica gel to get the desired compound (0.52 g, yield: 63.4%). ESI: [M+H]⁺=418.2

### (R)-4-(7-(3-(2-Methyl-2-nitropropyl)-2-oxopyrrolidin-1-yl)-9H-benzo[e]imidazo [2,1-c]pyrrolo[1,2-a][1,4]diazepin-12-yl)benzonitrile (43-B)

The solution of compound 43-A (0.52 g, 1.32 mmol), 2-nitropropane (0.59 g, 6.6 mmol,), DBU (1.00 g, 6.6 mmol) in THE (15 mL) was stirred at reflux for overnight under the N₂. The reaction mixture was concentrated under reduced pressure, the residue was purified by chromatography on silica gel to get the desired product (0.4 g, yield: 59.8%). ESI: [M+H]⁺=507.2

### (R)-4-(7-(3-(2-Amino-2-methylpropyl)-2-oxopyrrolidin-1-yl)-9H-benzo[e]imidazo[2,1-c]pyrrolo[1,2-a][1,4]diazepin-12-yl)benzonitrile (43)

To the solution of compound 43-B (50 mg, 0.10 mmol), NH₄Cl (26 mg, 0.50 mmol) in EtOH (8 mL), THE (4 mL), H₂O (4 mL) was added Fe (28 mg, 0.50 mmol). The reaction mixture was stirred overnight at 80° C. under N₂. The reaction mixture was cooled to room temperature and filtered through a pad of celite. The filtrate was concentrated, and the residue was purified by flash chromatography and then Prep-HPLC to give the desired product (14 mg, yield: 29.2%). ESI: [M+H]⁺=477.2; ¹H NMR (400 MHz, CD₃OD): δ 8.10 (s, 1H), 8.09 (s, 1H), 7.99 (dd, J=8.9, 2.5 Hz, 1H), 7.81-7.69 (m, 7H), 7.34 (d, J=1.8 Hz, 1H), 5.42 (s, 2H), 4.02-3.93 (m, 2H), 3.07-2.98 (m, 1H), 2.53 (s, 1H), 2.30-2.21 (m, 1H), 1.96 (dd, J=19.0, 7.0 Hz, 1H), 1.80 (dd, J=14.9, 6.1 Hz, 1H), 1.45 (s, 3H), 1.44 (s, 3H).

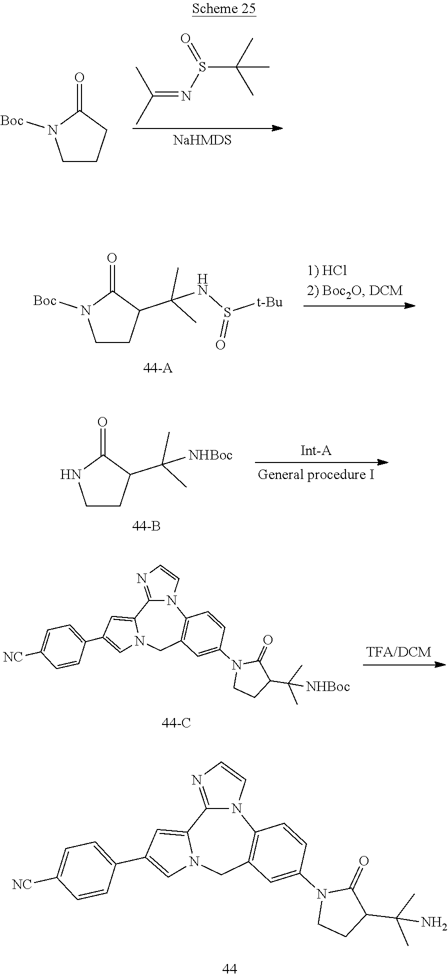

### tert-butyl 3-(2-(1,1-dimethylethylsulfinamido)propan-2-yl)-2-oxopyrrolidine-1-carboxylate (44-A)

To a solution of tert-butyl 2-oxopyrrolidine-1-carboxylate (926.1 mg, 5 mmol) in THE (20 mL) was added NaHMDS (2 M, 3 mL) at −78° C. The mixture was stirred at −78° C. for 0.5 h, and then the solution of 2-methyl-N-propan-2-ylidene-propane-2-sulfinamide (806.3 mg, 5 mmol) in THE (2 mL) was added at −78° C. The resulting solution was stirred at −78° C. for another 0.5 h, and the reaction was quenched by addition of saturated NH₄Cl aqueous solution. The mixture was extracted with EA three times. The combined organic layers were washed with brine, dried over anhydrous Na₂SO₄ and concentrated under reduced pressure to give the crude oil, which was purified by flash chromatography eluting with PE/EA (10/1-1/1) to give the desired product (470 mg, yield: 27.1%) as a white solid. ESI: [M+H]⁺=347.2

### tert-butyl (2-(2-oxopyrrolidin-3-yl)propan-2-yl)carbamate (44-B)

A solution of compound 44-A (447 mg, 1.29 mmol) in HCl (4N in dioxane, 3 mL) was stirred at 20° C. for 2 h, and then concentrated under reduced pressure to give the crude compound as HCl salt, which was used in next step directly. To a solution of above crude compound (185 mg, 1.3 mmol) and TEA (394 mg, 3.9 mmol) was added Boc₂O (283 mg, 1.3 mmol). The solution was stirred at 20° C. for 2 h before the addition of H₂O. The resulting mixture was extracted with ethyl acetate three times. The combined organic layers were washed with brine, dried over anhydrous Na₂SO₄ and concentrated under reduced pressure to dryness. The residue was purified by flash chromatography eluting with DCM/MeOH (100/1-20/1) to give the desired compound (190 mg, yield: 60.3%). ESI: [M+H]⁺=243.2

### tert-butyl (2-(1-(12-(4-cyanophenyl)-9H-benzo[e]imidazo[2,1-c]pyrrolo[1,2-a][1,4]diazepin-7-yl)-2-oxopyrrolidin-3-yl)propan-2-yl)carbamate (44-C)

Using the procedures described above, compound 44-C was synthesized. ESI: [M+H]⁺=563.3

### 4-(7-(3-(2-Aminopropan-2-yl)-2-oxopyrrolidin-1-yl)-9H-benzo[e]imidazo [2,1-c]pyrrolo[1,2-a][1,4]diazepin-12-yl)benzonitrile (44)

To a solution of compound 44-C (40.00 mg, 0.07 mmol) in DCM (10 mL) was added TFA (2 mL), and the resulting solution was stirred at 20° C. for 18 h. Sat'd NaHCO₃ aqueous solution (20 mL) was added slowly to the above solution; the resulting mixture was extracted with DCM three times. The combined organic layers were washed with brine, dried over anhydrous Na₂SO₄ filtered and concentrated to dryness. The residue was purified by Prep-HPLC to give the desired product (12 mg, yield: 36.5%). ESI: [M+H]⁺=463.2; ¹H-NMR (400 MHz, CD₃OD): δ 8.07 (s, 1H), 8.00 (m, 2H), 7.71 (m, 7H), 7.29 (d, J=4.2 Hz, 1H), 5.38 (s, 2H), 4.11-3.82 (m, 2H), 3.08 (m, 1H), 2.47-2.34 (m, 1H), 2.18-2.01 (m, 1H), 1.44 (s, 3H), 1.44 (s, 3H).

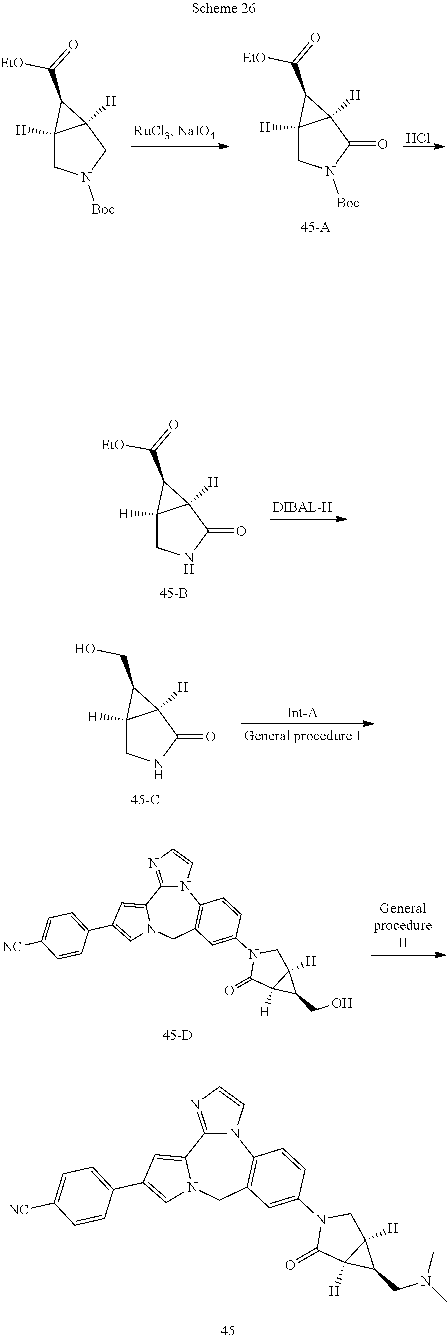

### 3-(tert-butyl) 6-ethyl (1S,5R,6R)-2-oxo-3-azabicyclo[3.1.0]hexane-3,6-dicarboxy late (45-A)

The mixture of NaIO₄ (1.68 g, 7.84 mmol) and RuCl₃·3H₂O (51 mg, 0.196 mmol) in water (20 mL) was stirred at rt for 30 min. 3-(tert-butyl) 6-ethyl (1R, 5S, 6s)-3-azabicyclo[3.1.0]hexane-3,6-dicarboxylate (1 g, 3.92 mmol) in EA (20 mL) was added to above the mixture. The resulting mixture was stirred at rt for 3 h and filtered. The EA layer in the filtrate was separated and concentrated to dryness. The residue was purified by silica gel chromatography eluting with PE/EA (10/1-3/1) to give the desired product (520 mg, yield: 49%). ESI: [M+H]⁺=270.1

### Ethyl (1S,5R,6R)-2-oxo-3-azabicyclo[3.1.0]hexane-6-carboxylate (45-B)

To a solution of compound 45-A (800 mg, 2.97 mmol) in DCM (20 mL) was added HCl in dioxane (4M, 2 mL) at rt and stirred for 1 h. The reaction solution was then concentrated to dryness to obtain the desired compound (500 mg, yield: 99%) as a solid.

### (1S,5S,6R)-6-(Hydroxymethyl)-3-azabicyclo[3.1.0]hexan-2-one (45-C)

To a solution of compound 45-B in dried THE under N₂ was added DIBAL-H (1.5 M in toluene, 4 mL) at 0° C. The resulting reaction solution was stirred at 0° C.-rt overnight before it was quenched by water slowly. After the filtration of the resulting mixture, the filtrate was concentrated to give the crude compound, which was purified by chromatography on silica gel (DCM:MeOH=10:1) to obtain the desired compound (120 mg, yield: 32%) as a yellow solid. ESI: [M+H]⁺=128.1

### 4-(7-((1S,5S,6R)-6-(Hydroxymethyl)-2-oxo-3-azabicyclo[3.1.0]hexan-3-yl)-9H-benzo[e]imidazo[2,1-c]pyrrolo[1,2-a][1,4]diazepin-12-yl)benzonitrile (45-D)

Using the procedures described above, compound 45-D was synthesized. ESI: [M+H]⁺=448.2

### 4-(7-((1R,5S,6R)-6-((Dimethylamino)methyl)-2-oxo-3-azabicyclo[3.1.0]hexan-3-yl)-9H-benzo[e]imidazo[2,1-c]pyrrolo[1,2-a][1,4]diazepin-12-yl)benzonitrile (45)

Using the procedures described above, compound 45 was synthesized. ESI: [M+H]+=475.2; ¹H NMR (400 MHz, CD₃OD): δ 8.25-8.02 (m, 2H), 7.93 (dd, J=8.9, 2.3 Hz, 1H), 7.83-7.63 (m, 7H), 7.33 (s, 1H), 5.40 (s, 2H), 4.28 (dd, J=11.6, 6.5 Hz, 1H), 3.92 (d, J=11.5 Hz, 1H), 3.42 (dd, J=13.6, 5.4 Hz, 1H), 3.03-2.86 (m, 7H), 2.63-2.41 (m, 2H), 1.82 (dd, J=8.2, 5.5 Hz, 1H).

Using the procedures described above, compound 46 in Table 6 was synthesized

TABLE 6

| | Ex. | | LC-MS |
|---|---|---|---|
| Structure | # | ¹H NMR | (M + H)⁺ |
| 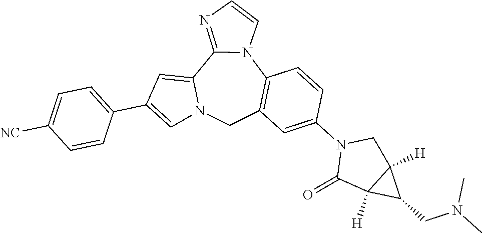 [SMILES: CN(C)C[C@H]1[C@@H]2CN(c3ccc4c(c3)Cn3cc(-c5ccc(C#N)cc5)cc3-c3nccn3-4)C(=O)[C@H]12] | 46 | (400 MHz, CD₃OD): δ 8.11(d, J = 2.0 Hz, 1H), 8.08 (s, 1H), 7.94-7.91(m, 1H), 7.80-7.69 (m, 7H), 7.34 (s, 1H), 5.40 (s, 2H), 4.22 (dd, J = 10.5, 5.4 Hz, 1H), 4.03 (d, J = 10.6 Hz, 1H), 3.28 − 3.15 (m, 2H), 2.97 (s, 6H), 2.44 − 2.21 (m, 2H), 1.83-1.80 (m, 1H). | 475.2 |

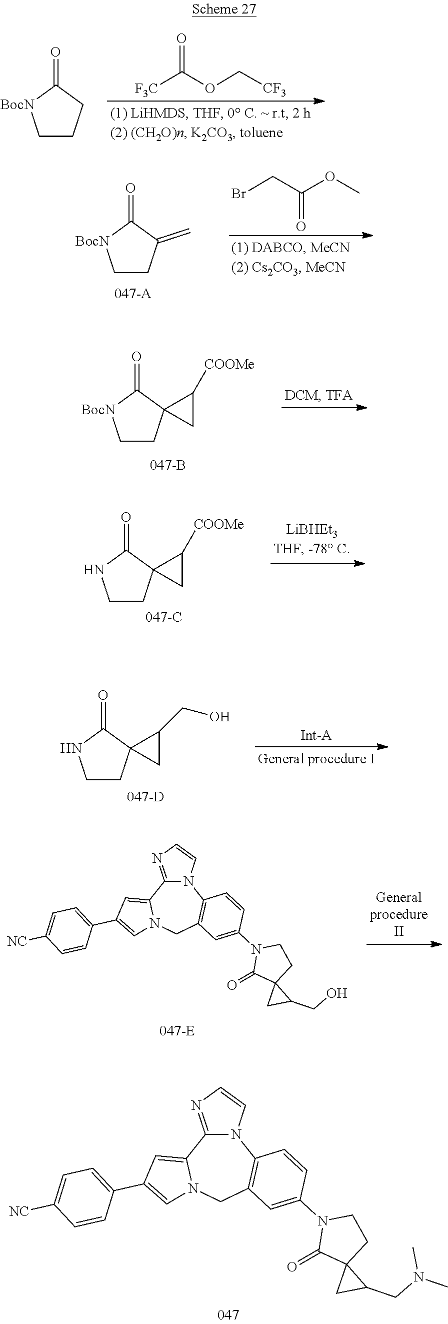

### tert-butyl 3-methylene-2-oxopyrrolidine-1-carboxylate (47-A)

To a solution of LiHMDS (1.0 M in THF, 55.4 mL, 55.4 mmol) in dry THE (10 mL) was added dropwise at −78° C. a solution of tert-butyl 2-oxopyrrolidine-1-carboxylate (5.0 g, 27.03 mmol) in dry THE (10 mL). The resulting mixture was stirred at room temperature for 40 minutes. Then 2,2,2-trifluoroethyl 2,2,2-trifluoroacetate (6.9 mL, 51.36 mmol) was added at room temperature dropwise and the resulting mixture was stirred at room temperature for another 20 minutes. After starting material was consumed, the reaction was quenched at 0° C. with sat'd NH₄Cl aqueous and extracted with EA (30 mL*3). The combined organic layers were washed with water, brine, dried over Na₂SO₄, filtered and concentrated. The crude product was dissolved in toluene (100 mL), (CH₂O)ₙ (4.1 g, 135.2 mmol) and K₂CO₃ (8.2 g, 59.5 mmol) were added to above solution. The resulting mixture was stirred at 108° C. for 2 h. After starting material was consumed, the reaction mixture was cooled to room temperature, diluted with water, and extracted with EA (30 mL*3). The combined organic layers were washed with water, brine, dried over Na₂SO₄, filtered and concentrated. The residue was purified by column chromatography on silica gel to give the desired product (1.1 g, yield: 20.4%) as a solid. ESI: [M+H]⁺=198; ¹H NMR (400 MHz, CDCl₃): δ 6.19 (t, J=2.8 Hz, 1H), 5.48 (t, J=2.4 Hz, 1H), 3.82-3.65 (m, 2H), 2.75 (tt, J=7.1, 2.7 Hz, 2H), 1.55 (s, 9H).

### 5-(tert-butyl) 1-methyl 4-oxo-5-azaspiro[2.4]heptane-1,5-dicarboxylate (47-B)

To a solution of methyl 2-bromoacetate (1.3 mL, 14.47 mmol) in dry MeCN (20 mL) was added DABCO (1.62 g, 14.47 mmol), and after 30 minutes, Cs₂CO₃ (4.71 g, 14.47 mmol) and a solution of compound 47-A (950 mg, 4.82 mmol) in dry MeCN (35 mL) were added. The resulting mixture was stirred at 80° C. for 22 h. The reaction mixture was cooled to room temperature, diluted with water, and extracted with EA (30 mL*3). The combined organic layers were washed with water, brine, dried over Na₂SO₄, filtered and concentrated. The residue was purified by column chromatography on silica gel to give the desired product (300 mg, yield: 23%) as a solid. ESI [M−56+H]⁺=214; ¹H NMR (400 MHz, CDCl₃): δ 3.86-3.78 (m, 2H), 3.76-3.65 (m, 3H), 2.40-2.26 (m, 1H), 2.25-2.16 (m, 2H), 1.69 (s, 2H), 1.61 (dd, J=8.9, 4.1 Hz, 1H), 1.57-1.49 (m, 9H), 1.43 (dd, J=6.1, 3.9 Hz, 2H), 1.31-1.22 (m, 1H).

### Methyl 4-oxo-5-azaspiro[2.4]heptane-1-carboxylate (47-C)

To a solution of compound 47-B (300 mg, 1.11 mmol) in dry DCM (3 mL) was added dropwise TFA (0.5 mL) at 0° C. The resulting mixture was stirred at room temperature for 2 h and then concentrated to dryness. The residue was purified by column chromatography on silica gel to give the desired product (174 mg, yield: 92.5%) as a solid. ESI [M−56+H]⁺=170.

### 1-(Hydroxymethyl)-5-azaspiro[2.4]heptan-4-one (47-D)

To a solution of compound 47-C (170 mg, 0.92 mmol) in dry THE (3 mL) was added dropwise LiBHEt₃ (1.0 M in THF, 4.6 mL, 4.62 mmol) at −78° C. The resulting mixture was stirred at −78° C. for 2 h. After which period, at 0° C. the reaction was quenched with sat'd NH₄Cl aqueous and extracted with EA (30 mL*3). The combined organic layers were washed with water, brine, dried over Na₂SO₄, filtered and concentrated to dryness. The residue was purified by column chromatography on silica gel to give the desired product (97 mg, yield: 74.6%) as an oil. ESI [M−56+H]+=142; ¹H NMR (400 MHz, MeOH-d₄) δ 3.79 (dd, J=11.7, 5.4 Hz, 1H), 3.48 (dd, J=7.9, 6.8 Hz, 2H), 3.34 (s, 2H), 2.45-2.29 (m, 1H), 2.10 (ddd, J=13.0, 12.3, 8.3 Hz, 1H), 1.62-1.48 (m, 1H), 1.14 (dd, J=9.2, 4.3 Hz, 1H), 0.62 (dd, J=6.3, 4.4 Hz, 1H).

### 4-(7-(1-(Hydroxymethyl)-4-oxo-5-azaspiro[2.4]heptan-5-yl)-9H-benzo[e]imidazo[2,1-c]pyrrolo[1,2-a][1,4]diazepin-12-yl)benzonitrile (47-E)

Using the procedures described above, compound 47-E was synthesized. ESI: [M+H]⁺=462

### 4-(7-(1-((Dimethylamino)methyl)-4-oxo-5-azaspiro[2.4]heptan-5-yl)-9H-benzo[e]imidazo[2,1-c]pyrrolo[1,2-a][1,4]diazepin-12-yl)benzonitrile (47)

Using the procedures described above, compound 47 as synthesized. ESI: [M+H]⁺=489; ¹H NMR (400 MHz, MeOH-d₄): δ 8.09 (d, J=2.4 Hz, 1H), 7.99 (d, J=1.9 Hz, 1H), 7.94 (dd, J=8.9, 2.5 Hz, 1H), 7.73-7.70 (m, 6H), 7.65 (d, J=1.8 Hz, 1H), 7.27 (d, J=1.8 Hz, 1H), 5.36 (s, 2H), 4.10 (dt, J=9.6, 6.5 Hz, 2H), 3.54 (dd, J=13.2, 4.9 Hz, 1H), 3.04-2.93 (m, 7H), 2.39 (dt, J=13.0, 9.0 Hz, 1H), 2.26 (ddd, J=12.8, 8.3, 4.6 Hz, 1H), 1.82-1.69 (m, 1H), 1.50 (dd, J=8.7, 4.7 Hz, 1H), 1.11-1.01 (m, 1H).

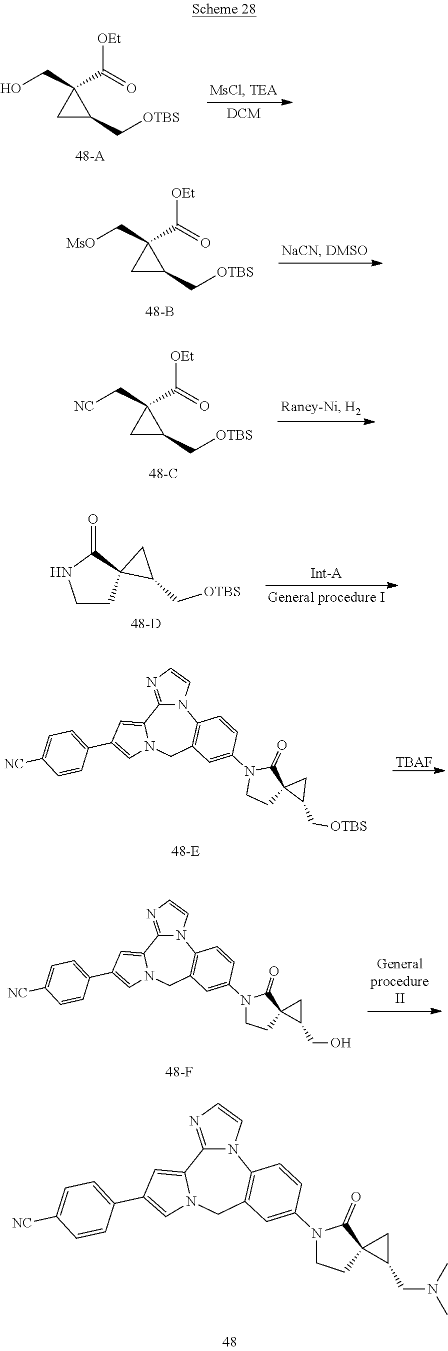

### Ethyl (1S,2S)-2-(((tert-butyldimethylsilyl)oxy)methyl)-1-(((methylsulfonyl)oxy) methyl)cyclopropane-1-carboxylate (48-B)

To a solution of compound 48-A (1.4 g, 4.86 m mol) in DCM (5 mL) as added TEA (1 mL) and MsCl (1.1 g, 9.72 mmol) at 0° C. The resulting mixture was stirred for 1 hour and then poured into NaHCO₃(aq.) (20 mL). The mixture was extracted with DCM (20 mL×2). The combined organic layers were washed with brine (20 mL×2), dried over Na₂SO₄, filtered, concentrated to dryness. The crude product was purified by chromatography on silica gel eluting with DCM:MeOH (20:1) to get the desired product (1.7 g, yield: 98%).

### Ethyl (1R,2S)-2-(((tert-butyldimethylsilyl)oxy)methyl)-1-(cyanomethyl) cyclopropane-1-carboxylate (48-C)

To a solution of compound 48-B (1.7 g, 4.6 mmol) in DMSO (5 mL) was added NaCN (455 mg, 9.29 mmol). The mixture was stirred at 95° C. for 3 h. The reaction mixture was cooled to room temperature, poured into NaHCO₃ (20 mL), and extracted with DCM (20 mL×2). The combined organic layers were washed with brine (20 mL×2), dried over Na₂SO₄, filtered and concentrated in vacuo. The crude product was purified by chromatography on silica gel to get the desired product (800 mg, yield: 61.5%).

### (1S,3R)-1-(((tert-butyldimethylsilyl)oxy)methyl)-5-azaspiro[2.4]heptan-4-one (48-D)

To a solution of compound 48-C (400 mg, 1.34 mmol) in MeOH (5 mL) was added Raney-Ni (455 mg, 9.29 mmol), and the resulting mixture was stirred under H2 at 65° C. for 3 h. The reaction mixture was cooled to room temperature and filtered through a pad of celite and concentrated to dryness. The residue was purified by silica gel chromatography eluting to get the desired product (100 mg, yield: 29.15%). ESI: [M+H]⁺=256.2

### 4-(7-((1S,3R)-1-(((tert-butyldimethylsilyl)oxy)methyl)-4-oxo-5-azaspiro[2.4]heptan-5-yl)-9H-benzo[e]imidazo[2,1-c]pyrrolo[1,2-a][1,4]diazepin-12-yl) benzonitrile (48-E)

Using the procedures described above, compound 48-E was synthesized. ESI: [M+H]+=576.3

### 4-(7-((1S,3R)-1-(hydroxymethyl)-4-oxo-5-azaspiro[2.4]heptan-5-yl)-9H-benzo [e]imidazo[2,1-c]pyrrolo[1,2-a][1,4]diazepin-12-yl)benzonitrile (48-F)

To a solution of compound 48-E (80 mg, 0.14 mmol) in THE (2 mL) was added TBAF (1 M in THF, 0.5 mL) at room temperature. The mixture was stirred for 3 hour and then poured into H₂O (20 mL). The resulting mixture was extracted with DCM (20 mL×2). The combined organic layers were washed with brine (20 mL×2), dried over Na₂SO₄, filtered, concentrated to dryness. The crude product was purified by chromatography on silica gel eluting to get the desired product (60 mg, yield: 93.75%). ESI: [M+H]⁺=462.2

### 4-(7-((1S,3R)-1-((dimethylamino)methyl)-4-oxo-5-azaspiro[2.4]heptan-5-yl)-9H-benzo[e]imidazo[2,1-c]pyrrolo[1,2-a][1,4]diazepin-12-yl)benzonitrile (48)

Using the procedures described above, compound 48 was synthesized. ESI: [M+H]⁺=489.2; ¹H NMR (400 MHz, MeOH-d₄) δ 8.10 (d, J=2.4 Hz, 1H), 8.03 (s, 1H), 7.95 (dd, J=8.9, 2.5 Hz, 1H), 7.84-7.64 (m, 7H), 7.30 (s, 1H), 5.38 (s, 2H), 4.16-4.09 (m, 2H), 3.54 (dd, J=13.2, 4.9 Hz, 1H), 2.47-2.33 (m, 1H), 2.29-2.23 (m, 1H), 1.84-1.66 (m, 1H), 1.50 (dd, J=8.7, 4.6 Hz, 1H), 1.12-1.03 (m, 1H).

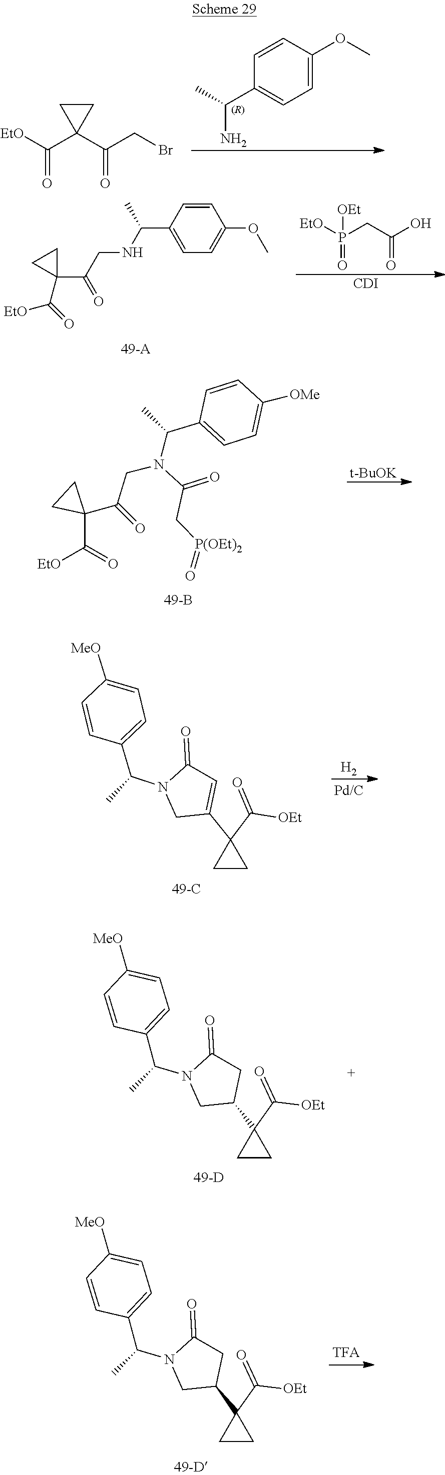

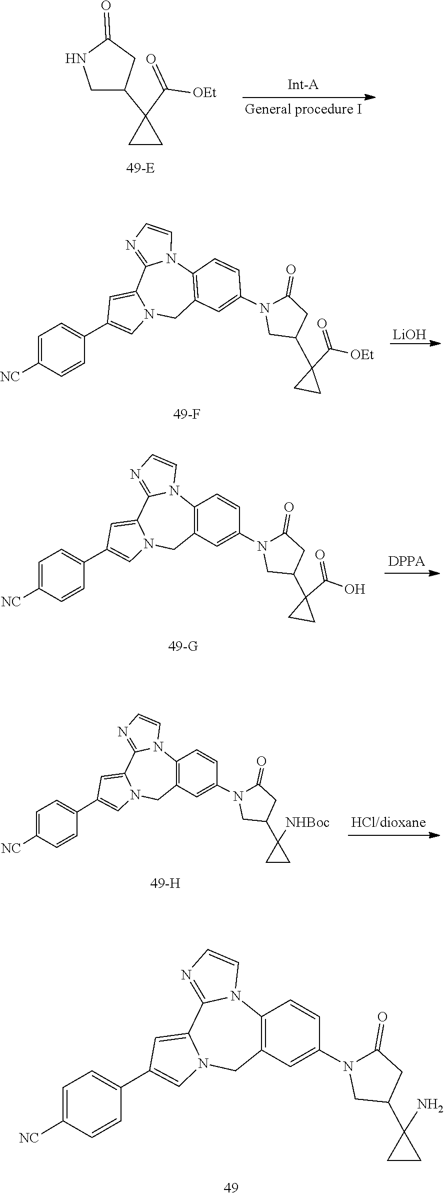

### Ethyl (R)-ethyl 1-(2-((1-(4-methoxyphenyl)ethyl)amino)acetyl)cyclopropane carboxylate (49-A)

To a solution of (R)-1-(4-methoxyphenyl)ethan-1-amine (6.45 g, 42.74 mmol) in CH₃CN (50 mL) was added a solution of ethyl 1-(2-bromoacetyl)cyclopropane-1-carboxylate (10 g, 42.74 mmol), TEA (6.5 mL, 47 mmol) in CH₃CN (10 mL) dropwise at 0° C. The mixture was stirred at 0° C. under N₂ for 3 h. After the reaction was completed, the mixture was concentrated and redissolved in EA (200 mL). The organic solution was extracted with 1N HCl (200 mL), the aqueous solution was washed with EA two times before neutralized with 1N NaOH (200 mL). The aq solution was extracted with EA. The organic layer was separated and dried over anhydrous Na₂SO₄, filtered, concentrated to give the desired product (6 g, yield: 45.9%). ESI: [M+H]⁺=306.2

### (R)-Ethyl 1-(2-(2-(diethoxyphosphoryl)-N-(1-(4-methoxyphenyl)ethyl) acetamido)acetyl)cyclopropanecarboxylate (49-B)

To a solution of 2-(diethoxyphosphoryl)acetic acid (4.6 g, 23.6 mmol) in THE (50 mL) was added CDI (4.1 g, 25.57 mmol) and stirred at 0° C. for 1 h. Compound 49-A (6 g, 19.67 mmol) in THE (10 mL) was added dropwise to above solution at 0° C., and the mixture was stirred at rt overnight. After the reaction was complete, the mixture was diluted with EA and washed with H₂O, 1N HCl (100 mL), NaHCO₃ (100 mL) and brine. The organic layer was dried over anhydrous Na₂SO₄, filtered and concentrated to dryness. The residue was purified by chromatography on silica gel eluting with PE:EA (10:1-1:1) to give the desired product (3.2 g, yield: 25.8%). ESI: [M+H]⁺=484.2.

### (R)-Ethyl 1-(1-(1-(4-methoxyphenyl)ethyl)-5-oxo-2,5-dihydro-1H-pyrrol-3-yl)cyclopropanecarboxylate (49-C)

To a solution of compound 49-B (3.2 g, 6.6 mmol) in toluene (30 mL) was added t-BuOK (1.48 g, 13.2 mmol) at 0° C. and the mixture was stirred at rt for 1 h. Citric acid (aq.) was added dropwise at 0° C. to adjust pH<7, and then extracted with EA three times. The combined organic layers were washed with NaHCO₃, brine, dried over anhydrous Na₂SO₄, filtered and concentrated to dryness. The residue was purified by chromatography on silica gel eluting with PE:EA (10:1-2:1) to give the desired product (1.3 g, yield: 59.8%). ESI[M+H]⁺=330.2

### Ethyl 1-((S)-1-((R)-1-(4-methoxyphenyl)ethyl)-5-oxopyrrolidin-3-yl) cyclopropane-1-carboxylate (049-D) and (049-D′)

To a solution of compound 49-C (1.3 g, 4 mmol) in EA (30 mL) was added Pd/C (200 mg) and the mixture was stirred at rt under H2 atmosphere overnight. The mixture was filtered through a pad of celite, concentrated and purified by chromatography on silica gel eluting with PE:EA (10:1-3:1) to give the desired product as mixture of two isomers (1 g, yield: 75%). ESI: [M+H]⁺=332.2. The two diastereomers were further separated by Prep-HPLC.

### Ethyl (S)-1-(5-oxopyrrolidin-3-yl)cyclopropane-1-carboxylate (49-E)

A solution of compound 49-D and 49-D′ (1:1, 470 mg, 1.42 mmol) in TFA (4 mL) was stirred at reflux for 3 h. The mixture was concentrated and purified by chromatography on silica gel eluting with DCM:MeOH (10:1-3:1) to give the desired product (250 mg, yield: 89.4%). ESI: [M+H]⁺=198.1

### Ethyl (R)-1-(1-(12-(4-cyanophenyl)-9H-benzo[e]imidazo[2,1-c]pyrrolo[1,2-a][1,4]diazepin-7-yl)-5-oxopyrrolidin-3-yl)cyclopropane-1-carboxylate (49-F)

Using the procedures described above, compound 49-F was synthesized. ESI: [M+H]⁺=518.2

### (R)-1-(1-(12-(4-cyanophenyl)-9H-benzo[e]imidazo[2,1-c]pyrrolo[1,2-a][1,4]diazepin-7-yl)-5-oxopyrrolidin-3-yl)cyclopropanecarboxylic acid (49-G)

To a solution of compound 49-F (100 mg, 0.19 mmol) in THE (5 mL) was added a solution of LiOH (46 mg, 1.9 mmol) in water (5 mL), and the mixture was stirred at 40° C. overnight. The reaction mixture was adjusted pH=2 with 6 M HCl and extracted with DCM/MeOH (10/1, 20 mL*3). The combined organic layers were washed with brine and dried over Na₂SO₄. The organic solution was filtered and concentrated in vacuo to give the desired product (90 mg, yield: 94.7%). ESI: [M+H]⁺=490.2

### tert-butyl (S)-(1-(1-(12-(4-cyanophenyl)-9H-benzo[e]imidazo[2,1-c]pyrrolo [1,2-a][1,4]diazepin-7-yl)-5-oxopyrrolidin-3-yl)cyclopropyl)carbamate (49-H)

To a solution of compound 49-G (90 mg, 0.18 mmol) in toluene (20 mL) was added TEA (37 mg, 0.39 mmol), DPPA (55 mg, 0.2 mmol) and stirred at reflux for 1.5 h. t-BuOH (5 mL) was added to above solution and stirred at 100° C. overnight. The reaction mixture was cooled to room temperature and concentrated to dryness. The residue was purified by Prep-TLC (DCM:MeOH=20:1) to give the desired compound (20 mg, yield: 19%). ESI: [M+H]⁺=561.3

### (S)-4-(7-(4-(1-aminocyclopropyl)-2-oxopyrrolidin-1-yl)-9H-benzo[e]imidazo [2,1-c]pyrrolo[1,2-a][1,4]diazepin-12-yl)benzonitrile (49)

To a solution of compound 49-G (20 mg, 0.035 mmol) in MeOH (5 mL) was added HCl-dioxane (4 M, 3 mL), the resulting solution was stirred at rt for 2 h. The mixture was concentrated and purified by Prep-HPLC to give the desired compound (11 mg, yield:68.5%). ESI: [M+H]⁺=461.5; ¹H NMR (400 MHz, CD₃OD): δ 8.12 (d, J=2.4 Hz, 1H), 8.03 (s, 1H), 7.84 (dd, J=8.9, 2.4 Hz, 1H), 7.78-7.69 (m, 7H), 7.29 (d, J=1.7 Hz, 1H), 5.38 (s, 2H), 4.08 (dd, J=9.8, 8.4 Hz, 1H), 3.81-3.69 (m, 1H), 3.04-2.93 (m, 1H), 2.80 (dd, J=17.0, 8.8 Hz, 1H), 2.57 (dd, J=17.0, 9.9 Hz, 1H), 1.10-1.02 (m, 4H).

Using the procedures described above, the following examples in Table 7 were synthesized.

TABLE 7

| | Ex. | | LC-MS |
|---|---|---|---|
| Structure | # | ¹H NMR | (M + H)⁺ |
| 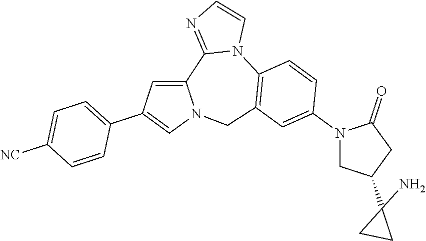 [SMILES: N#Cc1ccc(-c2cc3n(c2)Cc2cc(N4C[C@@H](C5(N)CC5)CC4=O)ccc2-n2ccnc2-3)cc1] | 50 | (400 MHz, MeOD): δ 8.17 (d, J = 2.2 Hz, 1H), 8.16 (d, J = 2.2 Hz, 1H), 7.95 (dd, J = 8.9, 2.1 Hz, 1H), 7.90 (d, J = 1.3 Hz, 1H), 7.86 (d, J = 2.1 Hz, 1H).7.81-7.70 (m, 5H), 7.39 (d, J = 1.3 Hz, 1H), 5.48 (s, 2H), 4.11 (t, J = 9.0 Hz, 1H), 3.80 (t, J = 9.1 Hz, 1H), 3.06 − 2.96 (m, 1H), 2.80 (dd, J = 17.0, 8.8 Hz, 1H), 2.57 (dd, J = | 461.5 |
| | | 17.0, 10.0 Hz, 1H), | |
| | | 1.10-1.05 (m, 4H). | |
| 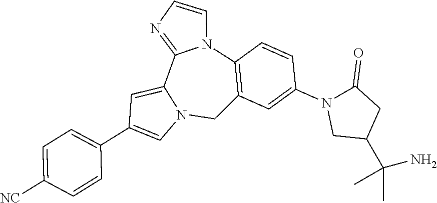 [SMILES: CC(C)(N)C1CC(=O)N(c2ccc3c(c2)Cn2cc(-c4ccc(C#N)cc4)cc2-c2nccn2-3)C1] | 51 | (400 MHz, CD₃OD): δ 8.13 (d, J = 2.2 Hz, 1H), 8.05 (d, J = 2.2 Hz, 1H), 7.89 (dd, J = 8.9, 2.5 Hz, 1H), 7.84 − 7.60 (m, 7H), 7.30 (d, J = 1.8 Hz, 1H), 5.39 (s, 2H), 4.18 − 4.00 (m, 1H), 4.00 − 3.82 (m, 1H), 2.95 − 2.80 (m, 1H), 2.80 − 2.61 (m, 2H), 1.45 (s, 3H), 1.42 (s, 3H), | 463.2 |
| 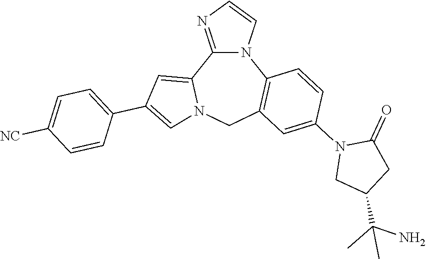 [SMILES: CC(C)(N)[C@H]1CC(=O)N(c2ccc3c(c2)Cn2cc(-c4ccc(C#N)cc4)cc2-c2nccn2-3)C1] | 52 | (400 MHz, CD₃OD) δ 8.13 (d, J = 2.3 Hz, 2H), 8.04 (d, J = 2.3 Hz, 2H), 7.92 (dd, J = 8.9, 2.5 Hz, 1H), 7.88 − 7.69 (m, 7H), 7.37 (d, J = 1.8 Hz, 1H), 5.43 (d, J = 6.4 Hz, 2H), 4.17 − 4.00 (m, 1H), 4.00 − 3.85 (m, 1H), 2.96 − 2.82 (m, 1H), 2.81 − 2.64 (m, 2H), 1.45 (s, 3H), 1.42 (s, 3H), | 463.2 |
| 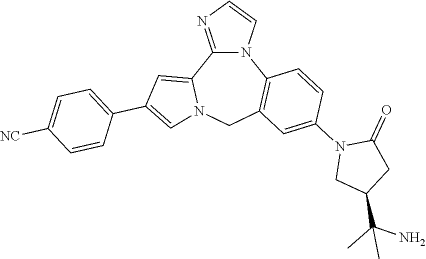 [SMILES: CC(C)(N)[C@@H]1CC(=O)N(c2ccc3c(c2)Cn2cc(-c4ccc(C#N)cc4)cc2-c2nccn2-3)C1] | 53 | (400 MHz, CD₃OD): δ 8.13 (d, J = 2.3 Hz, 2H), 8.04 (d, J = 2.3 Hz, 2H), 7.92 (dd, J = 8.9, 2.5 Hz, 1H), 7.88 − 7.69 (m, 7H), 7.37 (d, J = 1.8 Hz, 1H), 5.43 (d, J = 6.4 Hz, 2H), 4.17 − 4.00 (m, 1H), 4.00 − 3.85 (m, 1H), 2.96 − 2.82 (m, 1H), 2.81 − 2.64 (m, 2H), 1.45 (s, 3H), 1.42 (s, 3H), | 463.2 |

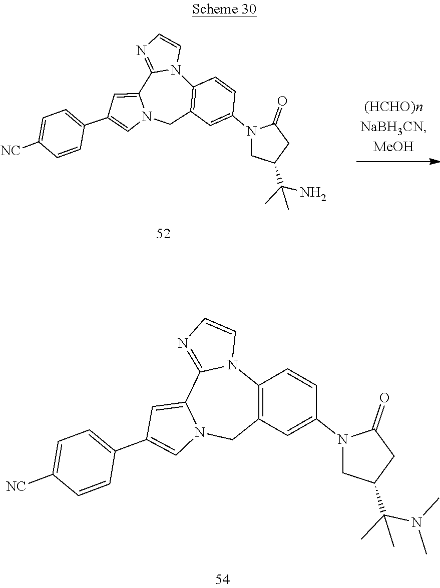 [SMILES: CC(C)(N)[C@H]1CC(=O)N(c2ccc3c(c2)Cn2cc(-c4ccc(C#N)cc4)cc2-c2nccn2-3)C1 ; CN(C)C(C)(C)[C@H]1CC(=O)N(c2ccc3c(c2)Cn2cc(-c4ccc(C#N)cc4)cc2-c2nccn2-3)C1]

### (S)-4-(7-(4-(2-aminopropan-2-yl)-2-oxopyrrolidin-1-yl)-9H-benzo[e]imidazo [2,1-c]pyrrolo[1,2-a][1,4]diazepin-12-yl)benzonitrile (54)

A solution of compound 52 (100 mg, 0.21 mmol) in 10 mL MeOH was charged with (HCHO)ₙ (63 mg 2.1 mmol) at 0° C. and then stirred at rt for 30 mins. NaBH₃CN (26 mg, 0.42 mmol) was added to the above mixture at rt and stirred for 1 h. The reaction mixture was concentrated and purified by Prep-HPLC to give the desired product (50 mg, yield: 50%) as a white solid. ESI: [M+H]⁺=491.2; ¹H NMR (400 MHz, DMSO-d₆): δ 8.00-7.89 (m, 2H), 7.81 (d, J=1.4 Hz, 1H), 7.79-7.67 (m, 5H), 7.58 (d, J=8.7 Hz, 1H), 7.23 (d, J=1.4 Hz, 1H), 7.04 (d, J=2.0 Hz, 1H), 5.24 (s, 2H), 3.93-3.68 (m, 2H), 2.87-2.70 (m, 1H), 2.56 (dd, J=17.5, 9.3 Hz, 3H), 2.16 (s, 6H), 0.93 (s, 3H), 0.87 (s, 3H).

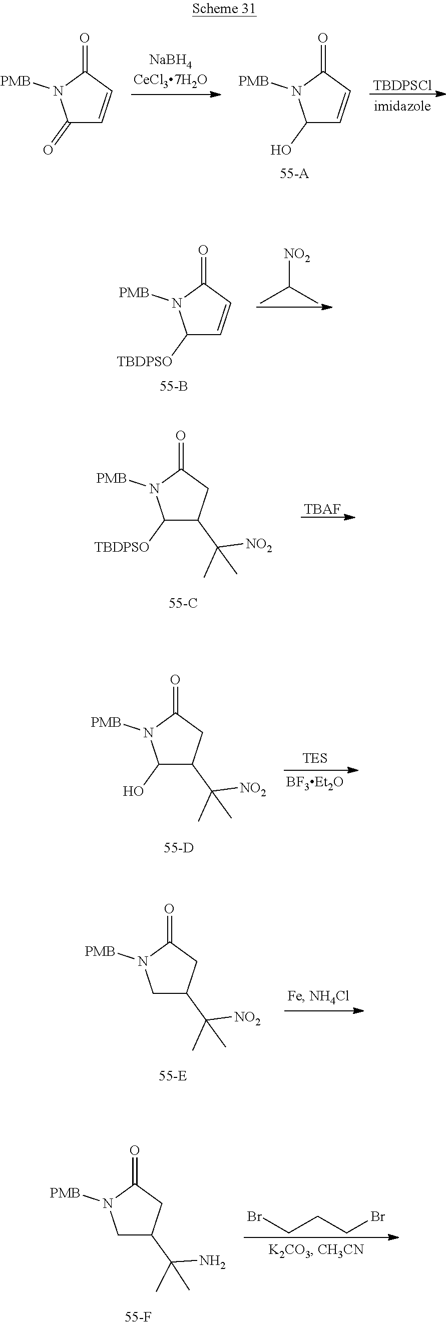

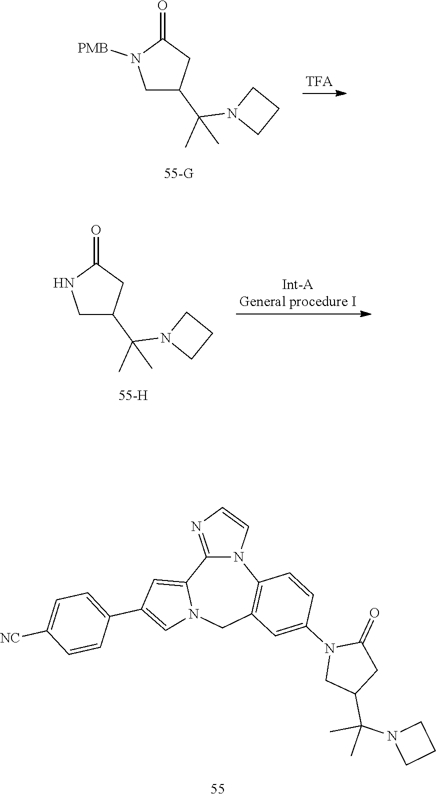

### 5-Hydroxy-1-(4-methoxybenzyl)-1,5-dihydro-2H-pyrrol-2-one (55-A)

To a solution of 1-(4-methoxybenzyl)-1H-pyrrole-2,5-dione (19 g, 87 mmol) in DCM/MeOH (5:1) (20 mL) was added NaBH₄ (3.97 g, 104 mmol) and CeCl₃·7H₂O (39 g, 104 mmol) at 0° C. The resulting mixture was stirred for 1 h before pouring into iced HCl (1M in water, 20 mL) and extracted with EA (300 mL*2). The combined organic layers were combined and washed with aqueous NaHCO₃ solution, brine, dried over Na₂SO₄, filtered, and concentrated to dryness. The residue was purified by chromatography on silica gel eluting with PE/EA (10/1-1/1) to obtain the desired compound as a white solid (13 g, yield: 68%). ESI: [M+H]⁺=220.1

### 5-((tert-butyldiphenylsilyl)oxy)-1-(4-methoxybenzyl)-1,5-dihydro-2H-pyrrol-2-one (55-B)

To a solution of compound 55-A (13 g, 86 mmol) a in DCM (200 mL) was added TBDPSCl (53.8 g, 95 mmol) and imidazole (8.8 g, 129 mmol) slowly at 0° C., and the resulting solution was stirred at 20° C. for 1 h. The mixture was quenched with water and extracted with DCM three times. The combined organic layers were washed with brine, dried over Na₂SO₄, filtered, and concentrated to dryness. The residue was purified by silica gel chromatography eluting with PE/EA (10/1-3/1) to give the desired compound (26 g, yield: 65%) as a colorless oil. ESI: [M+H]⁺=458.2

### 5-((tert-butyldiphenylsilyl)oxy)-1-(4-methoxybenzyl)-4-(2-nitropropan-2-yl)pyrrolidin-2-one (55-C)

To a solution of compound 52-B (2.0 g, 4.4 mmol) in 2-nitropropane (50 mL) was added DBU (1.3 g, 8.8 mmol), and the resulting solution was stirred at rt for 48 h. The mixture was concentrated and purified by chromatography on silica gel eluting with PE/EA (10/1-1/1) to give the desired compound (980 mg, yield: 42%). ESI: [M+H]⁺=547.3

### 5-Hydroxy-1-(4-methoxybenzyl)-4-(2-nitropropan-2-yl)pyrrolidin-2-one (55-D)

To a solution of compound 55-C (980 mg, 0.18 mmol) in THE (10 mL) was added TBAF in THE (1 M, 4 mL) at rt and the resulting solution was stirred at rt for 1 h. The reaction mixture was diluted with NH₄Cl solution and extracted with DCM (20 mL*2). The combined organic layers were dried over Na₂SO₄, filtered and concentrated to dryness. The residue was purified by silica gel chromatography eluting with PE/EA(10/1-1/1) to give the desired compound as a yellow oil (500 mg, yield: 89%). ESI: [M+H]⁺=309.1

### 1-(4-Methoxybenzyl)-4-(2-nitropropan-2-yl)pyrrolidin-2-one (55-E)

To a solution of compound 52-D (480 mg, 1.56 mmol) in DCM (30 mL) was added BF₃·Et₂O (444 mg, 3.12 mmol) and triethylsilane (364 mg, 3.12 mmol) at 0° C. under N₂ atmosphere, and the resulting solution was stirred at 20° C. for 8 h. The reaction was quenched with H₂O, and the mixture was extracted with EA (20 mL*2). The combined organic layers were dried over Na₂SO₄, filtered and concentrated to dryness. The residue was purified by chromatography on silica gel eluting with PE/EA (10/1-1/1) to give the desired product as a yellow oil (300 mg, yield: 66%). ESI: [M+H]⁺=293.1

### 4-(2-Aminopropan-2-yl)-1-(4-methoxybenzyl)pyrrolidin-2-one (55-F)

To a suspension of compound 55-E (600 mg, 2.1 mmol) in 10 mL of H₂O and 10 mL of EtOH was added Fe (1.2 g, 21 mmol,) and NH₄Cl (535 mg, 10.5 mmol). The reaction mixture was then stirred 80° C. for 2 h, cooled to room temperature, and filtered. The filtrate was concentrated, and the residue was purified by chromatography on silica gel to obtain the desired compound as a yellow solid (440 mg, yield: 80%). ESI: [M+H]⁺=263.2

### 4-(2-(Azetidin-1-yl)propan-2-yl)-1-(4-methoxybenzyl)pyrrolidin-2-one (55-G)

The mixture of compound 55-F (440 mg, 1.5 mmol), 1,3-dibromopropane (370 mg, 1.8 mmol) and K₂CO₃ (620 mg, 4.5 mmol) in 20 mL CH₃CN was stirred at 60° C. overnight. The reaction mixture was cooled to room temperature, filtered, and then concentrated to dryness. The residue (450 mg, crude) was used directly in the next step. ESI: [M+H]⁺=303.2

### 4-(2-(Azetidin-1-yl)propan-2-yl)pyrrolidin-2-one (55-H)

The mixture of compound 55-G (450 mg, crude) in TFA (10 mL) was placed at 140° C. for 2 h in a microwave reactor. The reaction mixture was concentrated to dryness under reduced pressure, and the residue was washed with Et₂O (10 mL*3) to give the desired product as a yellow solid (270 mg, yield: 99%). ESI: [M+H]⁺=183.1

### 4-(7-(4-(2-(Azetidin-1-yl)propan-2-yl)-2-oxopyrrolidin-1-yl)-9H-benzo[e]imidazo[2,1-c]pyrrolo[1,2-a][1,4]diazepin-12-yl)benzonitrile (55)

Using the procedures described above, compound 52 was synthesized as a white solid (2 mg, yield: 2%). ESI: [M+H]⁺=503.2; ¹H NMR (400 MHz, CD₃OD): δ 7.96 (d, J=2.2 Hz, 1H), 7.77 (dd, J=8.8, 2.2 Hz, 1H), 7.74-7.43 (m, 7H), 7.18 (d, J=8.7 Hz, 1H), 6.89 (d, J=8.7 Hz, 1H), 5.12 (s, 2H), 3.92-3.81 (m, 2H), 3.53 (t, J=6.7 Hz, 5H), 3.35 (s, 1H), 2.58 (s, 2H), 2.14 (dd, J=14.5, 7.2 Hz, 2H), 1.09 (s, 6H).

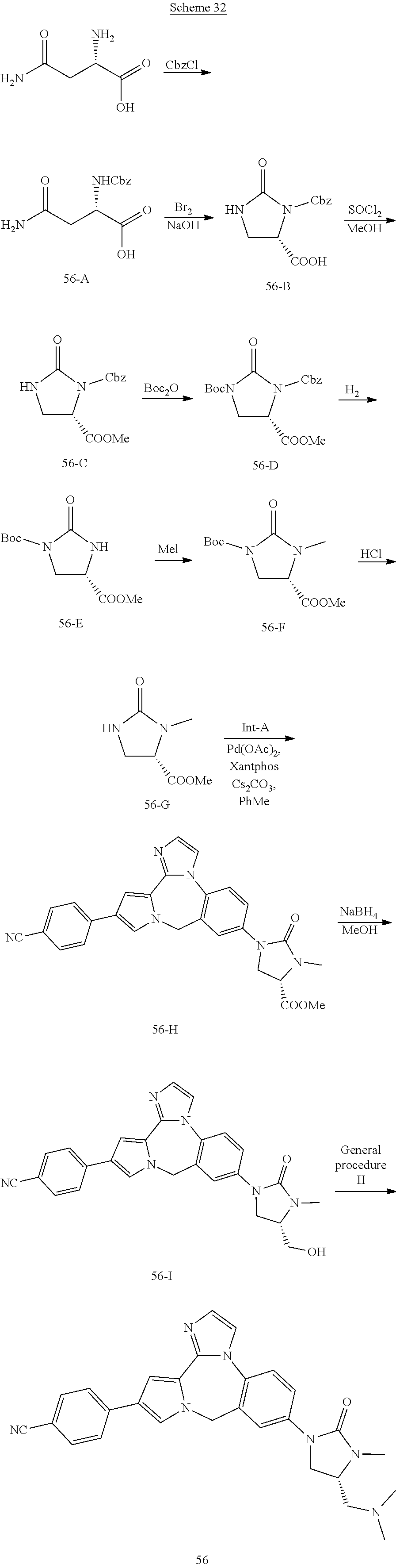

### ((Benzyloxy)carbonyl)-L-asparagine (056-A)

To a 0° C. solution of L-asparagine (11.2 g, 91.7 mmol) in 1,4-dioxane (112 mL) was added Na₂CO₃ (2N, 200 mL), then CbzCl (16.4 g, 96.3 mmol) was added dropwise. The resulting mixture was stirred at room temperature for 18 h and then washed with MTBE. The aqueous layer was acidified with HCl (4N) to adjust pH<1, the white solid was precipitated which was collected by filtration and dried in vacuo to give the desired compound (19 g, yield: 77.9%) as a white solid. ESI: [M+H]⁺=267.1

### (S)-3-((Benzyloxy)carbonyl)-2-oxoimidazolidine-4-carboxylic acid (56-B)

To a 0° C. solution of NaOH (2.98 g, 74.4 mmol) in H₂O (60 mL) was slowly added Br₂ (3.65 g, 24.3 mmol). Compound 56-A (6.0 g, 22.5 mmol) was added to above solution at 0° C., and the resulting mixture turned clear and colorless. The resulting solution was stirred at 55° C. for 3 h and cooled to room temperature. After washing with MTBE, the solution was acidified with HCl (4N) to adjust pH<1. A white solid was collected by filtration and dried in vacuo to give the desired compound (4 g, yield: 66.7%). ESI: [M+H]⁺=265.1

### (S)-1-Benzyl-5-methyl 2-oxoimidazolidine-1,5-dicarboxylate (56-C)

To a 0° C. solution of compound 56-B (2.0 g, 7.55 mmol) in MeOH (20 mL) was slowly added SOCl₂ (2.7 g, 22.65 mmol). The resulting mixture was stirred at 25° C. for 3 h. The mixture was quenched with sat'd NaHCO₃ aqueous solution and then extracted with EA three times. The combined organic layers were dried over anhydrous Na₂SO₄, filtered and concentrated under reduced pressure. The residue was purified by silica gel chromatography eluting with PE/EA (10/1-1/2) to give the desired compound as a white solid (1.7 g, yield: 81%). ESI: [M+H]⁺=279.1

### (S)-3-Benzyl-1-tert-butyl-4-methyl-2-oxoimidazolidine-1,3,4-tricarboxylate (56-D)

To a 0° C. solution of compound 56-C (0.23 g, 0.82 mmol) and DMAP (104 mg, 0.82 mmol) in CH₃CN (20 mL) was slowly added Boc₂O (0.36 g, 1.65 mmol). The resulting mixture was stirred at 50° C. for 3 h. After starting material was consumed, water was added and the mixture was extracted with EA three times. The combined organic layers were dried over anhydrous Na₂SO₄, filtered and concentrated under reduce pressure to dryness. The residue was purified by chromatography on silica gel eluting with PE/EA (10/1-1/5) to give the desired product as a white solid (0.26 g, yield: 83.6%). ESI [M+H]⁺=379.1

### (S)-1-tert-butyl 4-methyl-2-oxoimidazolidine-1,4-dicarboxylate (56-E)

To a solution of compound 56-D (0.26 g, 0.69 mmol) in MeOH (10 mL) was added Pd/C (10%, 50 mg). The resulting mixture was stirred at 50° C. for 16 h at 50 psi under H2 atmosphere. After starting material was consumed, the mixture was cooled and filtered through a pad of celite, and the filtrate was concentrated under reduced pressure to give the desired product as a solid (0.16 g, yield: 94.1%). ESI: [M+H]⁺=245.1

### (S)-1-tert-butyl 4-methyl 3-methyl-2-oxoimidazolidine-1,4-dicarboxylate (56-F)

To a 0° C. solution of compound 56-E (0.16 g, 0.65 mmol) in anhydrous THE (10 mL) was carefully added NaH (60%, 39 mg, 0.98 mmol). The resulting mixture was stirred at 0° C. for 0.5 h. MeI (139 mg, 0.98 mmol) was added dropwise to above solution, and the resulting mixture was stirred at room temperature for 2 h. After starting material was consumed, the mixture was quenched with sat'd NH₄Cl and extracted with EA three times. The combined organic layers were washed with brine, dried over anhydrous Na₂SO₄ and concentrated under reduced pressure to dryness. The residue was purified by silica gel chromatography eluting with PE/EA (10/1-1/5) to give the desired compound (0.12 g, yield: 75%). ESI: [M+H]⁺=259.1

### (S)-Methyl 3-methyl-2-oxoimidazolidine-4-carboxylate (56-G)

To a 0° C. solution of compound 56-G (0.5 g, 2 mmol) in MeOH (10 mL) was added HCl in 1,4-dioxane (4N, 10 mL). The resulting mixture was stirred at 20° C. for 6 h. The mixture was concentrated in vacuum to give the desired product as a yellow solid (0.28 g, yield: 88%). ESI: [M+H]⁺=159.1

### (S)-Methyl 1-(12-(4-cyanophenyl)-9H-benzo[e]imidazo[2,1-c]pyrrolo [1,2-a][1,4]diazepin-7-yl)-3-methyl-2-oxoimidazolidine-4-carboxylate (56-H)

To a solution of Int-A (120 mg, 0.3 mmol) in toluene (10 mL) was added 56-G (48 mg, 0.3 mmol), Pd(OAc)₂ (7 mg, 0.03 mmol), Xantphos (35 mg, 0.06 mmol) and Cs₂CO₃ (293 mg, 0.9 mmol). The reaction system was purged with argon gas three times and stirred for 18 h at 110° C. The reaction mixture was cooled to room temperature, diluted with DCM (30 mL), and filtered. The filtrate was concentrated to dryness and purified by flash chromatography eluting with DCM/MeOH (100/1-20/1) to give the desired compound (18 mg, yield: 12.5%). ESI: [M+H]⁺=479.2

### (S)-4-(7-(4-(Hydroxymethyl)-3-methyl-2-oxoimidazolidin-1-yl)-9H-benzo[e]imidazo[2,1-c]pyrrolo[1,2-a][1,4]diazepin-12-yl)benzonitrile (56-I)

To a 0° C. solution of compound 56-H (18 mg, 0.03 mmol) in EtOH (10 mL) was added NaBH₄ (6 mg, 0.15 mmol). The reaction was stirred at 20° C. for 2 h. After starting material was consumed, the mixture was quenched with NH₄Cl aqueous solution and extracted with EA. The organic layer was washed with brine, dried over Na₂SO₄, filtered and concentrated to dryness in vacuo. The residue was purified by flash chromatography eluting with DCM/MeOH (100/1-20/1) to give (the desired compound (15 mg, yield: 95%). ESI: [M+H]⁺=451.2

### (R)-4-(7-(4-((Dimethylamino)methyl)-3-methyl-2-oxoimidazolidin-1-yl)-9H-benzo[e]imidazo[2,1-c]pyrrolo[1,2-a][1,4]diazepin-12-yl)benzonitrile (56)

Using the procedures described above, compound 56 was synthesized. ESI: [M+H]⁺=478.2; ¹H-NMR (400 MHz, CD₃OD): δ 8.03 (m, 2H), 7.74 (m, 8H), 7.30 (s, 1H), 5.35 (s, 2H), 4.26 (t, J=9.2 Hz, 1H), 4.21-4.10 (m, 1H), 3.96-3.86 (m, 1H), 3.60 (m, 1H), 3.49-3.38 (m, 1H), 3.02 (s, 6H), 2.96 (s, 3H).

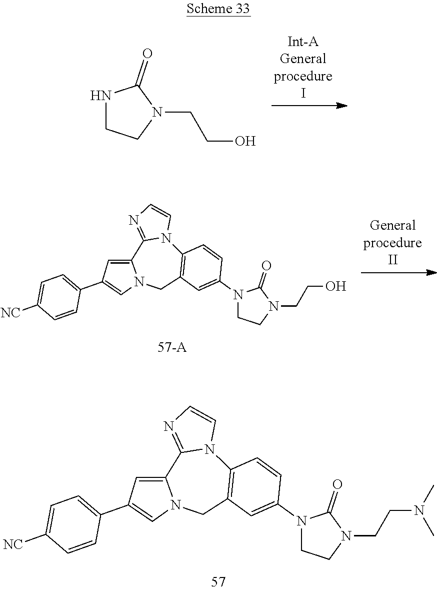 [SMILES: O=C1NCCN1CCO ; N#Cc1ccc(-c2cc3n(c2)Cc2cc(N4CCN(CCO)C4=O)ccc2-n2ccnc2-3)cc1 ; CN(C)CCN1CCN(c2ccc3c(c2)Cn2cc(-c4ccc(C#N)cc4)cc2-c2nccn2-3)C1=O]

### 4-(7-(3-(2-Hydroxyethyl)-2-oxoimidazolidin-1-yl)-9H-benzo[e]imidazo[2, 1-c]pyrrolo[1,2-a][1,4]diazepin-12-yl)benzonitrile (57-A)

Using the procedures described above, compound 57-A was synthesized. ESI: [M+H]⁺=451.2

### 4-(7-(3-(2-(Dimethylamino)ethyl)-2-oxoimidazolidin-1-yl)-9H-benzo[e]imidazo[2,1-c]pyrrolo[1,2-a][1,4]diazepin-12-yl)benzonitrile (57)

Using the procedures described above, compound 57 was synthesized. ESI: [M+H]⁺=478.2; ¹H NMR (400 MHz, CD₃OD): δ 8.05 (d, J=2.1 Hz, 1H), 8.00 (d, J=2.5 Hz, 1H), 7.84 (dd, J=9.0, 2.5 Hz, 1H), 7.79 (d, J=1.8 Hz, 1H), 7.78-7.70 (m, 6H), 7.70 (s, 1H), 7.31 (d, J=1.8 Hz, 1H), 5.37 (s, 2H), 4.01 (dd, J=9.2, 6.8 Hz, 2H), 3.73-3.69 (m, 2H), 3.66 (dd, J=9.1, 6.9 Hz, 2H), 3.44-3.39 (m, 3H), 2.99 (s, 6H).

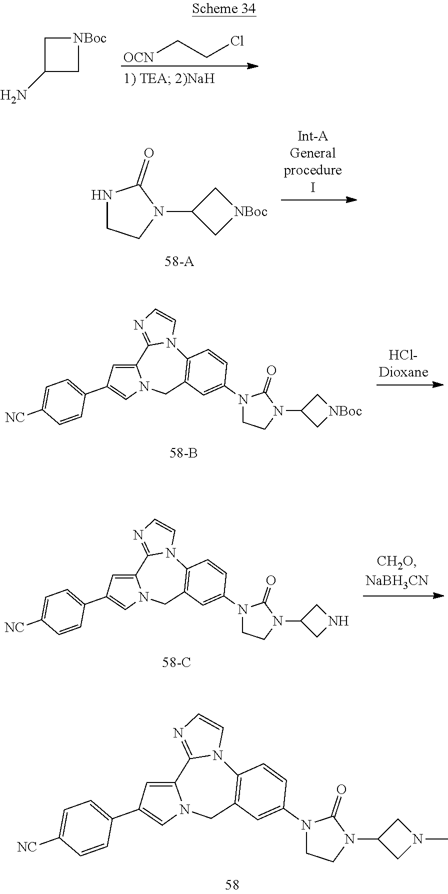

### tert-butyl 3-(2-oxoimidazolidin-1-yl)azetidine-1-carboxylate (58-A)

To a solution of tert-butyl 3-aminoazetidine-1-carboxylate (1 g, 5.8 mmol) and TEA (0.81 mL, 5.8 mmol) in dioxane (30 mL) was added 2-chloroethyl isocyanate (0.54 mL, 6.38 mmol) portion wise over a period of 3 minutes. This mixture was allowed to stir at ambient temperature for 18 h. Sodium hydride (60% dispersion in mineral oil, 350 mg) was then added to above solution. The resulting reaction mixture was stirred at ambient temperature for 48 h and quenched by the slow addition of water (5 mL). The mixture was concentrated under reduced pressure to 30 mL, and the resulting solution was directly purified by chromatography on silica gel (DCM:MeOH=10:1) to give the desired product (600 mg, yield: 43%). ESI: [M+H]⁺=242.1

### tert-butyl 3-(3-(12-(4-cyanophenyl)-9H-benzo[e]imidazo[2,1-c]pyrrolo [1,2-a][1,4]diazepin-7-yl)-2-oxoimidazolidin-1-yl)azetidine-1-carboxylate (58-B)

Using the procedures described above, compound 052 was synthesized. ESI: [M+H]⁺=562.2

### 4-(7-(3-(Azetidin-3-yl)-2-oxoimidazolidin-1-yl)-9H-benzo[e]imidazo[2, 1-c]pyrrolo[1,2-a][1,4]diazepin-12-yl)benzonitrile (58-C)

To a solution of compound 58-B (30 mg, 0.05 mmol) in MeOH (5 mL) was added HCl-dioxane (4N, 5 mL) and the resulting solution was stirred at 25° C. overnight. After the reaction was complete, the mixture was concentrated and redissolved in MeOH. The solution was neutralized by basic resin and concentrated to give the desired product (20 mg, yield: 81%). ESI: [M+H]⁺=462.2

### 4-(7-(3-(1-Methylazetidin-3-yl)-2-oxoimidazolidin-1-yl)-9H-benzo[e]imidazo[2,1-c]pyrrolo[1,2-a][1,4]diazepin-12-yl)benzonitrile (58)

Using the procedures described above, compound 58 was synthesized. ESI: [M+H]⁺=476.2; ¹H-NMR (400 MHz, CD₃OD): δ 8.06 (d, J=2.0 Hz, 1H), 7.98 (s, 1H), 7.79 (d, J=1.1 Hz, 1H), 7.74-7.69 (m, 7H), 7.32 (d, J=1.7 Hz, 1H), 5.38 (s, 2H), 4.69 (dt, J=15.6, 7.7 Hz, 2H), 4.54-4.52 (s, 2H), 4.29 (s, 1H), 4.01 (t, J=7.9 Hz, 2H), 3.76-3.66 (m, 2H), 3.05 (s, 3H).

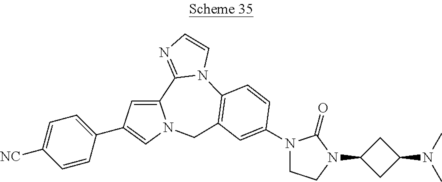 [SMILES: CN(C)[C@H]1C[C@@H](N2CCN(c3ccc4c(c3)Cn3cc(-c5ccc(C#N)cc5)cc3-c3nccn3-4)C2=O)C1]

Using the procedures described above, compound 59 was synthesized. ESI: [M+H]+=504.2; ¹H NMR (400 MHz, CD₃OD): δ 7.92 (s, 1H), 7.91 (s, 1H), 7.79 (dd, J=8.9, 2.6 Hz, 1H), 7.74 (d, J=8.6 Hz, 2H), 7.71-7.63 (m, 4H), 7.57 (s, 1H), 7.22 (d, J=1.7 Hz, 1H), 5.30 (s, 2H), 4.60-4.46 (m, 1H), 3.96 (dt, J=15.1, 6.8 Hz, 3H), 3.78-3.62 (m, 2H), 2.94 (s, 6H), 2.93-2.75 (m, 2H), 2.73-2.56 (m, 2H).

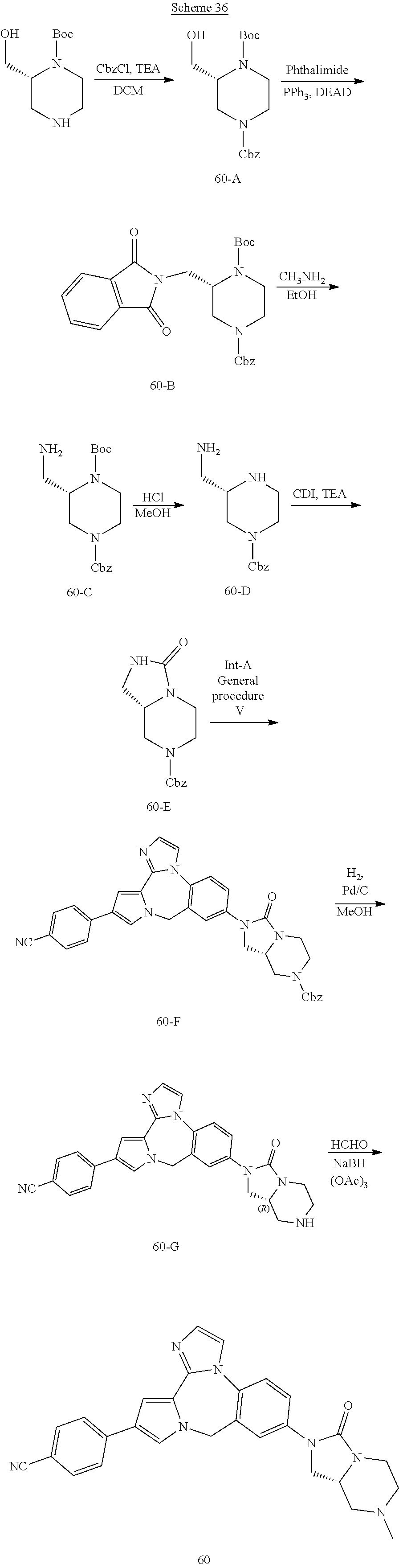

### (R)-4-Benzyl 1-tert-butyl 2-(hydroxymethyl)piperazine-1,4-dicarboxylate (60-A)

To a stirred solution of tert-butyl (R)-2-(hydroxymethyl)piperazine-1-carboxylate (500 mg, 2.31 mmol) and triethylamine (707 mg, 3.93 mmol) in DCM (20 mL) was added CbzCl (787 mg, 4.62 mmol) dropwise at 0° C. The mixture was stirred at room temperature overnight and then washed with brine, dried over Na₂SO₄, filtered and concentrated. The residue was purified by silica gel chromatography eluting with PE/EA (10/1-3/1) to give the desired compound (660 mg, yield: 82%). ESI: [M+H]⁺=351.2

### (S)-4-Benzyl 1-tert-butyl 2-((1,3-dioxoisoindolin-2-yl)methyl)piperazine-1,4-dicarboxylate (60-B)

To a stirred mixture of compound 60-A (660 mg, 1.88 mmol), PPh3 (1.5 g, 5.64 mmol), isoindoline-1,3-dione (550 mg, 3.76 mmol) in anhydrous THE (20 mL) was added DEAD (980 mg, 5.64 mmol) dropwise at 0° C. The mixture was stirred at room temperature for 1 h and then concentrated in vacuo. The resulting residue was purified by silica gel chromatography eluting with PE/EA (100/1-10/1) to give the desired product as a white solid (900 mg, yield: 99%). ESI: [M+H]⁺=480.

### (S)-4-Benzyl 1-tert-butyl 2-(aminomethyl)piperazine-1,4-dicarboxylate (60-C

To a stirred mixture of compound 60-B (900 mg, 1.88 mmol) in ethanol (20 mL) was added methanamine (580 mg, 18.8 mmol). The mixture was stirred at 90° C. for 3 h. The reaction mixture was concentrated to dryness, dissolved in ether and filtered. The filtrate was concentrated to give the desired compound as a white solid (650 mg, yield: 99%, crude). ESI: [M+H]⁺=350.2

### Benzyl (S)-3-(aminomethyl)piperazine-1-carboxylate (60-D)

To a stirred mixture of compound 60-C (650 mg, 1.88 mmol) in MeOH (20 mL) was added hydrochloric acid (2 mL, 24.0 mmol). The mixture was stirred at room temperature for 3 h and then concentrated to give the desired product as a hydrochloric acid salt as a white solid (470 mg, yield: 78%, crude). ESI: [M+H]⁺=250.2

### (S)-Benzyl 3-oxohexahydroimidazo[1,5-a]pyrazine-7(1H)-carboxylate (60-E)

To a stirred mixture of compound 60-D (470 mg, 1.47 mmol), TEA (575 mg, 5.64 mmol) in DCM (20 mL) was added CDI (550 mg, 3.39 mmol). The mixture was stirred at room temperature for 3 h and then concentrated. The residue was purified by silica gel chromatography eluting DCM/MeOH (100/1-10/1) to give the desired compound as a white solid (250 mg, yield: 62%). ESI: [M+H]⁺=276.1

### (S)-Benzyl-2-(12-(4-cyanophenyl)-9H-benzo[e]imidazo[2,1-c]pyrrolo[1,2-a][1,4]diazepin-7-yl)-3-oxohexahydroimidazo[1,5-a]pyrazine-7(1H)-carboxylate (60-F)

Using the procedures described above, compound 60-F was synthesized. ESI: [M+H]⁺=596.2

### (R)-4-(7-(3-Oxohexahydroimidazo[1,5-a]pyrazin-2(3H)-yl)-9H-benzo[e]imidazo [2,1-c]pyrrolo[1,2-a][1,4]diazepin-12-yl)benzonitrile (60-G)

To a stirred solution of compound 60-F (170 mg, 0.28 mmol) in MeOH (20 mL) was added Pd/C (30 mg). The mixture was stirred under hydrogen atmosphere at room temperature for 3 days. The reaction mixture was filtered, and the filtrate was concentrated to give the desired product (130 mg, yield: 99%, crude). ESI: [M+H]⁺=462.2

### (R)-4-(7-(7-Methyl-3-oxohexahydroimidazo[1,5-a]pyrazin-2(3H)-yl)-9H-benzo[e]imidazo[2,1-c]pyrrolo[1,2-a][1,4]diazepin-12-yl)benzonitrile (60)

To a stirred mixture of compound 60-G (130 mg, 0.28 mmol) in methanol (10 mL) was added paraformaldehyde (100 mg, 3.3 mmol) and sodium triacetoxyborohydride (190 mg, 0.89 mmol). The mixture was stirred at room temperature for 1 hour and then diluted with NaHCO₃ before extraction with DCM three times The combined organic layers were concentrated, and the residue was purified by Prep-HPLC to give the desired compound as a white solid (10 mg). ESI: [M+H]⁺=476.2; ¹H NMR (400 MHz, CD₃OD): δ 8.04 (d, J=2.0 Hz, 1H), 8.00 (d, J=2.5 Hz, 1H), 7.82 (dt, J=5.4, 2.7 Hz, 1H), 7.79 (t, J=2.6 Hz, 1H), 7.77-7.73 (m, 2H), 7.73-7.68 (m, 4H), 7.31 (d, J=1.8 Hz, 1H), 5.36 (s, 2H), 4.27-4.10 (m, 3H), 3.75 (dd, J=9.1, 3.4 Hz, 1H), 3.63 (t, J=13.1 Hz, 1H), 3.53 (d, J=12.0 Hz, 1H), 3.38 (m, 1H), 3.16-3.00 (m, 2H), 2.96 (s, 3H).

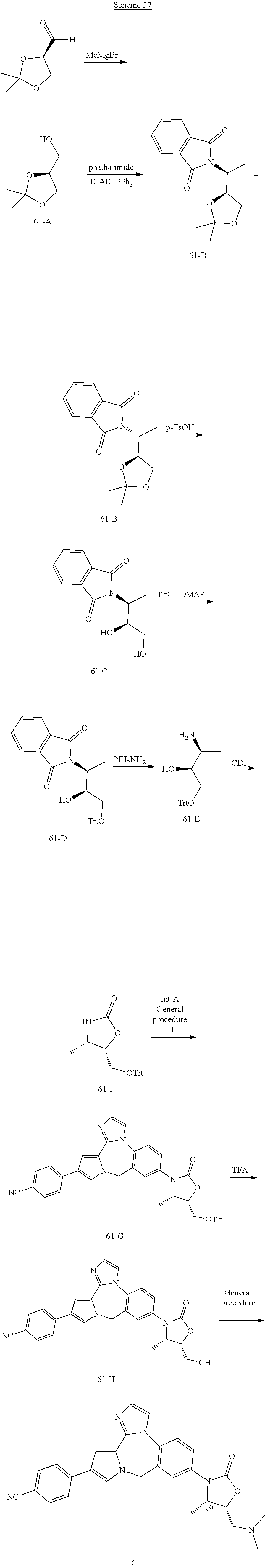

### 1-((R)-2,2-Dimethyl-1,3-dioxolan-4-yl)ethan-1-ol (61-A)

To a stirred mixture of (R)-2,2-dimethyl-1,3-dioxolane-4-carbaldehyde (12.0 g, 92.3 mmol) in tetrahydrofuran (200 mL) was added methylmagnesium bromide in diethyl ether (46 mL, 138 mmol, 3N) dropwise under ice-cooling, and the resulting mixture was stirred at room temperature for 16 h. The reaction mixture was quenched with aqueous saturated ammonium chloride solution and extracted with ethyl acetate. The combined organic layers were dried with sodium sulfate, filtered and concentrated to give the desired product (12 g, yield: 88%, crude).

### 2-((S)-1-((S)-2,2-Dimethyl-1,3-dioxolan-4-yl)ethyl)isoindoline-1,3-dione (61-B) and 2-((R)-1-((S)-2,2-dimethyl-1,3-dioxolan-4-yl)ethyl)isoindoline-1,3-dione (61-B′)

To a stirred mixture of isoindoline-1,3-dione (14.5 g, 97.0 mmol), compound 58-A (12 g, 82.0 mmol), PPh3 (25.4 g, 97.0 mmol) in THF (200 mL) was added DEAD (19.6 g, 97.0 mmol) dropwise at 0° C. The mixture was stirred at room temperature for 24 h. The reaction mixture was then diluted with water and extracted with ethyl acetate. The combined organic layers was dried with sodium sulfate, concentrated and purified by silica gel chromatography (PE:EA=1:1) to give compound 61-B′ (4 g, yield: 18%) and 61-B (1 g, yield: 4.5%).

### 2-((R)-1-((S)-2,2-dimethyl-1,3-dioxolan-4-yl)ethyl)isoindoline-1,3-dione (61-B′)

¹H NMR (400 MHz, CDCl₃): δ 7.83 (m, 2H), 7.73-7.68 (m, 2H), 4.76-4.67 (m, 1H), 4.40-4.28 (m, 1H), 4.14 (dd, J=8.6, 6.2 Hz, 1H), 3.83 (dd, J=8.6, 5.1 Hz, 1H), 1.40 (t, J=5.7 Hz, 3H), 1.37 (s, 4H), 1.29 (s, 3H).

### 2-((S)-1-((S)-2,2-dimethyl-1,3-dioxolan-4-yl)ethyl)isoindoline-1,3-dione (61-B)

¹H NMR (400 MHz, CDCl₃): δ 7.87-7.80 (m, 2H), 7.76-7.69 (m, 2H), 4.72 (dt, J=9.2, 6.0 Hz, 1H), 4.33 (dq, J=9.2, 6.9 Hz, 1H), 3.96 (dd, J=8.4, 6.2 Hz, 1H), 3.68 (dt, J=13.7, 6.9 Hz, 1H), 1.56 (d, J=6.9 Hz, 3H), 1.44 (s, 3H), 1.38 (d, J=6.4 Hz, 3H).

### 2-((2S,3S)-3,4-Dihydroxybutan-2-yl)isoindoline-1,3-dione (061-C)

To a stirred mixture of compound 61-B (1 g, 3.63 mmol) in methanol (20 mL) was added p-toluenesulfonic acid (1.60 g, 9.08 mmol). The mixture was stirred at 60° C. for 4 h and then cooled to room temperature. The reaction mixture was diluted with water and extracted with ethyl acetate. The combined organic layers were dried with sodium sulfate, concentrated and purified by silica gel chromatography (PE:EA=1:10) to give the desired product (850 mg, yield: 99%). ESI: [M+H]⁺=236.1

### 2-((2S,3S)-3-Hydroxy-4-(trityloxy)butan-2-yl)isoindoline-1,3-dione (61-D)

To a stirred mixture of compound 61-C, (chloromethanetriyl)tribenzene (1.27 g, 4.59 mmol), TEA (780 mg, 7.64 mmol) in DCM (30 mL) was added DMAP (24 mg, 0.20 mmol). The mixture was stirred at room temperature for 16 h. The reaction mixture was diluted with EA and washed with water The organic layer was dried with sodium sulfate, concentrated and purified by silica gel chromatography (PE:EA=1:1) to give the desired product (1.5 g, yield: 87%). ESI: [M+H]⁺=478.2

### (2S,3S)-3-Amino-1-(trityloxy)butan-2-ol (61-E)

To a stirred mixture of compound 61-D (1.5 g, 3.14 mmol) in ethanol (50 mL) was added hydrazine hydrate (1.5 g, 31.4 mmol). The mixture was stirred at 80° C. for 1 h and cooled to room temperature. The reaction mixture was filtered, and the filtrate was dried with sodium sulfate, filtered and concentrated to give the desired product (1 g, yield: 92%). ESI: [M+H]⁺=348.2

### (4S,5S)-4-Methyl-5-((trityloxy)methyl)oxazolidin-2-one (61-F)

To a stirred mixture of compound 61-E (1.0 g, 2.88 mmol) in tetrahydrofuran (30 mL) was added N,N′-carbonyldiimidazole (740 mg, 4.32 mmol). The mixture was stirred at 65° C. for 1 h and then concentrated to dryness. The residue was purified by silica gel chromatography (PE:EA=1:10) to give the desired compound (700 mg, yield: 65%). ESI: [M+H]⁺=374.2

### 4-(7-((4S,5S)-4-Methyl-2-oxo-5-((trityloxy)methyl)oxazolidin-3-yl)-9H-benzo[e]imidazo[2,1-c]pyrrolo[1,2-a][1,4]diazepin-12-yl)benzonitrile (61-G)

Using the procedures described above, compound 061-F was synthesized. ESI: [M+H]+=694.3

### 4-(7-((4S,5S)-5-(Hydroxymethyl)-4-methyl-2-oxooxazolidin-3-yl)-9H-benzo [e]imidazo[2,1-c]pyrrolo[1,2-a][1,4]diazepin-12-yl)benzonitrile (61-H)

To a stirred solution of compound 61-G (200 mg, 0.29 mmol) in DCM (10 mL) was added TFA (160 mg, 1.44 mmol). The mixture was stirred at room temperature for 1 hour and then diluted with DCM. The organic solution was washed with water, dried with sodium sulfate, filtered and concentrated to dryness. The residue was purified by silica gel chromatography (DCM:CH₃OH=10:1) to give the desired compound (100 mg, yield: 77%). ESI: [M+H]⁺=452.2

### 4-(7-((4S,5R)-5-((Dimethylamino)methyl)-4-methyl-2-oxooxazolidin-3-yl)-9H-benzo[e]imidazo[2,1-c]pyrrolo[1,2-a][1,4]diazepin-12-yl)benzonitrile (61)

Using the procedures described above, compound 61 was synthesized. ESI: [M+H]⁺=479.2; ¹H NMR (400 MHz, CD₃OD): δ 8.01 (d, J=2.2 Hz, 1H), 7.99 (d, J=2.0 Hz, 1H), 7.78-7.64 (m, 8H), 7.28 (d, J=1.7 Hz, 1H), 5.38 (s, 2H), 5.29-5.20 (m, 1H), 4.97-4.89 (m, 1H), 3.85-3.74 (m, 1H), 3.60 (d, J=13.8 Hz, 1H), 3.04 (s, 6H), 1.28 (d, J=6.4 Hz, 3H).

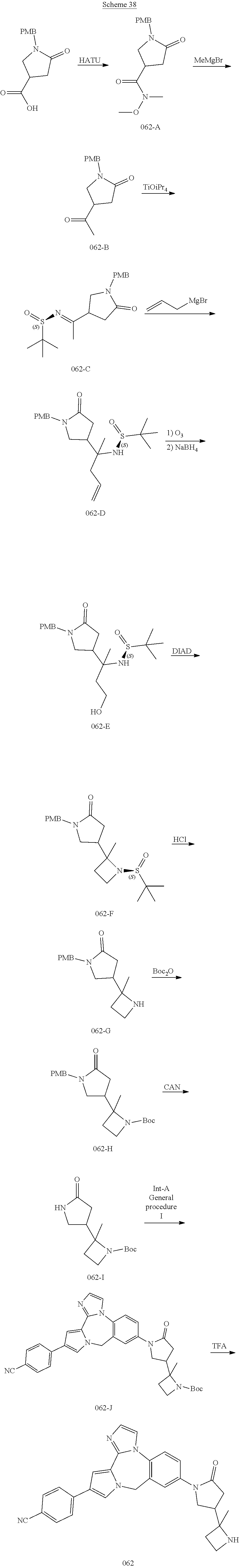

### N-Methoxy-1-(4-methoxybenzyl)-N-methyl-5-oxopyrrolidine-3-carboxamide (62-A)

To a solution of 1-(4-methoxybenzyl)-5-oxopyrrolidine-3-carboxylic acid (15.2 g, 60.8 mmol), N,O-dimethylhydroxylamine (6 g, 60.8 mmol), DIPEA (23.6 g, 182.4 mmol) in dry DMF (100 mL) was added HATU (23.1 g, 60.8 mmol) at 0° C. The resulting mixture was stirred at room temperature for 2 h and diluted with EA. The organic layer was washed with brine, dried over Na₂SO₄, filtered and concentrated to dryness. The crude product was purified by silica gel chromatography to give the desired product as a oil (16 g, yield: 90%). ESI: [M+H]⁺=293.1

### 4-Acetyl-1-(4-methoxybenzyl)pyrrolidin-2-one (62-B)

To a solution of compound 62-A (11.7 g, 40 mmol) in dry THE (120 mL) was added MeMgBr (60 mmol, 20 mL) at 0° C. The resulting mixture was stirred at room temperature for 3 h before pouring into 1N HCl solution at 0° C. Following extraction with EA, the combined organic layers were washed with brine, dried over Na₂SO₄, filtered and concentrated in vacuo. The residue was purified by silica gel chromatography eluting with PE/EA (1/1-1/20) to give the desired product as a yellow oil (7.3 g, yield: 73.9%). ESI: [M+H]⁺=248.1

### (S,E)-N-(1-(1-(4-Methoxybenzyl)-5-oxopyrrolidin-3-yl)ethylidene)-2-methyl propane-2-sulfinamide (62-C)

To a 0° C. solution of compound 62-B (6.3 g, 25.4 mmol) in dry THE (100 mL) was added (S)-2-methylpropane-2-sulfinamide (4.6 g, 38.1 mmol) and Ti(O-iPr)₄ (21.6 g, 76.2 mmol). The resulting mixture was stirred at 20° C. for 24 h. Sat'd NaHCO₃ aqueous solution was added at 0° C. to above solution to quench the reaction. The resulting mixture was filtered through a pad of celite and the filtrate was extracted with EA. The organic phase was washed with brine, dried over Na₂SO₄, filtered and concentrated. The residue was purified by chromatography on silica gel eluting with PE/EA (1/1-1/10) to give the desired product (7.7 g, yield: 86.5%). ESI: [M+H]⁺=351.2

### (S)—N-(2-(1-(4-Methoxybenzyl)-5-oxopyrrolidin-3-yl)pent-4-en-2-yl)ethane sulfinamide (62-D)

To a 0° C. solution of compound 62-C (6.0 g, 17.1 mmol) in dry THE (50 mL) was added allylmagnesium bromide (25.6 mmol, 25.6 mL). The resulting reaction mixture was stirred at 20° C. for 3 h, then quenched by addition of 1N NH₄Cl at 0° C. and extracted with EA three times. The combined organic layers were washed with brine, dried over Na₂SO₄, filtered and concentrated. The residue was purified by chromatography on silica gel eluting with PE/EA (1/1-1:20) to give the desired compound as two isomers as an oil (1 g, P1; 1.8 g P2, yield: 42.5%). ESI: [M+H]⁺=393

### (S)—N-(2-(1-(4-Methoxybenzyl)-5-oxopyrrolidin-3-yl)pent-4-en-2-yl)ethane sulfinamide (62-E)

A −78° C. solution of compound 62-D (1.8 g, 4.5 mmol) in dry MeOH (30 mL) was purged with 03 until the resulting mixture turned blue. After starting material was consumed, the solution was purged with N₂. NaBH₄ (684 mg, 18 mmol) was added to solution, and the resulting solution was stirred at 20° C. for 30 mins before addition of NH₄Cl aqueous solution and extraction with EA. The combined organic layers were washed with brine, dried over Na₂SO₄, filtered and concentrated in vacuo. The residue was purified by flash chromatography eluting with DCM/MeOH (100/1-20/1) to give the desired product as a white solid (810 mg, yield: 44%). ESI: [M+H]⁺=397.2

### 4-(1-((S)-tert-butylsulfinyl)-2-methylazetidin-2-yl)-1-(4-methoxybenzyl) pyrrolidin-2-one (62-F)

To a solution of compound 62-E (0.7 g, 1.76 mmol) and PPh3 (2.31 g, 8.83 mmol) in dry THE (50 mL) was added DIAD (2.13 g, 10.56 mmol) at 20° C. The reaction was stirred at 70° C. for 16 h and then concentrated in vacuo. The residue was purified by silica gel chromatography eluting with DCM/MeOH (100/1-10/1) to give the desired product as a yellow solid (0.5 g, yield: 75%). ESI: [M+H]⁺=379.2

### 1-(4-Methoxybenzyl)-4-(2-methylazetidin-2-yl)pyrrolidin-2-one (62-G)

To a solution of compound 62-F (0.5 g, 1.32 mmol) in 1,4-dioxane (10 mL) was added HCl (12 N, 2 mL) at 0° C. The reaction was stirred at 20° C. for 2 h and then concentrated in vacuo to give the desired compound as HCl salt, which was used in next step directly (0.41 g, yield: 99%). ESI: [M+H]⁺=275.2

### tert-butyl-2-(1-(4-methoxybenzyl)-5-oxopyrrolidin-3-yl)-2-methylazetidine-1-carboxylate (62-H)

To a solution of compound 62-G (0.4 g, 1.32 mmol) and TEA (404 mg, 4.0 mmol) in DCM (10 mL) at 0° C. was added Boc₂O (0.29 g, 1.32 mmol) slowly. The resulting mixture was stirred at 20° C. for 2 h. The mixture was diluted with water and extracted with EA three times. The organic layer was dried over anhydrous Na₂SO₄ and concentrated under reduced pressure. The residue was purified by silica gel chromatography eluting with PE/EA (10/1-1/10) to give the desired product as a white solid (0.33 g, yield: 66.7%). ESI: [M+H]⁺=375.2

### tert-butyl 2-methyl-2-(5-oxopyrrolidin-3-yl)azetidine-1-carboxylate (62-I)

To a solution of compound 62-H (0.1 g, 0.27 mmol) in CH₃CN (10 mL) was added CAN (740 mg, 1.35 mmol) at 0° C. The resulting mixture was stirred at 20° C. for 2 h and diluted with water. After the extraction of EA three times, the organic layers were dried over anhydrous Na₂SO₄, filtered and concentrated under reduced pressure. The residue was purified by silica gel chromatography eluting with DCM/MeOH (100/1-10/1) to give the desired compound (40 mg, yield: 58%). ESI: [M+H]⁺=255.2

### tert-butyl2-(1-(12-(4-cyanophenyl)-9H-benzo[e]imidazo[2,1-c]pyrrolo [1,2-a][1,4]diazepin-7-yl)-5-oxopyrrolidin-3-yl)-2-methylazetidine-1-carboxylate (62-J)

Using the procedures described in General procedure I, compound 62-J was synthesized. ESI: [M+H]⁺=575.3

### 4-(7-(4-(2-methylazetidin-2-yl)-2-oxopyrrolidin-1-yl)-9H-benzo[e]imidazo[2,1-c]pyrrolo[1,2-a][1,4]diazepin-12-yl)benzonitrile (62)

To a solution of compound 62-J (28 mg, 0.05 mmol) in anhydrous DCM (10 mL) was added TFA (2 mL). The resulting reaction solution was stirred for at room temperature for 3 h before the addition of saturated NaHCO₃ aqueous to adjust pH>7. The mixture was then extracted with DCM; the combined organic layers were washed with brine, dried over Na₂SO₄, filtered and concentrated in vacuo. The residue was purified by Prep-HPLC to give the desired compound (8 mg, yield: 33.3%). ESI: [M+H]⁺=475.2

¹H NMR (400 MHz, CD₃OD): δ 8.13 (d, 2.4 Hz, 1H), 8.05 (s, 1H), 7.91 (m, 1H), 7.74 (m, 7H), 7.30 (s, 1H), 5.39 (s, 2H), 4.28-3.69 (m, 5H), 2.91 (m, 1H), 2.68-2.53 (m, 2H), 2.44 (m, 1H), 1.63 (s, 3H).

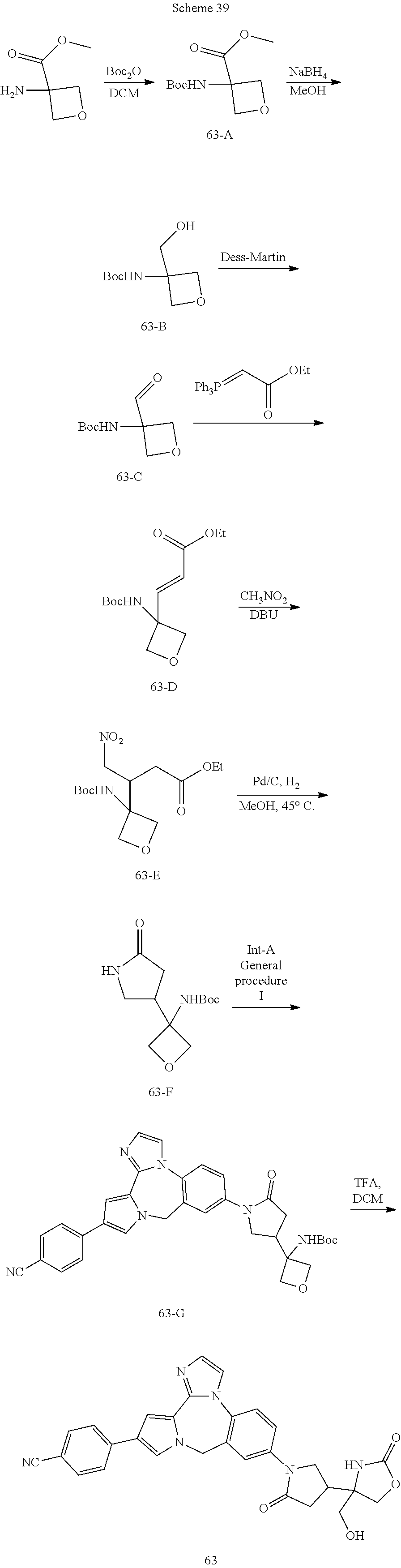

### Methyl 3-((tert-butoxycarbonyl)amino)oxetane-3-carboxylate (63-A)

To a solution of methyl 3-aminooxetane-3-carboxylate (1.7 g, 13 mmol) and di-tert-butyl dicarbonate (3.1 g, 14.3 mmol) in DCM (17 mL) was added TEA (2.63 g, 26 mmol), and the mixture was stirred at 20° C. for 4 h under nitrogen atmosphere before the dilution with DCM. The resulting solution was washed with water, brine, concentrated under reduced pressure. The residue was purified by silica gel chromatography eluting with PE/EA (10/1) to give the desired compound (1.0 g, yield: 33%). ESI: [M+H]⁺=232.2

### tert-butyl (3-(hydroxymethyl)oxetan-3-yl)carbamate (63-B)

To a solution of compound 63-A (2.0 g, 8.7 mmol) in MeOH (20 mL) was added NaBH₄ (1.0 g, 26.1 mmol), and the mixture was stirred at 20° C. for overnight under nitrogen atmosphere before the addition of aqueous NH₄Cl solution to quench to reaction. The resulting mixture was extracted with EtOAc three times. The combined organic layers were washed with brine, concentrated under reduced pressure. The residue was purified by silica gel chromatography eluting with PE/EA (1/1) to give the desired compound (1.7 g, yield: 94%). ESI: [M+H]⁺=204.1; ¹H NMR (400 MHz, CDCl₃): δ 5.09 (s, 1H), 4.66 (d, J=6.7 Hz, 2H), 4.54 (d, J=6.5 Hz, 2H), 4.03 (s, 2H), 1.45 (s, 9H).

### tert-butyl (3-formyloxetan-3-yl)carbamate (63-C)

To a solution of compound 63-B (1.7 g, 8.4 mmol) in DCM (20 mL) was added Dess-Martin periodinane (5.3 g, 12.6 mmol), and the mixture was stirred at 20° C. overnight under nitrogen atmosphere. The mixture was then quenched with NaHCO₃ and Na₂SO₃ aqueous, extracted with DCM. The organic phase was washed with brine, concentrated under reduced pressure. The residue was purified by silica gel chromatography eluting with PE/EtOAc (1/1) to give the desired compound (1.4 g, yield: 82%). ESI: [M+H]⁺=202.1

### Ethyl-3-(3-((tert-butoxycarbonyl)amino)oxetan-3-yl)acrylate (63-D)

The solution of 63-C (1.4 g, 7.0 mmol) and ethyl 2-(triphenyl-15-phosphanylidene)acetate (3.7 g, 10.5 mmol) in toluene (20 mL) was stirred at 110° C. overnight under nitrogen atmosphere. After cooling to room temperature, the mixture was concentrated under reduced pressure, and the residue was purified by silica gel chromatography eluting with PE/EA (5/1) to give the desired compound (1.0 g, yield 53%). ESI: [M+H]⁺=272.1; ¹H NMR (400 MHz, CDCl₃): δ 7.33 (d, J=15.7 Hz, 1H), 6.00 (d, J=15.7 Hz, 1H), 5.29 (s, 1H), 4.77 (d, J=6.5 Hz, 2H), 4.64 (s, 2H), 4.22 (q, J=7.1 Hz, 2H), 1.44 (s, 9H), 1.31 (t, J=7.1 Hz, 3H).

### Ethyl 3-(3-((tert-butoxycarbonyl)amino)oxetan-3-yl)-4-nitrobutanoate (63-E)

To a solution of compound 63-D (0.794 g, 2.93 mmol) in nitromethane (8 mL) was added DBU (2.3 g, 14.7 mmol), and the mixture was stirred at 70° C. for 3 h under nitrogen atmosphere. The mixture was the diluted with DCM and washed with water, brine and concentrated to dryness under reduced pressure. The residue was purified by silica gel chromatography eluting with PE/EtOAc (3/1) to give the desired product (0.86 g, yield: 89%). ESI: [M+H]⁺=333.2

### tert-butyl (3-(5-oxopyrrolidin-3-yl)oxetan-3-yl)carbamate (63-F)

To a solution of compound 63-E (0.39 g, 1.1 mmol) in MeOH (5 mL) was added Pd/C 10% on Carbon (0.05 g, 0.44 mmol), and the mixture was stirred at 45° C. for 1 h under a H2 balloon. The mixture was then cooled to room temperature and filtered through celite, the filtrate was concentrated under reduced pressure. The residue was purified by silica gel chromatography eluting with PE/EtOAc (1/1) to give the desired product (0.2 g, yield: 67%). ESI: [M+H]⁺=257.1

### tert-butyl(3-(1-(12-(4-cyanophenyl)-9H-benzo[e]imidazo[2,1-c]pyrrolo[1, 2-a][1,4]diazepin-7-yl)-5-oxopyrrolidin-3-yl)oxetan-3-yl)carbamate (63-G)

Using the procedures described in General procedure I, compound 063-G was synthesized. ESI: [M+H]⁺=577.2

### 4-(7-(4-(4-(Hydroxymethyl)-2-oxooxazolidin-4-yl)-2-oxopyrrolidin-1-yl)-9H-benzo[e]imidazo[2,1-c]pyrrolo[1,2-a][1,4]diazepin-12-yl)benzonitrile (63)

To a solution of compound 63-G (3 mg, 0.005 mmol) in DCM (2 mL) was added TFA (0.5 mL), and the mixture was stirred at 20° C. for 1 h and then concentrated under reduced pressure. The residue was purified by Prep-HPLC to give the titled compound (1.4 mg, yield: 52%). ESI: [M+H]⁺=521.2; ¹H NMR (400 MHz, CD₃OD): δ 8.11-8.06 (m, 2H), 7.98-7.91 (m, 1H), 7.80 (s, 1H), 7.75-7.70 (m, 7H), 7.31 (s, 1H), 5.40 (s, 2H), 4.45-4.27 (m, 2H), 4.11-4.01 (m, 1H), 3.97-3.83 (m, 1H), 3.67-3.54 (m, 2H), 3.0-2.90 (m, 1H), 2.68 (dd, J=9.5, 7.1 Hz, 2H).

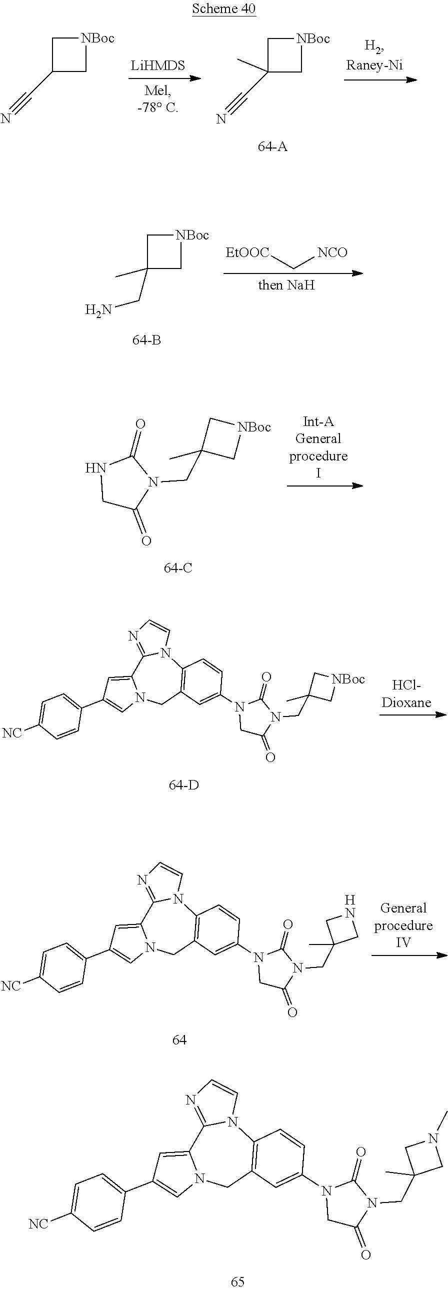

### tert-butyl 3-cyano-3-methylazetidine-1-carboxylate (64-A)

To a solution of tert-butyl 3-cyanoazetidine-1-carboxylate (1.5 g, 8.2 mmol) in THE (30 mL) was added LiHMDS (1.0M solution in THF, 9.0 mL, 9.0 mmol) at −78° C. dropwise over 25 min. The yellow solution was stirred at this temperature for 30 min, and then iodomethane (0.78 mL, 12.5 mmol) was slowly added. The reaction mixture was stirred at this temperature for another 30 min and then warmed to room temperature over 1 h. The reaction mixture was quenched with 10 mL of saturated NH₄Cl aqueous and diluted with 10 mL of water then extracted with ˜100 mL of EtOAc twice. The combined organic layers were washed with 20 mL of water and 20 mL brine, then dried over sodium sulfate, filtered and concentrated. The residue was chromatographed over silica gel with EtOAc/hexanes, (gradient: 0-30% EtOAc) to give the desired product (1.2 g, yield: 74.6%). ESI: [M+H]⁺=197.1

### tert-butyl 3-(aminomethyl)-3-methylazetidine-1-carboxylate (64-B)

To a solution of compound 64-A (1.2 g, 6.12 mmol) in MeOH (20 mL) was added Raney Ni (3 g) and stirred at rt under H2 overnight. The reaction mixture was then filtered, the filtrate was concentrated to give the desired product (900 mg, yield: 73.5%). ESI: [M+H]⁺=201.2

### tert-butyl 3-((2,5-dioxoimidazolidin-1-yl)methyl)-3-methylazetidine-1-carboxylate (064-C)

To a solution of compound 64-B (900 mg, 4.5 mmol) in dry DCM (20 mL) was added ethyl 2-isocyanatoacetate (580 mg, 4.5 mmol) at 0° C. and the resulting solution was stirred at rt for 2 h before the addition of NaH (216 mg, 9 mmol). The reaction mixture was stirred at rt for additional 1 h, and then MeOH (10 mL) was added. The mixture was concentrated, and the residue was purified by chromatographed over silica gel with (MeOH/DCM=1/10) to give the desired product (1 g, yield: 78.4%).

ESI: [M+H]⁺=284.2.

### tert-butyl 3-((3-(12-(4-cyanophenyl)-9H-benzo[e]imidazo[2,1-c]pyrrolo [1,2-a][1,4]diazepin-7-yl)-2,5-dioxoimidazolidin-1-yl)methyl)-3-methylazetidine-1-carboxylate (64-D)

Using the procedures described in General procedure I; the desired compound was synthesized. ESI: [M+H]⁺=604.3

### 4-(7-(3-((3-Methylazetidin-3-yl)methyl)-2,4-dioxoimidazolidin-1-yl)-9H-benzo[e]imidazo[2,1-c]pyrrolo[1,2-a][1,4]diazepin-12-yl)benzonitrile (64)

To a solution of compound 064-D (350 mg, 0.57 mmol) in MeOH (15 mL) was added HCl-dioxane (4 M, 5 mL), and the resulting solution was stirred at rt for 1 h and then concentrated. The residue was purified by chromatography on silica gel to give the desired compound (264 mg, yield: 92.1%). ESI: [M+H]⁺=504.2; ¹H NMR (400 MHz, CD₃OD): δ 8.07 (d, J=2.3 Hz, 1H), 8.05 (d, J=2.3 Hz, 1H), 7.95 (dd, J=8.9, 2.6 Hz, 1H), 7.80-7.69 (m, 7H), 7.30 (d, J=1.8 Hz, 1H), 5.40 (s, 2H), 4.59 (s, 2H), 4.23 (d, J=11.5 Hz, 2H), 3.89-3.67 (m, 4H), 1.43 (s, 3H).

### 4-(7-(3-((1,3-Dimethylazetidin-3-yl)methyl)-2,4-dioxoimidazolidin-1-yl)-9H-benzo[e]imidazo[2,1-c]pyrrolo[1,2-a][1,4]diazepin-12-yl)benzonitrile (65)

Using the procedures described in General procedure IV, the desired compound was synthesized. ESI: [M+H]⁺=518.2; ¹H NMR (400 MHz, CD₃OD): δ 8.06 (s, 1H), 8.05 (s, 1H), 7.96 (d, J=8.8 Hz, 1H), 7.78-7.31 (m, 7H), 7.31 (d, J=1.5 Hz, 1H), 5.39 (s, 2H), 4.59 (s, 2H), 4.50 (d, J=11.0 Hz, 1H), 4.14 (d, J=10.8 Hz, 1H), 4.00 (d, J=10.6 Hz, 1H), 3.85-3.84 (m, 3H), 2.94 (s, 3H), 1.43 (s, 3H).

Using the procedures described above, the following examples Table 8 were synthesized.

TABLE 8

| | Ex. | | LC-MS |
|---|---|---|---|
| Structure | # | ¹H NMR | (M + H)⁺ |
| 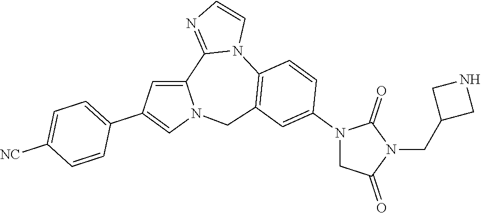 [SMILES: N#Cc1ccc(-c2cc3n(c2)Cc2cc(N4CC(=O)N(CC5CNC5)C4=O)ccc2-n2ccnc2-3)cc1] | 66 | (400 MHz, CD₃OD): δ 8.07 (d, J = 2.1 Hz, 1H), 8.05 (d, J = 2.1 Hz, 1H), 7.95 (dd, J = 8.9, 2.6 Hz, 1H), 7.80-7.69 (m, 7H), 7.32 (d, J = 1.4 Hz, 1H), 5.40 (s, 2H), 4.53 (d, J = 9.5 Hz, 2H), 4.22 − 4.11 (m, 2H), 4.11 − 3.98 (m, 2H), 3.89 (d, J = 6.6 Hz, 2H), | 490.2 |
| | | 3.29 − 3.23 (m, 1H). | |
| 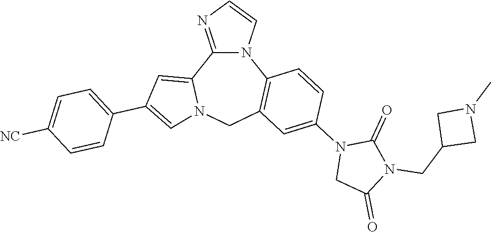 [SMILES: CN1CC(CN2C(=O)CN(c3ccc4c(c3)Cn3cc(-c5ccc(C#N)cc5)cc3-c3nccn3-4)C2=O)C1] | 67 | (400 MHz, CD₃OD): δ 8.07 (d, J = 2.1 Hz, 1H), 8.05 (d, J = 2.1 Hz, 1H), 7.96 (d, J = 9.0 Hz, 1H), 7.81 − 7.66 (m, 7H), 7.32 (d, J = 1.6 Hz, 1H), 5.40 (s, 2H), 4.53 (s, 2H), 4.35-4.28 (m, 2H), 4.12 − 3.82 (m, 4H), 3.20 (s, 1H), 2.93 (s, 3H). | 504.2 |
| 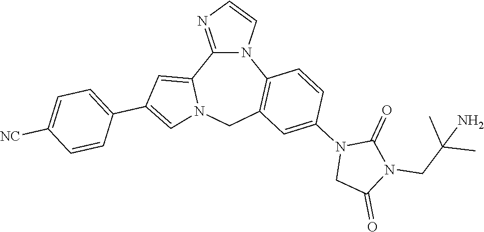 [SMILES: CC(C)(N)CN1C(=O)CN(c2ccc3c(c2)Cn2cc(-c4ccc(C#N)cc4)cc2-c2nccn2-3)C1=O] | 68 | (400 MHz, CD₃OD): δ 8.05 (d, J = 2.6 Hz, 1H), 8.05-7.96 (m, 2H), 7.77 − 7.68 (m, 6H), 7.62 (s, 1H), 7.26 (s, 1H), 5.37 (s, 2H), 4.59 (s, 2H), 3.78 (s, 2H), 1.42 (s, 6H) | 492.2 |
| 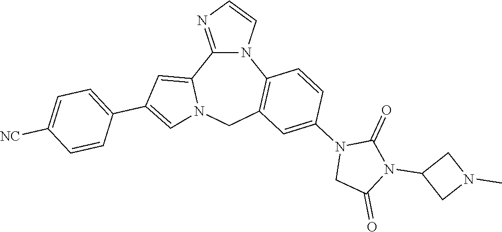 [SMILES: CN1CC(N2C(=O)CN(c3ccc4c(c3)Cn3cc(-c5ccc(C#N)cc5)cc3-c3nccn3-4)C2=O)C1] | 69 | (400 MHz, CD₃OD): δ 8.05- 7.95 (m, 3H), 7.78- 7.66 (m, 7H), 7.28 (s, 1H), 5.38 (s, 2H), 5.28-5.20 (m, 1H), 4.83-4.80 (m, 1H), 4.73-4.61 (m, 2H), 4.56 (d, J = 9.1 Hz, 2H), 4.44- 4.39 (m, 1H), 3.15(s, 1.5H), 3.02 (s, 1.5H). | 490.2 |

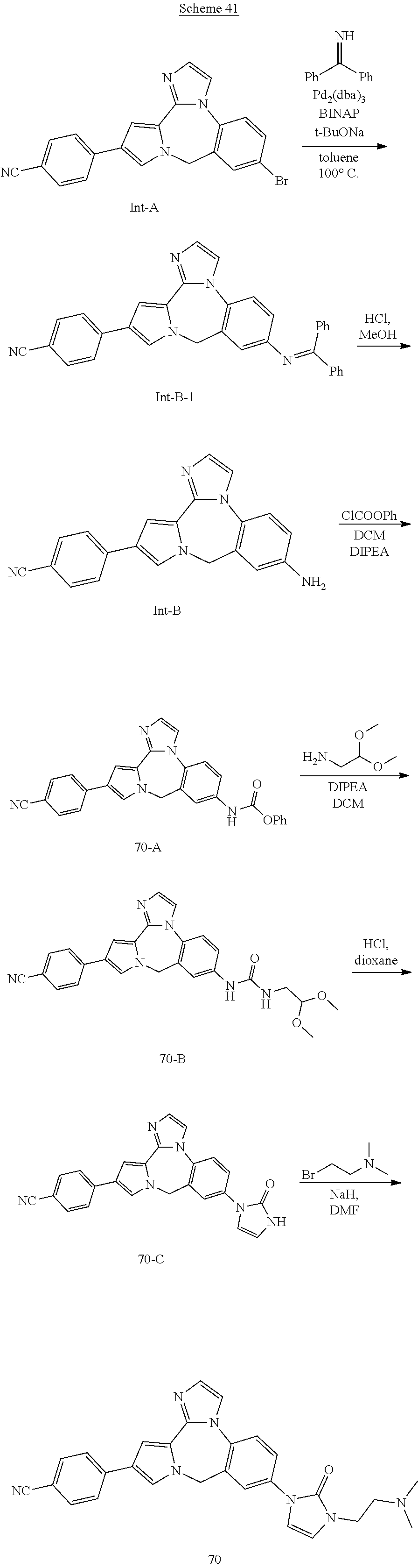

### 4-(7-((Diphenylmethylene)amino)-9H-benzo[e]imidazo[2,1-c]pyrrolo[1,2-a][1,4]diazepin-12-yl)benzonitrile (Int-B-1)

A mixture of compound Int-A (1 g, 2.5 mmol), diphenylmethanimine (905 mg, 5 mmol), Pd₂(dba)₃ (566 mg, 0.36 mmol), BINAP (738 mg, 1.25 mmol) and t-BuONa (736 mg, 7.15 mmol) in toluene (20 mL) was stirred at 100° C. under N₂ atmosphere for 16 h. The mixture was cooled to room temperature and diluted with DCM (50 mL) and MeOH (5 mL). The organic solution was washed with water (50 mL), brine and dried over Na₂SO₄. After filtration and concentrate, the residue was purified by silica gel chromatography to give the desired compound (1.1 g, yield: 88.1%). ESI: [M+H]⁺=502.2

### 4-(7-Amino-9H-benzo[e]imidazo[2,1-c]pyrrolo[1,2-a][1,4]diazepin-12-yl) benzonitrile (Int-B)

To a solution of compound Int-B-1 (1.1 g, 2.19 mmol) in MeOH (20 mL) was added HCl aq. (1M, 10 ml.), the resulting solution was stirred at room temperature for 2-3 hrs. The solid formed in the reaction system was collected by filtration and dried to give desired product as HCl salt (700 mg, yield: 94.7%). ESI: [M+H]⁺=338.1

### Phenyl(12-(4-cyanophenyl)-9H-benzo[e]imidazo[2,1-c]pyrrolo[1,2-a][1,4]diazepin-7-yl)carbamate (70-A)

To a solution of compound Int-A (100 mg, 0.30 mmol) and DIPEA (0.5 mL) in DCM (5 mL) was added phenyl carbonochloridate (70 mg, 0.44 mmol) at room temperature. The resulting solution was stirred for 2-3 h and then concentrated to give the desired crude compound, which was used directly to next step. ESI: [M+H]⁺=458.2

### 1-(12-(4-Cyanophenyl)-9H-benzo[e]imidazo[2,1-c]pyrrolo[1,2-a][1,4]diazepin-7-yl)-3-(2,2-dimethoxyethyl)urea (70-B)

To a solution of compound 70-A (crude, 0.30 mmol) in DCM (5 mL) was added 2,2-dimethoxyethan-1-amine (32 mg, 0.31 mmol) at room temperature, and the resulting solution was stirred for 16 h. The reaction solution was diluted with DCM (200 mL) and washed with H₂O (20 mL), brine, dried over Na₂SO₄. After filtration and concentration, the residue was purified by chromatography on silica gel to give the desired product as a solid (53 mg, yield: 74.6%). ESI: [M+H]⁺=469.2

### 4-(7-(2-Oxo-2,3-dihydro-1H-imidazol-1-yl)-9H-benzo[e]imidazo[2,1-c]pyrrolo[1,2-a][1,4]diazepin-12-yl)benzonitrile (70-C)

To a solution of compound 70-B (104 mg, 0.22 mmol) in dioxane (5 mL) was added HCl (aq.) (1M, 4 mL) and stirred at 85° C. for 2-3 brs. After cooling to the room temperature, the solid formed in the reaction mixture was collected by filtration and dried to give the desired product (50 mg, yield: 56.2%). ESI: [M+H]⁺=405.1

### 4-(7-(3-(2-(Dimethylamino)ethyl)-2-oxo-2,3-dihydro-1H-imidazol-1-yl)-9H-benzo[e]imidazo[2,1-c]pyrrolo[1,2-a][1,4]diazepin-12-yl)benzonitrile (70)

To a solution of compound 70-C (50 mg, 0.12 mmol) in DMF (2 mL) was added NaH (20 mg, 0.5 mmol) at 0° C., and the resulting solution was stirred for 30 mins before the addition of 2-bromo-N,N-dimethylethan-1-amine (28 mg, 0.18 mmol) at room temperature. Then the solution was stirred for 2 h at room temperature and then quenched with NH₄Cl (20 mL); the resulting mixture was extracted with DCM (20 mL). The organic phase was washed with brine, dried over Na₂SO₄, filtered and concentrated to dryness. The residue was purified by Prep-TLC to give the desired compound as a solid (13 mg, yield: 22.4%). ESI: [M+H]⁺=476.2; ¹H NMR (400 MHz, CD₃OD): δ 8.11 (d, J=2.4 Hz, 1H), 8.06 (d, J=2.0 Hz, 1H), 7.96 (dd, J=8.8, 2.5 Hz, 1H), 7.82 (d, J=8.8 Hz, 1H), 7.79-7.67 (m, 6H), 7.30 (d, J=1.8 Hz, 1H), 7.12 (d, J=3.2 Hz, 1H), 6.82 (d, J=3.2 Hz, 1H), 4.15 (t, J=5.8 Hz, 2H), 3.55 (t, J=5.8 Hz, 2H), 2.99 (s, 6H).

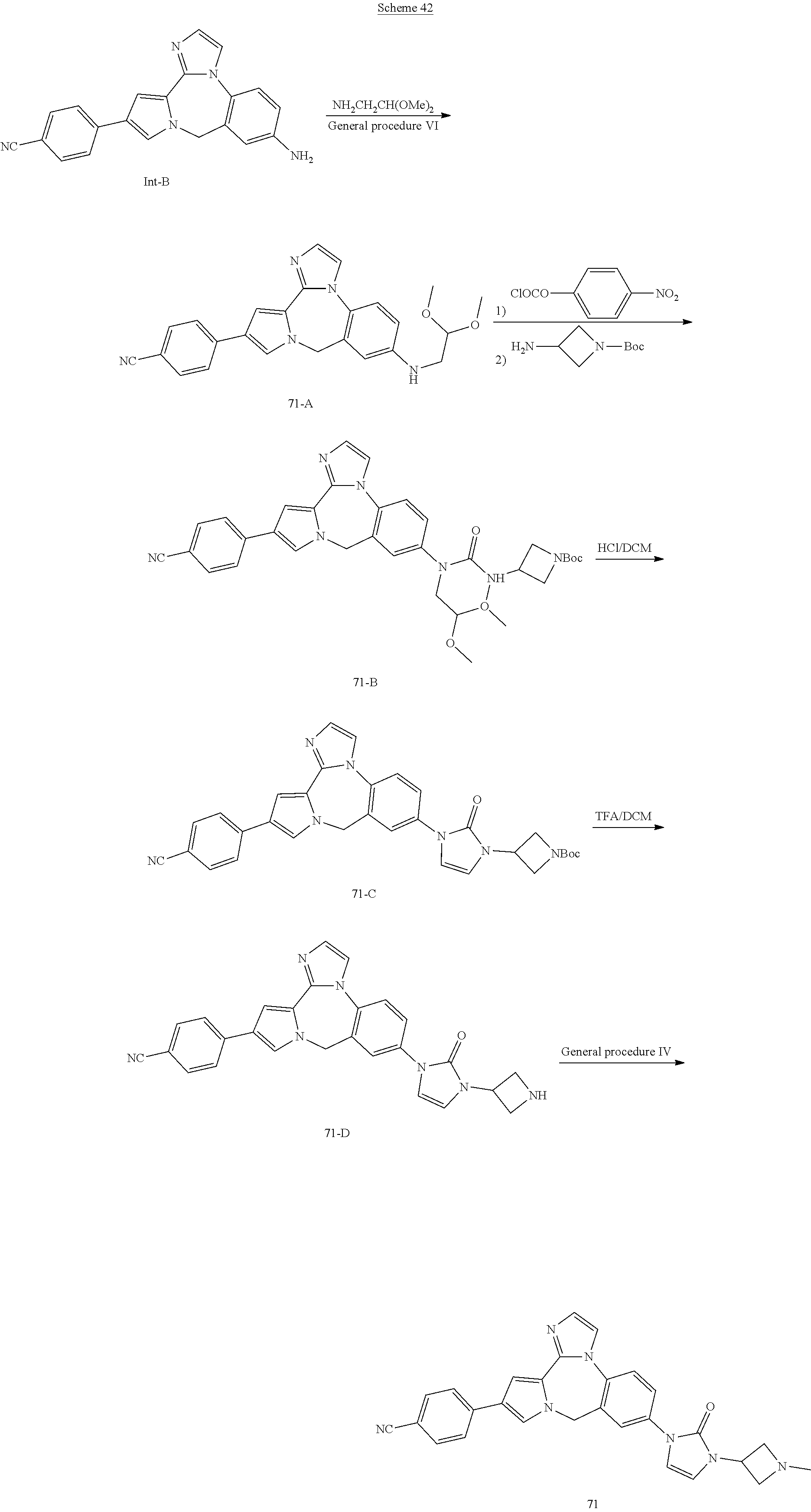

### 4-(7-((2,2-Dimethoxyethyl)amino)-9H-benzo[e]imidazo[2,1-c]pyrrolo[1,2-a][1,4]diazepin-12-yl)benzonitrile (71-A)

Using the procedures described above, compound 071-A was synthesized.

### tert-butyl 3-(3-(12-(4-cyanophenyl)-9H-benzo[e]imidazo[2,1-c]pyrrolo [1,2-a][1,4]diazepin-7-yl)-3-(2,2-dimethoxyethyl)ureido)azetidine-1-carboxylate (71-B)

To a solution of compound 71-A (59 mg, 0.14 mmol) in CH₃CN (10 mL) at 0° C. was added 4-nitrophenyl carbonochloridate (30 mg, 0.15 mmol) slowly. The resulting mixture was stirred at 20° C. for 3 h and then concentrated under reduced pressure to give crude product (82 mg), which was used in next step directly ESI: [M+H]⁺=591.2 To a solution of above compound (82 mg, 0.14 mmol) in DMAc (3 mL) at 20° C. was added tert-butyl 3-aminoazetidine-1-carboxylate (72 mg, 0.42 mmol) slowly. The resulting mixture was placed at 120° C. in a microwave reactor for 3 h. The reaction mixture was diluted with DCM and washed with brine, dried over Na₂SO₄. After filtration, the organic solution was concentrated in vacuo to give the desired crude compound (87 mg), which was used in next step directly. ESI: [M+H]⁺=624.3

### tert-butyl-3-(3-(12-(4-cyanophenyl)-9H-benzo[e]imidazo[2,1-c]pyrrolo [1,2-a][1,4]diazepin-7-yl)-2-oxo-2,3-dihydro-1H-imidazol-1-yl)azetidine-1-carboxylate (71-C)

To a solution of compound 71-B (87 mg, 0.14 mmol) in DCM (20 mL) at 0° C. was added HCl in dioxane (4N, 0.5 mL) slowly. The resulting mixture was stirred at 20° C. for 3 h and then quenched with sat'd NaHCO₃ aqueous solution to adjust pH>7. The resulting mixture was extracted with DCM three times. The combined organic layers were washed with brine, dried over Na₂SO₄, filtered and concentrated to dryness. The residue was purified by Prep-TLC to give the desired compound as a yellow solid (10 mg, yield: 12%). ESI: [M+H]⁺=560.2

### 4-(7-(3-(Azetidin-3-yl)-2-oxo-2,3-dihydro-1H-imidazol-1-yl)-9H-benzo[e]imidazo[2,1-c]pyrrolo[1,2-a][1,4]diazepin-12-yl)benzonitrile (71-D)

To a solution of compound 71-C (10 mg, 0.02 mmol) in DCM (5 mL) was added TFA (1 mL). The reaction solution was stirred at room temperature for 1 hour. The reaction was quenched with sat'd NaHCO₃ aqueous solution to adjust pH>7, and the mixture was extracted with DCM three times. The combined organic layers were washed with brine, dried over Na₂SO₄, concentrated in vacuo to give the desired crude product (9 mg). ESI: [M+H]⁺=460.2

### 4-(7-(3-(1-Methylazetidin-3-yl)-2-oxo-2,3-dihydro-1H-imidazol-1-yl)-9H-benzo[e]imidazo[2,1-c]pyrrolo[1,2-a][1,4]diazepin-12-yl)benzonitrile (71)

Using the procedures described above, compound 71 was synthesized (2 mg, yield: 22.1%). ESI: [M+H]=474.2; ¹H NMR (400 MHz, CD₃OD): δ 8.07 (d, J=2.4 Hz, 1H), 7.93 (s, 1H), 7.78-7.66 (m, 5H), 7.54 (s, 1H), 7.48 (m, 1H), 7.35 (m, 1H), 7.22 (d, J=1.5 Hz, 1H), 7.08 (d, J=3.2 Hz, 1H), 6.81 (s, 1H), 5.35 (s, 2H), 5.11-4.99 (m, 1H), 4.72-4.30 (m, 4H), 3.12 (s, 3H).

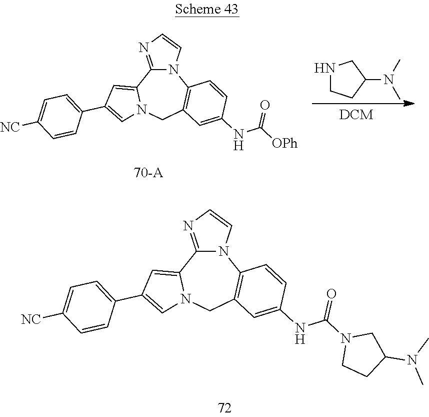

### N-(12-(4-Cyanophenyl)-9H-benzo[e]imidazo[2,1-c]pyrrolo[1,2-a][1,4]diazepin-7-yl)-3-(dimethylamino)cyclopentane-1-carboxamide (72)

To a solution of compound 66-A (50 mg, 0.11 mmol) in DCM (5 mL) was added N,N-dimethylpyrrolidin-3-amine (15 mg, 0.13 mmol). The reaction mixture was stirred at room temperature overnight and then diluted with DCM (100 mL). The organic phase was washed with H₂O (20 mL), brine, dried over Na₂SO₄ and filtered. The solution was concentrated, and the residue was purified by Prep-HPLC to give the desired compound as a white solid (23 mg, yield: 45.1%). ESI: [M+H]⁺=478.2; ¹H NMR (400 MHz, CD₃OD): δ 8.04-8.03 (d, J=2.2 Hz, 1H), 7.90-7.89 (d, J=2.0 Hz, 1H), 7.80-7.79 (d, J=4 Hz, 1H), 7.80-7.63 (m, 8H), 7.31-7.30 (d, J=4 Hz, 1H), 5.33 (s, 2H), 4.05-3.94 (m, 2H), 3.82-3.80 (m, 1H), 3.66-3.59 (m, 2H), 2.98 (s, 6H), 2.54-2.53 (m, 1H), 2.30-2.23 (m, 1H).

Using the procedure described above, the following examples were Table 9 were synthesized.

TABLE 9

| | Ex. | | LC-MS |
|---|---|---|---|
| Structure | # | ¹H NMR | (M + H)⁺ |
| 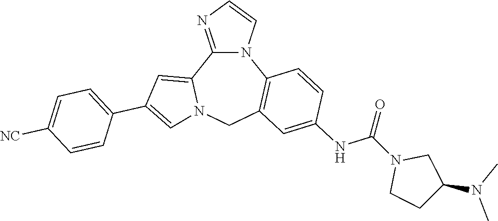 [SMILES: CN(C)[C@H]1CCN(C(=O)Nc2ccc3c(c2)Cn2cc(-c4ccc(C#N)cc4)cc2-c2nccn2-3)C1] | 73 | (400 MHz, CD₃OD): δ 8.04 (d, J = 4 Hz, 1H), 7.90 (d, J = 4 Hz, 1H), 7.80 (d, J = 4 Hz, 1H), 7.80 − 7.63 (m, 7H), 7.3 (d, J = 4 Hz, 1H), 5.33 (s, 2H), 4.05 − 3.94 (m, 2H), 3.82 − 3.80 (m, 1H), 3.66 − 3.59 (m, 2H), 2.98 (s, 6H), 2.54 − 2.53 (m, 1H), 2.30 − 2.23 (m, 1H). | 478.2 |
| 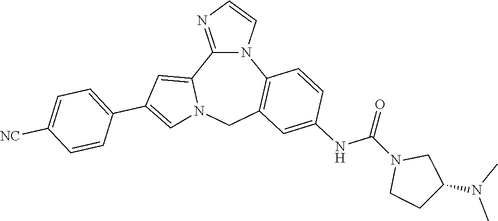 [SMILES: CN(C)[C@@H]1CCN(C(=O)Nc2ccc3c(c2)Cn2cc(-c4ccc(C#N)cc4)cc2-c2nccn2-3)C1] | 074 | (400 MHz, CD₃OD): δ 8.04 (d, J = 4 Hz, 1H), 7.90 (d, J = 4 Hz, 1H), 7.79 (d, J = 4 Hz, 1H), 7.80 − 7.63 (m, 7H), 7.31 − 7.30 (d, J = 4 Hz, 1H), 5.33 (s, 2H), 4.05 − 3.94 (m, 2H), 3.82 − 3.80 (m, 1H), 3.66 − 3.59 (m, 2H), 2.98 (s, 6H), 2.54 − 2.53 (m, 1H), 2.30 − 2.23(m, 1H). | 478.2 |

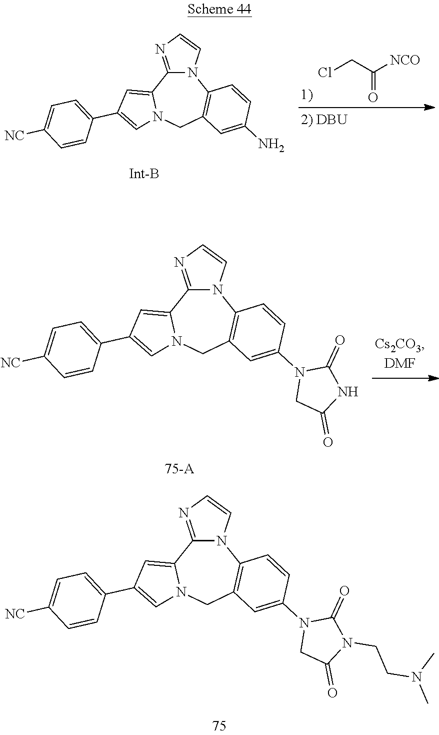

### 4-(7-(2,4-Dioxoimidazolidin-1-yl)-9H-benzo[e]imidazo[2,1-c]pyrrolo[1,2-a][1,4]diazepin-12-yl)benzonitrile (75-A)

To a stirred solution of compound Int-B (300 mg, 0.89 mmol), TEA (180 mg, 1.80 mmol) in DMF (10 mL) was added 2-chloroacetyl isocyanate (127 mg, 1.07 mmol) under ice-cooling, and the mixture was stirred at room temperature for 1 h. 1,8-Diazabicyclo[5.4.0]undec-7-ene (270 mg, 1.80 mmol) was added, and the resulting solution was stirred at room temperature for 1 h. The mixture was diluted with DCM, washed with water and brine, dried over Na₂SO₄. After filtration and concentration, the residue was purified by Prep-HPLC to give the desired product as white solid (250 mg, yield: 52%). ESI: [M+H]⁺=421.1

### 4-(7-(3-(2-(Dimethylamino)ethyl)-2,4-dioxoimidazolidin-1-yl)-9H-benzo[e]imidazo[2,1-c]pyrrolo[1,2-a][1,4]diazepin-12-yl)benzonitrile (75)

A mixture of compound 75-A (70 mg, 0.13 mmol), cesium carbonate (156 mg, 0.48 mmol) and 2-bromo-N,N-dimethylethan-1-amine (75 mg, 0.48 mmol) in DMF (10 mL) was stirred at 100° C. for 1 h. After cooling, the reaction mixture was filtered. The filtrate was concentrated, and the residue was purified by Prep-HPLC to give the desired compound as a trifloroacetic acid salt as a white solid (25 mg, yield: 32%). ESI: [M+H]⁺=492.2; ¹H NMR (400 MHz, CD₃OD): δ 8.05 (d, J=2.4 Hz, 1H), 8.01 (s, 1H), 7.96 (m, 1H), 7.78-7.73 (m, 4H), 7.70 (d, J=8.5 Hz, 2H), 7.66 (s, 1H), 7.28 (s, 1H), 5.38 (s, 2H), 4.56 (s, 2H), 4.05-3.98 (m, 2H), 3.54-3.44 (m, 2H), 3.02 (s, 6H).

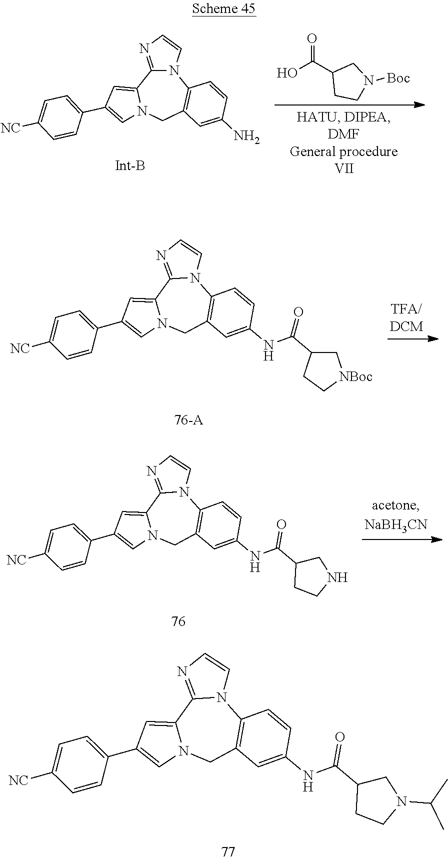 [SMILES: N#Cc1ccc(-c2cc3n(c2)Cc2cc(N)ccc2-n2ccnc2-3)cc1 ; CC(C)(C)OC(=O)N1CCC(C(=O)O)C1 ; CC(C)(C)OC(=O)N1CCC(C(=O)Nc2ccc3c(c2)Cn2cc(-c4ccc(C#N)cc4)cc2-c2nccn2-3)C1 ; N#Cc1ccc(-c2cc3n(c2)Cc2cc(NC(=O)C4CCNC4)ccc2-n2ccnc2-3)cc1 ; CC(C)N1CCC(C(=O)Nc2ccc3c(c2)Cn2cc(-c4ccc(C#N)cc4)cc2-c2nccn2-3)C1]

General Procedure VII:

### tert-butyl 3-((12-(4-cyanophenyl)-9H-benzo[e]imidazo[2,1-c]pyrrolo [1,2-a][1,4]diazepin-7-yl)carbamoyl)pyrrolidine-1-carboxylate (76-A)

A solution of compound Int-B (100 mg, 0.27 mmol) in DMF (10 mL), DIPEA (1 mL) and HATU (205 mg, 0.54 mmol) was stirred at 0° C. 1-(tert-butoxycarbonyl)pyrrolidine-3-carboxylic acid (116 mg, 0.54 mmol) was added to above solution. The resulting reaction solution was stirred at room temperature for 3 h and then quenched by addition of NaHCO₃ solution (10 mL). The mixture was extracted with DCM/MeOH (10 mL/2 mL) three times. The combined organic layers were washed with brine and dried over Na₂SO₄. After filtration, the solution was concentrated and then purified by Prep-TLC (DCM:MeOH=15:1) to obtain the desired product (103 mg, yield: 71.5%). ESI: [M+H]⁺=535.2

### N-(12-(4-Cyanophenyl)-9H-benzo[e]imidazo[2,1-c]pyrrolo[1,2-a][1,4]diazepin-7-yl)pyrrolidine-3-carboxamide (76)

To a solution of compound 76-A (103 mg, 0.19 mmol) in DCM (8 mL) was added TFA (3 mL) dropwise. The reaction solution stirred for 2 h, and then the reaction was quenched with sat'd NaHCO₃ (10 mL). The mixture was extracted with DCM/MeOH (10 mL/2 mL) three times. The combined organic layers were washed with brine and dried over Na₂SO₄. After filtration, the solution was concentrated and purified by Prep-TLC (DCM:MeOH=15:1) to obtain the desired product as a light yellow solid (76 mg, yield: 90.5%). ESI: [M+H]⁺=435.2; ¹H NMR (400 MHz, CD₃OD): δ 8.08 (d, J=2.2 Hz, 2H), 7.87-7.67 (m, 8H), 7.34 (d, J=1.4 Hz, 1H), 5.38 (s, 2H), 3.68-3.64 (m, 1H), 3.53-3.35 (m, 4H), 2.48-2.39 (m, 1H), 2.31-2.21 (m, 1H).

### N-(12-(4-Cyanophenyl)-9H-benzo[e]imidazo[2,1-c]pyrrolo[1,2-a][1,4]diazepin-7-yl)-1-isopropylpyrrolidine-3-carboxamide (77)

To a solution of compound 76 (37 mg, 0.09 mmol) in THE (3 mL) was added acetone (3 mL) and NaBH₃CN (16.1 mg, 0.26 mmol). The reaction mixture was stirred for 3 h at room temperature and quenched with aqueous NaHCO₃ solution. The resulting mixture was extracted with DCM three times. The combined organic layers were washed with brine, dried over Na₂SO₄, filtered and concentrated to dryness. The residue was purified by Prep-HPLC to give the desired product as a white solid (7.3 mg, yield: 18.7%). ESI: [M+H]⁺=477.2; ¹H NMR (400 MHz, CD₃OD): δ 8.05 (d, J=6.2 Hz, 1H), 7.96 (s, 1H), 7.79-7.61 (m, 8H), 7.25 (d, J=1.6 Hz, 1H), 5.32 (s, 2H), 3.95-3.60 (m, 5H), 2.68-2.55 (m, 1H), 2.45-2.35 (m, 1H), 2.25-2.15 (m, 1H), 1.40 (d, J=4.0 Hz, 6H).

Using the procedure described above, following example in Table 10 was synthesized.

TABLE 10

| | Ex. | | LC-MS |
|---|---|---|---|
| Structure | # | ¹H NMR | (M + H)⁺ |
| 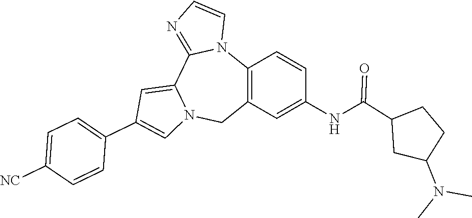 [SMILES: CN(C)C1CCC(C(=O)Nc2ccc3c(c2)Cn2cc(-c4ccc(C#N)cc4)cc2-c2nccn2-3)C1] | 78 | (400 MHz, CD₃OD): δ 8.05 (dd, J = 5.6, 2.3 Hz, 2H), 7.81 − 7.63 (m, 8H), 7.31 (s, 1H), 5.35 (s, 2H), 3.84 − 3.64 (m, 1H), 3.22 − 3.04 (m, 1H), 2.94 (s, 6H), 2.55 − 1.77 (m, 6H). | 477.2 |

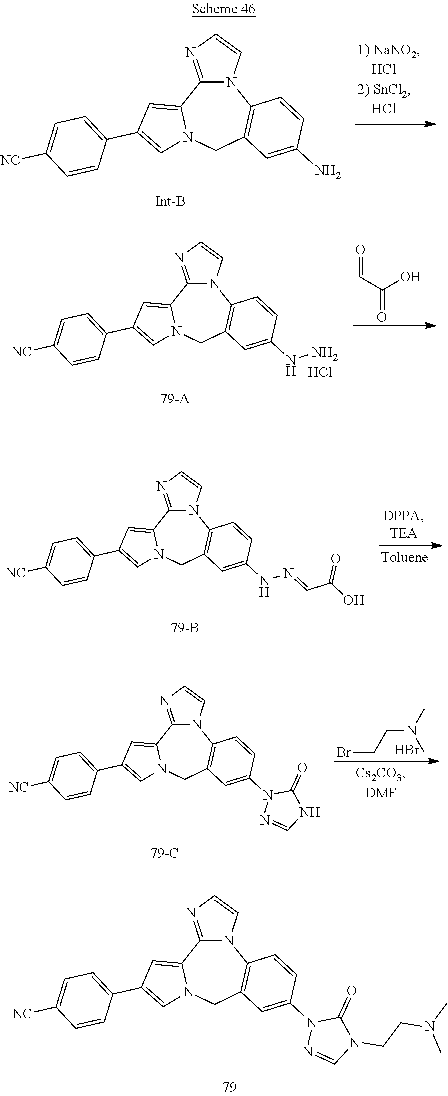

### 4-(7-Hydrazinyl-9H-benzo[e]imidazo[2,1-c]pyrrolo[1,2-a][1,4]diazepin-12-yl) benzonitrile hydrochloride (79-A)

To a mixture of compound 79-A (150 mg, 0.45 mmol) in HCl (2 mL) was added sodium nitrite (30 mg, 0.45 mmol) under ice-cooling, and the resulting mixture was stirred for 40 minutes followed by the addition of SnCl₂ (300 mg, 1.35 mmol) under ice-cooling. The reaction mixture was then stirred for another 2 h and filtered. The filter cake was washed with water and dried to give the desired product as a yellow solid (150 mg, yield: 68%).

### (E)-2-(2-(12-(4-Cyanophenyl)-9H-benzo[e]imidazo[2,1-c]pyrrolo[1,2-a][1,4]diazepin-7-yl)hydrazono)acetic acid (79-B)

To a mixture of compound 79-A (150 mg, 0.31 mmol) in concentrated HCl (20 mL) was added 2-oxoacetic acid (80 mg, 0.54 mmol, 50% wt). The mixture was stirred at room temperature for 2 h and filtered. The filter cake was washed with water and dried to give the desired product as a solid (130 mg, yield: 99%). ESI: [M+H]⁺=409.1

### 4-(7-(5-Oxo-4,5-dihydro-1H-1,2,4-triazol-1-yl)-9H-benzo[e]imidazo[2,1-c]pyrrolo[1,2-a][1,4]diazepin-12-yl)benzonitrile (79-C)

To a mixture of compound 79-B (110 mg, 0.27 mmol) in dry toluene (5 mL) was added TEA (82 mg, 0.81 mmol) and DPPA (110 mg, 0.40 mmol). The mixture was heated to reflux for 3 h. After cooling down to room temperature, the reaction mixture was concentrated to remove toluene, and the residue was dissolved in DCM. The resulting solution was washed with water, brine and dried with sodium sulfate. After filtration, the solution was concentrated and purified by silica gel chromatography (DCM:CH₃OH=10:1) to give the desired compound as a solid (40 mg, yield: 30%). ESI: [M+H]⁺=406.1

### 4-(7-(4-(2-(Dimethylamino)ethyl)-5-oxo-4,5-dihydro-1H-1,2,4-triazol-1-yl)-9H-benzo[e]imidazo[2,1-c]pyrrolo[1,2-a][1,4]diazepin-12-yl)benzonitrile (79)

To a mixture of 79-C (40 mg, 0.10 mmol) in DMF (1.5 mL) was added 2-bromo-N,N-dimethylethan-1-amine hydrobromide (100 mg, 0.5 mmol) and Cs₂CO₃ (160 mg, 0.5 mmol). The mixture was placed at 100° C. for 2 h in a microwave reactor. The reaction mixture was concentrated and purified by Prep-HPLC (TFA) to give the desired compound as a trifluoroacetate salt as a white solid (2 mg, yield: 4%). ESI: [M+H]+=477.2; ¹H NMR (400 MHz, CD₃OD): δ 8.36 (d, J=2.4 Hz, 1H), 8.26 (dd, J=8.9, 2.4 Hz, 1H), 8.12 (s, 1H), 8.02 (s, 1H), 7.81 (d, J=8.9 Hz, 1H), 7.78-7.73 (m, 3H), 7.72-7.63 (m, 3H), 7.28 (s, 1H), 5.41 (s, 2H), 4.23 (t, J=5.8 Hz, 2H), 3.60 (t, J=5.8 Hz, 2H), 3.01 (d, J=12.3 Hz, 6H).

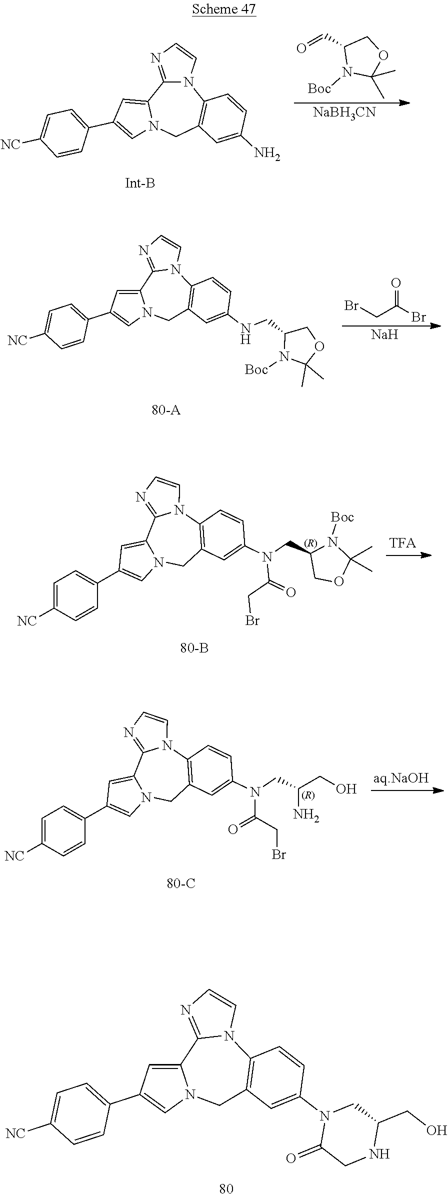

### tert-butyl (R)-4-(((12-(4-cyanophenyl)-9H-benzo[e]imidazo[2,1-c]pyrrolo[1,2-a][1,4]diazepin-7-yl)amino)methyl)-2,2-dimethyloxazolidine-3-carboxylate (80-A)

The solution of compound Int-B (800 mg, 2.37 mmol), tert-butyl (S)-4-formyl-2,2-dimethyloxazolidine-3-carboxylate (700 mg, 3.56 mmol) in methanol (40 mL) was stirred at room temperature for 1 hour, and then sodium cyanoborohydride (700 mg, 4.85 mmol) was added. The reaction mixture was stirred at room temperature for 1 hour and quenched by addition of aqueous NaHCO₃. The mixture was extracted with DCM three times. The combined organic layers were dried over Na₂SO₄, filtered and concentrated to dryness. The residue was purified by silica gel chromatography (DCM:CH₃OH=10:1) to give the desired compound as a white solid (300 mg, yield: 23%). ESI: [M+H]⁺=551.3

### tert-butyl (R)-4-((2-bromo-N-(12-(4-cyanophenyl)-9H-benzo[e]imidazo[2,1-c]pyrrolo[1,2-a][1,4]diazepin-7-yl)acetamido)methyl)-2,2-dimethyloxazolidine-3-carboxylate (80-B)

To a mixture of compound 80-A (100 mg, 0.18 mmol) in DCM (10 mL) was added sodium hydride (48 mg, 1.06 mmol) under ice-cooling, and the mixture was stirred at room temperature for 1 h. 2-bromoacetyl bromide (90 mg, 0.45 mmol) was added under ice-cooling before stirring at room temperature for 3 h. The reaction mixture was poured into iced 1N aq. NaOH and extracted with DCM three times. The combined organic layers were dried over Na₂SO₄, filtered and concentrated to give the crude product as a solid (80 mg), which was used directly to next step. ESI: [M+H]=671.2/673.2

### (R)—N-(2-Amino-3-hydroxypropyl)-2-bromo-N-(12-(4-cyanophenyl)-9H-benzo[e]imidazo[2,1-c]pyrrolo[1,2-a][1,4]diazepin-7-yl)acetamide (80-C)

To a solution of compound 80-B (80 mg, 0.12 mmol) in DCM (10 mL) was added TFA (100 mg, 0.87 mmol), and the resulting solution was stirred at room temperature for 1 h and concentrated to dryness. The residue was purified by Prep-HPLC (TFA) to give the desired compound as a trifluoroacetate salt as a white solid (70 mg, yield: 90%). ESI: [M+H]⁺=531.1/533.1

### (R)-4-(7-(5-(Hydroxymethyl)-2-oxopiperazin-1-yl)-9H-benzo[e]imidazo[2,1-c]pyrrolo[1,2-a][1,4]diazepin-12-yl)benzonitrile (80)

A biphasic solution of compound 80-C (60 mg, 0.09 mmol) in DCM (5 mL) and aq. NaOH (2 mL, 2N) was stirred at room temperature for 1 h. The reaction mixture was diluted with DCM and washed with water. The organic phase was dried over Na₂SO₄, filtered and concentrated to dryness, the residue was purified by Prep-HPLC (TFA) to give the desired compound as a trifluoroacetic acid salt as white solid (17 mg, yield: 34%). ESI: [M+H]⁺=451.2; ¹H NMR (400 MHz, CD₃OD): δ 8.02 (s, 1H), 7.81 (d, J=8.7 Hz, 1H), 7.75 (m, 4H), 7.70 (d, J=8.6 Hz, 2H), 7.66 (s, 1H), 7.64 (dd, J=8.6, 2.4 Hz, 1H), 7.28 (s, 1H), 5.38 (s, 2H), 4.11 (d, J=13.1 Hz, 2H), 4.05-3.98 (m, 1H), 3.99-3.89 (m, 3H), 3.85-3.75 (m, 1H).

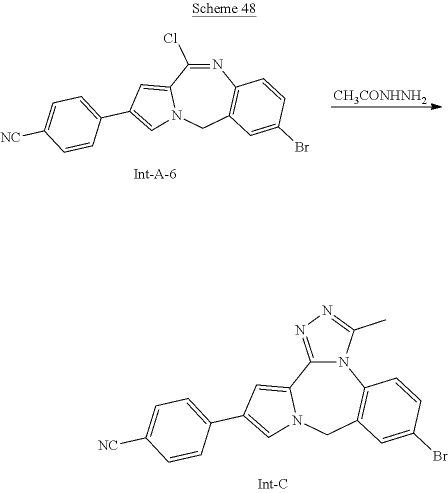

### 4-(7-Bromo-3-methyl-9H-benzo[e]pyrrolo[1,2-a][1,2,4]triazolo[3,4-c][1,4]diazepin-12-yl)benzonitrile (Int-C)

A solution of compound Int-A-6 (2.0 g, 5 mmol) and acetohydrazide (746 mg, 10 mmol) in dioxane (25 mL) was stirred at 110° C. for 16 h. The reaction mixture was concentrated under reduced pressure. The residue was diluted with ice-water (20 mL) and extracted with hot ethyl acetate/THF (2:1, 25 mL*3). The combined organic layers were washed with brine (30 mL), dried with anhydrous Na₂SO₄, filtered and concentrated in vacuum to afford black brown solid. The solid was triturated with MeOH (10 mL) to give the desired compound (1.30 g, crude) as a light yellow solid.

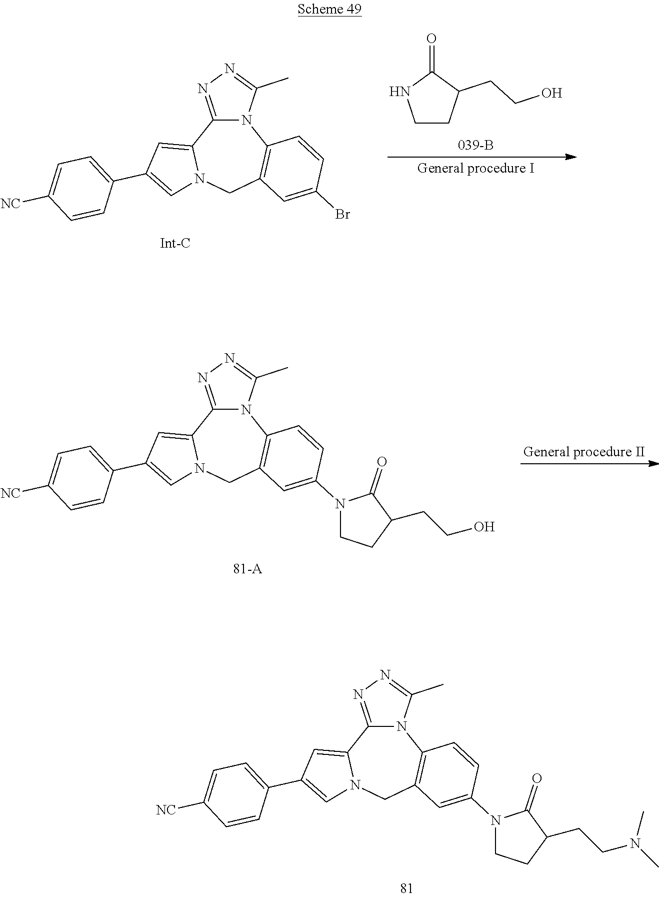 [SMILES: Cc1nnc2n1-c1ccc(Br)cc1Cn1cc(-c3ccc(C#N)cc3)cc1-2 ; O=C1NCCC1CCO ; Cc1nnc2n1-c1ccc(N3CCC(CCO)C3=O)cc1Cn1cc(-c3ccc(C#N)cc3)cc1-2 ; Cc1nnc2n1-c1ccc(N3CCC(CCN(C)C)C3=O)cc1Cn1cc(-c3ccc(C#N)cc3)cc1-2]

### 4-(7-(3-(2-Hydroxyethyl)-2-oxopyrrolidin-1-yl)-3-methyl-9H-benzo[e]pyrrolo [1,2-a][1,2,4]triazolo[3,4-c][1,4]diazepin-12-yl)benzonitrile (81-A)

Using the procedure described above, compound 081-A was synthesized from compound Int-C. ESI: [M+H]⁺=465.2

### 4-(7-(3-(2-(Dimethylamino)ethyl)-2-oxopyrrolidin-1-yl)-3-methyl-9H-benzo[e]pyrrolo[1,2-a][1,2,4]triazolo[3,4-c][1,4]diazepin-12-yl)benzonitrile (81)

Using the procedure described above, compound 081 was synthesized. ESI: [M+H]+=492.2; ¹H NMR (400 MHz, CD₃OD): δ 8.07 (dd, J=13.2, 2.2 Hz, 1H), 7.94-7.85 (m, 1H), 7.72 (d, J=8.4 Hz, 2H), 7.69-7.62 (m, 4H), 7.19 (d, J=1.8 Hz, 1H), 5.23 (s, 2H), 3.98-3.95 (m, 2H), 3.50-3.39 (m, 1H), 3.25 (s, 1H), 2.95 (d, J=2.2 Hz, 6H), 2.89-2.84 (m, 1H), 2.70 (s, 3H), 2.51-2.41 (m, 1H), 2.27-2.21 (m, 1H), 2.03-1.87 (m, 2H).

Using the procedure described above, the following examples in Table 11 were synthesized.

TABLE 11

| | Ex. | | LC-MS |
|---|---|---|---|
| Structure | # | ¹H NMR | (M + H)⁺ |
| 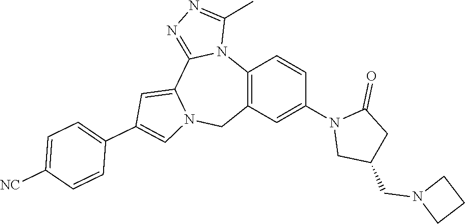 [SMILES: Cc1nnc2n1-c1ccc(N3C[C@@H](CN4CCC4)CC3=O)cc1Cn1cc(-c3ccc(C#N)cc3)cc1-2] | 82 | (400 MHz, CD₃OD): δ 8.11 (s, 1H), 7.73 − 7.71 (m, 3H), 7.67 − 7.63 (m, 4H), 7.19 − 7.18 (d, J = 4 Hz, 1H), 5.22 (s, 2H), 4.31 (m, 2H), 4.22 − 4.12 (m, 3H), 3.75 − 3.71 (m, 1H), 3.49 − 3.38 (m, 2H), 2.86 − 2.79 (m, 2H), 2.68 (s, 3H), 2.65 − 2.61 (m, 1H), 2.55 − 2.48 (m, 2H). | 490.2 |
| 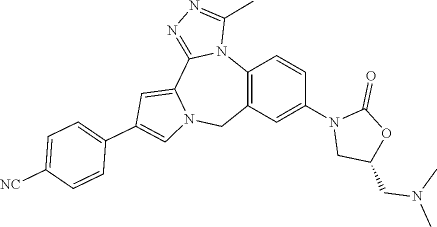 [SMILES: Cc1nnc2n1-c1ccc(N3C[C@@H](CN(C)C)OC3=O)cc1Cn1cc(-c3ccc(C#N)cc3)cc1-2] | 83 | (400 MHz, CD₃OD): δ 8.05-8.03 (d, J = 8 Hz,1H), 7.76 − 7.64 (m, 7H), 7.19 − 7.18 (m, 1H), 5.24 − 5.23 (d, J = 4 Hz, 2H), 4.40 − 4.35 (m, 1H), 3.91 − 3.87 (m, 1H), 3.74 − 3.68 (m, 1H), 3.54 − 3.47 (m, 2H), 3.30 (s, 6H), 2.68 (s, 3H) | 480.2 |
| 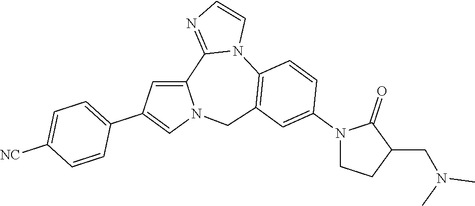 [SMILES: CN(C)CC1CCN(c2ccc3c(c2)Cn2cc(-c4ccc(C#N)cc4)cc2-c2nccn2-3)C1=O] | 84 | (400 MHz, CD₃OD): δ 8.06 (d, J = 7.6 Hz, 1H), 7.93 (s, 1H), 7.72 (d, J = 8.2 Hz, 2H), 7.69 − 7.60 (m, 4H), 7.19 (s, 1H), 5.23 (s, 2H), 4.01 (d, J = 8.7 Hz, 2H), 3.65-3.59 (m, 1H), 3.38-3.31 (m, 2H), 3.01 (s, 6H), 2.69 (s, 3H), 2.51-2.46 (m, 1H), 2.07 − 1.93 (m, 1H). | 478.2 |
| 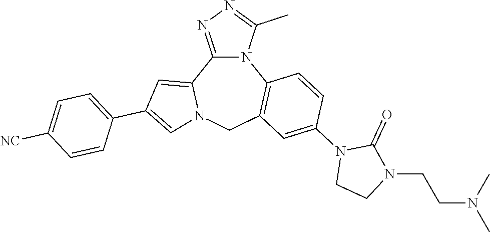 [SMILES: Cc1nnc2n1-c1ccc(N3CCN(CCN(C)C)C3=O)cc1Cn1cc(-c3ccc(C#N)cc3)cc1-2] | 85 | (400 MHz, CD₃OD): δ 8.01 (d, J = 2.6 Hz, 1H), 7.79 − 7.58 (m, 7H), 7.18 (d, J = 1.9 Hz, 1H), 5.20 (s, 2H), 4.08 − 3.93 (m, 2H), 3.78 − 3.57 (m, 4H), 3.41 (t, J = 5.7 Hz, 2H), 2.99 (s, 6H), 2.68 (s, 3H). | 493.2 |

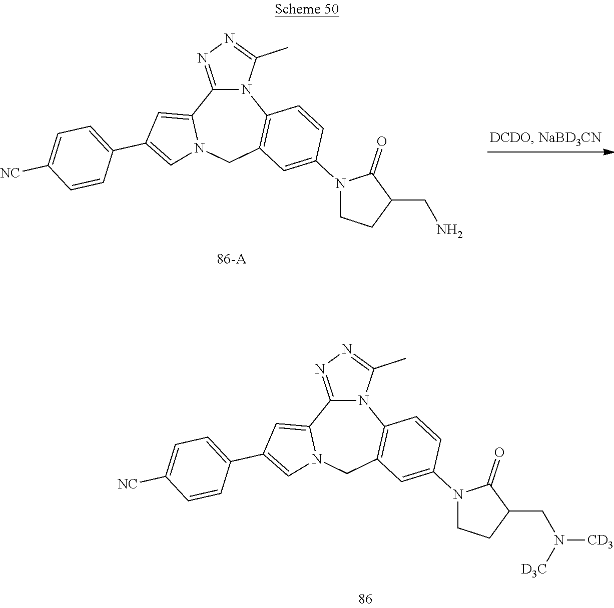 [SMILES: Cc1nnc2n1-c1ccc(N3CCC(CN)C3=O)cc1Cn1cc(-c3ccc(C#N)cc3)cc1-2 ; [2H]C([2H])([2H])N(CC1CCN(c2ccc3c(c2)Cn2cc(-c4ccc(C#N)cc4)cc2-c2nnc(C)n2-3)C1=O)C([2H])([2H])[2H]]

### 4-(7-(3-(Aminomethyl)-2-oxopyrrolidin-1-yl)-3-methyl-9H-benzo[e]pyrrolo[1,2-a] [1,2,4]triazolo[3,4-c][1,4]diazepin-12-yl)benzonitrile (86-A)

Using the procedure described above, compound 86-A was synthesized. ESI: [M+H]⁺=450.2

### 4-(7-(3-((Bis(methyl-d₃)amino)methyl)-2-oxopyrrolidin-1-yl)-3-methyl-9H-benzo [e]pyrrolo[1,2-a][1,2,4]triazolo[3,4-c][1,4]diazepin-12-yl)benzonitrile (86)

To a mixture of compound 86-A (30 mg, 0.07 mmol), DCDO (30 mg, 0.94 mmol) in CD₃OD (2 mL) was added NaBD₃CN (30 mg, 0.94 mmol). The mixture was stirred at room temperature for 1 hour. The reaction mixture was washed with water and extracted with dichloromethane. The organic phase w as dried with sodium sulfate, concentrated and purified by Prep-HPLC (TFA) to give the desired compound as a solid (12 mg, yield: 30%). ESI: [M+H]⁺=484.3; ¹H NMR (400 MHz, CD₃OD): δ 8.07 (d, J=5.8 Hz, 1H), 7.93 (s, 1H), 7.73-7.62 (m, 6H), 7.19 (d, J=1.7 Hz, 1H), 5.23 (s, 2H), 4.01 (d, J=8.5 Hz, 2H), 3.64-3.58 (m, 1H), 3.38-3.31 (m, 2H), 2.69 (s, 3H), 2.57-2.46 (m, 1H), 2.04-1.94 (m, 1H)

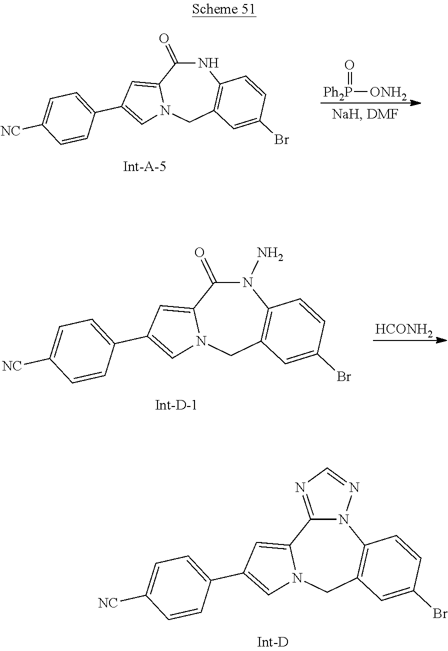

### 4-(10-Amino-7-bromo-11-oxo-10,11-dihydro-5H-benzo[e]pyrrolo[1,2-a][1,4]diazepin-2-yl)benzonitrile (Int-D-1)

To a solution of compound Int-A-5 (1 g, 2.64 mmol) in DMF (20 mL) was added NaH (169 mg, 4.2 mmol, 60% purity) at 0° C. After stirred for 1 h, (aminooxy) diphenylphosphine oxide (738 mg, 3.2 mmol) was added and the mixture was stirred at 20° C. for 1 h. The mixture was poured into ice-water (100 mL) and extracted with EtOAc (300 mL*3). The organic layer was washed with brine (100 mL), dried over MgSO4 and concentrated in vacuo. The residue was taken up in MTBE (20 mL) and stirred for 1 h. The precipitate was collected by filtration to give the desired product (1.0 g, crude) as a yellow solid, which was used into the next step without further purification. ESI [M+H]⁺=393.1/395.1

### 4-(7-Bromo-9H-benzo[e]pyrrolo[1,2-a][1,2,4]triazolo[5,1-c][1,4]diazepin-12-yl) benzonitrile (Int-D)

A solution of compound Int-D-1 (900 mg, 2.29 mmol) in formamide (10 mL) was stirred at 200° C. for 1.5 h. The mixture was cooled to room temperature, poured into ice-water (20 mL) and filtered. The filter cake was triturated with EtOH (10 mL) for 1 h and then collected by filtration to give the desired compound (600 mg) as a yellow solid. ESI [M+H]⁺=402.1/404.1; ¹H NMR (400 MHz, DMSO-d₆): δ 8.34 (br. s., 1H), 7.93 (br. s., 1H), 7.78 (br. s., 6H), 7.40-7.11 (m, 2H), 5.38 (br. s., 2H).

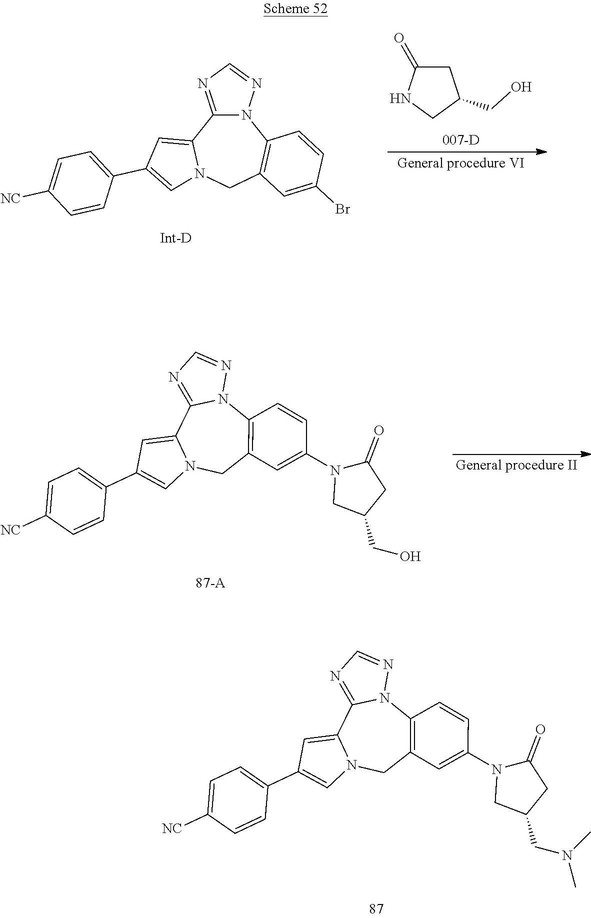 [SMILES: N#Cc1ccc(-c2cc3n(c2)Cc2cc(Br)ccc2-n2ncnc2-3)cc1 ; O=C1C[C@H](CO)CN1 ; N#Cc1ccc(-c2cc3n(c2)Cc2cc(N4C[C@@H](CO)CC4=O)ccc2-n2ncnc2-3)cc1 ; CN(C)C[C@H]1CC(=O)N(c2ccc3c(c2)Cn2cc(-c4ccc(C#N)cc4)cc2-c2ncnn2-3)C1]

### (S)-4-(7-(4-(Hydroxymethyl)-2-oxopyrrolidin-1-yl)-9H-benzo[e]pyrrolo [1,2-a][1,2,4]triazolo[5,1-c][1,4]diazepin-12-yl)benzonitrile (87-A)

Using the procedure described above, compound 87-A was synthesized as a light white solid. ESI: [M+H]⁺=437.2

### (R)-4-(7-(4-((Dimethylamino)methyl)-2-oxopyrrolidin-1-yl)-9H-benzo[e]pyrrolo [1,2-a][1,2,4]triazolo[5,1-c][1,4]diazepin-12-yl)benzonitrile (87)

Using the procedure described above, compound 87 was synthesized. ESI: [M+H]⁺=464.2; ¹H NMR (400 MHz, CD₃OD): δ 8.13 (s, 1H), 8.00 (d, J=2.4 Hz, 1H), 7.83 (d, J=8.9 Hz, 1H), 7.67-7.62 (m, 3H), 7.61-7.56 (m, 3H), 7.17 (d, J=1.8 Hz, 1H), 5.23 (s, 2H), 4.09 (dd, J=9.6, 7.2 Hz, 1H), 3.69 (dd, J=9.6, 7.2 Hz, 1H), 3.31 (d, J=7.3 Hz, 2H), 3.05-2.95 (m, 1H), 2.89 (s, 6H), 2.81 (dd, J=16.9, 8.3 Hz, 1H), 2.48 (dd, J=16.9, 8.3 Hz, 1H).

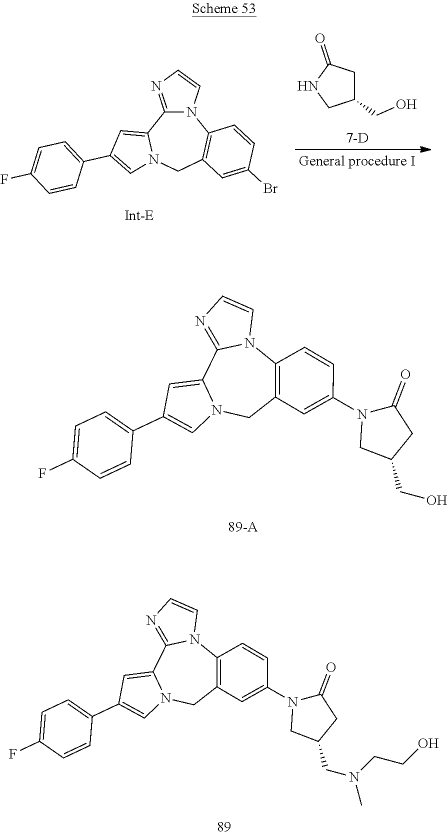 [SMILES: Fc1ccc(-c2cc3n(c2)Cc2cc(Br)ccc2-n2ccnc2-3)cc1 ; O=C1C[C@H](CO)CN1 ; O=C1C[C@H](CO)CN1c1ccc2c(c1)Cn1cc(-c3ccc(F)cc3)cc1-c1nccn1-2 ; CN(CCO)C[C@H]1CC(=O)N(c2ccc3c(c2)Cn2cc(-c4ccc(F)cc4)cc2-c2nccn2-3)C1]

### (S)-1-(12-(4-Fluorophenyl)-9H-benzo[e]imidazo[2,1-c]pyrrolo[1,2-a][1,4]diazepin-7-yl)-4-(hydroxymethyl)pyrrolidin-2-one (89-A)

Int-E was synthesized analogously to that of Int-A. Using the procedure described above, compound 89-A was synthesized. ESI: [M+H]⁺=429.2

### (R)-1-(12-(4-Fluorophenyl)-9H-benzo[e]imidazo[2,1-c]pyrrolo[1,2-a][1,4]diazepin-7-yl)-4-(((2-hydroxyethyl)(methyl)amino)methyl)pyrrolidin-2-one (89)

Using the procedure described above, compound 89 was synthesized. ESI: [M+H]+=486.2; ¹H NMR (400 MHz, CD₃OD): δ 8.21-7.89 (m, 3H), 7.74 (m, 2H), 7.67-7.46 (m, 3H), 7.20 (d, J=1.8 Hz, 1H), 7.15-6.99 (m, 2H), 5.37 (s, 2H), 4.22 (dd, J=9.7, 6.0 Hz, 1H), 3.94 (t, J=5.3 Hz, 2H), 3.77 (d, J=5.2 Hz, 1H), 3.48-3.46 (m, 4H), 3.29-3.24 (m, 1H), 3.05 (s, 3H), 2.56-2.54 (m, 1H), 2.14-1.93 (m, 1H).

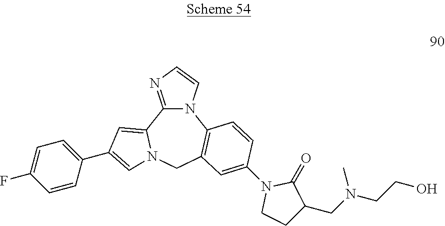 [SMILES: CN(CCO)CC1CCN(c2ccc3c(c2)Cn2cc(-c4ccc(F)cc4)cc2-c2nccn2-3)C1=O]

### (R)-1-(12-(4-Fluorophenyl)-9H-benzo[e]imidazo[2,1-c]pyrrolo[1,2-a][1,4]diazepin-7-yl)-3-(((2-hydroxyethyl)(methyl)amino)methyl)pyrrolidin-2-one (90)

Using the procedure described above, the title example was synthesized. ESI: [M+H]⁺=486.2; ¹H NMR (400 MHz, CD₃OD): δ 8.04 (m, 2H), 7.85 (dd, J=8.9, 2.4 Hz, 1H), 7.74 (d, J=8.9 Hz, 1H), 7.66 (d, J=1.6 Hz, 1H), 7.56 (td, J=5.3, 3.0 Hz, 3H), 7.17 (d, J=1.7 Hz, 1H), 7.12-6.99 (m, 2H), 5.34 (s, 2H), 4.20 (dd, J=9.7, 8.2 Hz, 1H), 3.91 (t, J=5.1 Hz, 2H), 3.82 (dd, J=9.7, 7.3 Hz, 1H), 3.48 (d, J=1.6 Hz, 1H), 3.35 (d, J=4.2 Hz, 2H), 3.28-3.25 (m, 1H), 3.21-3.08 (m, 1H), 3.05-3.01 (m, 3H), 2.94-2.92 (m, 1H), 2.63-2.60 (m, 1H).

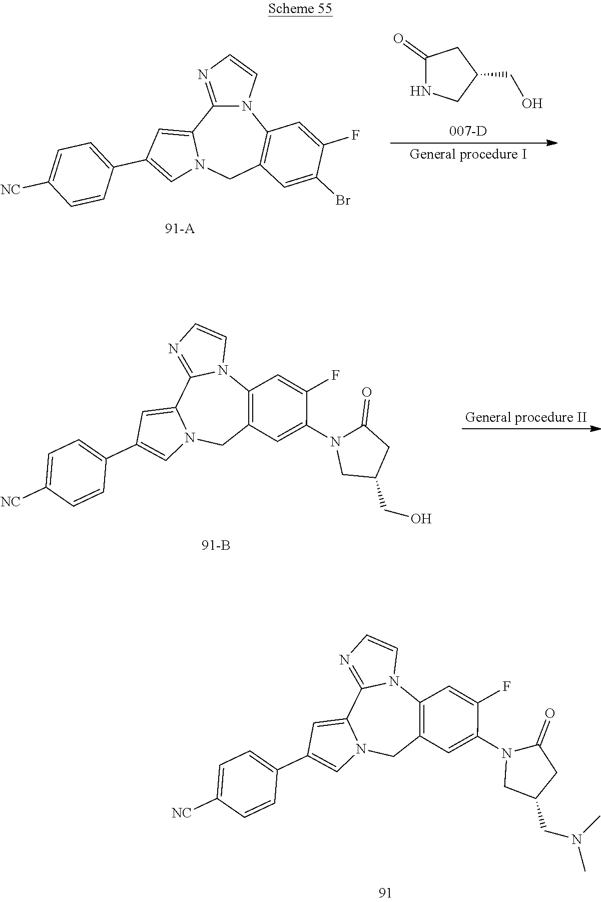 [SMILES: N#Cc1ccc(-c2cc3n(c2)Cc2cc(Br)c(F)cc2-n2ccnc2-3)cc1 ; O=C1C[C@H](CO)CN1 ; N#Cc1ccc(-c2cc3n(c2)Cc2cc(N4C[C@@H](CO)CC4=O)c(F)cc2-n2ccnc2-3)cc1 ; CN(C)C[C@H]1CC(=O)N(c2cc3c(cc2F)-n2ccnc2-c2cc(-c4ccc(C#N)cc4)cn2C3)C1]

### 4-(7-Bromo-6-fluoro-9H-benzo[e]imidazo[2,1-c]pyrrolo[1,2-a][1,4]diazepin-12-yl) benzonitrile (91-B)

Compound 91-A was synthesized analogously to that of Int-A. Using the procedure described above, compound 91-B was synthesized. ESI: [M+H]⁺=419.0/421.0

### (S)-4-(6-Fluoro-7-(4-(hydroxymethyl)-2-oxopyrrolidin-1-yl)-9H-benzo[e]imidazo [2,1-c]pyrrolo[1,2-a][1,4]diazepin-12-yl)benzonitrile (91)

Using the procedure described above, compound 91 was synthesized. ESI: [M+H]⁺=481.2; ¹H NMR (400 MHz, CD₃OD): δ 7.74 (d, J=7.8 Hz, 1H), 7.72-7.69 (m, 3H), 7.67-7.63 (m, 2H), 7.55 (d, J=11.2 Hz, 1H), 7.51 (d, J=1.9 Hz, 1H), 7.27 (d, J=1.5 Hz, 1H), 7.07 (d, J=1.9 Hz, 1H), 5.18 (s, 2H), 3.97 (dd, J=9.6, 7.7 Hz, 1H), 3.65 (dd, J=9.6, 6.1 Hz, 1H), 2.86 (dt, J=14.6, 7.4 Hz, 1H), 2.74 (dd, J=17.0, 8.6 Hz, 1H), 2.55 (qd, J=12.3, 7.5 Hz, 2H), 2.39 (dd, J=17.0, 7.1 Hz, 1H), 2.33 (s, 6H).

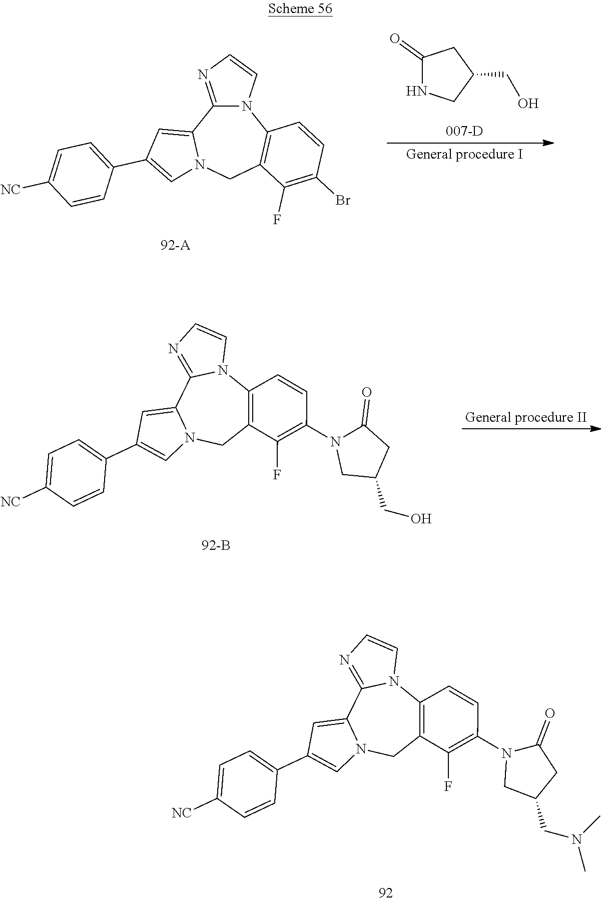 [SMILES: N#Cc1ccc(-c2cc3n(c2)Cc2c(ccc(Br)c2F)-n2ccnc2-3)cc1 ; O=C1C[C@H](CO)CN1 ; N#Cc1ccc(-c2cc3n(c2)Cc2c(ccc(N4C[C@@H](CO)CC4=O)c2F)-n2ccnc2-3)cc1 ; CN(C)C[C@H]1CC(=O)N(c2ccc3c(c2F)Cn2cc(-c4ccc(C#N)cc4)cc2-c2nccn2-3)C1]

### (R)-4-(7-(4-((Dimethylamino)methyl)-2-oxopyrrolidin-1-yl)-8-fluoro-9H-benzo[e]imidazo[2,1-c]pyrrolo[1,2-a][1,4]diazepin-12-yl)benzonitrile (92)

Using the procedures described above, compound 92 was synthesized. ESI: [M+H]⁺=481.2; ¹H NMR (400 MHz, CD₃OD): δ 8.04 (d, J=1.7 Hz, 1H), 7.81 (d, J=1.7 Hz, 1H), 7.78-7.66 (m, 6H), 7.65-7.60 (m, 1H), 7.31 (d, J=1.7 Hz, 1H), 5.49 (s, 2H), 4.08 (dd, J=9.6, 8.0 Hz, 1H), 3.76 (dd, J=9.5, 7.4 Hz, 1H), 3.41 (d, J=7.6 Hz, 2H), 3.23-3.10 (m, 1H), 2.97 (s, 6H), 2.86 (dd, J=17.0, 8.7 Hz, 1H), 2.54 (dd, J=17.0, 8.4, 1H).

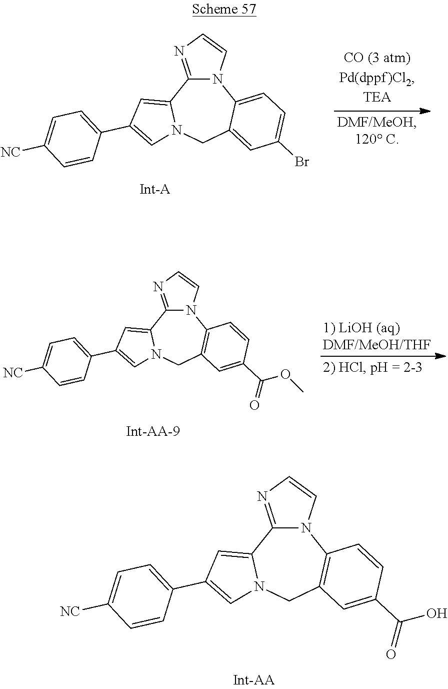 [SMILES: N#Cc1ccc(-c2cc3n(c2)Cc2cc(Br)ccc2-n2ccnc2-3)cc1 ; COC(=O)c1ccc2c(c1)Cn1cc(-c3ccc(C#N)cc3)cc1-c1nccn1-2 ; N#Cc1ccc(-c2cc3n(c2)Cc2cc(C(=O)O)ccc2-n2ccnc2-3)cc1]

### Methyl 12-(4-cyanophenyl)-9H-benzo[e]imidazo[2,1-c]pyrrolo[1,2-a][1,4]diazepine-7-carboxylate (Int-AA-9)

A suspension of Int-A (2.4 g, 6 mmol, 1.0 eq), Pd(dppf)Cl₂ (439 mg, 0.6 mmol, 0.05 eq) and TEA (1.8 g, 18 mmol, 3.0 eq) in DMF/MeOH (100 mL, 1:1) was de-gassed and then heated to 80˜100° C. for 12 h. under CO atmosphere (100 psi). The mixture was then cooled down to room temperature and concentrated under reduced pressure. The resulting residue was portioned between ethyl acetate (300 mL) and brine (300 mL). The organic layer was dried over anhydrous Na₂SO₄, filtered and concentrated to give crude product (2.4 g), which was used in the next step without further purification.

### 12-(4-cyanophenyl)-9H-benzo[e]imidazo[2,1-c]pyrrolo[1,2-a][1,4]diazepine-7-carboxylic acid (Int-AA)

To a mixture of crude compound Int-AA-9 (1.9 g, 5 mmol, 1.0 eq) in DMF/MeOH/THF (20 mL: 10 mL: 20 mL) was added 10% LiOH to adjust pH=10. The mixture was stirred for 1 h at room temperature. 1N HCl was added to adjust pH to 2˜3 and the resulting precipitate was collected by filtration to give desired acid (1.7 g) as white solid.

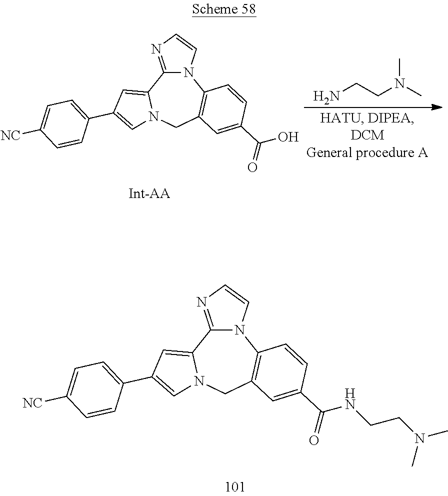

General Procedure A:

### 12-(4-Cyanophenyl)-N-(2-(dimethylamino)ethyl)-9H-benzo[e]imidazo[2,1-c]pyrrolo[1,2-a][1,4]diazepine-7-carboxamide (101)

To a solution of compound Int-AA (160 mg, 0.44 mmol), DIPEA (170 mg, 1.32 mmol) and N,N-dimethylethane-1,2-diamine (39 mg, 0.48 mmol) in DCM (5 mL) was added HATU (167 mg, 0.44 mmol) and the mixture was stirred at 20° C. for 2 h under nitrogen atmosphere until TLC indicated no remaining SM. The reaction mixture was quenched with water (20 mL), extracted with DCM (20 mL×2). The combined organic layers were washed with brine (20 mL) and dried over anhydrous Na₂SO₄. After filtration, the solvent was concentrated under reduced pressure, and the residue was purified by silica gel chromatography eluting with DCM/MeOH (100/1-20/1) to give the desired compound as a yellow solid (56 mg, yield: 29%). ESI: [M+H]⁺=437.2; ¹H NMR (400 MHz, CD₃OD): δ 8.21 (s, 1H), 8.08 (dd, J=7.8, 5.6 Hz, 2H), 7.83 (d, J=8.5 Hz, 1H), 7.78-7.72 (m, 3H), 7.70 (d, J=8.3 Hz, 3H), 7.30 (s, 1H), 5.43 (s, 2H), 3.80 (t, J=5.9 Hz, 2H), 3.41 (t, J=5.8 Hz, 2H), 3.00 (s, 6H).

### Examples 102-111

Compound 102-111 in Table 12 were synthesized by using procedures described above.

TABLE 12

| | Ex. | | LC-MS |
|---|---|---|---|
| Structure | # | ¹H NMR | (M + H)⁺ |
| 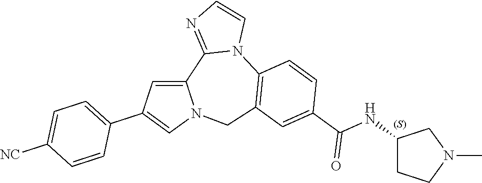 | 102 | (400 MHz, CD₃OD): δ 8.19 (d, J = 1.9 Hz, 1H), 8.10 − 8.03 (m, 2H), 7.82 (d, J = 8.4 Hz, 1H), 7.76 − 7.66 (m, 6H), 7.29 (d, J = 1.8 Hz, 1H), 5.43 (s, 2H), 4.60 (s, 1H), 3.86 (m, 2H), 3.53 − 3.42 (m, 1H), 3.18 − 2.92 (m, 4H), 2.29 | 449.2 |
| | | (s, 2H) | |
| 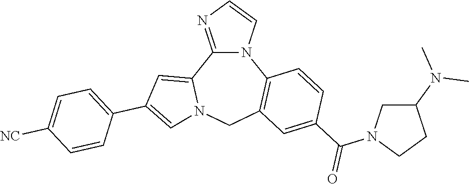 [SMILES: CN(C)C1CCN(C(=O)c2ccc3c(c2)Cn2cc(-c4ccc(C#N)cc4)cc2-c2nccn2-3)C1] | 103 | (400 MHz, CD₃OD): δ 8.07 (s, 1H), 7.90 (s, 1H), 7.82 − 7.69 (m, 8H), 7.31 (s, 1H), 5.44 (s, 2H), 3.89-3.80 (m, 5H), 3.02 − 2.87 (m, 6H), 2.48 (s, 1H), 2.22(s, 1H). | 463.2 |
| 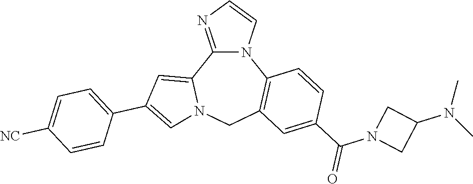 [SMILES: CN(C)C1CN(C(=O)c2ccc3c(c2)Cn2cc(-c4ccc(C#N)cc4)cc2-c2nccn2-3)C1] | 104 | (400 MHz, CD₃OD): δ 8.07 − 7.98 (m, 2H), 7.89 − 7.68 (m, 8H), 7.29 (s, 1H), 5.44 (s, 2H), 4.72 − 4.35 (m, 4H), 4.26-4.18 (m, 1H), 2.91 (s, 6H). | 449.2 |
| 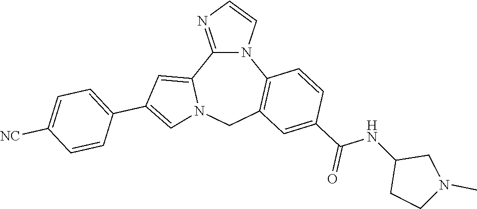 [SMILES: CN1CCC(NC(=O)c2ccc3c(c2)Cn2cc(-c4ccc(C#N)cc4)cc2-c2nccn2-3)C1] | 105 | (400 MHz, CD₃OD): δ 8.20 (d, J = 1.9 Hz, 1H), 8.08 (d d, J = 8.4, 2.0 Hz, 1H), 8.05 (d, J = 1.8 Hz, 1H), 7.82 (d, J = 8.4 Hz, 1H), 7.77 − 7.72 (m, 3H), 7.70 (d, J = 8.6 Hz, 2H), 7.67 (d, J = 1.6 Hz, 1H), 7.29 (d, J = 1.7 Hz, 1H), 5.43 (s, 2H), 4.65 | 449.2 |
| | | (s, 1H), 4.06-3.98 | |
| | | (m, 1H), 3.80 (s, 1H), | |
| | | 3.25 − 3.09 (m, | |
| | | 2H), 3.00 (s, 3H), | |
| | | 2.59-2.54 (m, 1H), | |
| | | 2.29 (s, 1H). | |
| 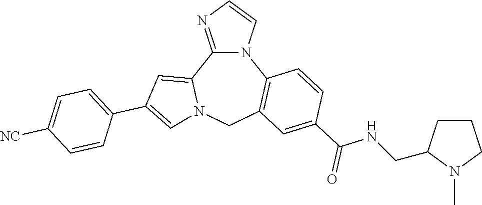 [SMILES: CN1CCCC1CNC(=O)c1ccc2c(c1)Cn1cc(-c3ccc(C#N)cc3)cc1-c1nccn1-2] | 106 | (400 MHz, CD₃OD): δ 8.22 (s, 1H), 8.12 − 8.03 (m, 2H), 7.84 (d, J = 8.4 Hz, 1H), 7.71 (m, 6H), 7.28 (s, 1H), 5.43 (s, 2H), 3.98- 3.92 (m, 1H), 3.74- 3.66 (m, 2H), 3.24 − 3.05 (m, 5H), 2.35 − 1.98 (m, 4H). | 463.2 |
| 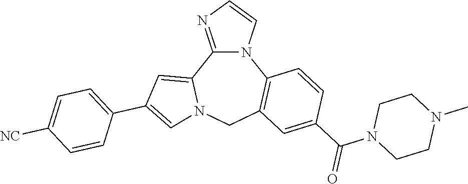 [SMILES: CN1CCN(C(=O)c2ccc3c(c2)Cn2cc(-c4ccc(C#N)cc4)cc2-c2nccn2-3)CC1] | 107 | (400 MHz, DMSO-d₆) δ 10.00 (s, 1H), 8.08 (s, 1H), 7.82 − 7.74 (m, 7H), 7.68 (dd, J = 8.3, 1.8 Hz, 1H), 7.59 (s, 1H), 7.22 (d, J = 1.5 Hz, 1H), 5.39 (s, 2H), 3.44 − 3.09 (m, 8H), 2.83 (s, 3H). | 449.2 |
| 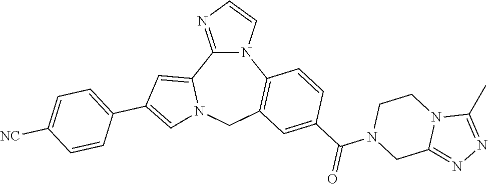 [SMILES: Cc1nnc2n1CCN(C(=O)c1ccc3c(c1)Cn1cc(-c4ccc(C#N)cc4)cc1-c1nccn1-3)C2] | 108 | (400 MHz, CD₃OD) δ 8.15 (s, 1H), 7.88 (d, J = 8.2 Hz, 2H), 7.82 − 7.78 (m, 3H), 7.76 (d, J = 8.6 Hz, 2H), 7.71 (d, J = 8.5 Hz, 2H), 7.35 (s, 1H), 5.47 (s, 2H), 4.85- 4.78 (m, 4H), 4.12 (s, 2H), 2.48(s, 3H). | 487.2 |
| 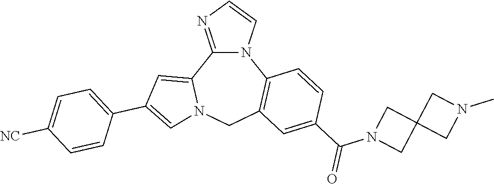 [SMILES: CN1CC2(C1)CN(C(=O)c1ccc3c(c1)Cn1cc(-c4ccc(C#N)cc4)cc1-c1nccn1-3)C2] | 109 | (400 MHz, CD₃OD) δ 8.07 (s, 1H), 7.97 (s, 1H), 7.89 − 7.85 (m, 1H), 7.82 (d, J = 8.4 Hz, 1H), 7.78 − 7.73 (m, 3H), 7.73 − 7.68 m, 3H), 7.30 (s, (1H), 5.43 (s, 2H), 4.66-4.60 (m, 2H), 4.55-4.47 (m, 2H), 4.45-4.37 (m, 2H), | 461.2 |
| | | 4.26-4.18 (m, 2H), | |
| | | 2.91 (s, 3H). | |
| 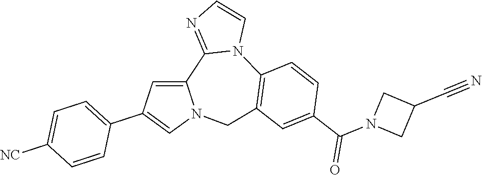 [SMILES: N#Cc1ccc(-c2cc3n(c2)Cc2cc(C(=O)N4CC(C#N)C4)ccc2-n2ccnc2-3)cc1] | 110 | (400 MHz, CDCl₃) δ 7.85 (d, J = 1.8 Hz, 1H), 7.68 − 7.53 (m, 5H), 7.49 (d, J = 8.3 Hz, 1H), 7.42 (d, J = 1.4 Hz, 1H), 7.35 (d, J = 1.4 Hz, 1H), 7.19 (d, J = 1.9 Hz, 1H), 7.14 (d, J = 1.7 Hz, 1H), 5.16 − 5.07 | 431.2 |
| | | (m, 2H), 4.59-4.52 | |
| | | (m, 4H), 3.64-3.57 | |
| | | (m, 1H). | |
| 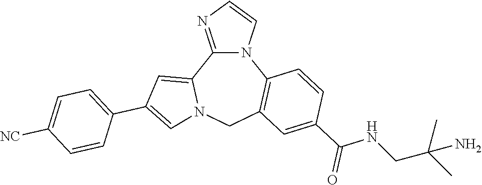 [SMILES: CC(C)(N)CNC(=O)c1ccc2c(c1)Cn1cc(-c3ccc(C#N)cc3)cc1-c1nccn1-2] | 111 | (400 MHz, CD₃OD) δ 8.21 (d, J = 1.7 Hz, 1H), 8.10 (d, d, J = 8.4, 2.0 Hz, 1H), 8.04 (s, 1H), 7.83 (d, J = 8.4 Hz 1H), 7.75 (dd, J = 6.2, 2.3 Hz, 3H), 7.72 − 7.67 (m, 2H), 7.65 (s, 1H), 7.28 (s, 1H), 5.43 (s, 2H), 3.57 (s, 2H), 1.40 (s, 6H). | 437.2 |

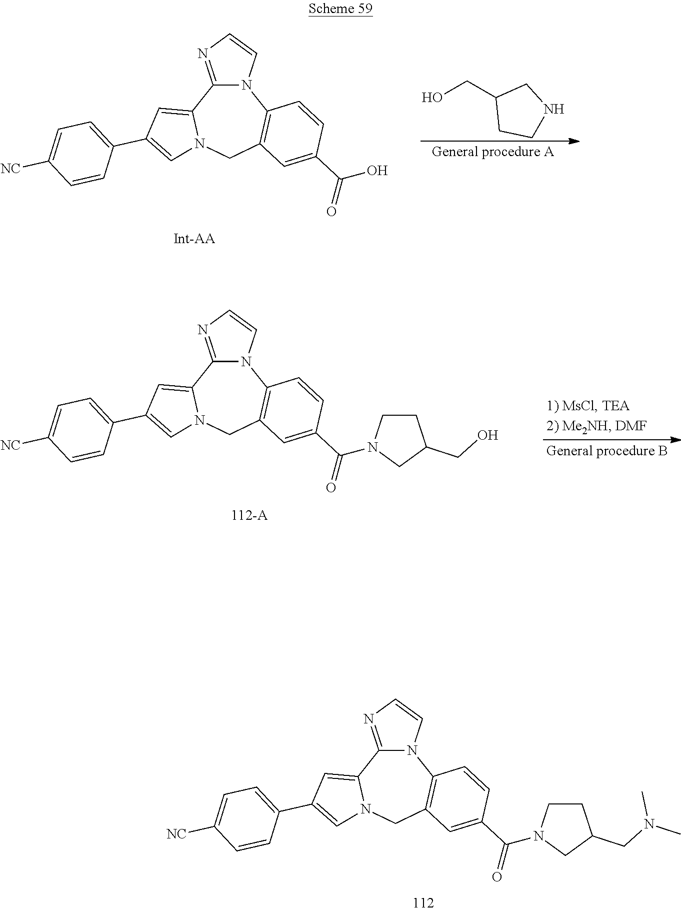 [SMILES: N#Cc1ccc(-c2cc3n(c2)Cc2cc(C(=O)O)ccc2-n2ccnc2-3)cc1 ; OCC1CCNC1 ; N#Cc1ccc(-c2cc3n(c2)Cc2cc(C(=O)N4CCC(CO)C4)ccc2-n2ccnc2-3)cc1 ; CN(C)CC1CCN(C(=O)c2ccc3c(c2)Cn2cc(-c4ccc(C#N)cc4)cc2-c2nccn2-3)C1]

### 4-(7-(3-(Hydroxymethyl)pyrrolidine-1-carbonyl)-9H-benzo[e]imidazo[2,1-c]pyrrolo[1,2-a][1,4]diazepin-12-yl)benzonitrile (112-A)

Compound 112-A was prepared by using General procedure A (45 mg, yield: 36.9%). ESI: [M+H]⁺=450.2.

General Procedure B:

### 4-(7-(3-((dimethylamino)methyl)pyrrolidine-1-carbonyl)-9H-benzo[e]imidazo [2,1-c]pyrrolo[1,2-a][1,4]diazepin-12-yl)benzonitrile (112)

To a solution of 112-A (45 mg, 0.1 mol) and TEA (1 mL) in DCM (4 mL) was added MsCl (20 mg, 0.17 mol) at 0° C. under N₂ atmosphere. The mixture was stirred for 1 h until TLC indicated no remaining SM. The mixture was poured into saturated NaHCO₃ aqueous (20 mL) and then extracted with DCM (20 mL×2). The combined organic layers were washed with brine (20 mL), dried over Na₂SO₄ and filtered. The organic layer was concentrated under vacuum. The residue was purified by silica gel chromatography eluting with DCM/MeOH (100/1-20/1) to afford the desired product as a solid (30 mg, yield: 57.7%). ESI [M+H]⁺=528.2. To a solution of above alcohol (30 mg, 0.06 mol) in DMF (1 mL) was added dimethylamine in THE (1 mol/L, 1 mL) under microwave condition at 100° C. for 1 h. The mixture was poured into water (20 mL) and then extracted with DCM (20 mL×2). The combined organic layers were washed with brine (20 mL×2), dried over Na₂SO₄ and filtered. The organic layer was concentrated under vacuum. The residue was purified by prep-HPLC to get the desired compound 112 as a solid (8.4 mg, yield: 31.1%). ESI: [M+H]⁺=477.2; ¹H NMR (400 MHz, CD₃OD): δ 8.01-7.99 (d, J=10.7 Hz, 1H), 7.88-7.86 (d, J=7.0 Hz, 1H), 7.79-7.68 (m, 7H), 7.64 (m, 1H), 7.27 (s, 1H), 5.40-5.39 (d, J=4.7 Hz, 2H), 3.81-3.61 (m, 4H), 3.17 (m, 2H), 2.97 (s, 3H), 2.85 (s, 3H), 2.74 (m, 1H), 2.28-2.19 (m, 1H), 1.78-1.76 (m, 1H).

### Example 113-118

Compound 113-118 in Table 13 were synthesized by using similar procedures described above.

TABLE 13

| | Ex. | | LC-MS |
|---|---|---|---|
| Structure | # | ¹H NMR | (M + H)⁺ |
| 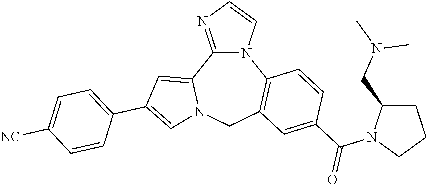 [SMILES: CN(C)C[C@H]1CCCN1C(=O)c1ccc2c(c1)Cn1cc(-c3ccc(C#N)cc3)cc1-c1nccn1-2] | 113 | (400 MHz, CD₃OD) δ 8.00 − 7.98 (m, 1H), 7.91 (s, 1H), 7.84-7.78 (m, 2H), 7.75 − 7.68 (m, 5H), 7.62 − 7.56 (m, 1H), 7.25 (s, 1H), 7.33 (s, 1H), 5.39 (s, 2H), 4.60 − 4.58 (m, 1H), 3.67 − 3.48 (m, 4H), 3.10 (s, 3H), 2.98 (s, 3H), 2.38-2.31 (m, 1H), 1.99-1.92 (m, | 477.2 |
| | | 1H), 1.82 − 1.79 (m, 2H). | |
| 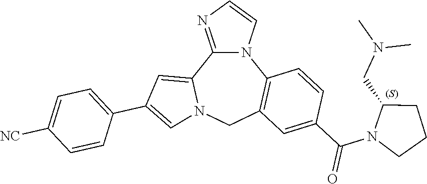 | 114 | (600 MHz, CD₃OD): δ 8.02 (s, 1H), 7.93 (d, J = 1.4 Hz, 1H), 7.83-7.66 (m, 7H), 7.63 (s, 1H), 7.27 (s, 1H), 5.41 (s, 2H), 4.63-4.0 (m, 1H), 3.72 − 3.62 (m, 1H), 3.61-3.49 (m, 2H), 3.36 − 3.33 (m, 1H), 3.11 (s, 3H), 2.97 (s, 3H), 2.40 − 2.28 (m, 1H), 2.03 − 1.92 (m, 1H), 1.92 − 1.73 (m, 2H). | 477.2 |
| 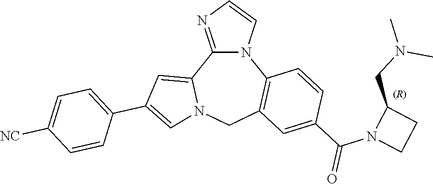 | 115 | (400 MHz, CD₃OD): δ 8.05-8.04 (d, J = 4 Hz, 2H), 7.95 − 7.93 (m, 1H), 7.83 −7.81 (m, 1H), 7.75 − 7.67 (m, 6H), 7.29 − 7.28 (d, J = 4 Hz, 1H), 5.44 (s, 2H) , 5.09 − 5.04 (m, IH), 4.51 − 4.45 (m, 1H), 4.41 − 4.35 (m, 1H), 3.81 − 3.72 (m, 1H), 3.44 − 3.40 (m, 1H), 3.10 (s, 3H), 3.01 (s, 3H), 2.66 − 2.57 (m, 1H), 2.25 − 2.17 (m, 1H). | 463.2 |
| 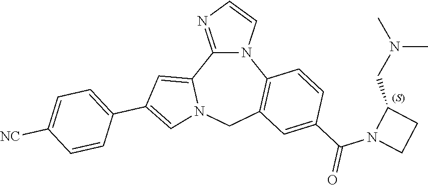 | 116 | (400 MHz, CD₃OD): δ 8.10 − 8.04 (m, 2H), 7.95 (dd, J = 8.4, 1.9 Hz, 1H), 7.84 (d, J = 8.4 Hz, 1H), 7.78 − 7.68 (m, 6H), 7.32 (s, 1H), 5.45 (s, 2H), 5.10 − 5.02 (m, 1H), 4.53 − 4.35 (m, 2H), 3.82- 3.76 (m, 1H), 3.46- 3.40 (m, 1H), 3.10 (s, 3H), 3.01 (s, 3H), 2.63 − | 463.2 |
| | | 2.60 (m, 1H), 2.27 − | |
| | | 2.16 (m, 1H). | |
| 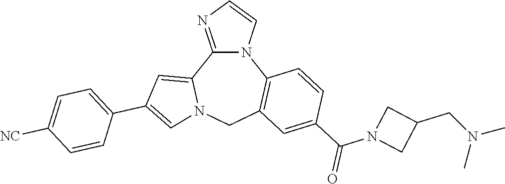 [SMILES: CN(C)CC1CN(C(=O)c2ccc3c(c2)Cn2cc(-c4ccc(C#N)cc4)cc2-c2nccn2-3)C1] | 117 | (400 MHz, CD₃OD): δ 8.05 (d, J = 2.0 Hz, 1H), 7.99 (d, J = 1.8 Hz, 1H), 7.88 (dd, J = 8.4, 1.9 Hz, 1H), 7.81 (d, J = 8.4 Hz, 1H), 7.75-7.66 (m, 6H), 7.30 (d, J = 1.8 Hz, 1H), 5.43 (s, 2H), 4.61 (t, J = 8.6 Hz, 1H), 4.41 (t, J = 9.5 Hz, 1H), 4.25 (s, 1H), 4.07 − | 463.2 |
| | | 3.96 (m, 1H), 3.57 − | |
| | | 3.44 (m, 2H) , 3.26 − | |
| | | 3.21 (m, 1H), 2.89 (s, 6H). | |
| 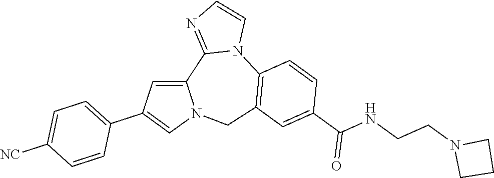 [SMILES: N#Cc1ccc(-c2cc3n(c2)Cc2cc(C(=O)NCCN4CCC4)ccc2-n2ccnc2-3)cc1] | 118 | (400 MHz, CD₃OD): δ 8.19 (d, J = 1.9 Hz, 1H), 8.07 (dd, J = 8.4, 1.9 Hz, 1H), 8.00 (s, 1H), 7.81 (d, J = 8.5 Hz, 1H), 7.74-7.66 (m, 5H), 7.61 (s, 1H), 7.26 (s, 1H), 5.40 (s, 2H), 4.36-4.29 (m, 2H), 4.22-4.15 (m, 2H), 3.70 − 3.61 (m, 2H), 3.48 − 3.42 (m, | 449.2 |
| | | 2H), 2.66 − 2.36 (m, 2H). | |

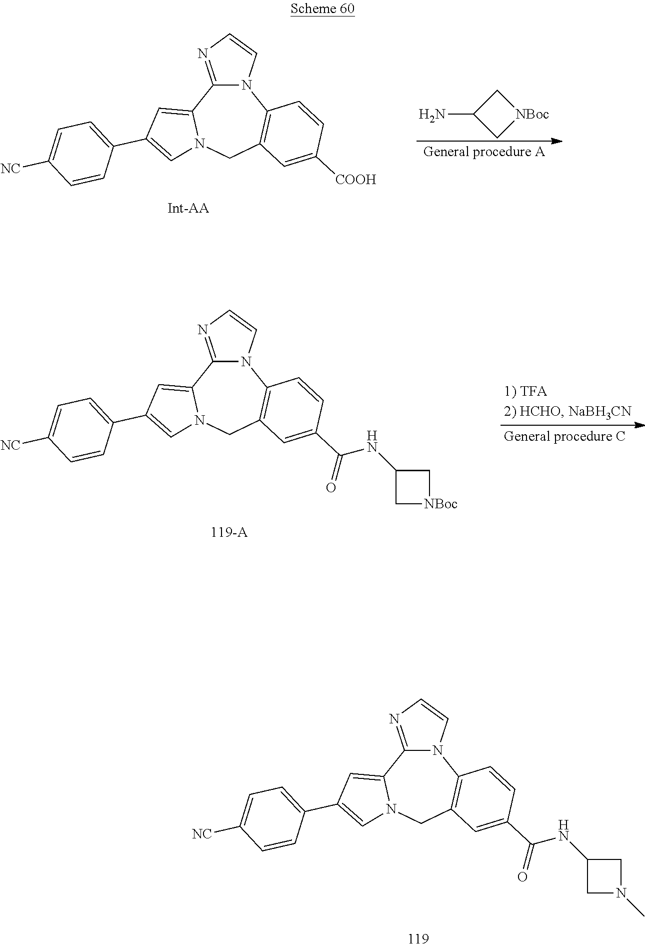 [SMILES: N#Cc1ccc(-c2cc3n(c2)Cc2cc(C(=O)O)ccc2-n2ccnc2-3)cc1 ; CC(C)(C)OC(=O)N1CC(N)C1 ; CC(C)(C)OC(=O)N1CC(NC(=O)c2ccc3c(c2)Cn2cc(-c4ccc(C#N)cc4)cc2-c2nccn2-3)C1 ; CN1CC(NC(=O)c2ccc3c(c2)Cn2cc(-c4ccc(C#N)cc4)cc2-c2nccn2-3)C1]

### tert-butyl-3-(12-(4-cyanophenyl)-9H-benzo[e]imidazo[2,1-c]pyrrolo[1,2-a][1,4]diazepine-7-carboxamido)azetidine-1-carboxylate (119-A)

Compound 119-A was prepared by using General procedure A (30 mg, yield: 21%). ESI: [M+H]⁺=521.2.

General Procedure C:

### 2-(4-Cyanophenyl)-N-(1-methylazetidin-3-yl)-9H-benzo[e]imidazo[2,1-c]pyrrolo [1,2-a][1,4]diazepine-7-carboxamide (119)

To a stirred mixture of compound 119-A (30 mg, 0.06 mmol) in DCM (2 mL) was added TFA (1 mL), and the reaction solution was stirred at room temperature for 1 h until TLC indicated no remaining SM. The reaction mixture was concentrated under vacuum. The residue was dissolved in DCM (10 mL) and saturated NaHCO₃ aqueous (20 mL) was added to adjust pH>7. The mixture was extracted with DCM (20 mL×2). The combined organic layers were washed with brine (20 mL), dried over Na₂SO₄ and filtered. The solution was concentrated under vacuum to give the amine (20 mg, crude). ESI: [M+H]⁺=421.2.

To a solution of above compound (20 mg, 0.05 mmol) and paraformaldehyde (15 mg, 0.5 mmol) in DCM (3 mL) and MeOH (3 mL) was added NaBH₃CN (15 mg, 0.25 mmol), and the reaction mixture was stirred at room temperature for 1 h. The mixture was poured into saturated NaHCO₃ aqueous (10 mL) and then extracted with DCM (10 mL×2). The combined organic layers were washed with brine (10 mL), dried over Na₂SO₄ and filtered. The organic solution was concentrated under vacuum, and the residue was purified by Prep-HPLC to give the desired compound as a white solid (10 mg, yield: 38%). ESI: [M+H]⁺=435.2; ¹H NMR (400 MHz, CD₃OD): δ 8.20 (s, 1H), 8.08 (s, 1H), 8.01 (s, 1H), 7.83 (d, J=8.5 Hz, 1H), 7.72 (m, 5H), 7.63 (s, 1H), 7.26 (s, 1H), 5.42 (s, 2H), 4.84 (m, 1H), 4.64 (m, 2H), 4.35 (m, 1H), 4.27 (m, 1H), 3.03 (d, J=16.1 Hz, 3H).

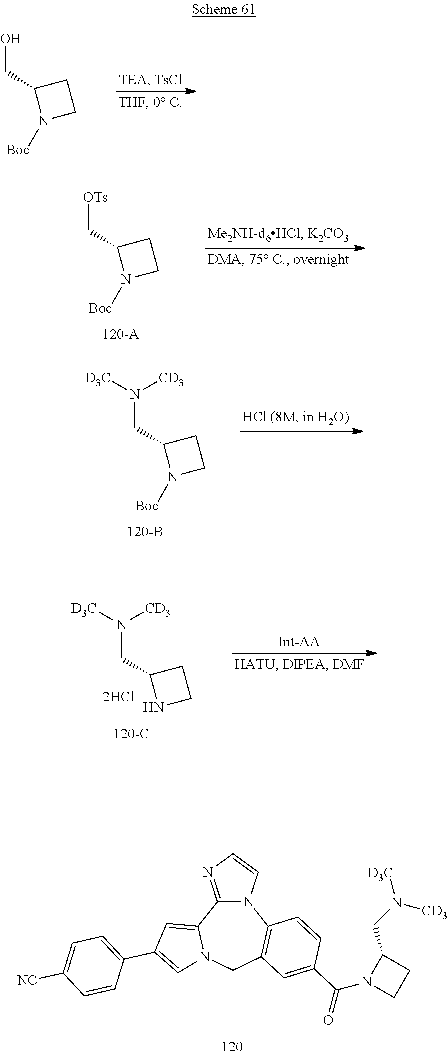 [SMILES: CC(C)(C)OC(=O)N1CC[C@H]1CO ; Cc1ccc(S(=O)(=O)OC[C@@H]2CCN2C(=O)OC(C)(C)C)cc1 ; [2H]C([2H])([2H])N(C[C@@H]1CCN1C(=O)OC(C)(C)C)C([2H])([2H])[2H] ; [2H]C([2H])([2H])N(C[C@@H]1CCN1)C([2H])([2H])[2H] ; [2H]C([2H])([2H])N(C[C@@H]1CCN1C(=O)c1ccc2c(c1)Cn1cc(-c3ccc(C#N)cc3)cc1-c1nccn1-2)C([2H])([2H])[2H]]

### tert-butyl (S)-2-(((methylsulfonyl)oxy)methyl)azetidine-1-carboxylate (120-A)

To a solution of tert-butyl (S)-2-(hydroxymethyl)azetidine-1-carboxylate (10 g, 53.4 mmol) in THE (100 mL) was added TEA (6.47 g, 64 mmol) and TsCl (11.7 g, 61.4 mmol) at 0° C. The mixture was stirred at room temperature overnight. The mixture was filtered, and the filtrate was concentrated and purified by chromatography on silica gel eluting with PE:EA=10:1 to get the desired product (17.9 g) as light a yellow oil.

### tert-butyl (S)-2-((bis(methyl-d₃)amino)methyl)azetidine-1-carboxylate (120-B)

To a solution of compound 120-A (2.53 g, 7.35 mmol) in DMA (10 mL) was added dimethylamine-d₆ Hydrochloride (3 g, 36.8 mmol) and K₂CO₃ (5.08 g, 36.8 mmol) at 25° C., and the mixture was stirred at room temperature for 30 min. The supernatant in above mixture was transferred to a sealed tube and stirred at 75° C. overnight. After cooling to room temperature, the solution was then concentrated under reduced pressure. The residue was purified by chromatography on silica gel eluting with DCM:MeOH=20:1 to get the desired product (1.24 g, 76.8%) as light a yellow oil. ¹H NMR (400 MHz, CDCl₃): δ 4.29 (d, J=8.0 Hz, 1H), 3.88-3.78 (m, 2H), 2.76-2.72 (m, 1H), 2.57-2.51 (m, 1H), 2.34-2.33 (m, 1H), 2.06-1.98 (m, 1H), 1.44 (s, 9H).

### (S)—N-(Azetidin-2-ylmethyl)-N-(methyl-d₃)methanamine-d₃ (120-C)

To a solution of compound 13 (1.24 g) was added HCl (8M, 10 mL) at 0° C. The resulting solution was stirred at room temperature overnight, and then washed with DCM (20 mL×2) and MTBE (20 mL×2). The aqueous layer was freeze-dried to get the desired product (700 mg, 99%) in its HCl salt form as a light a yellow solid

### (S)-4-(7-(2-((Bis(methyl-d₃)amino)methyl)azetidine-1-carbonyl)-9H-benzo[e]imidazo[2,1-c]pyrrolo[1,2-a][1,4]diazepin-12-yl)benzonitrile (120)

A suspension of compound Int-AA (1.26 g, 3.45 mmol), compound 120-C (700 mg, 3.62 mmol), HATU (1.44 g, 3.79 mmol) and DIPEA (2.4 g, 18.63 mmol) in DMA (10 mL) was stirred at 0° C. for 10 min. After the reaction was complete, the mixture was concentrated and purified by chromatography on silica gel eluting with DCM:MeOH=20:1 to get the desired product (840 mg, 52.2%) as a white solid. ESI [M+H]⁺=469.3

¹H NMR (400 MHz, CDCl₃): δ 7.83 (d, J=1.5 Hz, 1H), 7.69 (d, J=8.1 Hz, 1H), 7.60 (d, J=8.5 Hz, 2H), 7.56 (d, J=8.5 Hz, 2H), 7.45 (d, J=8.3 Hz, 1H), 7.42 (d, J=1.3 Hz, 1H), 7.33 (d, J=1.3 Hz, 1H), 7.26 (s, 1H), 7.17 (d, J=1.8 Hz, 1H), 7.11 (d, J=1.8 Hz, 1H), 5.10 (s, 2H), 4.78-4.62 (m, 1H), 4.42-4.30 (m, 1H), 4.09 (dd, J=15.2, 9.0 Hz, 1H), 3.02-2.87 (m, 1H), 2.76-2.63 (m, 1H), 2.58-2.46 (m, 1H), 2.28-2.15 (s, 1H).

### Example 121-131

Compounds 121-131 in Table 14 were synthesized by using similar procedures as described above.

TABLE 14

| | Ex. | | LC-MS |
|---|---|---|---|
| Structure | # | ¹H NMR | (M + H)⁺ |
| 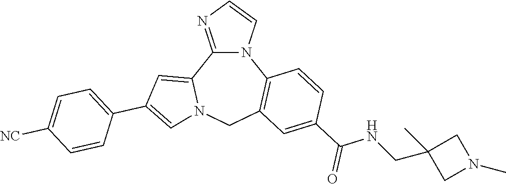 [SMILES: CN1CC(C)(CNC(=O)c2ccc3c(c2)Cn2cc(-c4ccc(C#N)cc4)cc2-c2nccn2-3)C1] | 121 | (400 MHz, CD₃OD): δ 8.18 (s, 1H), 8.07 (d, J = 8.5 Hz, 1H), 8.02 (d, J = 1.8 Hz, 1H), 7.82 (d, J = 8.4 Hz, 1H), 7.74-7.65 (m, 5H), 7.63 (d, J = 1.6 Hz, 1H), 7.27 (d, J = 1.8 Hz, 1H), 5.42 (s, 2H), 4.37 (d, J = 11.5 Hz, 1H), 4.02 | 463.2 |
| | | (d, J = 25.0, 11.1 | |
| | | Hz, 2H), 3.82 (d, J = | |
| | | 11.6 Hz, 1H), 3.67 | |
| | | (s, 1H), 3.55 (s, 1H), | |
| | | 2.92 (d, J = 17.5 Hz, | |
| | | 3H), 1.44 (d, J = 9.9 | |
| | | Hz, 3H) | |
| 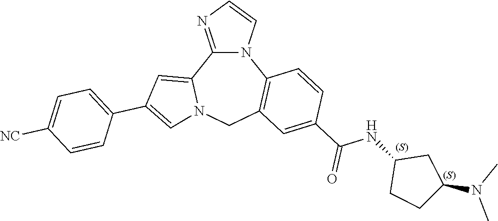 | 122 | (400 MHz, CD₃OD): δ 8.16- 8.15 (d, J = 4 Hz, 1H), 8.05 − 8.03 (m, 2H), 7.81 − 7.99 (d, J = 8.4 Hz, 1H), 7.76 − 7.73 (m, 3H), 7.70 − 7.65 (m, 3H), 7.28 (s, 1H), 5.41 (s, 2H), 4.52 − 4.49 (m, 1H), 3.83 − 3.80 (m, 1H), 2.92 (s, | 477.2 |
| | | 6H), 2.36 − 2.19 | |
| | | (m, 4H), 1.82 − | |
| | | 1.79 (m, 2H) | |
| 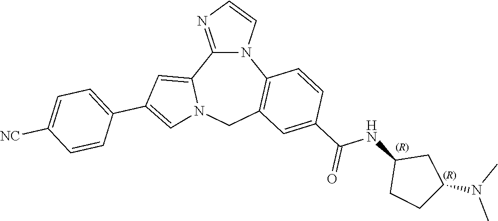 | 123 | (400 MHz, CD₃OD): δ 8.16- 8.15 (d, J = 4 Hz, 1H), 8.05-8.03 (m, 2H), 7.81-7.99 (d, J = 8.5 Hz, 1H), 7.76 − 7.73 (m, 3H), 7.70 − 7.65 (m, 3H), 7.28 (s, 1H), 5.41 (s, 2H), 4.52 − 4.49 (m, 1H), 3.83 − 3.80 (m, 1H), 2.92 (s, 6H), 2.36 − 2.19 (m, 4H), 1.82 − 1.79 (m, 2H). | 477.2 |
| 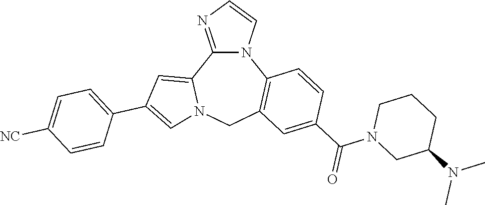 [SMILES: CN(C)[C@@H]1CCCN(C(=O)c2ccc3c(c2)Cn2cc(-c4ccc(C#N)cc4)cc2-c2nccn2-3)C1] | 124 | (400 MHz, CD₃OD): δ 8.07 (s, 1H), 7.83 (d, J = 8.5 Hz, 2H), 7.77 − 7.68 (m, 7H), 7.31 (s, 1H), 5.43 (s, 2H), 4.69 − 4.43 (m, 1H), 3.72-3.65 (m, 1H), 3.43 (d, J = 7.9 Hz, 2H), 3.03-2.96 (m, 7H), 2.27-2.20 (m, 1H), 1.91-1.86 (m, 2H), 1.68-1.62 (m, 1H). | 477.2 |
| 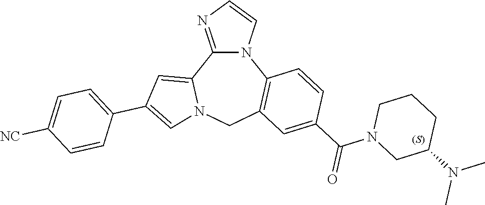 | 125 | (400 MHz, CD₃OD): δ 8.04 (s, 1H), 7.84 − 7.79 (m, 2H), 7.76 −7.67 (m, 7H), 7.28 (d, J = 1.7 Hz, 1H), 5.41 (s, 2H), 4.74 − 4.61 (m, 1H), 3.75 − 3.58 (m, 1H), 3.45 − 3.36 (m, 3H), 2.99 (s, 6H), 2.28-2.23 | 477.2 |
| | | (m, 1H), 1.93- | |
| | | 1.84 (m, 2H), | |
| | | 1.69-1.62 (m, 1H). | |
| 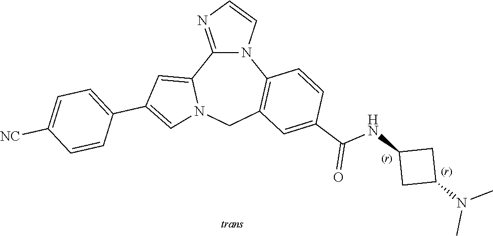 [SMILES: CN(C)[C@H]1C[C@H](NC(=O)c2ccc3c(c2)Cn2cc(-c4ccc(C#N)cc4)cc2-c2nccn2-3)C1] | 126 | (400 MHz, CD₃OD): δ 8.19 (d, J = 1.9 Hz, 1H), 8.11 − 8.01 (m, 2H), 7.77- 7.69 (m, 7H), 7.31 (d, J = 1.6 Hz, 1H), 5.44 (s, 2H), 4.60 − 4.40 (m, 1H), 4.06 − 3.86 (m, 1H), 2.86 (s, 6H), 2.80 − 2.69 (m, 2H), 2.66-2.62 (m, 2H). | 463.2 |
| 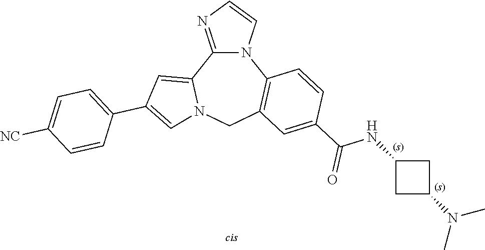 [SMILES: CN(C)[C@H]1C[C@@H](NC(=O)c2ccc3c(c2)Cn2cc(-c4ccc(C#N)cc4)cc2-c2nccn2-3)C1] | 127 | (400 MHz, CD₃OD): δ 8.19 − 8.18 (d, J = 4 Hz, 1H), 8.11 − 8.07 (m, 2H), 7.84 − 7.82 (m, 1H), 7.78 − 7.77 (d, J = 4 Hz, 1H), 7.75 − 7.73 (m, 3H), 7.70 − 7.68 (m, 2H), 7.33 (s, 1H), 5.44 (s, 2H), 4.34 − 4.26 (m, 1H), 3.60 − 3.52 (m, 1H), 2.91 − 2.86 (m, 2H), | 463.2 |
| | | 2.84 (s, 6H), 2.47 − | |
| | | 2.39 (m, 2H). | |
| 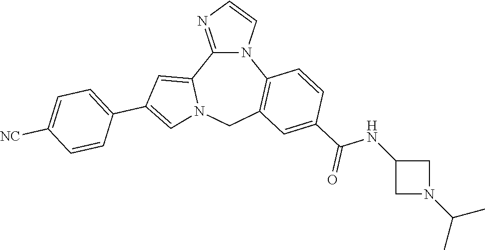 [SMILES: CC(C)N1CC(NC(=O)c2ccc3c(c2)Cn2cc(-c4ccc(C#N)cc4)cc2-c2nccn2-3)C1] | 128 | (400 MHz, CD₃OD) δ 8.21 (d, J = 11.7 Hz, 1H), 8.07 (d, J = 8.5 Hz, 1H), 8.00 (s, 1H), 7.82 (d, J = 8.4 Hz, 1H), 7.72 (m, 5H), 7.60 (s, 1H), 7.26 (s, 1H), 5.41 (s, 2H), 4.55 − 4.43 (m, 2H), 4.35-4.30(m, 1H), 3.64 − 3.48 (m, 1H), 1.29 (d, J = 6.5 Hz, 6H). | 463.2 |
| 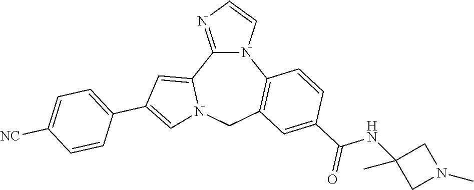 [SMILES: CN1CC(C)(NC(=O)c2ccc3c(c2)Cn2cc(-c4ccc(C#N)cc4)cc2-c2nccn2-3)C1] | 129 | (400 MHz, CD₃OD) δ 8.20- 8.18 (m, 1H), 8.05 (s, 1H), 8.01 (s, 1H), 7.82 (d, J = 8.2 Hz, 1H), 7.83-7.68 (m, 5H), 7.63-7.62 (m, 1H), 7.26 (s, 1H), 5.41 (s, 2H), 4.78 (d, J = 11.5 Hz, 1H), 4.54 (d, | 449.2 |
| | | J = 11.5 Hz, 1H), | |
| | | 4.28 (d, J = 11.6 | |
| | | Hz, 1H), 4.12 (d, | |
| | | J = 12.4 Hz, 1H), | |
| | | 3.04 (s, 3H), 1.73 | |
| | | (s, 3H). | |
| 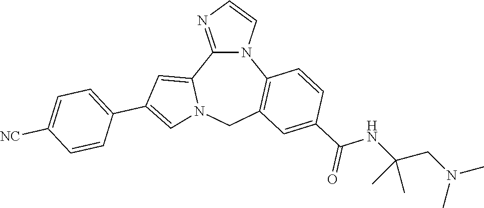 [SMILES: CN(C)CC(C)(C)NC(=O)c1ccc2c(c1)Cn1cc(-c3ccc(C#N)cc3)cc1-c1nccn1-2] | 130 | (400 MHz, CD₃OD) δ 8.21 (d, J = 1.9 Hz, 1H), 8.09 (dd, J = 8.4, 2.0 Hz, 2H), 7.77-7.69 (m, 7H), 7.31 (s, 1H), 5.44 (s, 2H), 3.71 (s, 2H), 2.99 (s, 6H), 1.58 (s, 6H). | 465.2 |
| 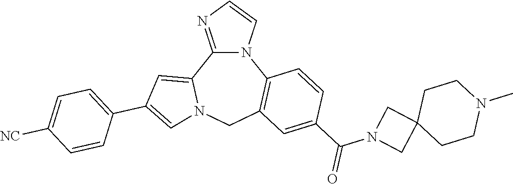 [SMILES: CN1CCC2(CC1)CN(C(=O)c1ccc3c(c1)Cn1cc(-c4ccc(C#N)cc4)cc1-c1nccn1-3)C2] | 131 | (400 MHz, CD₃OD) δ 8.07 (s, 1H), 8.01 (s, 1H), 7.90 (d, J = 8.3 Hz, 1H), 7.82 (d, J = 8.4 Hz, 1H), 7.78 − 7.73 (m, 3H), 7.70 (m, 3H), 7.31 (d, J = 1.7 Hz, 1H), 5.44 (s, 2H), 4.23 (d, J = 30.7 Hz, 2H), | 489.2 |
| | | 4.02 (d, J = 38.0 | |
| | | Hz, 2H), 3.52- | |
| | | 3.46 (m, 2H), | |
| | | 3.08 (t, J = | |
| | | 12.0 Hz , 1H), | |
| | | 2.96(t, J = | |
| | | 12.0 Hz , 1H),, | |
| | | 2.88 (s, 1.5H), | |
| | | 2.83(s, 1.5H), | |
| | | 2.25 (d, J = 14.1 | |
| | | Hz, 2H), 2.05 − | |
| | | 1.92 (m, 2H). | |

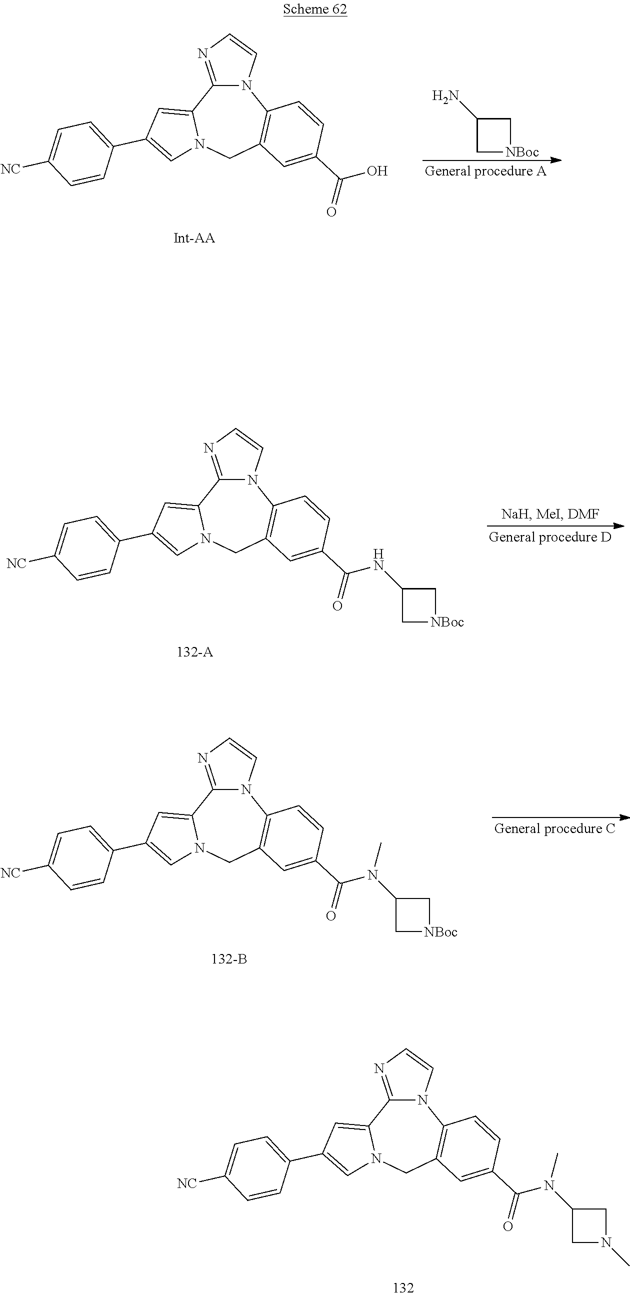 [SMILES: N#Cc1ccc(-c2cc3n(c2)Cc2cc(C(=O)O)ccc2-n2ccnc2-3)cc1 ; CC(C)(C)OC(=O)N1CC(N)C1 ; CC(C)(C)OC(=O)N1CC(NC(=O)c2ccc3c(c2)Cn2cc(-c4ccc(C#N)cc4)cc2-c2nccn2-3)C1 ; CN(C(=O)c1ccc2c(c1)Cn1cc(-c3ccc(C#N)cc3)cc1-c1nccn1-2)C1CN(C(=O)OC(C)(C)C)C1 ; CN1CC(N(C)C(=O)c2ccc3c(c2)Cn2cc(-c4ccc(C#N)cc4)cc2-c2nccn2-3)C1]

### tert-butyl 3-(12-(4-cyanophenyl)-9H-benzo[e]imidazo[2,1-c]pyrrolo [1,2-a][1,4]diazepine-7-carboxamido)azetidine-1-carboxylate (132-A)

Compound 132-A was prepared by using General procedure A as a solid (85 mg, yield: 64%). ESI: [M+H]f=521.2

General Procedure D:

### tert-butyl-3-(12-(4-cyanophenyl)-N-methyl-9H-benzo[e]imidazo[2,1-c]pyrrolo [1,2-a][1,4]diazepine-7-carboxamido)azetidine-1-carboxylate (132-B)

To a solution of compound 132-A (85 mg, 0.16 mmol) in DMF (5 mL) was added sodium hydride (15 mg, 0.64 mmol) at 0° C., and the resulting mixture was stirred for 30 min before the addition of MeI (40 mg, 0.28 mmol). The reaction mixture was stirred at 0° C. for 1 h and then quenched by addition of saturated NH₄Cl aqueous (20 mL). The resulting mixture was extracted with EA (20 mL×2); the combined organic layers were washed with brine (20 mL), dried over anhydrous Na₂SO₄. After filtration, the organic solution was concentrated under vacuum. The residue was purified by silica gel chromatography eluting with DCM/MeOH (100/1-20/1) to give the desired compound (50 mg, yield: 57%). ESI [M+H]⁺=535.2.

### 12-(4-Cyanophenyl)-N-methyl-N-(1-methylazetidin-3-yl)-9H-benzo[e]imidazo [2,1c]pyrrolo[1,2-a][1,4]diazepine-7-carboxamide (132)

Compound 132 was prepared by using General procedure C as a solid (2 mg, yield: 22%). ESI [M+H]⁺=448.2; ¹H-NMR (400 MHz, CD₃OD) δ 8.09 (s, 1H), 7.84 (d, J=8.5 Hz, 2H), 7.79-7.66 (m, 8H), 7.33 (s, 1H), 5.44 (s, 2H), 4.71-4.16 (m, 4H), 3.03 (s, 6H).

### Example 133-135

Compounds 133-135 in Table 15 were synthesized by using similar procedure described above.

TABLE 15

| | Ex. | | LC-MS |
|---|---|---|---|
| Structure | # | ¹H NMR | (M + H)⁺ |
| 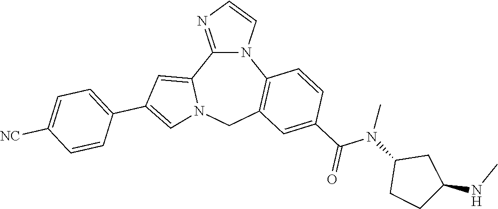 [SMILES: CN[C@H]1CC[C@H](N(C)C(=O)c2ccc3c(c2)Cn2cc(-c4ccc(C#N)cc4)cc2-c2nccn2-3)C1] | 133 | (400 MHz, CD₃OD): δ 8.10 − 8.09 (d, J = 4 Hz, 1H), 7.84 − 7.69 (m, 9H), 7.34 − 7.33 (d, J = 4 Hz, 1H), 5.44 (s, 2H), 3.72 (ds, 1H), 2.96 (s, 3H), 2.72 (m, 3H), 2.33 − 1.95 (m, 5H), 1.71 − 1.29 (m,2H). | 477.2 |
| 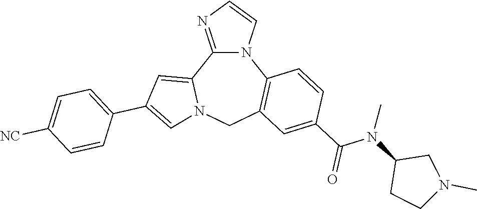 [SMILES: CN1CC[C@@H](N(C)C(=O)c2ccc3c(c2)Cn2cc(-c4ccc(C#N)cc4)cc2-c2nccn2-3)C1] | 134 | (400 MHz, CD₃OD): δ 8.10 − 8.07 (m, 1H), 7.84 − 7.82 (m, 2H), 7.78 − 7.69 (m, 7H), 7.33 − 7.31 (m, 1H), 5.44 − 5.43 (d, J = 4 Hz, 2H), 4.59-4.52 (m, 1H), 3.99-3.94 (m, 2H), 3.47- 3.41 (m, 1H), 3.07 (s, 3H), 2.98 (s, 3H) 2.69-2.62 (m, 1H), 2.47-2.40 (m, 2H). | 463.2 |
| 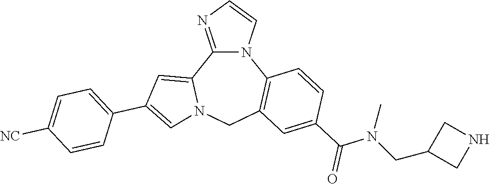 [SMILES: CN(CC1CNC1)C(=O)c1ccc2c(c1)Cn1cc(-c3ccc(C#N)cc3)cc1-c1nccn1-2] | 135 | (400 MHz, CD₃OD): δ 8.08 (s, 1H), 7.82 (t, J = 5.7 Hz, 2H), 7.78 − 7.68 (m, 7H), 7.32 (d, J = 1.4 Hz, 1H), 5.43 (s, 2H), 4.19 (t, J = 9.5 Hz, 2H), 4.13 − 4.03 (m, 2H), 3.85 (d, J = 6.9 Hz, 2H), 3.30 (s, 1H), 3.01 (s, 3H). | 449.2 |

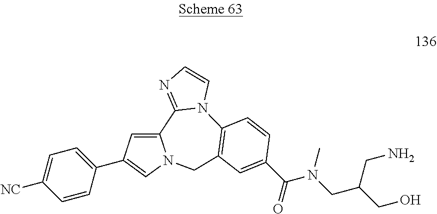 [SMILES: CN(CC(CN)CO)C(=O)c1ccc2c(c1)Cn1cc(-c3ccc(C#N)cc3)cc1-c1nccn1-2]

Compound 136 was the by-product during the synthesis of compound 135. ESI: [M+H]⁺=467.2; ¹H-NMR (400 MHz, CD₃OD): δ 8.43 (d, J=1.9 Hz, 1H), 8.29 (dt, J=10.6, 5.3 Hz, 1H), 8.07 (t, J=4.0 Hz, 1H), 7.85 (d, J=8.5 Hz, 1H), 7.78-7.65 (m, 6H), 7.31 (d, J=1.7 Hz, 1H), 5.48 (s, 2H), 4.55 (d, J=4.4 Hz, 2H), 3.34 (t, J=7.6 Hz, 2H), 3.26 (d, J=6.9 Hz, 2H), 2.81 (d, J=10.1 Hz, 3H), 2.71-2.64 (m, 1H).

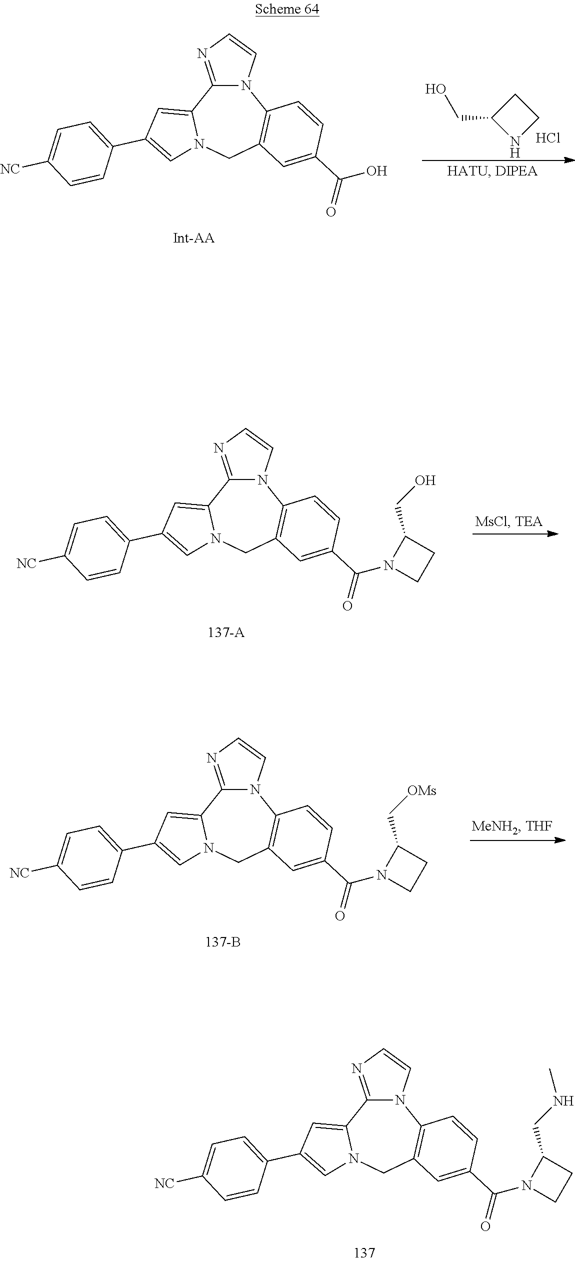 [SMILES: N#Cc1ccc(-c2cc3n(c2)Cc2cc(C(=O)O)ccc2-n2ccnc2-3)cc1 ; OC[C@@H]1CCN1 ; Cl ; N#Cc1ccc(-c2cc3n(c2)Cc2cc(C(=O)N4CC[C@H]4CO)ccc2-n2ccnc2-3)cc1 ; CS(=O)(=O)OC[C@@H]1CCN1C(=O)c1ccc2c(c1)Cn1cc(-c3ccc(C#N)cc3)cc1-c1nccn1-2 ; CNC[C@@H]1CCN1C(=O)c1ccc2c(c1)Cn1cc(-c3ccc(C#N)cc3)cc1-c1nccn1-2]

### (S)-4-(7-(2-(Hydroxymethyl)azetidine-1-carbonyl)-9H-benzo[e]imidazo[2,1-c]pyrrolo[1,2-a][1,4]diazepin-12-yl)benzonitrile (137-A)

To a solution of (S)-azetidin-2-ylmethanol hydrochloride (605 mg, 4.9 mmol, 1.2 eq), compound Int-AA (1.5 g, 4.1 mmol, 1.0 eq.) in dry DCM (20 mL) was added DIPEA (1 mL) and HATU (1.55 g, 4.1 mmol, 1.0 eq.). The mixture was stirred at rt overnight. After the reaction was completed, the mixture was concentrated in vacuum. The residue was purified by flash chromatography (MeOH/DCM=0-5%) to give the desired product as a solid (1.05 g, yield: 58%). ESI: [M+H]⁺=436

### (S)-(1-(12-(4-Cyanophenyl)-9H-benzo[e]imidazo[2,1-c]pyrrolo[1,2-a][1,4]diazepine-7-carbonyl)azetidin-2-yl)methyl methanesulfonate (137-B)

To a solution of compound 137-A (1.05 g, 2.4 mmol, 1.0 eq.) in dry DCM (20 mL) was added TEA (0.67 mL, 4.8 mmol, 2.0 eq.) and MsCl (0.28 mL, 3.6 mmol, 1.5 eq.) at 0° C. The mixture was stirred at rt for 1 h. After the reaction was complete, the mixture was quenched with ice-water (20 mL) and extracted with DCM (20 mL). The separated organic layer was washed with brine, dried over anhydrous Na₂SO₄, filtered. After filtration and concentration, the crude compound was purified by flash chromatography (MeOH/DCM=0%-5%) to give the desired product as a solid (800 mg, yield: 65%). ESI: [M+H]⁺=513

### (S)-4-(7-(2-((Methylamino)methyl)azetidine-1-carbonyl)-9H-benzo[e]imidazo [2,1-c]pyrrolo[1,2-a][1,4]diazepin-12-yl)benzonitrile (137)

The mixture of Compound 137-B (200 mg, 0.39 mmol, 1.0 eq.) and MeNH₂ in THE (20 mL, 40 mmol) was stirred in a sealed tube at 70° C. for 16 hours. After the reaction was complete, the mixture was concentrated under reduced pressure. The resulting residue was purified by flash chromatography (MeOH/DCM=0%-5%) and then Prep-HPLC to give the desired product as a white solid (100 mg, yield: 56%). ESI: [M+H]⁺=449; ¹H-NMR (400 MHz, DMSO-d₆): δ 7.90 (d, J=9.6 Hz, 2H), 7.78-7.72 (m, 6H), 7.68-7.63 (m, 2H), 7.26 (s, 1H), 7.06 (d, J=1.8 Hz, 1H), 5.30 (s, 2H), 4.59-4.51 (m, 1H), 4.39-4.30 (m, 1H), 4.08-4.01 (m, 1H), 2.92-2.80 (m, 2H), 2.43-2.27 (m, 4H), 2.14-2.03 (m, 2H).

B. Biological Assays

Materials and Reagents for Measuring Inhibition of MK2 Biochemical Activity:
1) Assay plate: 384 Well Low Volume Black Round Bottom Polystyrene NBS Microplate (Corning, Cat.No. 4514)
2) MK2 kinase assay kit: Z' LYTE KINASE ASSAY KIT (ThermoFisher, Cat.No. pv3177)
3) MAPKAPK2 Recombinant Human Protein (ThermoFisher, Cat.No. pv3317)
4) DMSO (Vetec, Cat.No. V900090)
5) DTT (Sigma, Cat.No. D0632)

MK2 Kinase Assay Procedure:
1) Dispense 5 μl Kinase/Peptide Mixture to each wells of 384-well plate
2) Add 2.5 μl 4× Test Compound to each well
3) Add 2.5 μl 4× ATP Solution to each well
4) After 90 minutes incubation at room temperature, add 5 μl Development Solution to each well
5) After 60 minutes incubation at room temperature, add 5 μl stop Solution to each well
6) Measure the Coumarin (Ex. 400 nm, Em. 445 nm) and Fluorescein (Ex. 400 nm, Em. 520 nm) emission signals on an Envision plate reader (PerkinElmer)

Materials and Reagents for Measuring Activity in THP-1 Cells:
1) THP-1 cells (Cell bank of Shanghai)
2) PMA (Sigma, Cat.No. P1585)
3) LPS (Sigma, Cat.No. L4516)
4) 96-well tissue-culture treated plate (Grenier, Cat.No. 655180)
5) FBS (Thermofisher, Cat.No. 10100147)
6) Complete EDTA-free protease inhibitor Cocktail (Roche, Cat.No. 4693132001)
7) PhosSTOP (Roche, Cat.No. 4906845001)
8) Phosphor-Hsp27 (Ser78) Sandwich Elisa kit (CST, Cat.No. 7295)

Cell Assay Procedure:
1) Day 0: THP-1 cells are seeded at 50K/well in complete media (1640+10% FBS+50 μM β-ME)+20 ng/mL PMA in 96-well plate
2) Day 1: PMA containing media is removed after 24 h incubation (37° C./5% CO₂), and replaced with complete growth media. Cells are incubated an additional 48 h to recover.
3) Day 3: Cells are incubated with or without compounds at indicated concentrations for 60 min, prior to addition of 20 ng/mL LPS. Cells are stimulated for 30 min with LPS, washed 1× with ice cold PBS, lysed on ice in Lysis buffer provided in ELISA kit with Roche protease & phosphatase inhibitors. Extracts are clarified by refrigerated centrifugation & stored at −80° C. until pHsp27/total Hsp27 ELISAs
4) Day 3 or Day 4: Cell Signaling Technology ELISA is run for pSer78-Hsp27 on lysates diluted to approximately 2.5 μg/well.
5) The absorbance at 450 nm was read in a SpectraMax plus plate reader (Molecular Devices). Data was processed using GraphPad prism 8.0.

Using the procedures above, the following biological activity in Table 16 was determined:

TABLE 16

| | | |
|---|---|---|
| Example | MK2 Activity | THP-1 IC₅₀ |

| | | | | |
|---|---|---|---|---|
| 1 | <50 | nM | <50 | nM |
| 2 | 50-100 | nM | | |
| 3 | <50 | nM | <50 | nM |
| 4 | <50 | nM | <50 | nM |
| 5 | <50 | nM | <50 | nM |
| 6 | <50 | nM | <50 | nM |
| 7 | <50 | nM | <50 | nM |
| 8 | <50 | nM | 300-400 | nM |
| 9 | <50 | nM | 50-100 | nM |
| 10 | <50 | nM | 100-200 | nM |
| 11 | 50-100 | nM | 100-200 | nM |
| 12 | <50 | nM | 100-200 | nM |
| 13 | <50 | nM | 100-200 | nM |
| 14 | 50-100 | nM | 100-200 | nM |
| 15 | <50 | nM | <50 | nM |
| 16 | 50-100 | nM | 400-500 | nM |
| 17 | 50-100 | nM | 200-300 | nM |
| 18 | 50-100 | nM | | |
| 19 | <50 | nM | 50-100 | nM |
| 20 | <50 | nM | | |
| 21 | <50 | nM | <50 | nM |
| 22 | <50 | nM | 1000-1500 | nM |
| 23 | <50 | nM | 200-300 | nM |
| 24 | <50 | nM | <50 | nM |
| 25 | 50-100 | nM | 300-400 | nM |
| 26 | 50-100 | nM | 100-200 | nM |
| 27 | <50 | nM | <50 | nM |
| 28 | <50 | nM | 500-1000 | nM |
| 29 | <50 | nM | 50-100 | nM |
| 30 | 50-100 | nM | 200-300 | nM |
| 31 | 50-100 | nM | | |
| 32 | 50-100 | nM | | |
| 33 | <50 | nM | 400-500 | nM |
| 34 | <50 | nM | <50 | nM |
| 35 | <50 | nM | <50 | nM |
| 36 | <50 | nM | <50 | nM |
| 37 | 50-100 | nM | 50-100 | nM |
| 38 | <50 | nM | | |
| 39 | 100-200 | nM | | |
| 40 | <50 | nM | | |
| 41 | <50 | nM | <50 | nM |
| 42 | <50 | nM | <50 | nM |
| 43 | <50 | nM | 300-400 | nM |
| 44 | 50-100 | nM | | |
| 45 | 50-100 | nM | | |
| 46 | <50 | nM | 200-300 | nM |
| 47 | <50 | nM | 100-200 | nM |
| 48 | 50-100 | nM | 200-300 | nM |
| 49 | <50 | nM | | |
| 50 | <50 | nM | 50-100 | nM |
| 51 | <50 | nM | | |
| 52 | <50 | nM | | |
| 53 | <50 | nM | 300-400 | nM |
| 54 | <50 | nM | <50 | nM |
| 55 | <50 | nM | <50 | nM |
| 56 | 50-100 | nM | 200-300 | nM |
| 57 | <50 | nM | <50 | nM |
| 58 | <50 | nM | <50 | nM |
| 59 | <50 | nM | 50-100 | nM |
| 60 | <50 | nM | | |
| 61 | 50-100 | nM | 300-400 | nM |
| 62 | <50 | nM | | |
| 63 | 50-100 | nM | | |
| 64 | <50 | nM | | |
| 65 | <50 | nM | <50 | nM |
| 66 | <50 | nM | >1000 | nM |
| 67 | <50 | nM | >1000 | nM |
| 68 | <50 | nM | <50 | nM |
| 69 | <50 | nM | <50 | nM |
| 70 | <50 | nM | 500-1000 | nM |
| 71 | 50-100 | nM | 200-300 | nM |
| 72 | <50 | nM | <50 | nM |
| 73 | <50 | nM | <50 | nM |
| 74 | <50 | nM | 50-100 | nM |
| 75 | <50 | nM | <50 | nM |
| 76 | <50 | nM | 1000-1500 | nM |
| 77 | 50-100 | nM | 100-200 | nM |
| 78 | 50-100 | nM | | |
| 79 | 50-100 | nM | 100-200 | nM |
| 80 | 50-100 | nM | | |
| 81 | 50-100 | nM | | |
| 82 | 50-100 | nM | 300-400 | nM |
| 83 | 50-100 | nM | | |
| 84 | 50-100 | nM | | |
| 85 | <50 | nM | | |
| 86 | <50 | nM | 300-400 | nM |
| 87 | <50 | nM | 100-200 | nM |
| 89 | 50-100 | nM | 50-100 | nM |
| 90 | <50 | nM | 50-100 | nM |
| 91 | 50-100 | nM | 300-400 | nM |
| 92 | 50-100 | nM | | |
| 101 | 50-100 | nM | 100-200 | nM |
| 102 | <50 | nM | | |
| 103 | 200-300 | nM | | |
| 104 | 200-300 | nM | | |
| 105 | <50 | nM | 500-1000 | nM |
| 106 | 100-200 | nM | | |
| 107 | 200-300 | nM | | |
| 108 | 200-300 | nM | | |
| 109 | 50-100 | nM | 200-300 | nM |
| 110 | <50 | nM | 50-100 | nM |
| 111 | 200-300 | nM | | |
| 112 | 50-100 | nM | | |
| 113 | 200-300 | nM | | |
| 114 | 100-200 | nM | | |
| 115 | <50 | nM | 200-300 | nM |
| 116 | <50 | nM | <50 | nM |
| 117 | 50-100 | nM | 500-1000 | nM |
| 118 | 100-200 | nM | | |
| 119 | <50 | nM | 500-1000 | nM |
| 120 | | | <50 | nM |
| 121 | 50-100 | nM | | |
| 122 | 300-400 | nM | | |
| 123 | 300-400 | nM | | |
| 124 | 50-100 | nM | | |
| 125 | 50-100 | nM | | |
| 126 | <50 | nM | 500-1000 | nM |
| 127 | 100-200 | nM | | |
| 128 | <50 | nM | 500-1000 | nM |
| 129 | 100-200 | nM | | |
| 130 | 100-200 | nM | | |
| 131 | 50-100 | nM | | |
| 132 | 50-100 | nM | | |
| 133 | 200-300 | nM | | |
| 134 | 200-300 | nM | | |
| 135 | 100-200 | nM | | |
| 136 | 50-100 | nM | | |
| 137 | | | 200-300 | nM |

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A compound having a structure of Formula (I) or a pharmaceutically acceptable salt thereof: 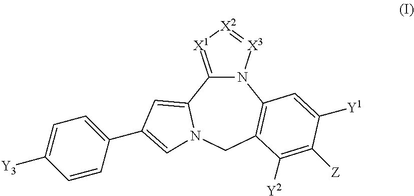
wherein
X¹, X², and X³ are each independently CH, C-(alkyl), or N;
Y¹ and Y² are each independently H or halogen;
Y³ is CN or halogen;
Z is —C(O)NZ¹Z², —NHC(O)NZ³Z⁴, —NHC(O)C(HZ⁵Z⁶), or —NZ⁷Z⁸;
Z¹ is H or alkyl;
Z² is optionally substituted alkyl, aminoalkyl, cycloalkyl, heterocyclyl, or heterocycloalkyl; or
Z¹ and Z² together with the N to which they are bound combine to form an optionally substituted 4-, 5-, or 6-membered heterocyclic ring or an optionally substituted heterobicyclic ring;
Z³ and Z⁴ together with the N to which they are bound combine to form an optionally substituted 4-, 5-, or 6-membered heterocyclic ring;
Z⁵ and Z⁶ together with the C to which they are bound combine to form an optionally substituted 4-, 5-, or 6-membered heterocyclic ring; and
Z⁷ and Z⁸ together with the N to which they are bound combine to form an optionally substituted 5- or 6-membered cyclic amide, cyclic urea, or cyclic carbamate.

2. The compound of claim 1, wherein at least one of X¹, X², and X³ is N.

3. The compound of claim 1 or 2, wherein Z is —C(O)NZ¹Z².

4. The compound of claim 3, wherein Z¹ is H or CH₃.

5. The compound of any one of claims 1-4, wherein Z² is substituted alkyl or substituted aminoalkyl.

6. The compound of any one of claims 1-5, wherein Z² is a substituted linear C₁-C₆ alkyl.

7. The compound of any one of claims 1-5, wherein Z² is a substituted branched C₂-C₆ alkyl.

8. The compound of any one of claims 5-7, wherein Z² is substituted with alkylamino.

9. The compound of claim 8, wherein Z² is substituted with methylamino or dimethylamino.

10. The compound of any one of claims 5-7, wherein Z² is substituted with hydroxyalkyl.

11. The compound of any one of claims 1-4, wherein Z is 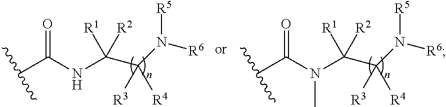
n is 0, 1 or 2;
R¹, R², R³, and R⁴ are, independently for each occurrence, H, alkyl, or hydroxyalkyl;
and R⁵ and R⁶ are, independently for each occurrence, H or alkyl.

12. The compound of claim 11, wherein R¹, R², R³, and R⁴ are, independently for each occurrence, H or CH₃; and each occurrence of R⁵ and R⁶ is CH₃.

13. The compound of claim 11, wherein each occurrence of R¹, R², R³, and R⁴ is H; and each occurrence of R⁵ and R⁶ is CH₃.

14. The compound of claim 11, wherein Z is

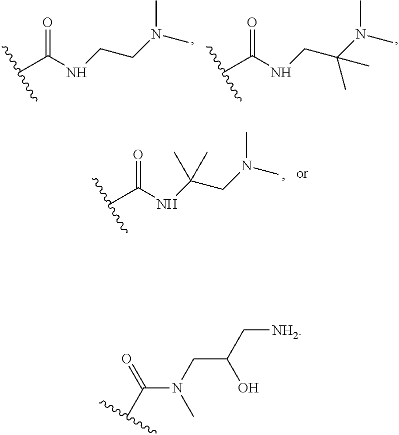

15. The compound of any one of claims 1-4, wherein Z² is optionally substituted heterocycloalkyl.

16. The compound of claim 15, wherein the heterocycloalkyl comprises an azetidinyl, pyrrolidinyl, or piperidinyl.

17. The compound of claim 15 or 16, wherein the azetidinyl, pyrrolidinyl, or piperidinyl, when substituted, is substituted with alkyl.

18. The compound of claim 15, wherein Z is

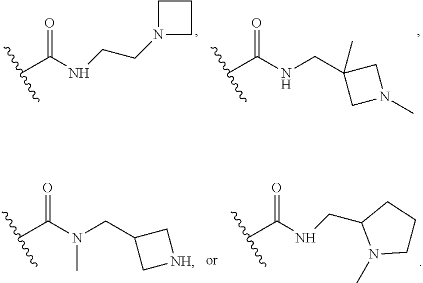

19. The compound of any one of claims 1-4, wherein Z₂ is optionally substituted cycloalkyl.

20. The compound of claim 19 wherein Z₂ is an optionally substituted cyclobutyl, cyclopentyl, or cyclohexyl.

21. The compound of claim 20, wherein the cyclobutyl, cyclopentyl or cyclohexyl, when substituted, is substituted with alkylamino.

22. The compound of claim 21, wherein the alkylamino is methylamino or dimethylamino.

23. The compound of claim 19, wherein Z is

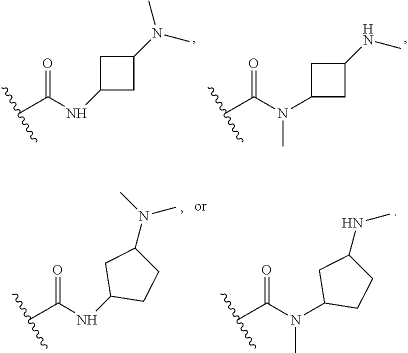

24. The compound of any one of claims 1-4, wherein Z² is optionally substituted heterocyclyl.

25. The compound of claim 24, wherein the Z² is an optionally substituted azetidinyl, pyrrolidinyl, or piperidinyl.

26. The compound of claim 25, wherein the azetidinyl, pyrrolidinyl, or piperidinyl, when substituted, is substituted with alkyl.

27. The compound of claim 26, wherein the alkyl is methyl or isopropyl.

28. The compound of claim 24, wherein Z is

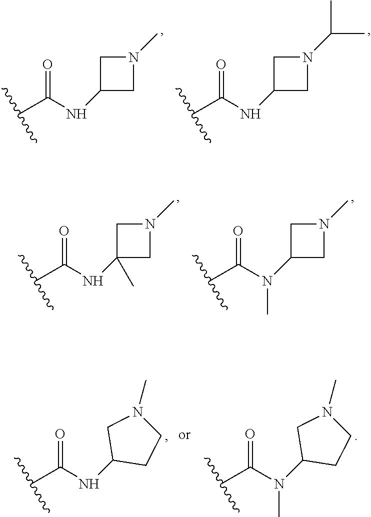

29. The compound of any one of claims 1-3, wherein Z¹ and Z², together with the N to which they are bound, combine to form an optionally substituted 4-, 5-, or 6-membered heterocyclic ring.

30. The compound of claim 29, wherein
Z is 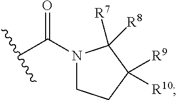
R⁷ and R⁸ are each independently H, CN, halo, alkyl, aminoalkyl, or alkylaminoalkyl; and
R⁹ and R¹⁰ are each independently H, CN, halo, hydroxyl, amino, alkyl, aminoalkyl, alkylamino, or alkylaminoalkyl.

31. The compound of claim 30, wherein R⁷ and R⁸ are each independently H, alkyl, or alkylaminoalkyl; and R⁹ and R¹⁰ are each independently H, alkyl, alkylamino, or alkylaminoalkyl.

32. The compound of claim 30 or 31, wherein the alkylamino is methylamino or dimethylamino.

33. The compound of claim 30 or 31, wherein the alkylaminoalkyl is methylaminoalkyl or dimethylaminoalkyl.

34. The compound of claim 29, wherein
Z is 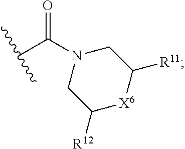
X⁶ is CH₂, NH, or N(alkyl);
R¹¹ and R¹² are each independently H, CN, halo, hydroxyl, amino, alkyl, aminoalkyl, alkylamino, or alkylaminoalkyl; and
when X⁶ is NH or N(alkyl), then R¹¹ and R¹² are not hydroxyl, amino, or alkylamino.

35. The compound of claim 33, wherein R¹¹ and R¹² are each independently H, CN, alkyl, alkylamino, or alkylaminoalkyl.

36. The compound of claim 34 or 35, wherein the alkylamino is methylamino or dimethylamino.

37. The compound of claim 34 or 35, wherein the alkylaminoalkyl is methylaminoalkyl or dimethylaminoalkyl.

38. The compound of any one of claim 30 or 34, wherein Z is

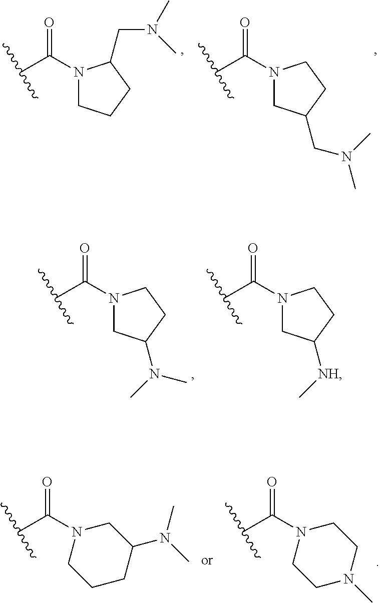

39. The compound of claim 29, wherein
Z is 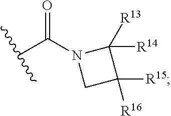
R¹³ and R¹⁴ are each independently H, CN, halo, alkyl, aminoalkyl, or alkylaminoalkyl; and
R¹⁵ and R¹⁶ are each independently H, CN, halo, hydroxyl, amino, alkyl, aminoalkyl, alkylamino, or alkylaminoalkyl, or
R¹⁵ and R¹⁶ combine to form an optionally substituted 4-, 5-, or 6-membered nitrogen containing heterocyclic ring.

40. The compound of claim 39, wherein
R¹³ and R¹⁴ are each independently H, CN, alkyl, aminoalkyl, or alkylaminoalkyl.

41. The compound of claim 39 or 40, wherein R¹⁵ and R¹⁶ are each independently H, CN, alkyl, aminoalkyl, alkylamino, or alkylaminoalkyl.

42. The compound of claim 40 or 41, wherein the alkylamino is methylamino or dimethylamino.

43. The compound of claim 40 or 41, wherein the alkylaminoalkyl is methylaminoalkyl or dimethylaminoalkyl.

44. The compound of claim 43, wherein R¹³ and R¹⁴ are each H; and R¹⁵ and R¹⁶ combine to form an optionally substituted 4-, 5-, or 6-membered nitrogen containing heterocyclic ring.

45. The compound of claim 44, wherein the optionally substituted 4-, 5-, or 6-membered nitrogen containing heterocyclic ring is an azetidine, pyrrolidine, or piperidine.

46. The compound of claim 45, wherein the azetidine, pyrrolidine, or piperidine, when substituted, is substituted with alkyl.

47. The compound of any one of claim 39, wherein Z is

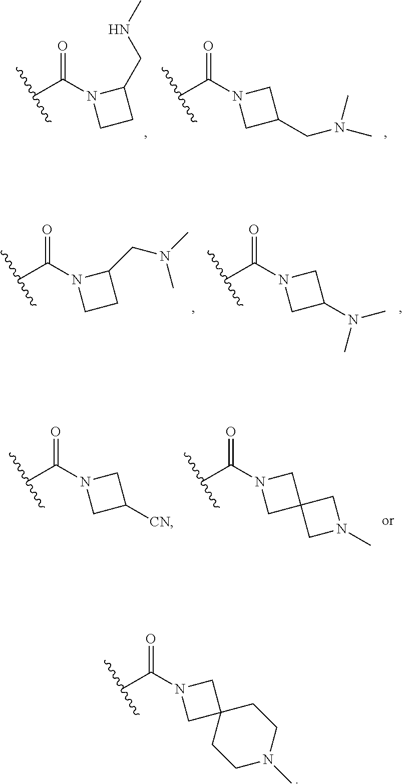

48. The compound of any one of claims 1-3, wherein Z¹ and Z² together with the N to which they are bound combine to form an optionally substituted heterobicyclic ring.

49. The compound of claim 48, wherein Z is 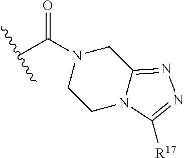
wherein R⁷ is H, CN, halo, hydroxyl, amino, alkyl, aminoalkyl, alkylamino, or alkylaminoalkyl.

50. The compound of claim 49, wherein Z is

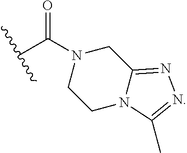 [SMILES: *C(=O)N1CCn2c(C)nnc2C1]

51. The compound of claim 1 or 2 having the structure: 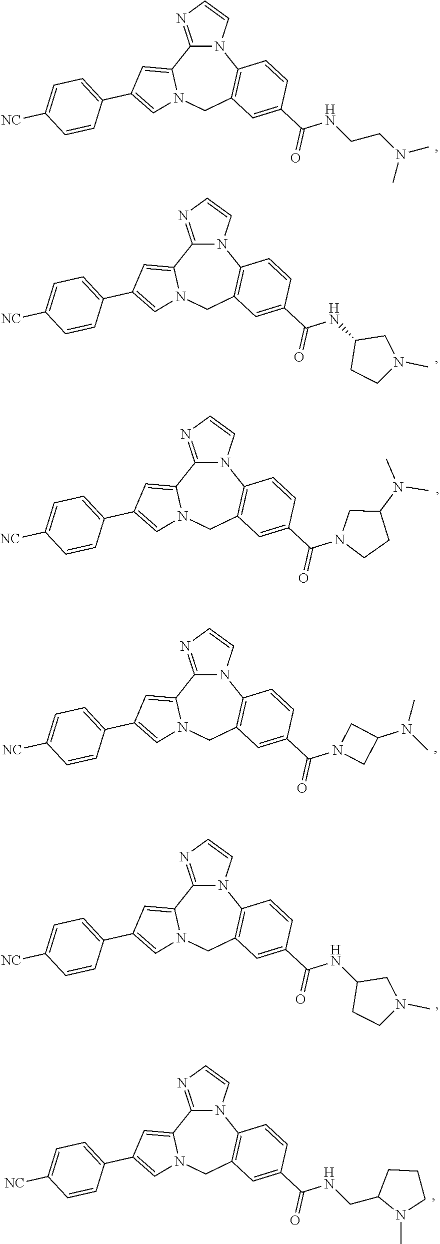 [SMILES: CN(C)CCNC(=O)c1ccc2c(c1)Cn1cc(-c3ccc(C#N)cc3)cc1-c1nccn1-2 ; CN1CC[C@H](NC(=O)c2ccc3c(c2)Cn2cc(-c4ccc(C#N)cc4)cc2-c2nccn2-3)C1 ; CN(C)C1CCN(C(=O)c2ccc3c(c2)Cn2cc(-c4ccc(C#N)cc4)cc2-c2nccn2-3)C1 ; CN(C)C1CN(C(=O)c2ccc3c(c2)Cn2cc(-c4ccc(C#N)cc4)cc2-c2nccn2-3)C1 ; CN1CCC(NC(=O)c2ccc3c(c2)Cn2cc(-c4ccc(C#N)cc4)cc2-c2nccn2-3)C1 ; CN1CCCC1CNC(=O)c1ccc2c(c1)Cn1cc(-c3ccc(C#N)cc3)cc1-c1nccn1-2] 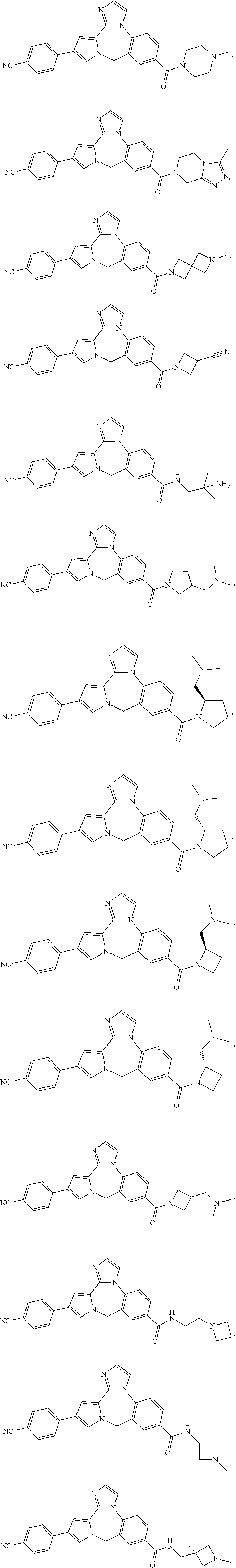 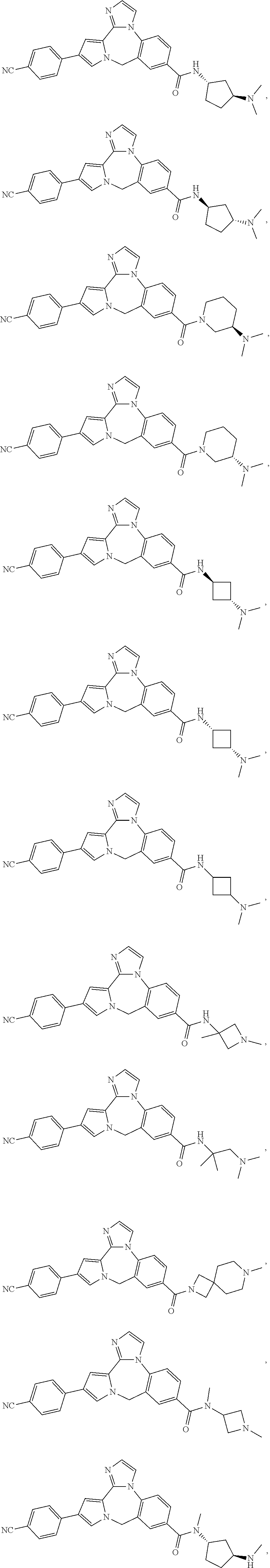 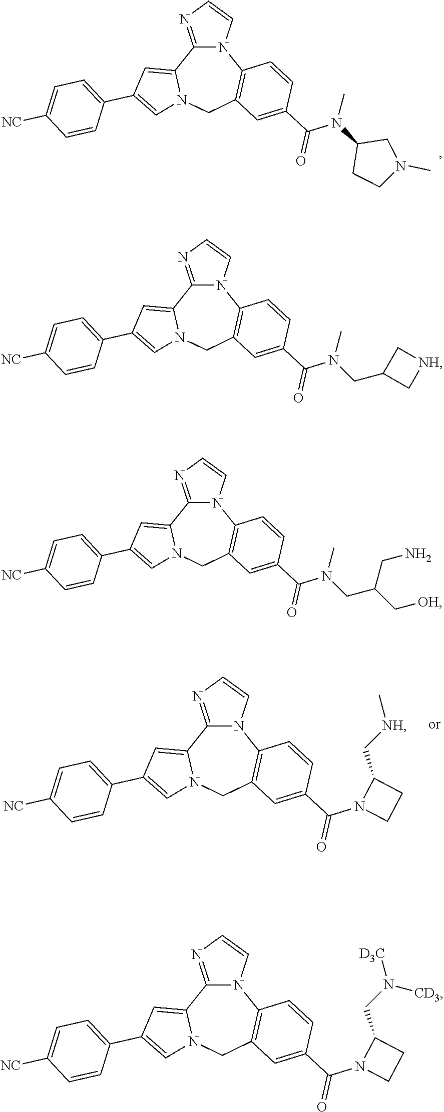 [SMILES: CN1CC[C@@H](N(C)C(=O)c2ccc3c(c2)Cn2cc(-c4ccc(C#N)cc4)cc2-c2nccn2-3)C1 ; CN(CC1CNC1)C(=O)c1ccc2c(c1)Cn1cc(-c3ccc(C#N)cc3)cc1-c1nccn1-2 ; CN(CC(CN)CO)C(=O)c1ccc2c(c1)Cn1cc(-c3ccc(C#N)cc3)cc1-c1nccn1-2 ; CNC[C@@H]1CCN1C(=O)c1ccc2c(c1)Cn1cc(-c3ccc(C#N)cc3)cc1-c1nccn1-2 ; "or" ; [2H]C([2H])([2H])N(C[C@@H]1CCN1C(=O)c1ccc2c(c1)Cn1cc(-c3ccc(C#N)cc3)cc1-c1nccn1-2)C([2H])([2H])[2H]]
or a pharmaceutically acceptable salt of the compound.

52. The compound of claim 1 or 2, wherein Z is —NHC(O)NZ³Z⁴, and Z³ and Z⁴ together with the N to which they are bound combine to form an optionally substituted 5-membered heterocyclic ring.

53. The compound of claim 52, wherein
Z is 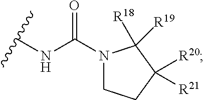
R¹⁸ and R¹⁹ are each independently H, CN, halo, alkyl, aminoalkyl, or alkylaminoalkyl; and
R²⁰ and R²¹ are each independently H, CN, halo, hydroxyl, amino, alkyl, aminoalkyl, alkylamino, or alkylaminoalkyl.

54. The compound of claim 53, wherein R²⁰ and R²¹ are each independently H or alkylamino.

55. The compound of claim 53 or 54, wherein the alkylamino is methylamino or dimethylamino.

56. The compound of claim 53, wherein Z is

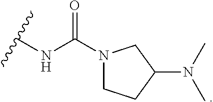 [SMILES: *NC(=O)N1CCC(N(C)C)C1 ; "."]

57. The compound of claim 1 or 2, wherein Z is —NHC(O)C(HZ⁵Z⁶); and Z⁵ and Z⁶ together with the C to which they are bound combine to form an optionally substituted 5-membered heterocyclic ring.

58. The compound of claim 57, wherein
Z is 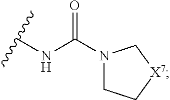
X⁷ is CHR²² or NR²³;
R²² is H, CN, halo, hydroxyl, amino, alkyl, aminoalkyl, alkylamino, or alkylaminoalkyl; and
R²³ is H, alkyl, alkylamino, or alkylaminoalkyl.

59. The compound of claim 58, wherein X⁷ is CHR²²; and R²² is alkylamino.

60. The compound of claim 59, wherein the alkylamino is methylamino or dimethylamino.

61. The compound of claim 58, wherein X⁷ is NR²³; and R²³ is H or alkyl.

62. The compound of claim 52 or 58, wherein Z is

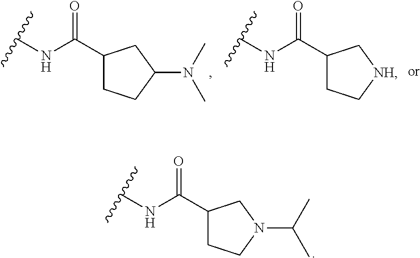

63. The compound of claim 1 or 2 having the structure: 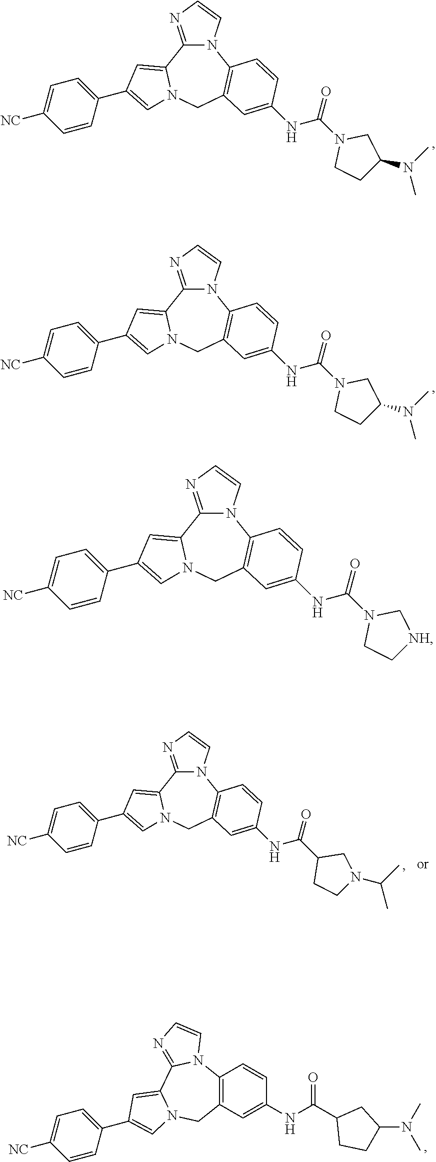
or a pharmaceutically acceptable salt of the compound.

64. The compound of claim 1 or 2, wherein Z is —NZ⁷Z⁸.

65. The compound of claim 64, wherein Z⁷ and Z⁸ together with the N to which they are bound combine to form an optionally substituted 5- or 6-membered cyclic amide, cyclic urea, or cyclic carbamate.

66. The compound of claim 65, wherein
Z is 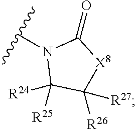
X⁸ is CR²⁸R²⁹, NR³⁰ or O;
R²⁴ and R²⁶ are each independently H or alkyl;
R²⁵ is H, or an optionally substituted alkyl, aminoalkyl, alkylaminoalkyl, heterocycloalkyl, or heterocyclyl;
R²⁷ is H, or an optionally substituted alkyl, amino, alkylamino, aminoalkyl, alkylaminoalkyl, heterocycloalkyl, or heterocyclyl;
R²⁸ and R²⁹ each independently H, alkyl, alkylamino, aminoalkyl, or optionally substituted alkylaminoalkyl;
R³⁰ is H, alkyl, alkylaminoalkyl, or an optionally substituted cycloalkyl or heterocyclyl; and
when X⁸ is NR³⁰ or O, then R³⁰ is not amino, or alkylamino.

67. The compound of any one of claim 66, wherein X⁸ is NH or N—CH₃.

68. The compound of any one of claim 66, wherein X⁸ is CH₂.

69. The compound of any one of claim 66, wherein X⁸ is O.

70. The compound of any one of claims 66-69, wherein one of R²⁵ and R²⁷ is H and the other of R²⁵ and R²⁷ is 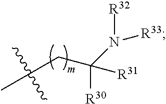
m is 0, 1 or 2;
R³⁰ and R³¹ are each independently H or alkyl, or R³⁰ and R³¹ together with the C to which they are bound form a carbocyclic ring; and
R³² and R³³ are each independently H, alkyl, hydroxyalkyl, optionally substituted cycloalkyl, or optionally substituted heterocyclyl, or together with the N to which they are bound form an optionally substituted heterocyclic ring.

71. The compound of claim 70, wherein R³⁰ and R³¹ are each H.

72. The compound of claim 70, wherein R³⁰ and R³¹ are each alkyl.

73. The compound of any one of claims 70-72, wherein R³² and R³³ are each H.

74. The compound of any one of claims 70-72, wherein R³² and R³³ are each alkyl.

75. The compound of any one of claims 70-72, wherein R³² is H or CH₃; and R³³ is alkyl, hydroxyalkyl, optionally substituted cycloalkyl, or optionally substituted heterocyclyl.

76. The compound of claim 75, wherein the optionally substituted cycloalkyl is a cyclopropyl or cyclobutyl.

77. The compound of claim 75, wherein the optionally substituted heterocyclyl is an azetidinyl, pyrrolidinyl, or piperidinyl.

78. The compound of claim 76 or 77, wherein the cycloalkyl or heterocyclyl, when substituted, is substituted with halo, alkyl, hydroxyl, hydroxyalkyl, or carbamate.

79. The compound of any one of claims 70-72, wherein R³² and R³³ combine to form an optionally substituted azetidine, pyrrolidine, or piperidine.

80. The compound of claim 79, wherein the azetidinyl, pyrrolidinyl, or piperidinyl, when substituted, is substituted with halo, alkyl, or hydroxyl.

81. The compound of claim 70, wherein Z is

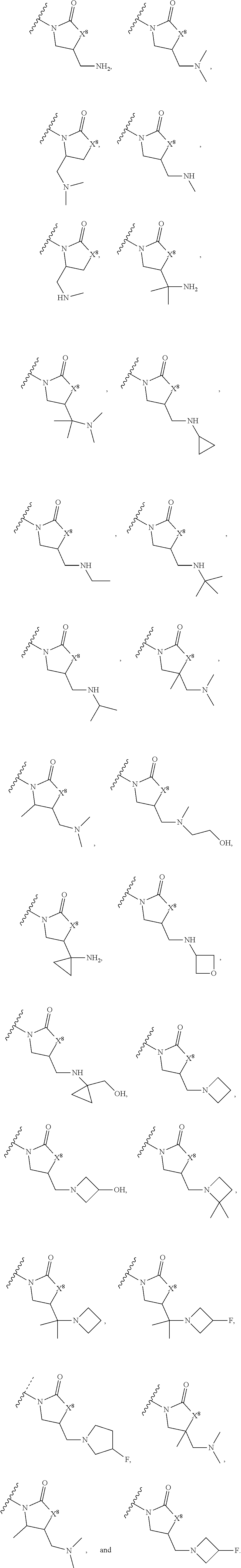

82. The compound of any one of claims 70-72, wherein one of R² is H and and R²⁷ is amino or optionally substituted heterocyclyl.

83. The compound of claim 82 wherein the optionally substituted heterocyclyl is an optionally substituted oxetanyl, azetidinyl, pyrrolidinyl, piperidinyl, or oxazolidinonyl.

84. The compound of claim 83, wherein the oxetanyl, azetidinyl, pyrrolidinyl, piperidinyl or oxazolidinonyl, when substituted, is substituted with alkyl, hydroxyalkyl, or carbamate.

85. The compound of claim 82, wherein Z is

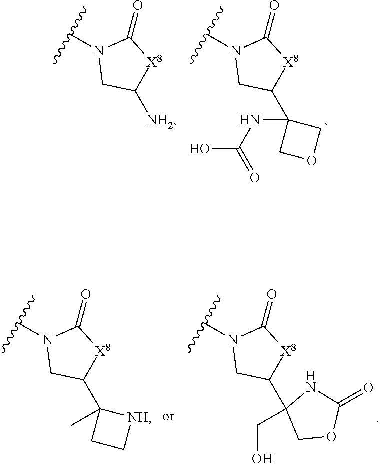

86. The compound of claim 66, wherein Z is

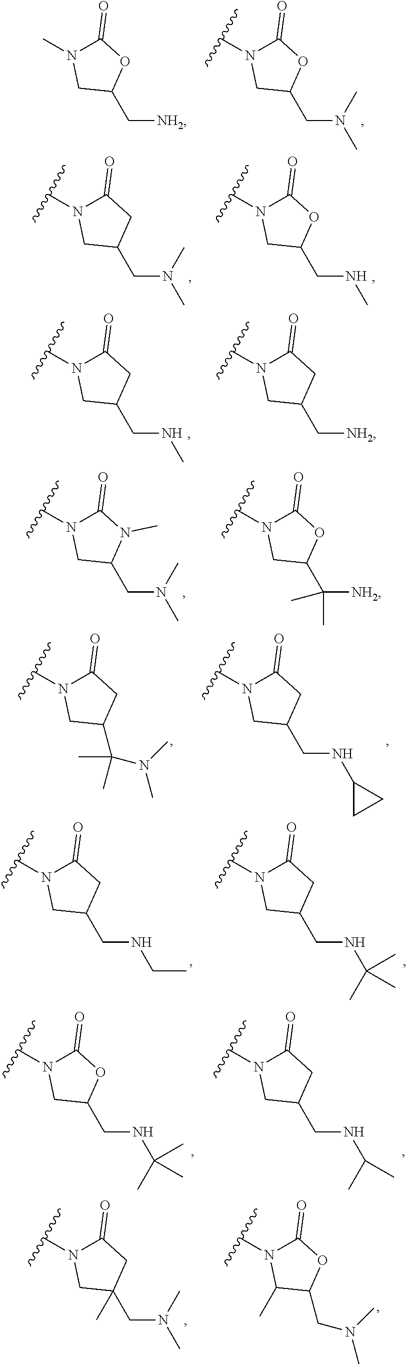

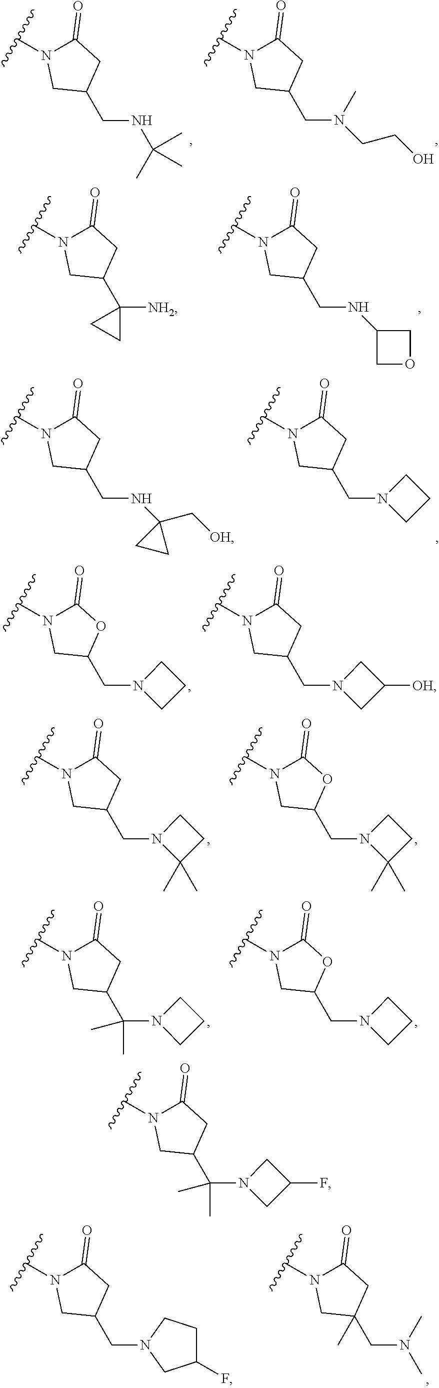

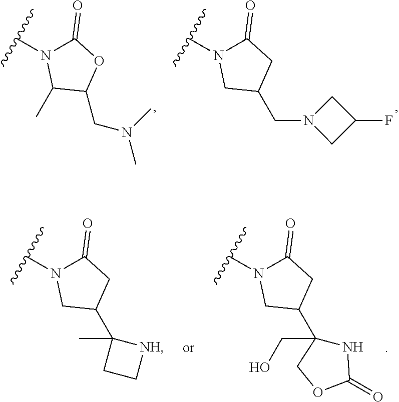 [SMILES: *N1C(=O)OC(CN(C)C)C1C ; *N1CC(CN2CC(F)C2)CC1=O ; *N1CC(C2(C)CCN2)CC1=O ; *N1CC(C2(CO)COC(=O)N2)CC1=O]

87. The compound of claim 66, wherein X⁸ is CR²⁸R²⁹; and R²⁸ and R²⁹ each independently H, alkyl, alkylamino, aminoalkyl, or optionally substituted alkylaminoalkyl; and R³⁰ is alkyl, alkylaminoalkyl, or an optionally substituted cycloalkyl or heterocyclyl.

88. The compound of claim 87, wherein R²⁴, R²⁵, R²⁶, and R²⁷ are each H.

89. The compound of claim 86 or 87, wherein one of R²⁸ and R²⁹ is H or CH₃ and the other of R²⁸ and R²⁹ is alkylamino, aminoalkyl, or optionally substituted alkylaminoalkyl.

90. The compound of claim 89, wherein the alkylaminoalkyl, when substituted, is substituted with a hydroxyl.

91. The compound of claim 86 or 87, wherein X⁸ is NR³¹; and R³¹ is alkylaminoalkyl, or an optionally substituted cycloalkyl or heterocyclyl.

92. The compound of claim 90, wherein the optionally substituted cycloalkyl is cyclopropyl or cyclobutyl.

93. The compound of claim 91, wherein the cyclopropyl or cyclobutyl, when substituted, is substituted with alkylamino.

94. The compound of claim 91, wherein the optionally substituted heterocyclyl is oxetanyl, azetidinyl, pyrrolidinyl, or piperidinyl.

95. The compound of claim 94, wherein the oxetanyl, azetidinyl, pyrrolidinyl, or piperidinyl, when substituted, is substituted with alkyl.

96. The compound of claim 87, wherein Z is

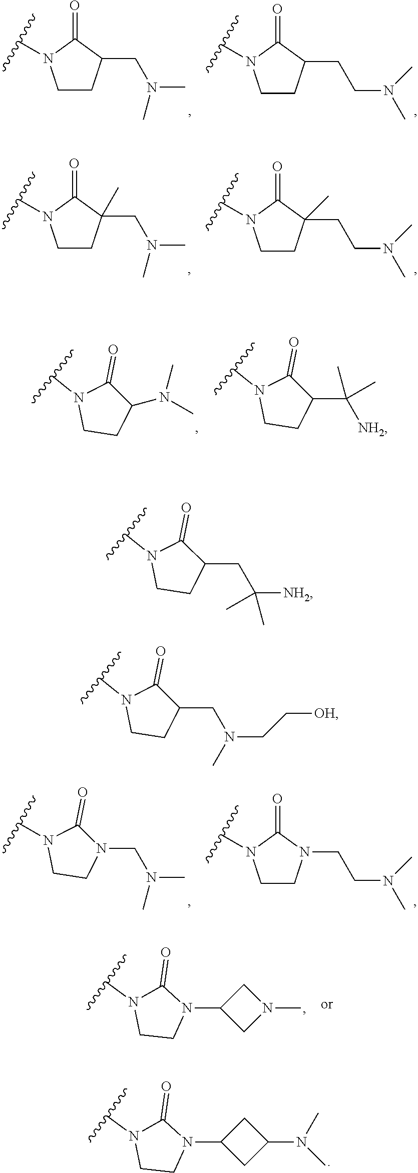

97. The compound of claim 65, wherein
Z is 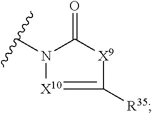
X⁹ is CR³⁶R³⁷ or NR³⁸;
X¹⁰ is N or CR³⁴;
R³⁴ and R³⁵ are each independently H or optionally substituted alkyl; and
R³⁶ and R³⁷ are each independently H, alkyl, alkylamino, aminoalkyl, alkylaminoalkyl, or optionally substituted heterocyclyl; and
R³⁸ is alkyl, aminoalkyl or alkylaminoalkyl.

98. The compound of claim 97, wherein Z is

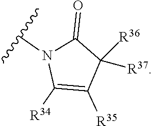

99. The compound of claim 98, wherein one of R³⁶ and R³⁷ is H or alkyl and the other of R³⁶ and R³⁷ is alkylaminoalkyl or optionally substituted heterocyclyl.

100. The compound of claim 99, wherein the optionally substituted heterocyclyl is oxetanyl, azetidinyl, pyrrolidinyl, or piperidinyl.

101. The compound of claim 99, wherein the oxetanyl, azetidinyl, pyrrolidinyl, or piperidinyl, when substituted, is substituted with alkyl.

102. The compound of claim 97, wherein Z is

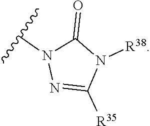

103. The compound of claim 102, wherein R³⁸ is alkylaminoalkyl.

104. The compound of claim 97, wherein Z is

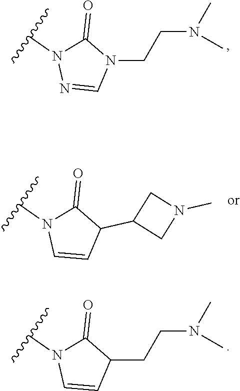

105. The compound of claim 65, wherein
Z is 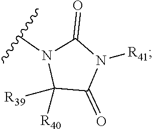
R³⁹ and R⁴⁰ are each independently H or optionally substituted alkyl; and
R⁴¹ is aminoalkyl, alkylaminoalkyl, or an optionally substituted heterocyclyl or heterocycloalkyl.

106. The compound of claim 105, wherein R⁴¹ is aminoalkyl or alkylaminoalkyl.

107. The compound of claim 105, wherein R⁴¹ is an optionally substituted heterocyclyl or heterocycloalkyl.

108. The compound of claim 107, wherein the heterocyclyl or heterocycloalkyl substituted alkyl, when substituted, is substituted with alkyl.

109. The compound of any one of claim 105, wherein Z is

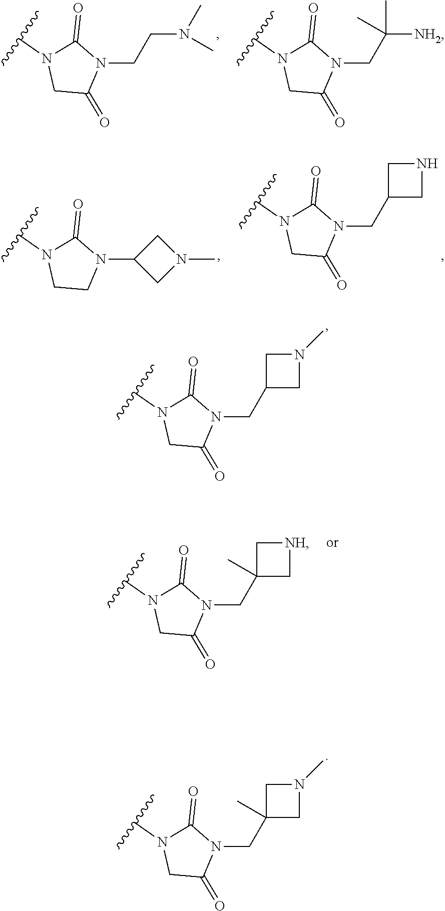

110. The compound of claim 65, wherein
Z is 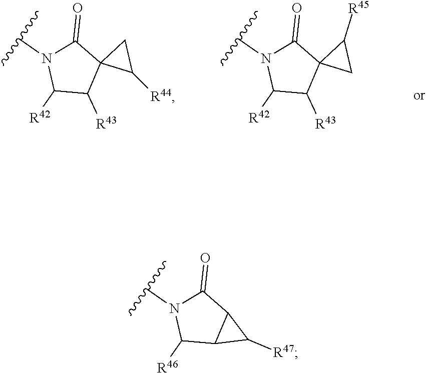
R⁴², R⁴³, and R⁴⁶ are each independently H or optionally substituted alkyl; and
R⁴⁴, R⁴⁵, and R⁴⁷ are each independently H, alkylamino, aminoalkyl, or alkylaminoalkyl.

111. The compound of claim 110, wherein R⁴², R⁴³, and R⁴⁶ are each H.

112. The compound of claim 110 or 111, wherein R⁴⁴, R⁴⁵, and R⁴⁷ are each alkylaminoalkyl.

113. The compound of claim 110, wherein Z is

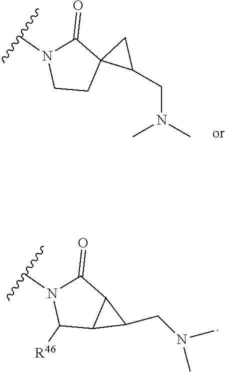

114. The compound of claim 65, wherein
Z is 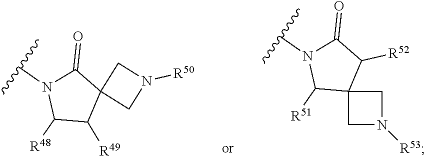
R⁴⁸, R⁴⁹, R⁵¹, and R⁵² are each independently H or optionally substituted alkyl; and
R⁵⁰ and R⁵³ are each independently H, alkyl, aminoalkyl, or alkylaminoalkyl.

115. The compound of claim 114, wherein R⁴⁸, R⁴⁹, R⁵¹, and R⁵² are each H.

116. The compound of claim 114 or 115, wherein R⁵⁰ and R⁵³ are each alkyl.

117. The compound of claim 113, wherein Z is

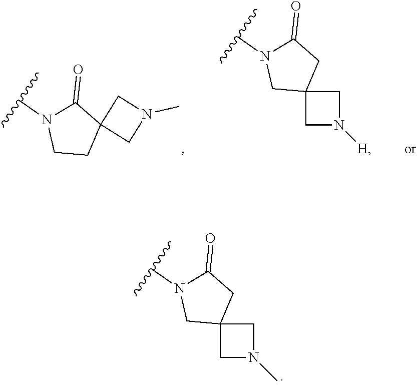

118. The compound of claim 65, wherein
Z is 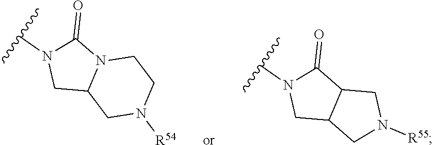
and
R⁵⁴ and R⁵⁵ are each independently H, alkyl, aminoalkyl, or alkylaminoalkyl.

119. The compound of claim 118, wherein R⁵⁴ and R⁵⁵ are each alkyl.

120. The compound of claim 118, wherein Z is

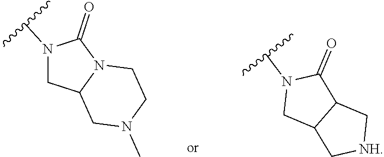 [SMILES: *N1CC2CN(C)CCN2C1=O ; "or" ; *N1CC2CNCC2C1=O]

121. The compound of claim 65, wherein
Z is 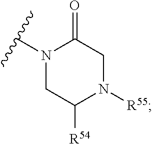
R⁵⁴ is H, or an optionally substituted alkyl or hydroxyalkyl; and
R⁵⁵ is H, alkyl, aminoalkyl, alkylaminoalkyl, or cycloalkyl.

122. The compound of claim 121, wherein R⁵⁴ is H or hydroxyalkyl.

123. The compound of claim 121 or 122, wherein R⁵⁵ is H or cycloalkyl.

124. The compound of any one of claim 121, wherein Z is

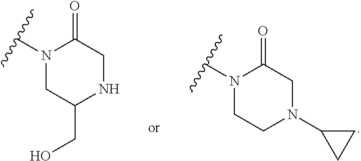 [SMILES: *N1CC(CO)NCC1=O ; "or" ; *N1CCN(C2CC2)CC1=O]

125. The compound of claim 1 or 2 having the structure: 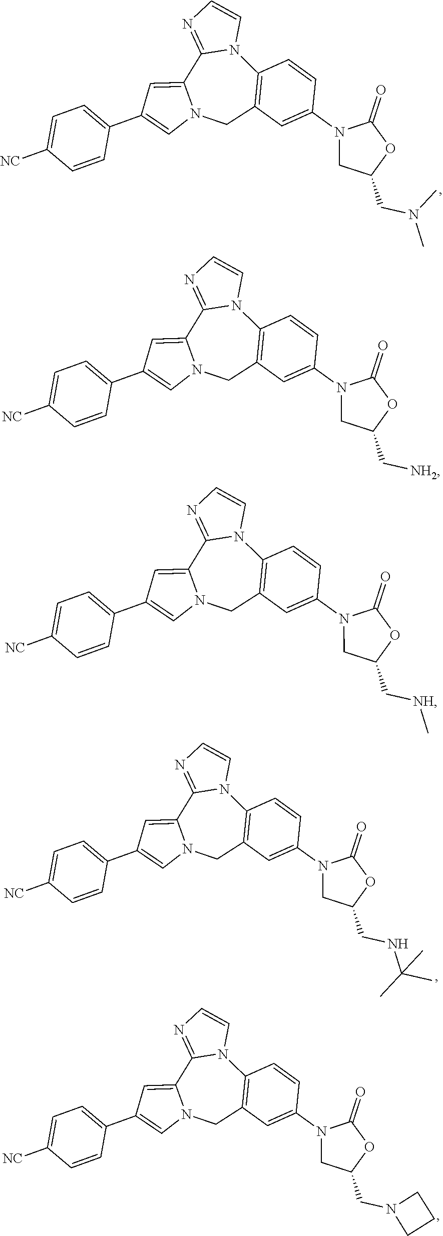 [SMILES: CN(C)C[C@@H]1CN(c2ccc3c(c2)Cn2cc(-c4ccc(C#N)cc4)cc2-c2nccn2-3)C(=O)O1 ; N#Cc1ccc(-c2cc3n(c2)Cc2cc(N4C[C@@H](CN)OC4=O)ccc2-n2ccnc2-3)cc1 ; CNC[C@@H]1CN(c2ccc3c(c2)Cn2cc(-c4ccc(C#N)cc4)cc2-c2nccn2-3)C(=O)O1 ; CC(C)(C)NC[C@@H]1CN(c2ccc3c(c2)Cn2cc(-c4ccc(C#N)cc4)cc2-c2nccn2-3)C(=O)O1 ; N#Cc1ccc(-c2cc3n(c2)Cc2cc(N4C[C@@H](CN5CCC5)OC4=O)ccc2-n2ccnc2-3)cc1] 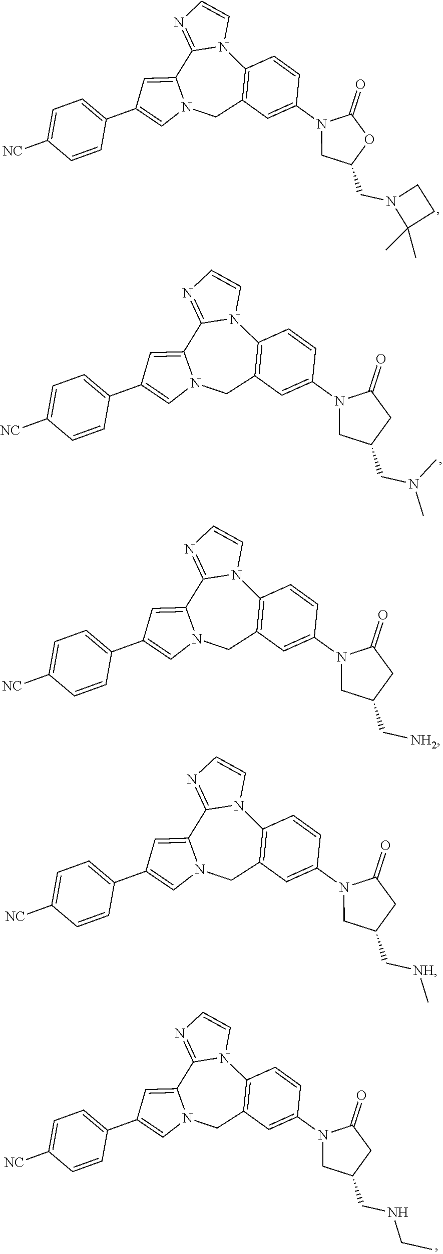 [SMILES: CC1(C)CCN1C[C@@H]1CN(c2ccc3c(c2)Cn2cc(-c4ccc(C#N)cc4)cc2-c2nccn2-3)C(=O)O1 ; CN(C)C[C@H]1CC(=O)N(c2ccc3c(c2)Cn2cc(-c4ccc(C#N)cc4)cc2-c2nccn2-3)C1 ; N#Cc1ccc(-c2cc3n(c2)Cc2cc(N4C[C@@H](CN)CC4=O)ccc2-n2ccnc2-3)cc1 ; CNC[C@H]1CC(=O)N(c2ccc3c(c2)Cn2cc(-c4ccc(C#N)cc4)cc2-c2nccn2-3)C1 ; CCNC[C@H]1CC(=O)N(c2ccc3c(c2)Cn2cc(-c4ccc(C#N)cc4)cc2-c2nccn2-3)C1] 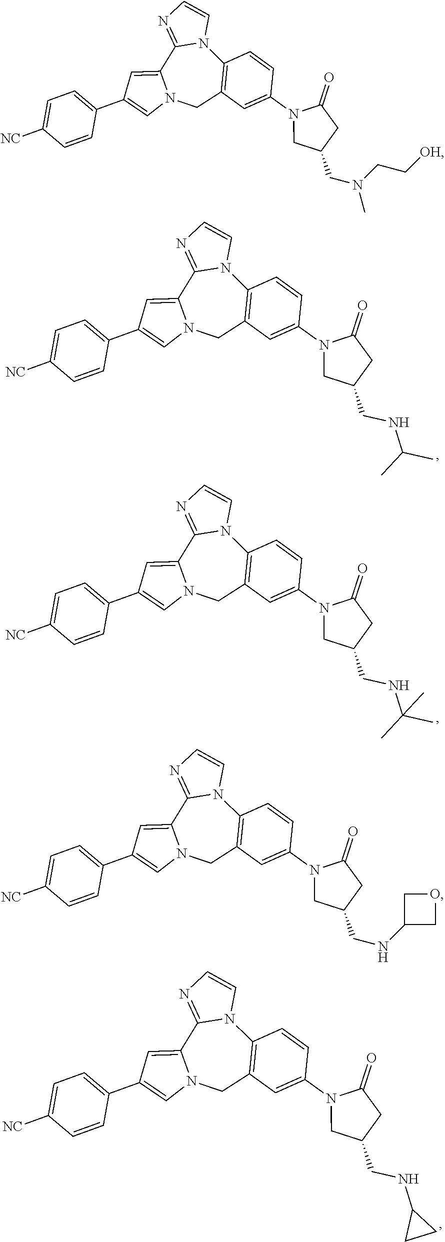 [SMILES: CN(CCO)C[C@H]1CC(=O)N(c2ccc3c(c2)Cn2cc(-c4ccc(C#N)cc4)cc2-c2nccn2-3)C1 ; CC(C)NC[C@H]1CC(=O)N(c2ccc3c(c2)Cn2cc(-c4ccc(C#N)cc4)cc2-c2nccn2-3)C1 ; CC(C)(C)NC[C@H]1CC(=O)N(c2ccc3c(c2)Cn2cc(-c4ccc(C#N)cc4)cc2-c2nccn2-3)C1 ; N#Cc1ccc(-c2cc3n(c2)Cc2cc(N4C[C@@H](CNC5COC5)CC4=O)ccc2-n2ccnc2-3)cc1 ; N#Cc1ccc(-c2cc3n(c2)Cc2cc(N4C[C@@H](CNC5CC5)CC4=O)ccc2-n2ccnc2-3)cc1] 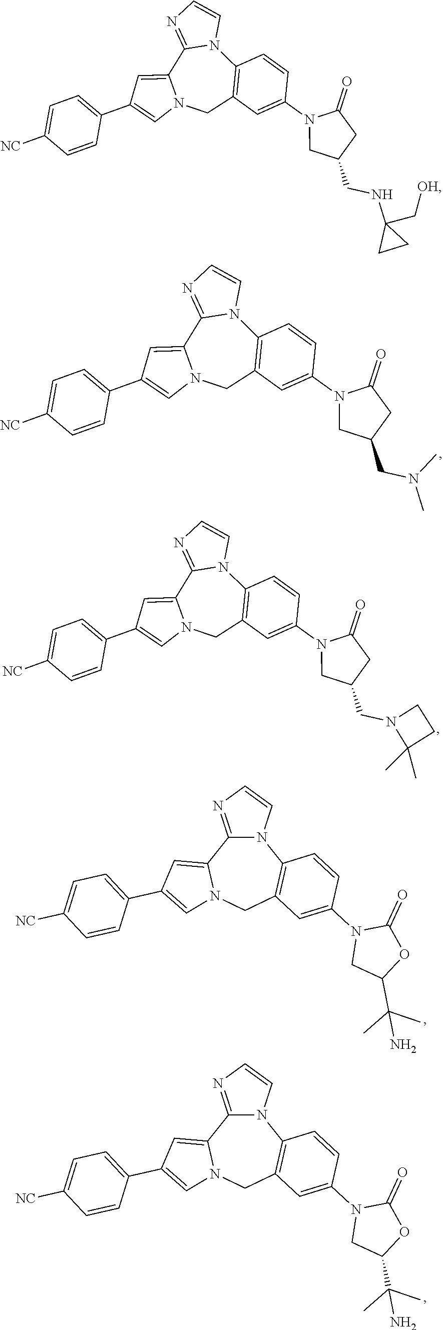 [SMILES: N#Cc1ccc(-c2cc3n(c2)Cc2cc(N4C[C@@H](CNC5(CO)CC5)CC4=O)ccc2-n2ccnc2-3)cc1 ; CN(C)C[C@@H]1CC(=O)N(c2ccc3c(c2)Cn2cc(-c4ccc(C#N)cc4)cc2-c2nccn2-3)C1 ; CC1(C)CCN1C[C@H]1CC(=O)N(c2ccc3c(c2)Cn2cc(-c4ccc(C#N)cc4)cc2-c2nccn2-3)C1 ; CC(C)(N)C1CN(c2ccc3c(c2)Cn2cc(-c4ccc(C#N)cc4)cc2-c2nccn2-3)C(=O)O1 ; CC(C)(N)[C@@H]1CN(c2ccc3c(c2)Cn2cc(-c4ccc(C#N)cc4)cc2-c2nccn2-3)C(=O)O1] 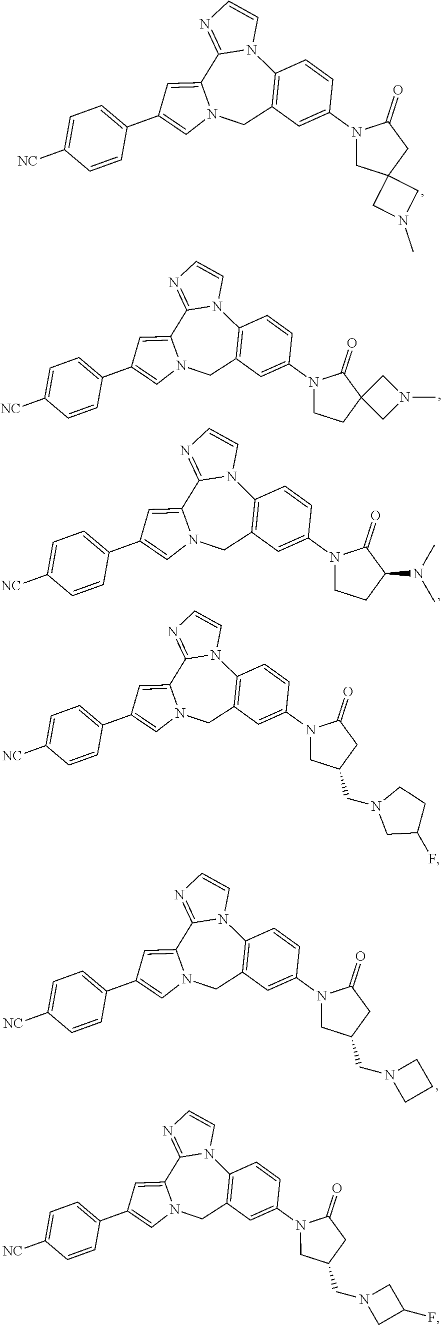 [SMILES: CN1CC2(CC(=O)N(c3ccc4c(c3)Cn3cc(-c5ccc(C#N)cc5)cc3-c3nccn3-4)C2)C1 ; CN1CC2(CCN(c3ccc4c(c3)Cn3cc(-c5ccc(C#N)cc5)cc3-c3nccn3-4)C2=O)C1 ; CN(C)[C@H]1CCN(c2ccc3c(c2)Cn2cc(-c4ccc(C#N)cc4)cc2-c2nccn2-3)C1=O ; N#Cc1ccc(-c2cc3n(c2)Cc2cc(N4C[C@@H](CN5CCC(F)C5)CC4=O)ccc2-n2ccnc2-3)cc1 ; N#Cc1ccc(-c2cc3n(c2)Cc2cc(N4C[C@@H](CN5CCC5)CC4=O)ccc2-n2ccnc2-3)cc1 ; N#Cc1ccc(-c2cc3n(c2)Cc2cc(N4C[C@@H](CN5CC(F)C5)CC4=O)ccc2-n2ccnc2-3)cc1] 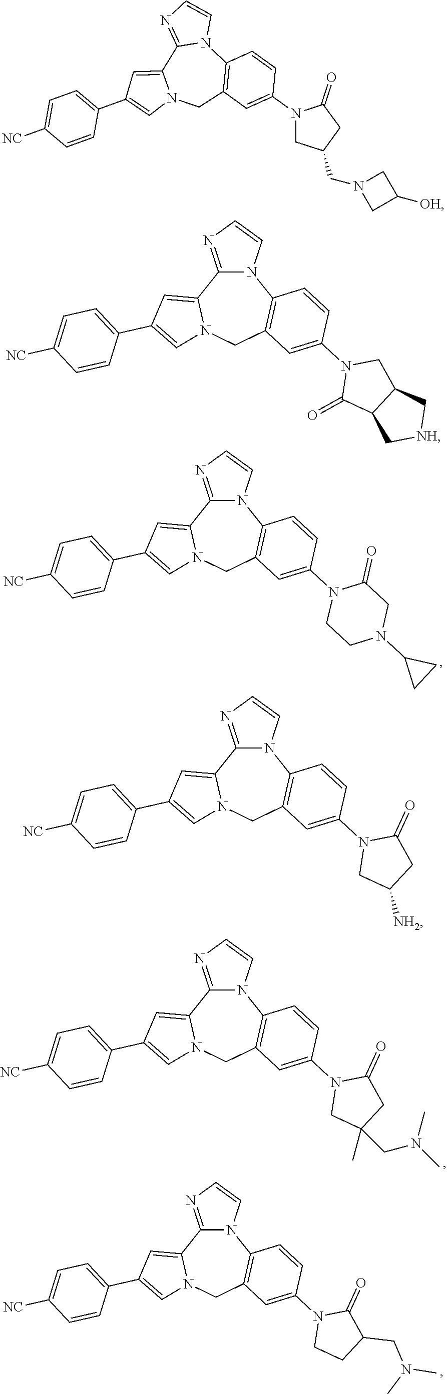 [SMILES: N#Cc1ccc(-c2cc3n(c2)Cc2cc(N4C[C@@H](CN5CC(O)C5)CC4=O)ccc2-n2ccnc2-3)cc1 ; N#Cc1ccc(-c2cc3n(c2)Cc2cc(N4C[C@H]5CNC[C@H]5C4=O)ccc2-n2ccnc2-3)cc1 ; N#Cc1ccc(-c2cc3n(c2)Cc2cc(N4CCN(C5CC5)CC4=O)ccc2-n2ccnc2-3)cc1 ; N#Cc1ccc(-c2cc3n(c2)Cc2cc(N4C[C@@H](N)CC4=O)ccc2-n2ccnc2-3)cc1 ; CN(C)CC1(C)CC(=O)N(c2ccc3c(c2)Cn2cc(-c4ccc(C#N)cc4)cc2-c2nccn2-3)C1 ; CN(C)CC1CCN(c2ccc3c(c2)Cn2cc(-c4ccc(C#N)cc4)cc2-c2nccn2-3)C1=O] 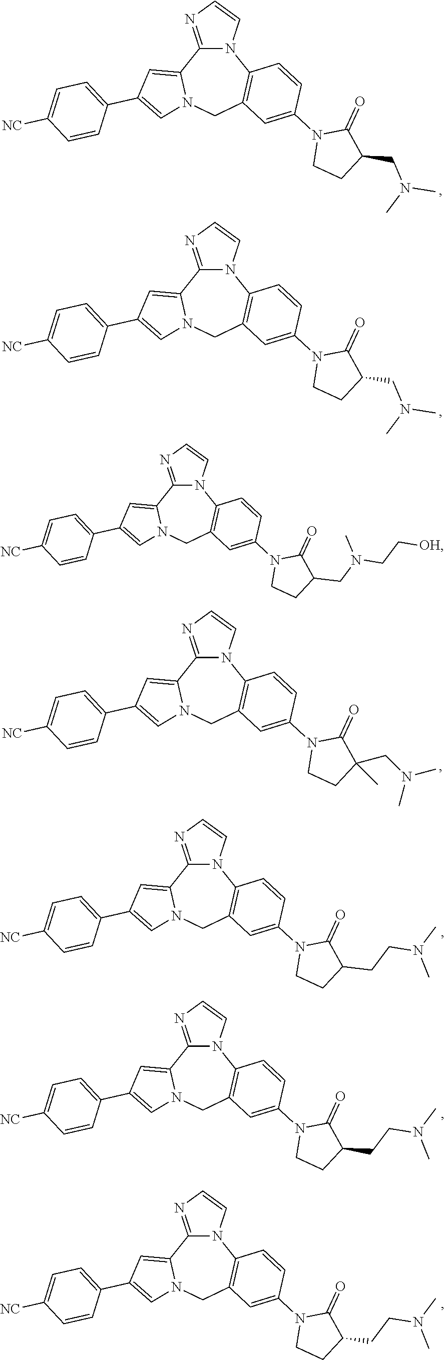 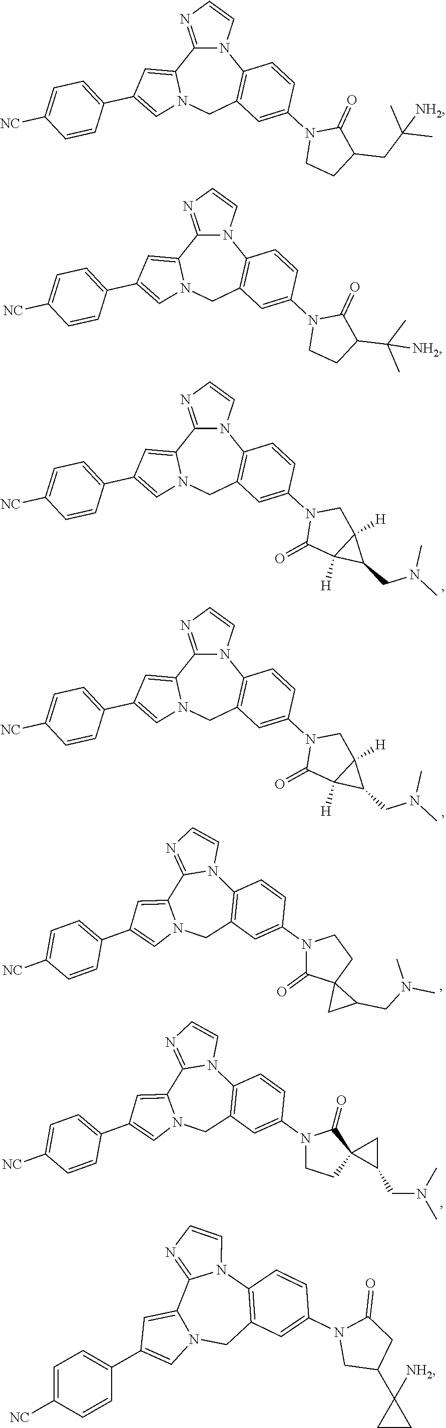 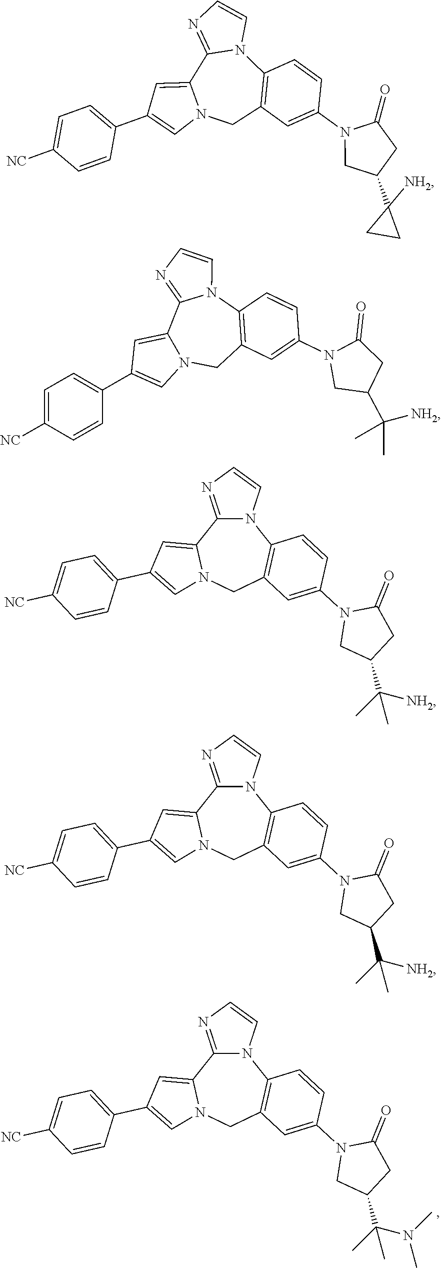 [SMILES: N#Cc1ccc(-c2cc3n(c2)Cc2cc(N4C[C@@H](C5(N)CC5)CC4=O)ccc2-n2ccnc2-3)cc1 ; CC(C)(N)C1CC(=O)N(c2ccc3c(c2)Cn2cc(-c4ccc(C#N)cc4)cc2-c2nccn2-3)C1 ; CC(C)(N)[C@H]1CC(=O)N(c2ccc3c(c2)Cn2cc(-c4ccc(C#N)cc4)cc2-c2nccn2-3)C1 ; CC(C)(N)[C@@H]1CC(=O)N(c2ccc3c(c2)Cn2cc(-c4ccc(C#N)cc4)cc2-c2nccn2-3)C1 ; CN(C)C(C)(C)[C@H]1CC(=O)N(c2ccc3c(c2)Cn2cc(-c4ccc(C#N)cc4)cc2-c2nccn2-3)C1] 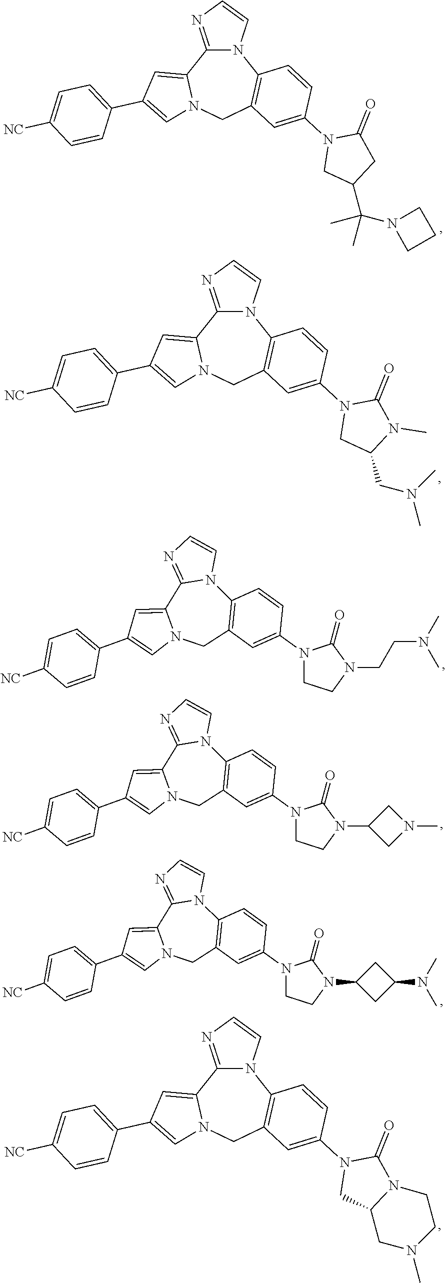 [SMILES: CC(C)(C1CC(=O)N(c2ccc3c(c2)Cn2cc(-c4ccc(C#N)cc4)cc2-c2nccn2-3)C1)N1CCC1 ; CN(C)C[C@@H]1CN(c2ccc3c(c2)Cn2cc(-c4ccc(C#N)cc4)cc2-c2nccn2-3)C(=O)N1C ; CN(C)CCN1CCN(c2ccc3c(c2)Cn2cc(-c4ccc(C#N)cc4)cc2-c2nccn2-3)C1=O ; CN1CC(N2CCN(c3ccc4c(c3)Cn3cc(-c5ccc(C#N)cc5)cc3-c3nccn3-4)C2=O)C1 ; CN(C)[C@H]1C[C@@H](N2CCN(c3ccc4c(c3)Cn3cc(-c5ccc(C#N)cc5)cc3-c3nccn3-4)C2=O)C1 ; CN1CCN2C(=O)N(c3ccc4c(c3)Cn3cc(-c5ccc(C#N)cc5)cc3-c3nccn3-4)C[C@H]2C1] 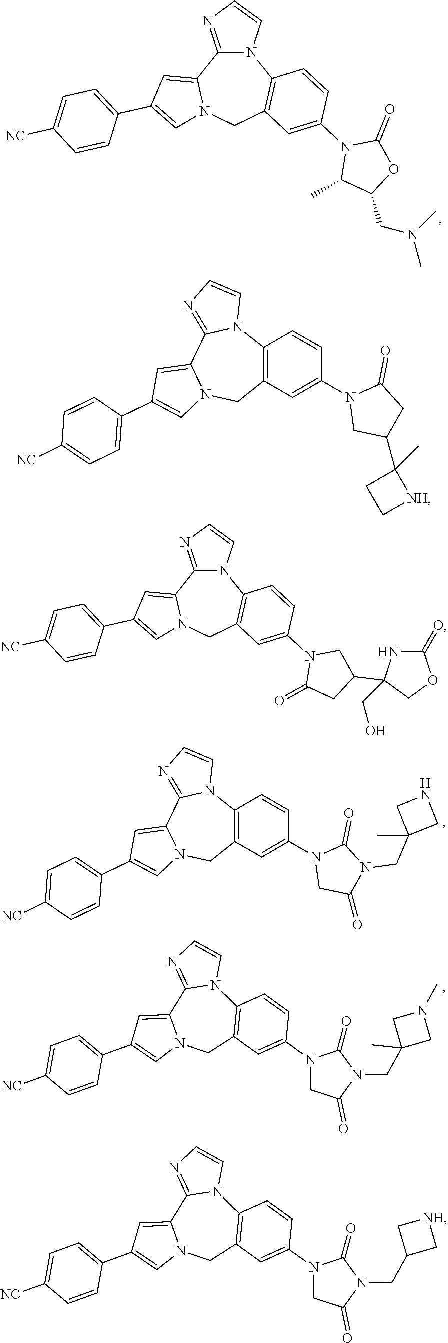 [SMILES: C[C@H]1[C@@H](CN(C)C)OC(=O)N1c1ccc2c(c1)Cn1cc(-c3ccc(C#N)cc3)cc1-c1nccn1-2 ; CC1(C2CC(=O)N(c3ccc4c(c3)Cn3cc(-c5ccc(C#N)cc5)cc3-c3nccn3-4)C2)CCN1 ; N#Cc1ccc(-c2cc3n(c2)Cc2cc(N4CC(C5(CO)COC(=O)N5)CC4=O)ccc2-n2ccnc2-3)cc1 ; CC1(CN2C(=O)CN(c3ccc4c(c3)Cn3cc(-c5ccc(C#N)cc5)cc3-c3nccn3-4)C2=O)CNC1 ; CN1CC(C)(CN2C(=O)CN(c3ccc4c(c3)Cn3cc(-c5ccc(C#N)cc5)cc3-c3nccn3-4)C2=O)C1 ; N#Cc1ccc(-c2cc3n(c2)Cc2cc(N4CC(=O)N(CC5CNC5)C4=O)ccc2-n2ccnc2-3)cc1] 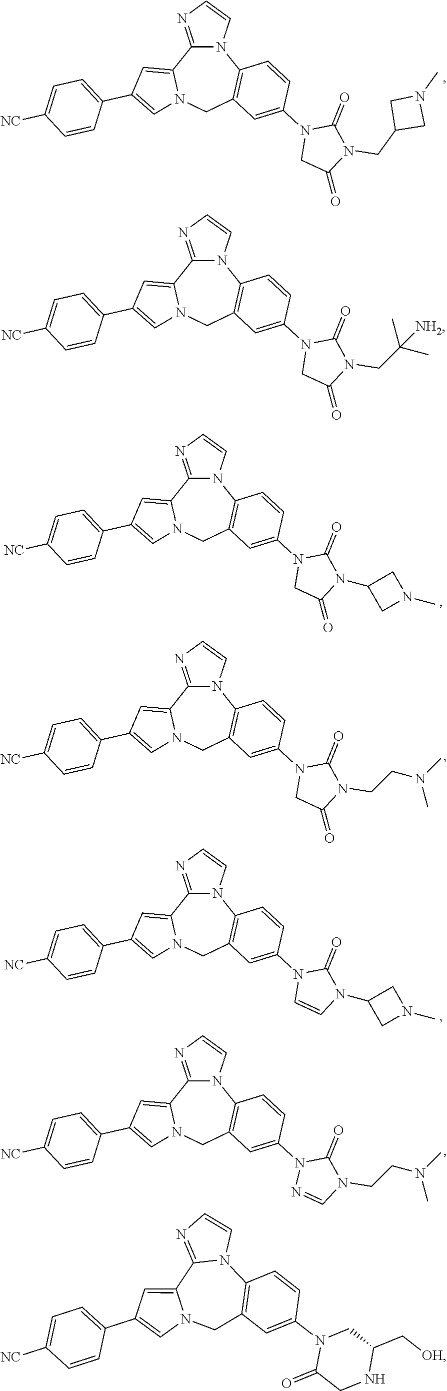 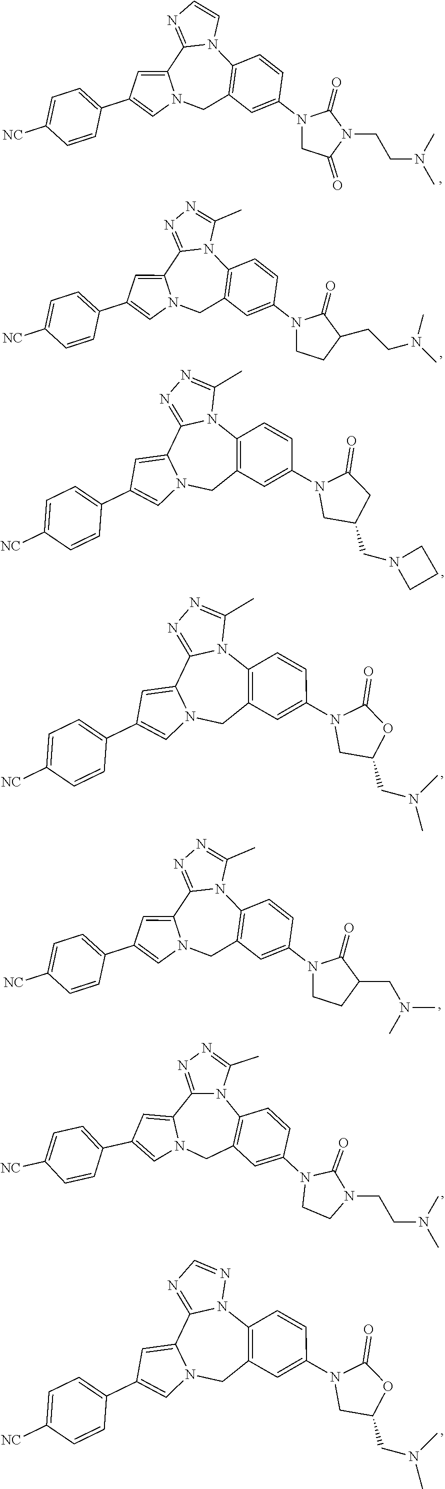 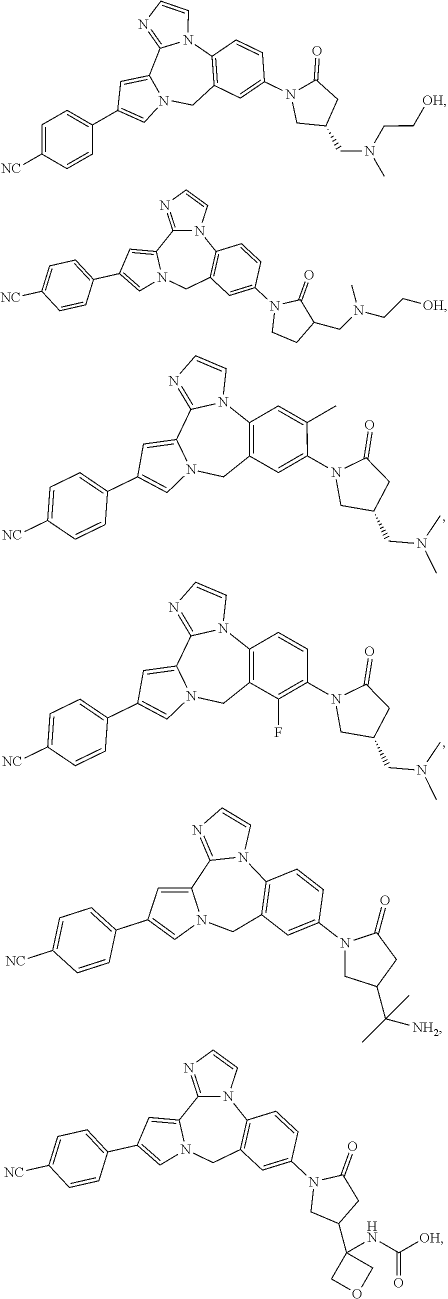 [SMILES: CN(CCO)C[C@H]1CC(=O)N(c2ccc3c(c2)Cn2cc(-c4ccc(C#N)cc4)cc2-c2nccn2-3)C1 ; CN(CCO)CC1CCN(c2ccc3c(c2)Cn2cc(-c4ccc(C#N)cc4)cc2-c2nccn2-3)C1=O ; Cc1cc2c(cc1N1C[C@@H](CN(C)C)CC1=O)Cn1cc(-c3ccc(C#N)cc3)cc1-c1nccn1-2 ; CN(C)C[C@H]1CC(=O)N(c2ccc3c(c2F)Cn2cc(-c4ccc(C#N)cc4)cc2-c2nccn2-3)C1 ; CC(C)(N)C1CC(=O)N(c2ccc3c(c2)Cn2cc(-c4ccc(C#N)cc4)cc2-c2nccn2-3)C1 ; N#Cc1ccc(-c2cc3n(c2)Cc2cc(N4CC(C5(NC(=O)O)COC5)CC4=O)ccc2-n2ccnc2-3)cc1] 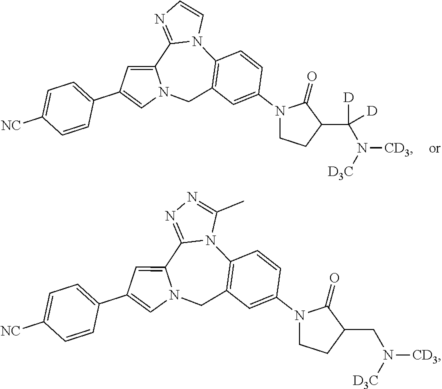 [SMILES: [2H]C([2H])([2H])N(C([2H])([2H])[2H])C([2H])([2H])C1CCN(c2ccc3c(c2)Cn2cc(-c4ccc(C#N)cc4)cc2-c2nccn2-3)C1=O ; [2H]C([2H])([2H])N(CC1CCN(c2ccc3c(c2)Cn2cc(-c4ccc(C#N)cc4)cc2-c2nnc(C)n2-3)C1=O)C([2H])([2H])[2H]]
or a pharmaceutically acceptable salt of the compound.

126. A pharmaceutical composition comprising a compound of any one of claims 1-125 and a pharmaceutically acceptable carrier.

127. A method of treating or preventing a mitogen-activated protein kinase activated protein kinase-2 (MK2) related disorder, comprising administering to a subject a compound of any one of claims 1-125 or pharmaceutical composition of claim 126 so as to thereby treat or prevent the MK2 related disorder in the subject.

128. The method of claim 127, wherein the MK2 related disorder is an inflamatory disorder.

129. The method of claim 127, wherein the MK2 related disorder is a cancer.

130. The method of claim 129, wherein the cancer is a KRAS- or BRAF-dependent cancer.

131. The method of claim 129 or 130, further comprising conjointly administering one or more additional chemotherapeutic agents or a combination therapy.

132. The method of claim 131, wherein the additional chemotherapeutic agent is a CHK1 inhibitor or an alkylating agent.

133. The method of claim 132, wherein the CHK1 inhibitor is PF477736 or LY2603618.

134. The method of claim 132, wherein the alkylating agent is cisplatin.

135. A method of inhibiting proliferation of a cancer cell, comprising contacting a cancer cell with a compound of any one of claims 1-125.

136. A method of inhibiting MK2 activity in a cell, comprising contacting a cell with a compound of any one of claim 125.

137. A method of treating or preventing a metabolic disorder, comprising administering to a subject a compound any one of claims 1-125 or pharmaceutical composition of claim 126, so as to thereby treat or prevent the metabolic disorder in the subject.

138. The method of claim 137, wherein the metabolic disorder is diabetes, insulin resistance, obesity, or metabolic syndrome.

139. The method of claim 138, wherein the diabetes is Type I, Type II, or gestational diabetes.

140. The method of any one of claims 137-139, wherein the treating or preventing affects glycogenolysis or gluconeogenesis in the subject.

141. The method of any one of claims 137-140, wherein the treating or preventing reduces hepatic glucose production, hyperglycemia, fatty liver, insulin resistance, insulin-resistance-associated inflammation, insulin resistance-associated dyslipidemia, or any combination thereof, in the subject.

142. The method of any one of claims 137-141, further comprising conjointly administering one or more additional antidiabetic agents.

143. The method of claim 142, wherein the one or more additional antidiabetic agents comprise metformin.
